(19) United States
(12) Patent Application Publication
(10) Pub. No.: US 2023/0203512 A1
(43) Pub. Date: Jun. 29, 2023

(54) COMPOSITIONS AND METHODS FOR AGROBACTERIUM MEDIATED TRANSFORMATION OF CHLOROPLASTS IN SEED PLANTS

(71) Applicant: RUTGERS, THE STATE UNIVERSITY OF NEW JERSEY, New Brunswick, NJ (US)

(72) Inventors: Pal Maliga, East Brunswick, NJ (US); Aki Matsuoka, Highland Park, NJ (US)

(73) Assignee: RUTGERS, THE STATE UNIVERSITY OF NEW JERSEY, New Brunswick, NJ (US)

(21) Appl. No.: 17/813,667

(22) Filed: Jul. 20, 2022

Related U.S. Application Data
(60) Provisional application No. 63/223,881, filed on Jul. 20, 2021.

(51) Int. Cl. C12N 15/82; C12N 1/20

(52) U.S. Cl. CPC C12N 15/8205; C12N 1/205; C12R 2001/01

(57) ABSTRACT

Compositions and methods for agrobacterium-mediated chloroplast transgene expression are provided.

### CROSS-REFERENCE TO RELATED APPLICATION

This application claims priority to U.S. Provisional Application No. 63/223,881, filed on Jul. 20, 2021, the entire disclosure of which is incorporated herein by reference as though set forth in full.

### INCORPORATION BY REFERENCE OF MATERIAL SUBMITTED IN ELECTRONIC FORM

The contents of the electronic sequence listing (RUT-102-US.xml; Size: 294,637 bytes; and Date of Creation: Jul. 20, 2022) is herein incorporated by reference in its entirety.

### GRANT STATEMENT

This invention was made with government support under grant numbers MCB 1716102 and IOS 2037155 awarded by the National Science Foundation. The government has certain rights in the invention.

### FIELD OF THE INVENTION

The invention relates to fields of transgenic plants and efficient means for expression of heterologous proteins and RNAs of interest. More specifically, the invention provides methods and compositions to increase the efficiency of plant cell transformation and to engineer desirable plant traits through Agrobacterium-mediated delivery of specific proteins and RNAs into targeted organelles (e.g., plastids, such as chloroplasts) in plants of interest.

### BACKGROUND OF THE INVENTION

Several publications and patent documents are cited throughout the specification in order to describe the state of the art to which this invention pertains. Each of these citations is incorporated herein by reference as though set forth in full.

Agrobacterium tumefaciens is a Gram-negative phytopathogenic bacterium considered a natural genetic engineer because it transforms plants and results in crown gall disease. The gall is caused by the transferred DNA (T-DNA) encoding genes for plant growth regulators, auxins and cytokinins, that trigger cell division. The T-DNA also encodes genes for the synthesis of the opines: octopine (ocs), mannopine (mas) and agropine (ags). Opines are metabolites only Agrobacterium can utilize, thus the objective of T-DNA transfer is towards creating an exclusive food source for the species. The T-DNA is defined by 25-bp direct repeats which form the T-DNA borders. The T-DNA and genes required for T-DNA transfer (vir genes) are encoded on mega-plasmids, such as pTiC58 in A. tumefaciens strain C58 (Wood et al., 2001, Gelvin, 2003).

The T-DNA transfer process is triggered by plant metabolites, such as sugars and phenolic compounds via a two-component sensory response system consisting of constitutively expressed bacterial VirA and VirG proteins. VirA detects plant signaling molecules and phosphorylates VirG, which activates transcription of other vir genes on the Ti plasmid. VirD2, an endonuclease, in concert with VirD1, nicks the left and right border sequences of the T-DNA, and VirD2 covalently attaches to the 5′ end of single-stranded T-DNA (T-strand). The VirD2/T-strand complex along with other effector proteins including VirE2, VirE3, VirF and VirD5 are translocated to the plant cell through a Type IV secretion system (T4SS) composed of VirB 1-11 and VirD4 (Gelvin, 2003, McCullen and Binns, 2006).

In the plant cytoplasm, VirE2 covers the T-strand of the VirD2/T-strand complex, forming the T-complex to protect it from degradation. The T-complex is directed to the nucleus by VirD2, which has nuclear localization signals. VirE2 also participates in the nuclear targeting of the complex and facilitates the movement of the T-complex through the nuclear pore. Plant proteins such as importin α, β, CAK2Ms kinase, protein phosphatase 2C and VirE2 interacting protein 1 (VIP1) form complexes with either VirD2 and/or VirE2 for nuclear targeting. Upon entry of the complex in the nucleus, VirF recognizes VIP1 which leads to the destabilization and degradation of VirE2 and VIP1 via the Skp-Cullin-F-box pathway. Subsequently, the T-strand integrates into the nuclear genome by a variant of double-strand break repair (Brunaud et al., 2002, Tzfira et al., 2004, Gelvin, 2017).

Arabidopsis (Arabidopsis thaliana) is the best characterized model plant and is used to study all aspects of basic science. A notable exception is that studies involving plastid genome engineering are also carried out in tobacco, the only vascular plant species in which plastome engineering is routine. Recently, high-frequency plastid transformation in Arabidopsis was achieved by using plants hyper-sensitive to spectinomycin, the selective agent used in chloroplast transformation. The current bottleneck of plastid transformation in Arabidopsis is the difficulty of obtaining fertile plants from transplastomic tissue culture cells. It is an objective of the invention to create an Agrobacterium based system for efficient transformation of chloroplasts in seed plants which avoids the need for tissue culture-based selection.

### SUMMARY OF THE INVENTION

Disarmed strains of Agrobacterium have been developed providing an efficient and practical gene transfer tool. By removing both the disease-causing and opine genes, the virulence plasmid is transformed to selectively aid the excision of T-DNA containing genes of interest from a modified binary plasmid. In one aspect, the invention provides a method for delivering a nucleic acid encoding a polypeptide or RNA of interest to plant cell chloroplasts. An exemplary method comprises providing an Agrobacterium variant strain lacking VirD2 in the Vir operon, introducing a binary vector plasmid encoding a polynucleotide fusion construct encoding a 1) VirD2 variant protein and chloroplast transit peptide operably linked to a promoter and terminator which drives expression in said Agrobacterium variant strain, and 2) a T DNA comprising a chloroplast sequence mediating homologous recombination into the chloroplast genome, a nucleic acid encoding a polypeptide or RNA of interest, and a nucleic acid encoding a selectable marker, wherein said VirD2 variant protein forms a complex with said T-DNA and delivers said complex to the chloroplast. Infected plant cells are then identified as those which survive in the presence of a selection agent, thereby identifying cells harboring the T-DNA and expressing said polynucleotide of interest. In certain embodiments the VirD2 variant protein expressed from the binary plasmid comprises a sequence selected from SEQ ID NO: 27, SEQ ID NO: 28, SEQ ID NO: 31, SEQ ID NO: 32, SEQ ID NO: 42 and SEQ ID NO: 43. The selectable marker in the binary vector can be encoded by a sequence selected from SEQ ID NO: 26, SEQ ID NO: 29, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO 40, SEQ ID NO: 41 and SEQ ID NO: 55.

The polynucleotide fusion construct can be present in a binary vector backbone selected from a pAGa vector of SEQ ID NO: 22, a pAGb vector of SEQ ID NO: 23, a pAGc vector of SEQ ID NO: 24, and a pAGd vector of SEQ ID NO: 25.

In a preferred embodiment, the pAG binary vector encoding said PtVirD2 variant is introduced into a XYA105ΔvirD2 strain for co-cultivation and chloroplast transformation in a plant. In yet another aspect, the pAG binary vector comprises a PtVirD2 variant encoded by SEQ ID NO: 29.

The invention also provides an isolated Agrobacterium strain XYA105Pt variant selected from XYA105Pt45, XYA105Pt46, and XYA105Pt47, comprising a reconstituted virD operon encoding a VirD2 variant selected from SEQ ID NO: 45, SEQ ID NO: 46 and SEQ ID NO: 47, said variant Agrobacterium strain optionally comprising a binary vector harboring a T-DNA encoding a nucleic acid of interest and a selectable marker, said T-DNA comprising a sequence which mediates homologous recombination in a chloroplast genome in said plant cell. In another aspect, the plant is infected with a XYA105ΔvirD2 Agrobacterium strain carrying said binary vector comprising Pt-VirD2 variant and said T-DNA.

In certain embodiments, the plant cell is a tobacco, Arabidopsis, corn, soybean, sorghum, wheat, rice, alfalfa, sunflower, or Brassica sp. cell. In certain embodiments, the selectable marker protein can confer a visibly selectable phenotype or it can confer resistance to a selection agent.

In a preferred embodiment, the polynucleotide fusion construct further comprises an operably linked polynucleotide encoding a transit polypeptide sequence, wherein said transit polypeptide sequence targets said VirD2 fusion polypeptide to a subcellular organelle other than the nucleus. In a particularly preferred embodiment, the subcellular organelle is a chloroplast.

The Agrobacterium can be a mutant Agrobacterium tumefaciens strain comprising a mutation in one or more of a virA, virG, virB, virD or virE gene. In certain embodiments, the plant cell is a monocot. In other embodiments, the plant cell is a dicot. The method described above can further include the step of growing the plant cell to produce a plant. In an alternative embodiment, the method can further comprise exposing a flower from the plant to the Agrobacterium, harvesting seed from said flower and optionally germinating said seed on selective media. In another aspect, the method further comprises exposing a flower from the plant to the Agrobacterium, harvesting seed from said flower and identifying transplastomic events by leaf phenotypic change.

In another aspect of the invention, a method for delivering a polynucleotide of interest to plant cell chloroplasts is disclosed. In this embodiment, the method comprises providing an Agrobacterium variant strain with a wild type Vir operon, introducing a binary vector plasmid encoding 1) a polynucleotide fusion construct encoding a VirD2 variant protein operably linked to a promoter and terminator and a chloroplast transit peptide which drives expression in said Agrobacterium variant strain, and 2) a T DNA comprising a chloroplast sequence mediating homologous recombination into the chloroplast genome, a nucleic acid encoding a polypeptide of interest, and a nucleic acid encoding a selectable marker, wherein said VirD2 variant protein forms a complex with said T-DNA and delivers said complex to the chloroplast, and selecting infected plant cells which survive in the presence of a selection agent, thereby identifying cells expressing said polynucleotide of interest. As above, the VirD2 variant protein can be expressed from a binary plasmid comprising a sequence selected from SEQ ID NO: 27, SEQ ID NO: 28, SEQ ID NO: 31, SEQ ID NO: 32, SEQ ID NO: 42 and SEQ ID NO: 43.

Also provided is a kit for practicing the methods of any of the preceding method claims.

### BRIEF DESCRIPTION OF THE DRAWINGS

FIG. 1. VirD2 and Pt-VirD2 protein domains. VirD2 contains a relaxase (endonuclease) domain, a domain of unknown function (DUF), a N-terminal monopartite type NLS (NLS1), a C-terminal bipartite type NLS2 and Y29, NLS 3 in in DUF region. Y29 is tyrosine 29, to which VirD2 links T-DNA. In the Pt-VirD2 protein TP is the Rubisco small subunit transit peptide, 5 aa are the five N-terminal amino acids of the Rubisco small subunit, R32T is the arginine to threonine mutation inactivating NLS 1.

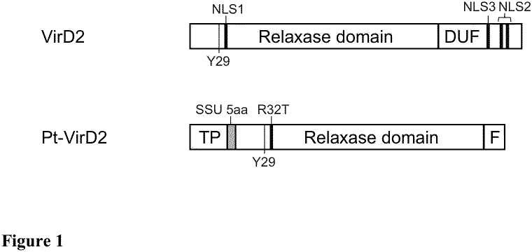

FIGS. 2A - 2B. Schematic diagram of bar^{au} gene excision by Agro-Int or Nuclear-Int yielding plastid genome Nt-pSS33Δ. FIG. 2A) Agro-Int is expressed in Agrobacterium from plasmid pJCD6B and exported to chloroplasts. The tobacco Rubisco small subunit transit peptide (TP) and the VirF (F) T4SS signal were fused at the Int N- and C-termini, respectively. PvE, Promoter virE; TvF, terminator virF. FIG. 2B) Co-cultivation of Agrobacterium carrying a binary plastid pKO117 (Lutz et al., 2004) is followed by the transfer of the T-complex to the nucleus, symbolized with a blue circle (VirD2) and a black line (T-strand). Int is expressed in a nuclear P2′/Tnos cassette and is fused with the pea Rubisco small subunit transit peptide for plastid targeting. T1-ptDNA and T2-ptDNA refer to transformed plastid genomes before and after bar^{au} excision.

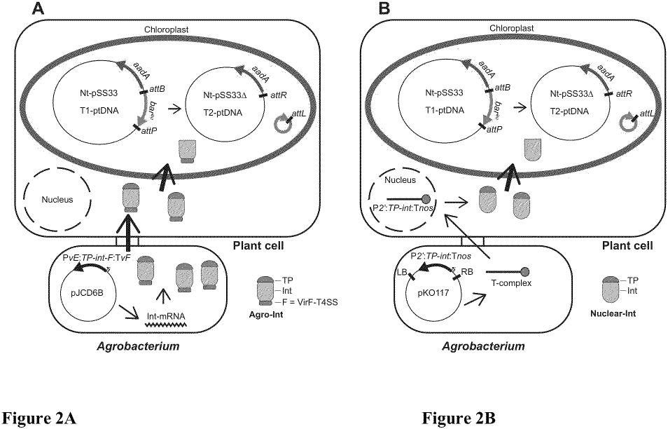

FIGS. 3A -3E. Integrase exported from Agrobacterium excises bar^{au} gene in the Nt-pSS33 chloroplast genome. FIG. 3A) Partial map of the wild-type plastid genome where arrow marks the site of transgene insertion. FIG. 3B) Partial map of the Nt-pSS33 plastid genome before and after the excision of bar^{au} gene. Shown are: the trnI, rrn16 and trnV plastid genes and of the transcription start site of 3’rps12/7 operon promoter; the attB, attP target sites and the attR recombination junction; the AM102 and AM103 primer sites; and the BamHI, EcoRI, and ApaI restriction sites. FIG. 3C), The minimal attB/attP sequence [SEQ ID NO 57, SEQ ID NO 58]: and the attL and attR recombination junctions (SEQ ID NO: 59, SEQ ID NO 60) (Groth et al., 2000). FIG. 3D) Tobacco leaf three days after Agroinfiltration. FIG. 3E) PCR amplification confirms target excision in syringe injected leaves. The AM102 and AM103 primers amplify a 1.9 kb fragment before, and a 0.9 kb fragment after excision of the bar^{au} gene.

FIG. 4. Schematic of the split GFP system to detect Pt-VirD2 fluorescence in plant chloroplasts.

FIG. 5. Plasmids for the split-GFP assay in E. coli. pAM305 is a chloroplast transformation vector carrying GFP1-10 and aadA genes under the chloroplast regulatory elements flanking with chloroplast homologous sequences. pAM31-36 vectors encode different configurations of Pt-VirD2 (TP:: virD2MRwt::F), GFP11, placed at either N- or C- terminus with or without the linker [L, (GGGS)x2; L*, GGGS], and the T4SS signal of VirF (F). pAM30 does not contain GFP11 and used as a complementation negative control. In pAM30-36, the genes can be induced by IPTG to determine the most efficient configuration.

FIG. 6. Fluorescence in E. coli confirms complementation of GFP1-10 and VirD2-11 proteins. Transformants were selected in E. coli on LB plates containing 100 mg/L spectinomycin and 50 mg/L kanamycin. Plates contained 1 mM IPTG. The positive control was plasmid pAM29 [SEQ ID NO 6], expressing sulfite-reductase-GFP11. The negative control was pAM30 [SEQ ID NO 7], Pt-VirD2 without GFP 11.

FIG. 7. Split-GFP assay in chloroplasts after split GFP expression from nuclear genes and transport to the chloroplasts.

FIG. 8. Plasmids for the split-GFP assay in chloroplasts for expression from nuclear genes. pAM407 binary vector encodes TP-GFP1-10 gene and kanamycin resistance gene. pJF7-12 binary vectors carry different configurations of Pt-VirD2 (TP:: virD2MRwt::F), GFP11, placed at either N- or C- terminus with or without the linker [L, (GGGS)x2; L*, GGGS], and the T4SS signal of VirF (F). Pt-virD2-11 genes expressed in an Arabidopsis ubiquitin promoter/terminator cassette (PAtUBQ/TAtUBQ).

FIG. 9. The T-DNA region of pAG binary vectors. Shown are the kanamycin resistance bacterial marker gene in the backbone, and the left border (LB) and right border (RB) repeats. Note duplicated 25 bp LB repeats, and the RB with the 24 nt overdrive (OD, gray box). To facilitate cloning of sequences for T-DNA transfer, we included the PacI, I-SecI and AscI restriction sites between the LB and RB. The a, b, c, and d derivatives differ in the position of multiple cloning sites (MCS).

FIG. 10. The T-DNA region of pAG-CpTEST (also referred to as Pwise in FIG. 13A) binary chloroplast transformation vector. The chloroplast targeting regions contains nucleotides 104,087-101,147 of the tobacco ptDNA (NC_001879.2) with the dicistronic aadA-gfp gene as marker. We show above the MCS a Pt-VirD2 gene that could be any gene listed in Table 1..

FIG. 11. The pAG-CHL-1 Agrobacterium binary vector for chloroplast transformation. The vector carries the aadA gene from vector pMRR13, an MCS and a Pt-VirD2gene. [SEQ ID NO 29). The Pt-VirtD2 gene can be any of the variants listed as SEQ ID NO 27, SEQ ID NO 28, SEQ ID NO 31, SEQ ID NO 32, SEQ ID NO 42, SEQ ID NO 43.

FIG. 12. Reconstituting the virD operon with Pt-virD2 replacing wild type virD2 gene in the operon. On top is sacB plasmid with kanamycin resistance as bacterial marker and part of the virD1-Pt-virD2-virD3 operon for insertion in the virulence plasmid. Cointegrate formation via the D1 (left) or D3 (right) genes is shown, followed by loss of duplicated structures via second homologous recombination event yielding the desired reconstituted operon, or restoration of the original virD2-lacking structure.

FIGS. 13A-13D. Agrobacterium strains for Agrobacterium-mediated chloroplast transformation. FIG. 13A) pWISE binary vector for Agrobacterium-mediated chloroplast transformation. pWISE vector carries the chloroplast transformation vector flanked by T-DNA borders and a plastid-targeted Pt-virD2 gene variant. The Pt-virD2 gene can be any gene listed in Table 1. The pXYA105ΔvirD2 virulence plasmid lacks virD2 gene in the virD operon. FIG. 13B) virD operon in pXYA105ΔvirD2 Agrobacterium strain. FIG. 13C) pWISE0 binary vector in XYA105Pt Agrobacterium strain. Note that binary plasmid pWISE0 lacks Pt-virD2 which is now part of virD operon on the virulence plasmid. FIG. 13D) Reconstituted virD operon in XYA105Pt Agrobacterium strain. Pt-virD2 can be any gene listed in Table 1.

### DETAILED DESCRIPTION OF THE INVENTION

Tissue culture limitations in Arabidopsis nuclear gene transformation were overcome by using Agrobacterium to directly transform the female gametocyte, and identifying nuclear transgenic events by germinating the resulting seedlings on a selective medium. Here we describe reengineering of an Agrobacterium for T-DNA chloroplast delivery to directly transform the plastids in the female gametocyte. Side-stepping the tissue culture process eliminates the need for specialized expertise to practice plastid transformation in Arabidopsis and other plant species. Therefore, the invention described herein enables widespread applications of Arabidopsis plastid genome engineering which, combined with the available extensive genomic resources, will have a major impact on basic science and applications in plant biotechnology.

Agrobacterium tumefaciens is a plant pathogen which delivers tumor-inducing genes encoded in the transferred DNA (T-DNA) to the plant nucleus. The T-DNA has been disarmed and converted into a vector commonly used to obtain transgenic plants. In conventional approaches, T-DNA is delivered by linkage to a virulence (VirD2) protein, which guides the T-DNA through linkage to a Type IV secretion signal sequence (T4SS). Subsequently, the T-DNA is integrated in the plant nucleus. As described herein, the Agrobacterium delivery system has been redesigned to transfer the T-DNA to chloroplasts. We demonstrate that proteins can be directly exported from Agrobacterium to chloroplasts provided a C-terminal Type IV secretion signal (T4SS) is present for export to the plant cell and an N-terminal transit peptide for import into chloroplasts. Targets for reengineering include Vir proteins involved in T-DNA transfer. In order to promote chloroplast delivery of VirD2, the strong nuclear localization (NLS) signals are removed for effective reengineering of the Vir proteins for chloroplast delivery of T-DNA.

In the examples below, compositions and methods are provided for the rapid and efficient reengineering of Agrobacterium and other plant species for T-DNA delivery to chloroplasts to directly transform the plastids in the female gametocyte. Direct transformation of plastids in the female gametocyte eliminates tissue culture as the bottleneck of chloroplast transformation.

### Definitions

By “T-DNA” is meant the transferred DNA or T-DNA of an Agrobacterium tumefaciens Ti plasmid or from an Agrobacterium rhizogenes Ri plasmid, or a derivative thereof. The T-DNA may comprise an entire T-DNA, but need only comprise the minimal sequences required in cis for transfer (i.e., the right and the left T-DNA border sequences). T-DNA delivery to germline cells would enable chloroplast transformation in all crops in which direct transformation of germline cells is feasible by the floral dip protocol.

The invention includes plant transformation vectors for use in Agrobacterium-mediated transformation of chloroplasts in plants. The vectors of the present invention have the pVS 1 replication origin (Heeb et al., 2000), but the replication origin may be replaced by other replication origins from the repABC family in Agrobacterium in certain embodiments. Also, vectors without an Agrobacterium-maintaining replication origin are capable of maintaining themselves in Agrobacterium with sequences in the T-DNA that can be integrated into either the Ti plasmid in A. tumefaciens, Ri plasmid in A. rhizogenes or Agrobacterium chromosome through homologous recombination. This results in the same effect of a reduction in non T-DNA sequence insertion and an increase in low copy number transformation events, since the chromosome, Ti or Ri plasmid is present in a single copy. When Rhizobia or non Agrobacterium species are used for plant transformation, T-DNA containing nucleic acid of interest can be integrated into the chromosome or into repABC plasmids in the host bacteria, which can produce the same effect as Agrobacterium does.

A vector of the present invention may comprise at least one of Agrobacterium Ti plasmid right border or left border region. The vectors may comprise at least one pair of borders. The vectors can include four pairs of borders, but the vector often comprise one or two pairs.

The vectors of the present invention may further comprise a coding region for a selectable marker for the maintenance in bacterial hosts. Coding regions for selectable markers include Spec/Strp that encodes for Shigella flexneri aminoglycoside adenyltransferase (aadA) conferring resistance to spectinomycin or streptomycin (Chinault et al. 1986), or a gentamycin (Gm, Gent) selectable marker gene from a Tn21-like multiresistance transposon (Wohlleben et al., 1989). Other resistance genes include carbenecillin, ampicillin, and kanamycin resistance genes. Others are known and may be readily used in the present invention by those of skill in the art.

The vectors of the present invention may also comprise a coding region for a plant selectable marker gene, which is typically located in T-DNA, to select transformed plant cells with the corresponding selection agent. The plant selectable marker may provide resistance to a positive selection compound, for example, antibiotic resistance (e.g., streptomycin/spectinomycin, kanamycin, chloramphenicol, tobramycin, gentamycin, etc.), or herbicide resistance (e.g., including but not limited to: glyphosate, Dicamba, glufosinate, sulfonylureas, imidazolinones, bromoxynil, dalapon, cyclohexanedione, protoporphyrinogen oxidase inhibitors, and isoxaflutole herbicides). Polynucleotide molecules encoding proteins involved in herbicide tolerance are known in the art and can also be introduced into target plant cells using the compositions and methods of the invention.

In addition to a plant selectable marker, in some embodiments it may be desirable to use a reporter gene. In some instances, a reporter gene may be used with or without a selectable marker. Reporter genes are genes that are typically not present in the recipient organism or tissue and typically encode for proteins resulting in some phenotypic change or enzymatic property.

Preferred reporter genes include the beta-glucuronidase (GUS) of the uidA locus of E. coli, the chloramphenicol acetyl transferase gene from Tn9 of E. coli, the green fluorescent protein from the bioluminescent jellyfish Aequorea victoria, and the luciferase genes from firefly Photinus pyralis or mScarlet. An assay for detecting reporter gene expression may then be performed at a suitable time after said gene has been introduced into recipient cells. A preferred such assay entails the use of split GFP.

In some embodiments, the vectors of the present invention comprise one replication origin for maintenance in E. coli. These origins of replication may be derived, for example, from pBR322 or from pUC. One example of such an origin of replication is ColE1. The vectors of the present invention can include any origin of replication which maintains at least 2, 3, 4, 5, 6, 7, 8, 9, 10, 15, 20, 25, 30, 35, 40, 45, 50, 60, 70, 80, 90, or 100 or more plasmid copies in E. coli. In some embodiments, the ori maintains a higher copy number in E. coli (e.g., greater than 5, 10, 15, 20, 25, or 30, and in some further embodiments, greater than 10, 15, 20, 25 or 30). Such high copy number vectors make it easier to amplify the amount of DNA available for transformation.

As used herein “operably linked” includes reference to a functional linkage between two sequences in a nucleic acid construct, for example, a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein “promoter” includes reference to a region of DNA involved in recognition and binding of RNA polymerase and other proteins to initiate transcription.

The invention discloses novel methods of delivering proteins of interest directly to plant organelles. Because the protein of interest is transferred directly into the plant cell, the potential negative effects which can result from the transient or stable introduction of genetic material are avoided. Another benefit of the invention is that a protein of interest can be directly delivered to a plant cell in conjunction with stable incorporation of a transgene. This allows for a broad range of utilities from increasing the efficiency of plant cell transformation to enhancing agroinfection.

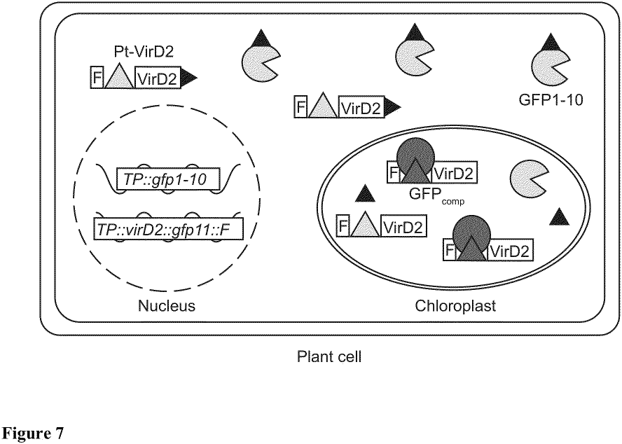

In addition, the methods of the invention can be used to test the efficacy of visible selectable markers by transferring chimeric VirD fusions with peptides derived from proteins such as GFP as described in the split GFP assay shown in FIG. 7. The visible markers could also be used in the system to test changes in protocols that would enhance transfer of molecules to various plant cells, or cells or tissues of recalcitrant species.

As used herein, the term “plant” includes reference to whole plants, plant organs (e.g., leaves, stems, roots, etc.), seeds and plant cells and progeny of same. Plant cell, as used herein includes, without limitation, plant cells within or isolated from seeds, suspension cultures, embryos, meristematic regions, callus tissue, leaves, roots, shoots, gametophytes, sporophytes, pollen, and microspores.

Proteins delivered to plant cells using the Agrobacterium-mediated method will generally be localized to the nucleus of the plant cell. Additional modifications to the protein could result in the targeting to other specific subcellular locations within the plant cell. For example, “signal” or “transit” peptides are capable of targeting a polypeptide to the chloroplast, as known in the art. It is recognized that in the Agrobacterium-mediated method of protein delivery to plant cells, the signal peptide would be added to the polypeptide of interest in the bacterium. More specifically, the modified Agrobacterium containing a replicon encoding a protein which has a T4SS at its C-terminus to facilitate the movement of proteins from Agrobacterium to the plant cell and an N-terminal “signal sequence” or “transit peptide” to target the polypeptide of interest to a specific subcellular location in the plant cell.

Known chloroplast transit peptides include, without limitation, the chloroplast small subunit of ribulose-1,5-bisphosphate carboxylase (Rubisco); 5-enolpyruvyl)shikimate-3-phosphate synthase (EPSPS); tryptophan synthase; plastocyanin; chorismate synthase; and the light harvesting chlorophyll a/b binding protein (LHBP).

The DNA construct of the present invention may be introduced into the genome of a desired plant host by a suitable Agrobacterium-mediated plant transformation method. Several Agrobacterium species mediate the transfer of T-DNA, described above, that can be genetically engineered to carry any desired piece of DNA into many plant species. Recently, non-Agrobacterium systems are coming to the fore for plant transformation, specifically Ensifer adhaerens, Ochrobactrum haywardense and Rhizobium etli (Rathore DS, Mullins E , 2018).

Other methods for introducing DNA into cells may also be used. These methods are well known to those of skill in the art and can include chemical methods and physical methods such as microinjection, electroporation, and micro-projectile.

Plant cell regeneration techniques rely on manipulation of certain phytohormones in a tissue culture growth medium, also typically relying on a biocide and/or herbicide marker that has been introduced together with the desired nucleotide sequences. Choice of methodology with suitable protocols being available for hosts from Leguminosae (alfalfa, soybean, clover, etc.), Umbelliferae (carrot, celery, parsnip), Cruciferae (cabbage, radish, canola/rapeseed, etc.), Cucurbitaceae (melons and cucumber), Gramineae (wheat, barley, rice, maize, etc.), Solanaceae (potato, tobacco, tomato, peppers), various floral crops, such as sunflower, and nut-bearing trees, such as almonds, cashews, walnuts, and pecans. Such regeneration techniques are known to those of skill in the art. Methods and compositions for transforming plants by introducing a transgenic DNA construct into a plant genome in the practice of this invention can include any of the well-known and demonstrated methods. For example, Agrobacterium-mediated transformation as illustrated in U.S. Pat. Nos. 5,824,877; 5,591,616; and 6,384,301, all of which are incorporated herein by reference.

The present invention may be used for delivery of proteins or modulatory DNA/RNAs to any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber ( C. sativus), cantaloupe ( C. cantalupensis), and musk melon ( C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida ), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pin us radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

### Kits Comprising for Practicing the Methods for Agrobacterium Mediated Transformation of Chloroplasts

The invention also provides a kit for comprising materials and reagents useful for practice of the methods of the present invention.

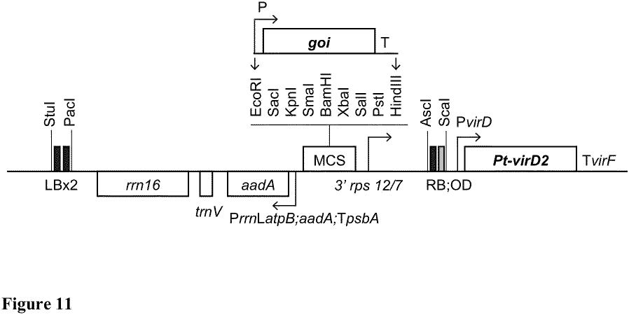

Suitable pAG-CHL binary vectors (FIG. 11) for practicing the invention are pAG-CHL-1 [SEQ ID NO 29], where the Pt-VirD2-1 [SEQ ID NO 27] is blunted to the right of RB; pAG-CHL- 2, where Pt-VirD2-2 [SEQ ID NO 28] is blunted to the right of RB; pAG-CHL- 3, where Pt-VirD2-3 [SEQ ID NO 30] is blunted to the right of RB; pAG-CHL- 4, where Pt-VirD2-4 [SEQ ID NO 31] is blunted in the to the right of RB.

A suitable Agrobacterium strain carrying a virulence plasmid lacking the T-DNA and a virD2 gene, is XYA105ΔvirD2. The Pt-VirD2 protein can nick the T-DNA at the borders and guide it to plastids (e.g. chloroplasts) subsequent to transfer to the plant cell. Pt-VirD2 plasmids are incorporated on the binary plasmid. These plasmids will be made part of the virD operon carried on the helper plasmid. The reconstituted virD operon will comprise Pt-VirD2 variants, such as [SEQ ID NO 1], [SEQ ID NO 31], [SEQ ID NO 42] and [SEQ ID NO 43].

Other reagents include, without limitation, media and selection agents, described herein useful for the production of the transplastomic plants of the invention.

The following materials and methods are provided to facilitate the practice of the present invention.

Plant materials and growth conditions. Nicotiana tabacum cv. Petit Havana Nt-pSS33 is a transplastomic tobacco plant, carrying the excisable, attB/attP flanked bar^{au} gene in the plastid genome (Tungsuchat-Huang et al., 2010). The plants were grown in the greenhouse with supplemental lighting (16 hrs).

Vector construction. Agro-Int gene in plasmid pJCD6B is based on a pPZP200 binary vector derivative (Hajdukiewicz et al., 1994), in which the BclI restriction site was converted into a StuI site. The T-DNA borders have been removed by digestion with StuI/ScaI restriction enzymes, and replaced with the Agro-Int gene consisting of the virE promoter (Winans et al., 1987) (GenBank: X06826.2), the tobacco Rubisco small subunit transit peptide (Gnanasambandam et al., 2007) fused with the phiC31 integrase (AJ006589.3) at the N-terminus and 37 amino acids of the virF T4SS signal at the C-terminus, followed by the virF terminator (Melchers et al., 1990, Vergunst et al., 2005). Removal of the transit peptide and both, the transit peptide and the T4SS signal, yielded binary plasmids pAMland pAM2, respectively.

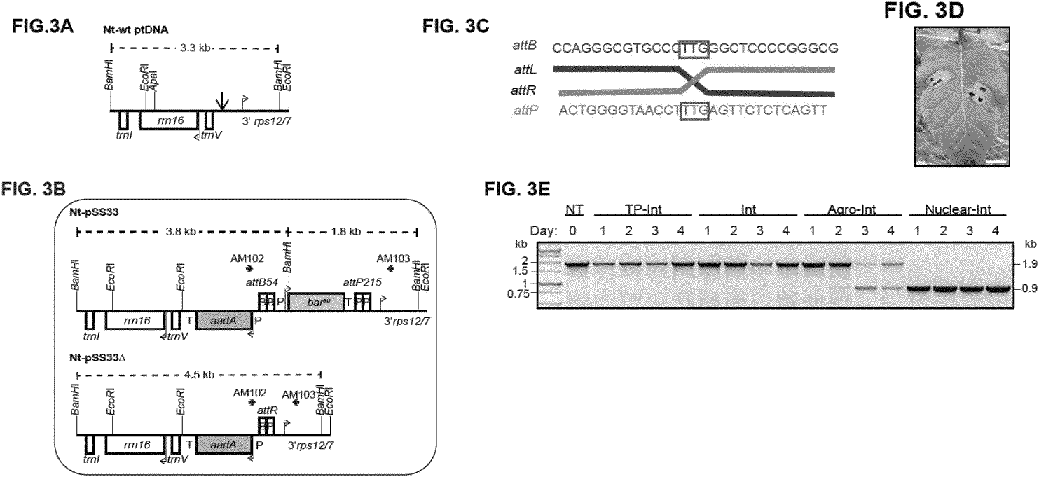

Agroinfiltration. Agrobacterium strain EHA105 (Hood et al., 1993) was transformed with the pAM1 (TP-Int), pAM2 (Int), or pJCD6B (Agro-Int) binary vectors described herein or pKO117 Nuclear-Int vector (Lutz et al., 2004). Infiltration of leaves with syringe was carried out as described (Sparkes et al., 2006). It was important to choose fully extended leaves of young plants before flower buds appeared. The Agrobacterium strains were inoculated into 30 mL YEB medium supplemented with 100 mg/L spectinomycin and 25 mg/L chloramphenicol and grown overnight at 28° C. to a final OD₆₀₀ of 0.5. The cultures were centrifuged at 4,200 g for 10 min. The cells were resuspended in 3 mL of the infiltration medium (250 mg D-glucose, 5 mL of 500 mM MES, 5 mL of Na₂PO₄·7H₂O, 50 µL of 100 µL of 100 mM acetosyringone, dH₂O up to 50 mL final volume). To wash the cells, the cell suspension was centrifuged again and then resuspended in 3 mL of the infiltration medium. The final OD₆₀₀ was adjusted to 1 for infiltration. Leaf sections (1 cm²) were collected daily for four days after infiltration (FIG. 3D.

PCR and DNA gel blot analyses. Total cellular DNA was isolated using the CTAB protocol. Excision of the target sequence was confirmed by PCR using total cellular DNA as template and primers AM102 (5′-GTTGAGTCGATACTTCGGCG-3′) (SEQ ID NO: 48) and AM103 (5′-GGCACCAGTCCTACATTTTG-3′) (SEQ ID NO: 49). The primer positions are shown in FIG. 3B. PCR amplicons were sequenced to verify the attR junction sequence (FIG. 3C). PCR was performed in a 20 µL reaction mixture containing 1.3 µg of total plant cellular DNA, 0.5 µM of primers AM102 and AM103, 1xJumpStart™REDTaq® ReadyMix™ Reaction Mix (SIGMA-ALDRICH, St. Louis, MO, Cat. No. P0982 ). PCR parameters were as follows: one cycle at 95° C. for 3 min, 30 cycles at 95° C. for 30 s, 57° C. for 30 s, 72° C. for 30 s, and one cycle at 72° C. for 10 min.

DNA gel blot analysis was also performed to detect excision of the target sequence (Tungsuchat-Huang et al., 2010). Total cellular DNA was digested with the BamHI restriction enzyme and probed with the 2.1 kb ApaI-BamHI ptDNA fragment.

The following examples are provided to illustrate certain embodiments of the invention. They are not intended to limit the invention in any way.

### Example I

### Split GFP Assay to Visualize Engineered Vird2 Movement to Chloroplasts

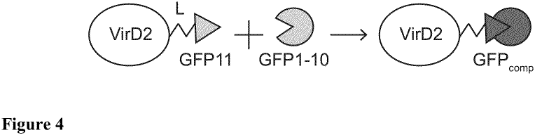

We have shown direct export of the integrase site-specific recombinase by the excision of target sequences in chloroplasts. As a follow-up, we confirmed that VirD2, the protein guiding the T-DNA from Agrobacterium to the plant nucleus can also be re-targeted to chloroplasts. For this, we have used the split GFP assay (Cabantous et al., 2005). In the assay a small part of GFP (GFP11; RDHMBLHEYBNAAGIT) (SEQ ID NO: 50) is incorporated in the VirD2 protein (VirD2-11) and when it is transported from Agrobacterium (or the cytoplasm) to chloroplasts it complements a GFP 1-10 protein [SEQ ID NO 5] lacking the small peptide (GFP 11) (FIG. 4). Localization of the engineered VirD2-11 to chloroplasts is detected by fluorescence under a confocal microscope of complementing GFP parts.

### Split GFP Complementation In E. Coli

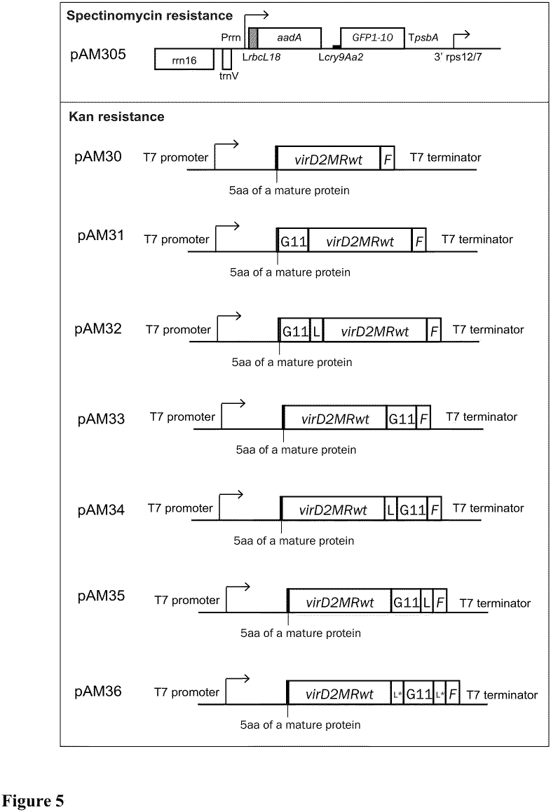

We tested complementation of VirD2-11 variants and GFP 1-10 in E. coli by expressing GFP 1-10 in a pUC plasmid carrying a spectinomycin resistance marker (pAM305 [SEQ ID NO 5]) and VirD2-11 in a PET28a vector carrying a kanamycin resistance marker. Selection for spectinomycin resistance and kanamycin resistance ensured that both plasmids were present in E. coli. The VirD2-11 plasmids expressed a variant lacking the transit peptide (TP) but had the five SSU amino acids at the N-terminus, the form that will be present when the TP is cleaved off after import in chloroplasts. Some of the VirD2 variants carried the GFP 11 repeat at the N-terminus between the 5 SSU amino acids and the VirD2 N-terminus (pAM31 [SEQ ID NO 8]; pM32 [SEQ ID NO 9]); others between the truncated VirD2 C-terminus and the VirF T4SS (pAM33 [SEQ ID NO 10], AM34 [SEQ ID NO 11], pAM35 [SEQ ID NO 12] and pAM36 [SEQ ID NO 13]). Plasmids pAM32, pAM34, pA35 and pAM36 carry a linker sequence adjacent to the 16 aa GFP 11 peptide. The schematic maps of the plasmids used for the split GFP assay in E. coli are shown in FIG. 5.

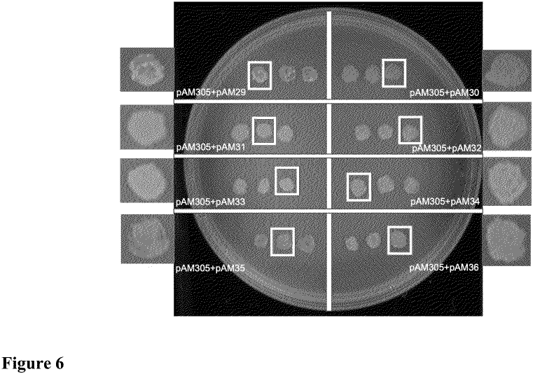

Each of the VirD2-11 proteins complemented GFP 1-10, indicated by fluorescence upon illumination with UV light (UVL-56, 366 nm Black-Ray Lamp, UVP Upland, CA 91786) (FIG. 6). The positive control was plasmid pAM29 [SEQ ID NO 6], expressing sulfite-reductase-GFP11 (Cabantous et al., 2005). The negative control was pAM30 [SEQ ID NO 7], Pt-VirD2 without GFP 11.

### Split-GFP Complementation in Chloroplasts From Nuclear Genes

A split-GFP assay in chloroplasts when both GFP-1-10 and the Pt-VirD2 variants are expressed from nuclear genes can also be performed. The assay is schematically shown in FIG. 7. GFP1-10 gene is cloned in a 35S promoter/ocs terminator cassette in plasmid pM407, a pBIN19 vector derivative [Seq. ID 14](Bevan, 1984). The GFP1-10 gene was introduced into the tobacco nucleus by standard protocols, selecting for kanamycin resistance. The gene was transcribed in the nucleus, its mRNA translated in the cytoplasm and the GFP 1-10 protein imported into chloroplasts where the transit peptide (RP) is cleaved off. GFP 1-10 is not fluorescent under UV light.

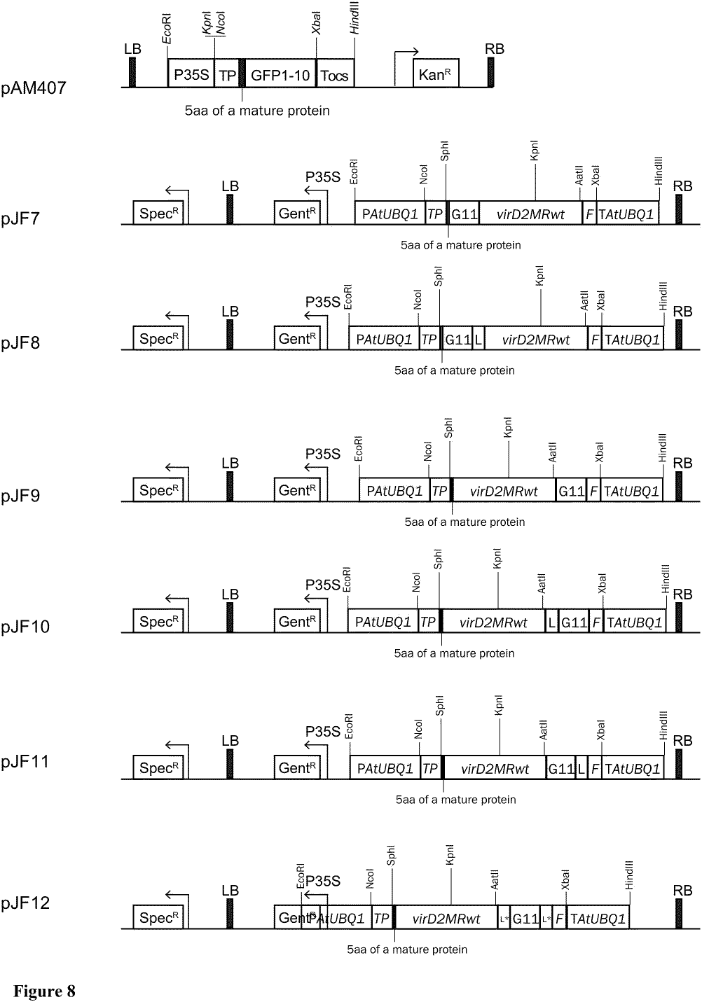

Pt-VirD2-11 plasmids are a series of plastid VirD2 variants, which carry a complementing GFP11 fragment in a pPZP 221 binary plasmid (Hajdukiewicz et al., 1994). The Pt-VirD2-11 genes encode proteins similar to those tested in XE. coli (FIG. 5), other than having the transit peptide sequence. The genes are cloned in a ubiquitin promoter/terminator cassette (Liu et al., 2015). The genes for the Pt-VirD2-11 variants are introduced into the tobacco nucleus of GFP 1-10 transgenic plants, where the genes are transcribed in the nucleus, their mRNA is translated in the cytoplasm and the Pt-VirD2-11 protein is imported in the chloroplasts where it complements GFP 1-10, indicating successful re-targeting of VirD2 to chloroplasts. The targeting region of Pt-VirD2-11 plasmids is shown in FIG. 8. The DNA sequence of plasmids is also available: pJF7 [SEQ ID NO 15], pJF8 [SEQ ID NO 16], pJF9 [SEQ ID NO 17], pJF10 [SEQ ID NO 18], pJF11 [SEQ ID NO 19] and pJF12 [SEQ ID NO 20].

### Agrobacterium-Mediated Chloroplast Transformation

To accomplish Agrobacterium-mediated chloroplast transformation we had to develop a new toolkit, because the markers in the current toolkit are not compatible with the Agrobacterium strain lacking VirD2. The new constructs we developed providing the new tools necessary for practice Agrobacterium-mediated chloroplast transformation are described in detail below.

### XYA105ΔvirD2 Agrobacterium Strain

In certain embodiments, to avoid competition between chloroplast and nuclear targeting, we utilize an Agrobacterium strain lacking the virD2 gene (XYA105ΔvirD2). This strain is the derivative of the EHA101 agrobacterium strain, a C58 nopalin strain carrying the pTiBo542 virulence plasmid lacking the transferred DNA (T-DNA) (Hood et al., 1986). DNA sequence of plasmid pTi_EHA101 is deposited in GenBank (KY000035.1). The kanamycin resistance gene in pEHA101 was deleted (Hood et al., 1993), then the left-T-DNA border still present on the virulence plasmid was removed to obtain plasmid XYA105 (Li et al., 2020). The virD2 gene in XYA105 was subsequently deleted to obtain XYA105ΔvirD2 (Li et al., 2020). The deleted sequence is shown as [SEQ ID NO 21].

### pAG Binary Backbone Plasmid Family

To adjust to the markers in XYA105, we constructed a new binary Agrobacterium binary vector family based on the pAG backbone with kanamycin resistance as the bacterial marker. The backbone of pAG serves as the foundation for both the chloroplast vector pAGC and the nuclear vector pAGN. The kanamycin resistance gene is described as part of pUK-S expression vector encoding APH(3′)-I family aminoglycoside 0-phosphotransferase (WP_099588326; AF053406.1)(Krieg et al., 1998). We modified the coding region by removing two NsiI restriction sites and one NruI restriction site in the coding region by silent mutagenesis and converted the kanR gene into an NsiI-StuI fragment by adding the restriction sites at the ends [SEQ ID NO 30]. The spectinomycin resistance gene in the pPZP200 binary vectors could be replaced with the synthetic StuI-NsiI fragment purchased from BlueHeron Biotechnology.

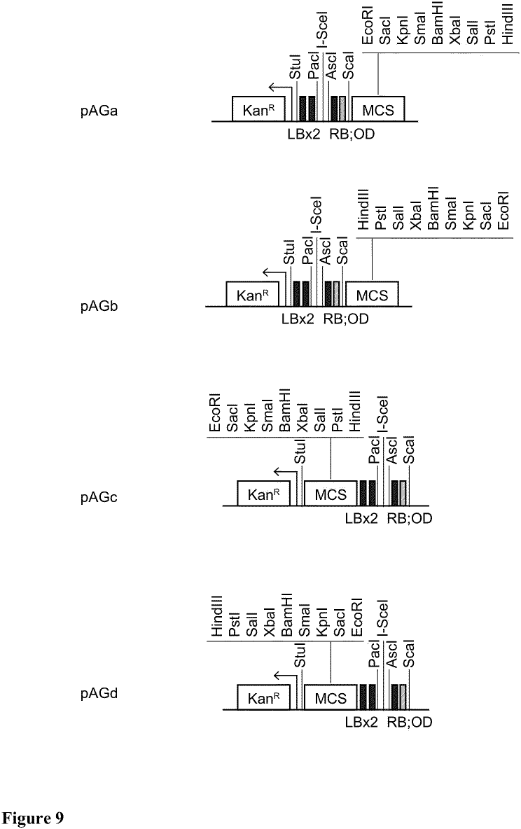

The pAG T-DNA region was designed as follows (FIG. 9). The LB is the 25 bp pTiT37 left border repeat, as in the pPZP vectors (Hajdukiewicz et al., 1994). We have used two 25-bp repeats [SEQ ID NO 51] with a spacer [SEQ ID NO 52], because two repeats improved the correct processing of the T-stand, reducing the transfer of vector sequences beyond the LB (Kuraya et al., 2004, Podevin et al., 2006, Thole et al., 2007). The RB also derives from pTiT37 [SEQ ID NO 53], as in the pPZP binary vectors (Hajdukiewicz et al., 1994). For efficient T-DNA transfer, sequences termed overdrive [SEQ ID NO 54], that are binding sites for VirC1, are also included (Toro et al., 1989). Because the pTiT37 plasmid does not contain a well-defined overdrive sequence, we have utilized the pTiA6 overdrive (Peralta et al., 1986, Thole et al., 2007). Between the LB and RB we included the PacI, I-SecI and AscI restriction sites to facilitate insertion of sequences for T-DNA transfer.

Four variants of the pAG backbone were created differing in the position of pUC multiple cloning sites relative to the T-DNA. In version a (pAGa) the multiple cloning site (MCS) is adjacent to the T-DNA right border (RB) and the restriction sites are as in pUC18 (From EcoRI to HindIII); in version b (pAGb) the MCS is also adjacent to the RB, but the restriction sites are in the reverse order (from HindIII- to EcoRI); in c (pAGc) and d (pAGd) the restriction sites are outside the LB and are arranged as in pUC18 (pAGc) and in reverse order (pAGd) for cloning flaxibility. The map of the T-DNA region of the four pGA vectors is shown in FIG. 9. The DNA sequences of the vectors are: pAGa [SEQ ID NO 22], pAGb [SEQ ID NO 23], pAGc [SEQ ID NO 24] and pAGd [SEQ ID NO 25].

### The pAGC Chloroplast Transformation Vector

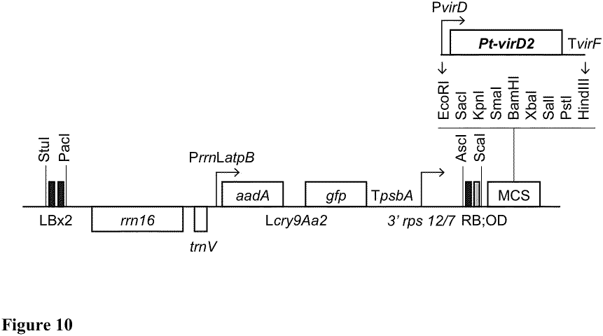

The pAGC chloroplast transformation backbone is based on the pAGa-pAGd vector backbones and targets insertion between the trnV gene and 3′-rpsl2/7 operon in the inverted repeat region of the tobacco chloroplast genome. The targeting regions contain nucleotides 104,087-101,147 of the tobacco ptDNA (NC_001879.2) where one of the HincII site was converted into a cloning site, using SnaBI and HpaI enzymes. The position of the insertion site is the same as in the pPRV chloroplast transformation vectors (Zoubenko et al., 1994). To facilitate identification of transplastomic events a dicistronic aadA-gfp operon (MF461355) was inserted into the cloning site, so that transplastomic events can be selected by spectinomycin resistance, and non-transplastomic spectinomycin resistant clones eliminated by the lack of GFP accumulation (Yu et al., 2017, Yu et al., 2020). Spectinomcyin resistance of non-transplastomic clones can be due to nuclear insertion of the marker gene cassette and spontaneous mutations in the 16S rRNA gene. The map of binary chloroplast transformation vector pAGCa carrying the dicistronic aad-gfp cassette [SEQ ID NO 26] is shown in FIG. 10. The schematic map of Pt-VirD2-1 gene in a VirD promoter/VirF terminator cassette [SEQ ID NO 27] is shown above the MCS. Pt-VirD2-2 can also be expressed in a VirE promoter/VirF terminator cassette [SEQ ID NO 28] for efficiency in chloroplast transformation.

### Chloroplast Transformation by Agrobacterium Using the pAG-CpTEST Chloroplast Transformation Vector

For successful chloroplast transformation by Agrobacterium, a Pt-VirD2 gene should be inserted in the multiple cloning site. To obtain a properly regulated plastid Pt-VirD2 gene, the Pt-VirD2 coding sequence can be inserted into a VirD operon promoter/VirF terminator cassette and the cassette cloned in the pAGCa multiple cloning site to obtain an agrobacterium binary vector, pAG-CpTEST [SEQ ID NO 26]. The vector can then be introduced by electroporation into a XYA105ΔvirD2 strain and used for cocultivation using a protocol modified for chloroplast transformation.

For chloroplast transformation by Agrobacterium, leaf sections of sterile plants are infiltrated with Agrobacterium, and then the leaf sections are transferred to a selective medium containing 500 mg/L spectinomycin to select for transplastomic events and 500 mg/L carbenicillin to control Agrobacterium proliferation.

Agroinfiltration of tobacco leaves is carried out as described (Kapila et al., 1997, Lutz et al., 2006). Briefly, Agrobacterium strain XYA105ΔvirD2 strain is transformed with vector pAGC-1. The Agrobacterium strains were inoculated into 100 mL yeast extract beef medium supplemented with 50 mg/L kanamycin and 25 mg/L chloramphenicol and grown overnight at 28° C. to a final OD₆₀₀ = 0.8. The cultures were sedimented by centrifugation, and resuspended in MMA medium (MS salts, 10 mM of MES, 20 g/L sucrose, pH 5.6, and 200 µM acetosyringone) to a final OD₆₀₀ of 2.4. The Agrobacterium suspension was kept at 22° C. for one hour, then used for Agroinfiltration of sterile leaves (1 cm²). It is important to select fully expanded leaves Agroinfiltration. Vacuum was applied at 5 Torr for 20 min. Leaf sections were placed on RMOP medium for co-cultivation. Placing the infiltrated leaf disks on a filter paper helps to control Agrobacterium growth during cocultivation. After 2 days of co-cultivation, leaves are transferred to RMOP medium supplemented with 500 mg/L carbenicillin to inhibit Agrobacterium cell division and 500 mg/L spectinomycin to select for transplastomic events. Transplastomic events are identified as green proliferating calli and shoots on a selective medium that suppresses growth and greening of wild type tobacco cells (Svab and Maliga, 1993).

### pAG-CHL Vectors for Introduction of Genes of Interest in the Plastid Genome

In vector pAG-CpTEST-1, (also referred to as Pwise herein) the Pt-VirD2-1 gene is flanked by restriction sites, so that alternative Pt-VirD2 variants can be readily cloned into the vector. The resulting vectors can also be tested for improving the efficiency T-DNA transfer. The tobacco SSU transit peptide and truncated VirD2 fusion protein has been shown to be efficiently processed by VirD2 in a nicking assay (Matsuoka, 2015), but T-DNA delivery to chloroplasts may be improved by using alternative transit peptide sequences (Armbruster et al., 2009, Sjuts et al., 2017). When suitable variants are identified, the Pt-VirD2 variant will be inserted without any major cloning sites, and the targeting region will be modified to include a multiple cloning site for the insertion of genes of interest (vector pAG-CHL-1; SEQ ID NO 29; FIG. 11).

### Floral Dip Protocol

The Arabidopsis thaliana floral dip protocol is caried out as described (Clough and Bent, 1998) and modified (Zhang et al., 2006). The Arabidopsis plants (~nine per pot) are grown in soil until they bolt and 80% start to produce first floral inflorescences. The first shoots are then cut down to 3 cm to provide a mechano-stimulus for a uniform second batch of shoots with more branching (7-10 days). When the second batch of shoots are 15-20 cm, they are ready for the floral dip. To prepare Agrobacterium cultures for floral dip, cultures are streaked on fresh LB medium with antibiotics. 2mL liquid LB is then inoculated with 2-3 colonies for overnight (~16 hours) culture at 28° C. with shaking at 220-230 rpm in a 1000 mL flask. Agrobacterium cells are collected by centrifugation, then resuspended in 1L ddH₂O, 50 g sucrose, 0.48 g MgCl₂, 300-500 µl Silwett L-77 added just prior to resuspending Agrobacterium). The flowers are briefly dipped (30-60 seconds) in the Agrobacterium solution with gentle swirling of the the inflorescences. The pots are then laid sideways and the plants covered with a plastic cover to preserve humidity overnight. The plants are placed upright in a clean tray and allowed to flower.

Seed is collected and germinated on RM medium modified for Arabidopsis (Yu et al., 2017; Yu et al., 2019). Seedlings with green sectors that fluoresce are then identified under UV light. The seedlings are then transferred to soil and seeds collected from branches with GFP sectors. The seed is then germinated in the presence of spectinomycin (50 mg/L) plants having uniformly transformed plastid genomes selected.

### Example II

### T-DNA Markers in Agrobacterium Including Antibiotic Resistance

The T-DNA in the T-complex is assumed to be single stranded (Stachel et al., 1986). As described in Example I, Agrobacterium binary vectors can deliver several kb-long T-strands. Smaller T-DNA strands are more likely to be imported into chloroplasts via the translocons that evolved for protein import from the cytoplasm. The shortest T-DNA strands that can be used as markers for chloroplast transformation is approximately 60 nt. Antibiotic resistance is a preferred marker; however, many other marker options are known to those skilled in the art.

### Selection for Antibiotic Resistance by Conversion of Native Plastid Genes With T-DNA Carrying Short Oligonucleotides

Early evidence for plastid transformation was obtained by selection for spectinomycin resistance encoded in 16S rRNA which is present in the plastid genome. The wild-type sensitive allele can be converted into a resistant one by a short single-stranded DNA transported by the Pt-VirD2 protein. There are several point mutations that disrupt a short stem structure in the 16S rRNA (Svab and Maliga, 1991). 60 nt-long synthetic oligonucleotides containing point mutations can be cloned as PacI/AscI fragments to replace the plastid targeting region in the pAG-CHL-1 Agrobacterium binary vector (FIG. 11). DNA sequences of PacI/AscI fragments with oligonucleotides conferring streptomycin resistance in the 16S rRNA include [SEQ ID NO 33] [SEQ ID NO 34] and [SEQ ID NO 35]. PacI/AscI fragments conferring spectinomycin resistance to the 16S rRNA include [SEQ ID NO 36], [SEQ ID NO 37], [SEQ ID NO 38], [SEQ ID NO 39] and [SEQ ID NO 40]. Single nucleotide exchanges occur randomly, yielding identical phenotypes. The Agrobacterium cocultivation and selection for spectinomycin (or streptomycin) resistance predominantly yielding the mutation that is present in the vector strongly supports gene conversion by T-DNA as the source of spectinomycin (streptomycin) resistance.

In an alternative approach, streptomycin resistance can also be conferred by introducing point mutations in rps12, a plastid-encoded ribosomal protein gene (Galili et al., 1989; Staub and Maliga, 1992; Hsu et al., 1993). Chloroplast transformants and spontaneous mutants could be distinguished via selection. The C to T mutation in codon 91 results in a Pro to Ser substitution, conferring streptomycin resistance in rps12. Silent mutations TTA to TTG or GGT to GGA in the adjacent codons do not result in amino acid substitutions. The point mutation resulting in streptomycin resistance will be included in the T-DNA, alongside with at least one of the adjacent silent mutations. Under these conditions, the presence of the silent mutation alongside with the mutation conferring streptomycin resistance. an event that is unlikely to occur spontaneously, provides irrefutable evidence that streptomycin resistance is due to gene conversion by the T-DNA. The sequence of the PacI/AscI fragment that yields streptomycin resistance and the TTA to TTG silent mutation in the adjacent codon is [SEQ ID NO: 41]. The vector with C271T changes Pro to Ser conferring streptomycin resistance and T276A is a silent mutation in [SEQ ID NO: 55]. An additional oligonucleotide that confers streptomycin resistance is SEQ ID NO 61, in which the C271T changes Pro to Ser conferring streptomycin resistance and two additional silent mutations create a KpnI site (Staub and Maliga, 1992).

### Alternative Transit Peptides to Facilitate the Import of T-Complex by Pt-VirD2 Variants

Transit peptides facilitate the import of proteins which are targeted to chloroplasts. These peptides are cleaved off by a stromal processing peptidase (SPP) upon entry into the stroma (Sjuts et al., 2017). In certain embodiments, the Pt-VirD2 constructs have the tobacco Rubisco small subunit transit peptide (TP) [SEQ ID NO 1]. The tobacco Rubisco SSU is relatively small (XM_016585367; 180 aa). Plastidic homomeric acetyl-CoA carboxylase (ACC2, AT1G36180.1) is one of the largest proteins (2355 aa) targeted to chloroplasts and its transit peptide, is provided on ppdb.tc.cornell.edu and the PeptideAtlas. The mature protein starts with C76 or F77. Inclusion of the 6 amino acids of the mature ACC2 N-terminus, renders information on the exact cleavage site is irrelevant. It is expected that the ACC2 TP which normally imports large proteins, (SEQ ID NO: 42) will be highly efficient. A third TP that shows promise is a non-canonical stromal targeted protein in which the transit peptides are not processed by a SPP. For example, Arabidopsis mitogen-activated protein kinase kinase4 (AtMKK4) has a non-canonical transit peptide; its first 65 residues are necessary for chloroplast targeting (GenBank: NP_175577, NM_104044) (Samuel et al., 2008). PtMKK4-VirD2 [SEQ ID NO 43] comprising the Arabidopsis MKK4 transit peptide can also be used.

### Construction of Agrobacterium Strains Carrying New Helper Plasmids With Pt-VirD2 as Part of the Vird Operon

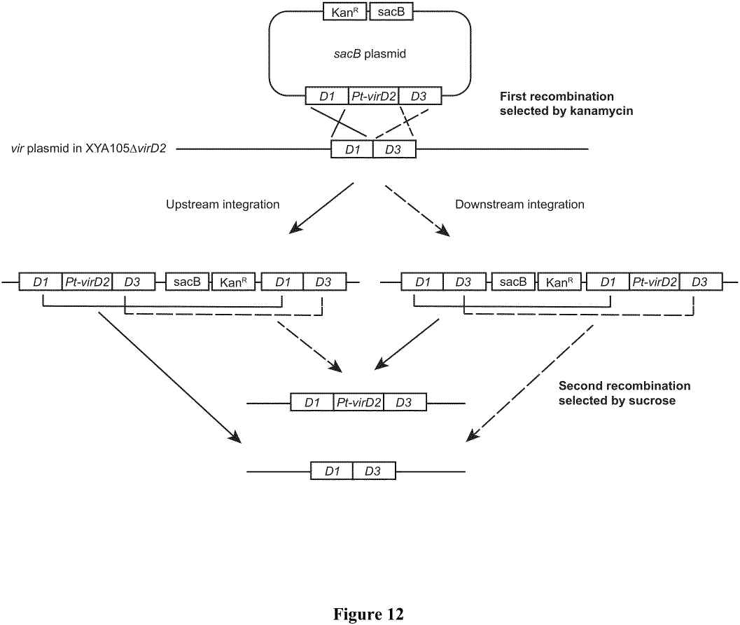

The Pt-VirD2 plasmids are incorporated on the binary plasmid. These plasmids can also be made part of the virD operon carried on the helper plasmid. This can be accomplished using a sacB-based gene replacement strategy as illustrated in FIG. 12 (Hoang et al., 1998; Huang and Wilks, 2017; Li et al., 2020).

First, a tetracycline resistance gene will be replaced with a kanamycin resistance gene in pEX18Tc (GenBank Accession No. AF047519) (Available on the world wide web at novoprolabs.com/vector/Vgyytina) yielding plasmid pEX18Kan [SEQ ID NO 44]. For insertion into the virD operon, Pt-VirD2 genes were flanked by part of the virD1 gene and virD3 gene in vector pEX18Kan. The pEX18Kan derivatives will be introduced into XYA105ΔvirD2 strain. Agrobacterium first will be selected for kanamycin resistance to obtain cointegrated forms, then grown on sucrose to recover derivatives in which Pt-VirD2 was incorporated in the virD operon (FIG. 12). The vector for insertion of Pt-VirD2 with the Rubisco SSU-TP is [SEQ ID NO 45], with the ACC2-TP is [SEQ ID NO 46] and with the MKK4-TP is [SEQ ID NO 47].

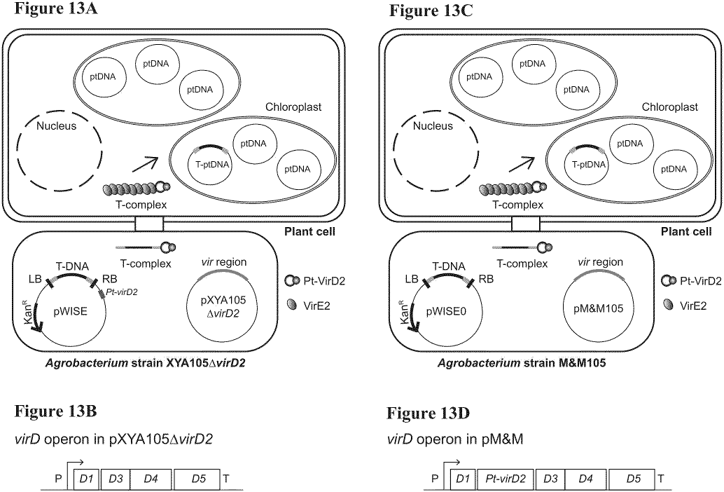

FIG. 13 depicts chloroplast transformation using Agrobacterium. (A) Chloroplast transformation in a tobacco cell with the XYA105ΔvirD2 stain, carrying a pWISE binary vector. (B) The virD2 operon on the XYA105ΔvirD2 virulence plasmid. (C) Chloroplast transformation in a tobacco cell with a pWISE0 binary vector, using a pM&M105 Agrobacterium strain. (D) The Pt-virD2 gene on the pM&M105 virulence plasmid.

In case of the pWISE vectors (FIG. 13A), the binary plasmid encodes both the T-DNA and the Pt-virD2. An example is SEQ ID NO 29 (FIG. 11). Similar WISE vectors can be obtained by combining the pAGa, pAGb, pAGc and pAGd backbones (FIG. 9) with the vector sequences in Table 2 and Pt-VirD2 genes in Table 1.

When pWISE0 vectors are used in pM&M105 Agrobacterium strains (FIG. 13C), the pWISE0 binary vector may encode the T-DNA sequence listed in Table 2 only, because Pt-VirD2 is part of the virD operon.

TABLE 1

SEQ ID OF Pt-virD2 genes

| SEQ ID NO | Promoter/Terminator Cassette | Chloroplast Transit Peptide | virD2 derivative | T4SS |
|---|---|---|---|---|
| 31 | PvirD/TvirF | Rubisco SSU+5aa | Pt-VirD2 | virF |
| 27 | PvirD/TvirF | Rubisco SSU+5aa | Pt-VirD2M | virF |
| 42 | PvirD/TvirF | ACC2+6aa | Pt-virD2 | virF |
| 43 | PvirD/TvirF | mKK4 | Pt-virD2 | virF |
| 32 | PvirE/TvirF | Rubisco SSU+5aa | Pt-VirD2 | virF |
| 28 | PvirE/TvirF | Rubisco SSU+5aa | Pt-VirD2M | virF |

TABLE 2

Chloroplast targeting region of pWISE vectors between PacI and AscI sites

| SEQ ID NO | Targeting seq. | Marker / selection | Reporter gene or goi |
|---|---|---|---|
| 26 | trnV/3′rps12/7 (long) | aadA | gfp |
| 29 | trnV/3′rps12/7 (long) | aadA | |
| 56 | trnV/3′rps12/7 (short) | aadA | |
| 33 | rrn16 | Streptomycin r. | |
| 34 | rrn16 | Streptomycin r. | |
| 35 | rrn16 | Streptomycin r. | |
| 36 | rrn16 | Spectinomycin r. | |
| 37 | rrn16 | Spectinomycin r. | |
| 38 | rrn16 | Spectinomycin r. | |
| 39 | rrn16 | Spectinomycin r. | |
| 40 | rrn16 | Spectinomycin r. | |
| 41 | rps12 | Streptomycin r. | |
| 55 | rps12 | Streptomycin r. | |
| 61 | rps12 | Streptomycin r. | |

Sequences useful for the practice of the present invention

| | |
|---|---|
| SEQ ID NO 1 | DNA sequence of Pt-VirD2. Coding sequence, tobacco SSU-TP+5aa fused with mutant minimal VirD2+T4SS signal |
| SEQ ID NO 2 | DNA sequence of integrase gene in binary vector pJCD6B. |
| SEQ ID NO 3 | Amino acid sequence of the phiC31 integrase in plasmid pJCD6. |
| SEQ ID NO 4 | DNA sequence of binary plasmid pJCD6B. |
| SEQ ID NO 5 | pAM305, with dicistronic aadA-gfp1-10 gene, for expression in chloroplasts. |
| SEQ ID NO 6 | pAM29. Sulfite-reductase-GFP11 fusion protein, in a T7 P/T cassette. |
| SEQ ID NO 7 | pAM30. Pt-VirD2 (without GFP1)1, in a T7 P/T cassette. |
| SEQ ID NO 8 | pAM31 |
| SEQ ID NO 9 | pAM32 |
| SEQ ID NO 10 | pAM33 |
| SEQ ID NO 11 | pAM34 |
| SEQ ID NO 12 | pAM35 |
| SEQ ID NO 13 | pAM36 |
| SEQ ID NO 14 | pAM407 |
| SEQ ID NO 15 | pJF7 |
| SEQ ID NO 16 | pJF8 |
| SEQ ID NO 17 | pJF9 |
| SEQ ID NO 18 | pJF10 |
| SEQ ID NO 19 | pJF11 |
| SEQ ID NO 20 | pJF12 |
| SEQ ID NO 21 | Deleted virD2 sequence in XYA105DvirD2 |
| SEQ ID NO 22 | DNA sequence of backbone vector pAGa |
| SEQ ID NO 23 | DNA sequence of backbone vector pAGb |
| SEQ ID NO 24 | DNA sequence of backbone vector pAGc |
| SEQ ID NO 25 | DNA sequence of backbone vector pAGd |
| SEQ ID NO 26 | DNA sequence of binary chloroplast transformation vector pAG-CpTEST Note dicistronic aadA-gfp marker genes. |
| SEQ ID NO 27 | The DNA sequence of Pt-VirD2 in a VirD promoter/VirF terminator cassette. The Pt-VirD2 has the tobacco SSU Transit peptide+5aa, the mutant minimal VirD2-T4SS signal. |
| SEQ ID NO 28 | The DNA sequence of Pt-VirD2 in a VirE promoter/VirF terminator cassette. The Pt-VirD2 has the tobacco SSU Transit peptide+5aa, the mutant minimal VirD2-T4SS signal. |
| SEQ ID NO 29 | DNA sequence of pAG-CHL-1 chloroplast binary transformation vector for introduction of transgenes (goi) into the plastid genome. The binary vector carries a Pt-VirD2 gene outside the T-DNA listed as SEQ ID No 27. |
| SEQ ID NO 30 | DNA sequence of kanamycin resistance gene in StuI-NsiI fragment. |
| SEQ ID NO 31 | The DNA sequence of Pt-VirD2-3 in a VirD promoter/VirF terminator cassette where Pt-VirD2 has the wild type NLS1. The Pt-VirD2-3 has the tobacco SSU Transit peptide+5aa, the wt minimal VirD2-T4SS signal. |
| SEQ ID NO 32 | The DNA sequence of Pt-VirD2-4 in a VirE promoter/VirF terminator cassette where Pt-VirD2 has the wild type NLS1. The Pt-VirD2-4 has the tobacco SSU Transit peptide+5aa, the wt minimal VirD2-T4SS signal. |
| SEQ ID NO 33 | DNA sequence introducing a C to T point mutation at nucleotide 473 in tobacco plastid 16S RNA coding sequence flanked by PacI and AscI conferring streptomycin resistance |
| SEQ ID NO 34 | DNA sequence introducing a C to A point mutation at nucleotide 861 in tobacco plastid 16S RNA coding sequence flanked by PacI and AscI conferring streptomycin resistance |
| SEQ ID NO 35 | DNA sequence introducing a C to T point mutation at nucleotide 861 in tobacco plastid 16S RNA coding sequence flanked by PacI and AscI conferring streptomycin resistance |
| SEQ ID NO 36 | DNA sequence introducing a G to A point mutation at nucleotide 1013 in tobacco plastid 16S RNA coding sequence flanked by PacI and AscI conferring spectinomycin resistance |
| SEQ ID NO 37 | DNA sequence introducing a A to C point mutation at nucleotide 1139 in tobacco plastid 16S RNA coding sequence flanked by PacI and AscI conferring spectinomycin resistance |
| SEQ ID NO 38 | DNA sequence introducing a C to T point mutation at nucleotide 1140 in tobacco plastid 16S RNA coding sequence flanked by PacI and AscI conferring spectinomycin resistance |
| SEQ ID NO 39 | DNA sequence introducing a G to A point mutation at nucleotide 1141 in tobacco plastid 16S RNA coding sequence flanked by PacI and AscI conferring spectinomycin resistance |
| SEQ ID NO 40 | DNA sequence introducing a G to A point mutation at nucleotide 1335 in tobacco plastid 16S RNA coding sequence flanked by PacI and AscI conferring spectinomycin resistance |
| SEQ ID NO 41 | DNA sequence introducing streptomycin resistance and a silent mutation in the tobacco rps12 coding sequence. Flanked by PacI and AscI restriction sites.. C271T changes Pro to Ser conferring streptomycin resistance. A270G is a silent mutation. |
| SEQ ID NO 42 | DNA sequence of Pt-ACC2-virD2 in a VirE promoter/VirF terminator cassette (P/T are in lower case) where Pt-ACC2-virD2 has A. thaliana ACC2 transit peptide+6aa fused with wt minimal VirD2 and T4SS signal. |
| SEQ ID NO 43 | DNA sequence of PtMKK4-virD2 in a VirE promoter/VirF terminator cassette (P/T are in lower case) where PtMKK4-virD2 has A. thaliana MKK4 transit peptide fused with wt minimal VirD2 and T4SS signal. |
| SEQ ID NO 44 | DNA sequence of pEX18Kan pEX18Tc (Accession no. AF047519) derivative with kanamycin resistance as selectable marker. |
| SEQ ID NO 45 | EcoRI-HindIII fragment of Pt-virD2 flanked with 500 bp of virD1 and 500 bp of virD3. The PtVirD2 has the tobacco SSU Transit peptide, the wt minimal VirD2-T4SS signal listed as SEQ ID NO 31. |
| SEQ ID NO 46 | EcoRI-HindIII fragment of PtACC2-virD2 flanked with 500 bp of virD1 and 500 bp of virD3. The PtACC2-VirD2 has the Arabidopsis ACC2 Transit peptide, the wt minimal VirD2-T4SS signal listed as SEQ ID NO 42. |
| SEQ ID NO 47 | EcoRI-HindIII fragment of PtMKK4-virD2 flanked with 500 bp of virD1 and 500 bp of virD3. The PtMKK4-VirD2 has the Arabidopsis MKK4 Transit peptide, the wt minimal VirD2-T4SS signal listed as SEQ ID NO 43. |
| SEQ ID NO 48 | AM 102 Primer |
| SEQ ID NO 49 | AM 102 Primer |
| SEQ ID NO 50 | GFP11 |
| SEQ ID NO 51 | Left Border sequence |
| SEQ ID NO 52 | Spacer between Left Border repeats |
| SEQ ID NO 53 | Right Border sequence |
| SEQ ID NO 54 | Overdrive sequence |
| SEQ ID NO 55 | DNA sequence introducing streptomycin resistance and a silent mutation in the tobacco rps12 coding sequence. Flanked by PacI and AscI restriction sites.. C271T changes Pro to Ser conferring streptomycin resistance. T276A is a silent mutation. |
| SEQ ID NO 56. | A variant of SEQ ID NO 29 (FIG. 11 ), with shorter plastid targeting sequences. Note that the PacI and AscI sites have been blunted. |
| SEQ ID NO 57 | The minimal attB sequence |
| SEQ ID NO 58 | The minimal attP sequence |
| SEQ ID NO 59 | The attL sequence |
| SEQ ID NO 60 | The attR sequence |
| SEQ ID NO 61 | Oligonucleotide confers streptomycin resistance and the silent mutations create a KpnI restriction site in the rps12 plastid gene. |

> SEQ ID NO 1 DNA sequence of Pt-VirD2. Coding sequence, tobacco SSU-TP+5aa fused with mutant minimal VirD2+T4SS signal

atggcttcctcagttctttcctccgcagcagttgccacccgcagcaatgt
tgctcaagctaacatggttgcacctttcactggccttaagtcagctgcct
cattccctgtttcaaggaagcaaaaccttgacatcacttccattgccagc
aacggcggaagagtgcaatgcatgCAGGTGTGGCCAGCGATGGCTCCCGA
TCGGGCCcaagtaatcattcgcattgtgccaggaggtggaaccaagaccc
ttcagcagataatcaatcagctggagtacctgtccacgaagggaaagctg
gaactccagcgttcagcccggcatctcgatattcccgttccgccggatca
aatccgtgagcttgcccaaagctgggttacggaggccgggatttatgacg
aaagtcagtcagacgatgacaggcaacaagacttaacaacacacattatt
gtgagcttccccgcaggtacggaccaaaccgcagcttatgaagcaagccg
ggaatgggcagccgagatgtttgggtcaggatacgggggtggccgctata
actatctgacagcctaccacgtggaccgcgatcatccacatttacatgtc
gtggtcaatcgtcgggaacttctggggcaggggtggctgaaaatatccag
gcgccatccccagctgaattatgacggcttacggaaaaagatggcagaga
tttcacttcgtcacggcatagtcctggatgcgacttcccgagcagaaagg
ggaatagcagagcgaccaatcacctatgctgaatatcgacgtCTTGAGCG
GATGCTCGAAGTTATGGCAGAAGTTCGGCCCATAGCCCGATCCATTAAAA
CGGCTCACGACGATGCGCGAGCGGAATTAATGTCGGCGGACAGACCTCGA
TCAACGCGCGGTCTATGA

>SEQ ID NO 2 DNA sequence of integrase gene in binary vector pJCD6B

tgcggaacatagaaagcgtcagagacgagacgcaccgtaagtgcgtgcta
cattgtcctcggccgcacctaaagaccggggcagttgaaatccgattgtg
atggcaactataagcgggtgacgaacagcacggattctagaatcatagac
cgcgcgttgatcgaggtctgtccgccgacattaattccgctcgcgcatcg
tcgtgagccgttttaatggatcgggctatgggccgaacttctgccataac
ttcgagggcgcccgccgcgacgtcttccgtgccgtcctgggcgtcgtctt
cgtcgtcgtcggtcggcggcttcgcccacgtgatcgaagcgcgcttctcg
atgggcgttccctgccccctgcccgtagtcgacttcgtgacaacgatctt
gtctacgaagagcccgacgaacacgcgcttgtcgtctactgacgcgcgcc
cccaccacgacttagggccggtcgggtcagcgtcggcgtcttcggggaac
cattggtcaaggggcagcttcggggcttcggcggcttcaagttcggcaag
ccgctcttccgccccttgctgccggagcgtcagcgctgcctgttgcttcc
ggaagtgcttcctgccaacgggtccgtcgtacgcgcctgccgcgcggtct
tcgtacagctcttcaagggcgttcagggcgtcggcgcgctccgcaacaag
gttcgcccgttcgccgctcttctcaggcgcctcagtgagcttgccgaagc
gtcgggcggcttcccacagaagcgccaacgtctcttcgtcgccttcggcg
tgcctgatcttgttgaagatgcgttccgcaacgaacttgtcgagtgccgc
catgctgacgttgcacgtgccttcgtgctgcccaggtgcggacgggtcga
ccaccttccggcgacggcagcggtaagagtccttgatcgattcttccccg
cgcttcgaagtcatgacggcgccacactcgcagtacagcttgtccatcgc
ggacagaatggcttgcccccgggaaagccccttgccgcgccccctgccgt
ccaaccacgcctgaagctcataccactcagcgggctcgatgatcggtccg
caatcaagctcgaccggccggagcgtgatcgggtcgcgctgaatgcggta
accctcaatcttcgtggtcggcgtgccgtccggcttcttcttgtagatca
cctcagcggcgaagcccgcaatacgcgggtcccgaaggattcgcataacg
gttgccgggtcccaggcgcttgaagcggtcttcttcccaatcgtctcgcc
ccgggtcggcacggcgtcagcgtccatgcgcttacaaagccccgtgatgc
tgcccgggtgaatggcggcttgactgcccggcttgaagggaaggtgtttg
tgcgtcttgatctcacgccaccaccaccggattacgtcgggctcgaactc
gaagggtccggtaaggggagtggtcgagtgcgccagcttgttgatgacga
cattgaccattcggccgttgcgcgtgatctccttcgtctccgaaacaagc
tcgaagccgtaaggcgccttcccgccgacgtacccgcccaattcgcgctg
aaggttcttcgtgtcgagaatcttcgccgacttcagcgaagattctttgt
gcgacgcgtcgagccgcataatcaggtgaatcaggtccatgacgtttccc
tgccggaagacgccttcctgagtggaaacaatcgtcacgcccagggcgag
caattccgagacaatcggaatcgcgtccatgaccttcaggcgcgagaagc
gcgacacgtcatagacaatgatcatgttgagccgcccggcgcggcattcg
ttcaggatgcgttcgaactccgggcgctccgccgtcccgaacgccgacgt
gcccggcgcttcgctgaaatgcccgacgaacctgaaccggcccccgtcgc
gctcgacttcgcgctgaaggtcggccgccttgtcttcgttggcgctacgc
tgtgtcgctgggcttgctgcgctcgaattttcgcgctcgcgcgactgacg
gtcgtaagcacccgcgtacgtgtccatgctagccatggctggccacacct
gcatgcattgcactcttccgccgttgctggcaatggaagtgatgtcaagg
ttttgcttccttgaaacagggaatgaggcagctgacttaaggccagtgaa
aggtgcaaccatgttagcttgagcaacattgctgcgggtggcaactgctg
cggaggaaagaactgaggaagccatatgttctctcctgcaaaattgcggt
tttccagaaaatcctctgcaacccgagataaagctgctttgctactttga
gcaaagccgggggcggaactgttttgatcagtgcgaccactagacgaccc
acttataaggtccgtcgtcagtaacctcttatcggaatttttaagtctcc
gaccgggaagtctggcaagtgctcatatctccaaagtaccatccatactc
gggtcttgaaacggatatcgcgtttcaactgcaatgagcaggcaataaat
gatgccacaatcgacctgaaagggaggaactggaaagactggacgcacgc
ggcctgactgtcgaggccatcgtatccctgtgagcattgttggtgacgag
cagccg

>SEQ ID NO 3 Amino acid sequence of the phiC31 integrase in plasmid pJCD6

MASSVLSSAAVATRSNVAQANMVAPFTGLKSAASFPVSRKQNLDITSIAS
NGGRVQCMQVWPAMASMDTYAGAYDRQSRERENSSAASPATQRSANEDKA
ADLQREVERDGGRFRFVGHFSEAPGTSAFGTAERPEFERILNECRAGRLN
MIIVYDVSRFSRLKVMDAIPIVSELLALGVTIVSTQEGVFRQGNVMDLIH
LIMRLDASHKESSLKSAKILDTKNLQRELGGYVGGKAPYGFELVSETKEI
TRNGRMVNVVINKLAHSTTPLTGPFEFEPDVIRWWWREIKTHKHLPFKPG
SQAAIHPGSITGLCKRMDADAVPTRGETIGKKTASSAWDPATVMRILRDP
RIAGFAAEVIYKKKPDGTPTTKIEGYRIQRDPITLRPVELDCGPIIEPAE
WYELQAWLDGRGRGKGLSRGQAILSAMDKLYCECGAVMTSKRGEESIKDS
YRCRRRKVVDPSAPGQHEGTCNVSMAALDKFVAERIFNKIRHAEGDEETL
ALLWEAARRFGKLTEAPEKSGERANLVAERADALNALEELYEDRAAGAYD
GPVGRKHFRKQQAALTLRQQGAEERLAELEAAEAPKLPLDQWFPEDADAD
PTGPKSWWGRASVDDKRVFVGLFVDKIVVTKSTTGRGQGTPIEKRASITW
AKPPTDDDEDDAQDGTEDVAAGALEVMAEVRPIARSIKTAHDDARAELMS
ADRPRSTRGL

>SEQ ID NO 4 DNA sequence of binary plasmid pJCD6B

agctttgcggaacatagaaagcgtcagagacgagacgcaccgtaagtgcg
tgctacattgtcctcggccgcacctaaagaccggggcagttgaaatccga
ttgtgatggcaactataagcgggtgacgaacagcacggattctagaatca
tagaccgcgcgttgatcgaggtctgtccgccgacattaattccgctcgcg
catcgtcgtgagccgttttaatggatcgggctatgggccgaacttctgcc
ataacttcgagggcgcccgccgcgacgtcttccgtgccgtcctgggcgtc
gtcttcgtcgtcgtcggtcggcggcttcgcccacgtgatcgaagcgcgct
tctcgatgggcgttccctgccccctgcccgtagtcgacttcgtgacaacg
atcttgtctacgaagagcccgacgaacacgcgcttgtcgtctactgacgc
gcgcccccaccacgacttagggccggtcgggtcagcgtcggcgtcttcgg
ggaaccattggtcaaggggcagcttcggggcttcggcggcttcaagttcg
gcaagccgctcttccgccccttgctgccggagcgtcagcgctgcctgttg
cttccggaagtgcttcctgccaacgggtccgtcgtacgcgcctgccgcgc
ggtcttcgtacagctcttcaagggcgttcagggcgtcggcgcgctccgca
acaaggttcgcccgttcgccgctcttctcaggcgcctcagtgagcttgcc
gaagcgtcgggcggcttcccacagaagcgccaacgtctcttcgtcgcctt
cggcgtgcctgatcttgttgaagatgcgttccgcaacgaacttgtcgagt
gccgccatgctgacgttgcacgtgccttcgtgctgcccaggtgcggacgg
gtcgaccaccttccggcgacggcagcggtaagagtccttgatcgattctt
ccccgcgcttcgaagtcatgacggcgccacactcgcagtacagcttgtcc
atcgcggacagaatggcttgcccccgggaaagccccttgccgcgccccct
gccgtccaaccacgcctgaagctcataccactcagcgggctcgatgatcg
gtccgcaatcaagctcgaccggccggagcgtgatcgggtcgcgctgaatg
cggtaaccctcaatcttcgtggtcggcgtgccgtccggcttcttcttgta
gatcacctcagcggcgaagcccgcaatacgcgggtcccgaaggattcgca
taacggttgccgggtcccaggcgcttgaagcggtcttcttcccaatcgtc
tcgccccgggtcggcacggcgtcagcgtccatgcgcttacaaagccccgt
gatgctgcccgggtgaatggcggcttgactgcccggcttgaagggaaggt
gtttgtgcgtcttgatctcacgccaccaccaccggattacgtcgggctcg
aactcgaagggtccggtaaggggagtggtcgagtgcgccagcttgttgat
gacgacattgaccattcggccgttgcgcgtgatctccttcgtctccgaaa
caagctcgaagccgtaaggcgccttcccgccgacgtacccgcccaattcg
cgctgaaggttcttcgtgtcgagaatcttcgccgacttcagcgaagattc
tttgtgcgacgcgtcgagccgcataatcaggtgaatcaggtccatgacgt
ttccctgccggaagacgccttcctgagtggaaacaatcgtcacgcccagg
gcgagcaattccgagacaatcggaatcgcgtccatgaccttcaggcgcga
gaagcgcgacacgtcatagacaatgatcatgttgagccgcccggcgcggc
attcgttcaggatgcgttcgaactccgggcgctccgccgtcccgaacgcc
gacgtgcccggcgcttcgctgaaatgcccgacgaacctgaaccggccccc
gtcgcgctcgacttcgcgctgaaggtcggccgccttgtcttcgttggcgc
tacgctgtgtcgctgggcttgctgcgctcgaattttcgcgctcgcgcgac
tgacggtcgtaagcacccgcgtacgtgtccatgctagccatggctggcca
cacctgcatgcattgcactcttccgccgttgctggcaatggaagtgatgt
caaggttttgcttccttgaaacagggaatgaggcagctgacttaaggcca
gtgaaaggtgcaaccatgttagcttgagcaacattgctgcgggtggcaac
tgctgcggaggaaagaactgaggaagccatatgttctctcctgcaaaatt
gcggttttccagaaaatcctctgcaacccgagataaagctgctttgctac
tttgagcaaagccgggggcggaactgttttgatcagtgcgaccactagac
gacccacttataaggtccgtcgtcagtaacctcttatcggaatttttaag
tctccgaccgggaagtctggcaagtgctcatatctccaaagtaccatcca
tactcgggtcttgaaacggatatcgcgtttcaactgcaatgagcaggcaa
taaatgatgccacaatcgacctgaaagggaggaactggaaagactggacg
cacgcggcctgactgtcgaggccatcgtatccctgtgagcattgttggtg
acgagcagccggaattactttgatccaacccctccgctgctatagtgcag
tcggcttctgacgttcagtgcagccgtcttctgaaaacgacatgtcgcac
aagtcctaagttacgcgacaggctgccgccctgcccttttcctggcgttt
tcttgtcgcgtgttttagtcgcataaagtagaatacttgcgactagaacc
ggagacattacgccatgaacaagagcgccgccgctggcctgctgggctat
gcccgcgtcagcaccgacgaccaggacttgaccaaccaacgggccgaact
gcacgcggccggctgcaccaagctgttttccgagaagatcaccggcacca
ggcgcgaccgcccggagctggccaggatgcttgaccacctacgccctggc
gacgttgtgacagtgaccaggctagaccgcctggcccgcagcacccgcga
cctactggacattgccgagcgcatccaggaggccggcgcgggcctgcgta
gcctggcagagccgtgggccgacaccaccacgccggccggccgcatggtg
ttgaccgtgttcgccggcattgccgagttcgagcgttccctaatcatcga
ccgcacccggagcgggcgcgaggccgccaaggcccgaggcgtgaagtttg
gcccccgccctaccctcaccccggcacagatcgcgcacgcccgcgagctg
atcgaccaggaaggccgcaccgtgaaagaggcggctgcactgcttggcgt
gcatcgctcgaccctgtaccgcgcacttgagcgcagcgaggaagtgacgc
ccaccgaggccaggcggcgcggtgccttccgtgaggacgcattgaccgag
gccgacgccctggcggccgccgagaatgaacgccaagaggaacaagcatg
aaaccgcaccaggacggccaggacgaaccgtttttcattaccgaagagat
cgaggcggagatgatcgcggccgggtacgtgttcgagccgcccgcgcacg
tctcaaccgtgcggctgcatgaaatcctggccggtttgtctgatgccaag
ctggcggcctggccggccagcttggccgctgaagaaaccgagcgccgccg
tctaaaaaggtgatgtgtatttgagtaaaacagcttgcgtcatgcggtcg
ctgcgtatatgatgcgatgagtaaataaacaaatacgcaaggggaacgca
tgaaggttatcgctgtacttaaccagaaaggcgggtcaggcaagacgacc
atcgcaacccatctagcccgcgccctgcaactcgccggggccgatgttct
gttagtcgattccgatccccagggcagtgcccgcgattgggcggccgtgc
gggaagatcaaccgctaaccgttgtcggcatcgaccgcccgacgattgac
cgcgacgtgaaggccatcggccggcgcgacttcgtagtgatcgacggagc
gccccaggcggcggacttggctgtgtccgcgatcaaggcagccgacttcg
tgctgattccggtgcagccaagcccttacgacatatgggccaccgccgac
ctggtggagctggttaagcagcgcattgaggtcacggatggaaggctaca
agcggcctttgtcgtgtcgcgggcgatcaaaggcacgcgcatcggcggtg
aggttgccgaggcgctggccgggtacgagctgcccattcttgagtcccgt
atcacgcagcgcgtgagctacccaggcactgccgccgccggcacaaccgt
tcttgaatcagaacccgagggcgacgctgcccgcgaggtccaggcgctgg
ccgctgaaattaaatcaaaactcatttgagttaatgaggtaaagagaaaa
tgagcaaaagcacaaacacgctaagtgccggccgtccgagcgcacgcagc
agcaaggctgcaacgttggccagcctggcagacacgccagccatgaagcg
ggtcaactttcagttgccggcggaggatcacaccaagctgaagatgtacg
cggtacgccaaggcaagaccattaccgagctgctatctgaatacatcgcg
cagctaccagagtaaatgagcaaatgaataaatgagtagatgaattttag
cggctaaaggaggcggcatggaaaatcaagaacaaccaggcaccgacgcc
gtggaatgccccatgtgtggaggaacgggcggttggccaggcgtaagcgg
ctgggttgtctgccggccctgcaatggcactggaacccccaagcccgagg
aatcggcgtgagcggtcgcaaaccatccggcccggtacaaatcggcgcgg
cgctgggtgatgacctggtggagaagttgaaggccgcgcaggccgcccag
cggcaacgcatcgaggcagaagcacgccccggtgaatcgtggcaagcggc
cgctgatcgaatccgcaaagaatcccggcaaccgccggcagccggtgcgc
cgtcgattaggaagccgcccaagggcgacgagcaaccagattttttcgtt
ccgatgctctatgacgtgggcacccgcgatagtcgcagcatcatggacgt
ggccgttttccgtctgtcgaagcgtgaccgacgagctggcgaggtgatcc
gctacgagcttccagacgggcacgtagaggtttccgcagggccggccggc
atggccagtgtgtgggattacgacctggtactgatggcggtttcccatct
aaccgaatccatgaaccgataccgggaagggaagggagacaagcccggcc
gcgtgttccgtccacacgttgcggacgtactcaagttctgccggcgagcc
gatggcggaaagcagaaagacgacctggtagaaacctgcattcggttaaa
caccacgcacgttgccatgcagcgtacgaagaaggccaagaacggccgcc
tggtgacggtatccgagggtgaagccttgattagccgctacaagatcgta
aagagcgaaaccgggcggccggagtacatcgagatcgagctagctgattg
gatgtaccgcgagatcacagaaggcaagaacccggacgtgctgacggttc
accccgattactttttgatcgatcccggcatcggccgttttctctaccgc
ctggcacgccgcgccgcaggcaaggcagaagccagatggttgttcaagac
gatctacgaacgcagtggcagcgccggagagttcaagaagttctgtttca
ccgtgcgcaagctgatcgggtcaaatgacctgccggagtacgatttgaag
gaggaggcggggcaggctggcccgatcctagtcatgcgctaccgcaacct
gatcgagggcgaagcatccgccggttcctaatgtacggagcagatgctag
ggcaaattgccctagcaggggaaaaaggtcgaaaaggtctctttcctgtg
gatagcacgtacattgggaacccaaagccgtacattgggaaccggaaccc
gtacattgggaacccaaagccgtacattgggaaccggtcacacatgtaag
tgactgatataaaagagaaaaaaggcgatttttccgcctaaaactcttta
aaacttattaaaactcttaaaacccgcctggcctgtgcataactgtctgg
ccagcgcacagccgaagagctgcaaaaagcgcctacccttcggtcgctgc
gctccctacgccccgccgcttcgcgtcggcctatcgcggccgctggccgc
tcaaaaatggctggcctacggccaggcaatctaccagggcgcggacaagc
cgcgccgtcgccactcgaccgccggcgcccacatcaaggcaccctgcctc
gcgcgtttcggtgatgacggtgaaaacctctgacacatgcagctcccgga
gacggtcacagcttgtctgtaagcggatgccgggagcagacaagcccgtc
agggcgcgtcagcgggtgttggcgggtgtcggggcgcagccatgacccag
tcacgtagcgatagcggagtgtatactggcttaactatgcggcatcagag
cagattgtactgagagtgcaccatatgcggtgtgaaataccgcacagatg
cgtaaggagaaaataccgcatcaggcgctcttccgcttcctcgctcactg
actcgctgcgctcggtcgttcggctgcggcgagcggtatcagctcactca
aaggcggtaatacggttatccacagaatcaggggataacgcaggaaagaa
catgtgagcaaaaggccagcaaaaggccaggaaccgtaaaaaggccgcgt
tgctggcgtttttccataggctccgcccccctgacgagcatcacaaaaat
cgacgctcaagtcagaggtggcgaaacccgacaggactataaagatacca
ggcgtttccccctggaagctccctcgtgcgctctcctgttccgaccctgc
cgcttaccggatacctgtccgcctttctcccttcgggaagcgtggcgctt
tctcatagctcacgctgtaggtatctcagttcggtgtaggtcgttcgctc
caagctgggctgtgtgcacgaaccccccgttcagcccgaccgctgcgcct
tatccggtaactatcgtcttgagtccaacccggtaagacacgacttatcg
ccactggcagcagccactggtaacaggattagcagagcgaggtatgtagg
cggtgctacagagttcttgaagtggtggcctaactacggctacactagaa
ggacagtatttggtatctgcgctctgctgaagccagttaccttcggaaaa
agagttggtagctcttgatccggcaaacaaaccaccgctggtagcggtgg
tttttttgtttgcaagcagcagattacgcgcagaaaaaaaggatctcaag
aagatcctttgatcttttctacggggtctgacgctcagtggaacgaaaac
tcacgttaagggattttggtcatgcatgatatatctcccaatttgtgtag
ggcttattatgcacgcttaaaaataataaaagcagacttgacctgatagt
ttggctgtgagcaattatgtgcttagtgcatctaatcgcttgagttaacg
ccggcgaagcggcgtcggcttgaacgaatttctagctagacattatttgc
cgactaccttggtgatctcgcctttcacgtagtggacaaattcttccaac
tgatctgcgcgcgaggccaagcgatcttcttcttgtccaagataagcctg
tctagcttcaagtatgacgggctgatactgggccggcaggcgctccattg
cccagtcggcagcgacatccttcggcgcgattttgccggttactgcgctg
taccaaatgcgggacaacgtaagcactacatttcgctcatcgccagccca
gtcgggcggcgagttccatagcgttaaggtttcatttagcgcctcaaata
gatcctgttcaggaaccggatcaaagagttcctccgccgctggacctacc
aaggcaacgctatgttctcttgcttttgtcagcaagatagccagatcaat
gtcgatcgtggctggctcgaagatacctgcaagaatgtcattgcgctgcc
attctccaaattgcagttcgcgcttagctggataacgccacggaatgatg
tcgtcgtgcacaacaatggtgacttctacagcgcggagaatctcgctctc
tccaggggaagccgaagtttccaaaaggtcgttgatcaaagctcgccgcg
ttgtttcatcaagccttacggtcaccgtaaccagcaaatcaatatcactg
tgtggcttcaggccgccatccactgcggagccgtacaaatgtacggccag
caacgtcggttcgagatggcgctcgatgacgccaactacctctgatagtt
gagtcgatacttcggcgatcaccgcttcccccatgatgtttaactttgtt
ttagggcgactgccctgctgcgtaacatcgttgctgctccataacatcaa
acatcgacccacggcgtaacgcgcttgctgcttggatgcccgaggcatag
actgtaccccaaaaaaacatgtcataacaagaagccatgaaaaccgccac
tgcgccgttaccaccgctgcgttcggtcaaggttctggaccagttgcgtg
acggcagttacgctacttgcattacagcttacgaaccgaacgaggcttat
gtccactgggttcgtgcccgaattgatccagg

>SEQ ID NO 5 pAM305, with dicistronic aadA-gfp1-10 gene, for expression in chloroplasts

GCTCCCCCGCCGTCGTTCAATGAGAATGGATAAGAGGCTCGTGGGATTGA
CGTGAGGGGGCAGGGATGGCTATATTTCTGGGAGAATTAACCGATCGACG
TGCaAGCGGACATTTATTTTaAATTCGATAATTTTTGCAAAAACATTTCG
ACATATTTATTTATTTTATTATTATGAGAATCAATCCTACTACTTCTGGT
TCTGGGGTTTCCACGgctactagcGAAGCGGTGATCGCCGAAGTATCGAC
TCAACTATCAGAGGTAGTTGGCGTCATCGAGCGCCATCTCGAACCGACGT
TGCTGGCCGTACATTTGTACGGCTCCGCAGTGGATGGCGGCCTGAAGCCA
CACAGTGATATTGATTTGCTGGTTACGGTGACCGTAAGGCTTGATGAAAC
AACGCGGCGAGCTTTGATCAACGACCTTTTGGAAACTTCGGCTTCCCCTG
GAGAGAGCGAGATTCTCCGCGCTGTAGAAGTCACCATTGTTGTGCACGAC
GACATCATTCCGTGGCGTTATCCAGCTAAGCGCGAACTGCAATTTGGAGA
ATGGCAGCGCAATGACATTCTTGCAGGTATCTTCGAGCCAGCCACGATCG
ACATTGATCTGGCTATCTTGCTGACAAAAGCAAGAGAACATAGCGTTGCC
TTGGTAGGTCCAGCGGCGGAGGAACTCTTTGATCCGGTTCCTGAACAGGA
TCTATTTGAGGCGCTAAATGAAACCTTAACGCTATGGAACTCGCCGCCCG
ACTGGGCTGGCGATGAGCGAAATGTAGTGCTTACGTTGTCCCGCATTTGG
TACAGCGCAGTAACCGGCAAAATCGCGCCGAAGGATGTCGCTGCCGACTG
GGCAATGGAGCGCCTGCCGGCCCAGTATCAGCCCGTCATACTTGAAGCTA
GACAGGCTTATCTTGGACAAGAAGAAGATCGCTTGGCCTCGCGCGCAGAT
CAGTTGGAAGAATTTGTCCACTACGTGAAAGGCGAGATCACCAAGGTAGT
gGGCAAAgaaCAAAAACTCATTTCTGAAGAAGACTTGTAACTGCAGATAA
CCCAAATAATGTTTTAAAATTTTAAAAATAATGTAGGAGGAAAAATTATG
AGCAAAGGAGAAGAACTTTTCACTGGAGTTGTCCCAATTCTTGTTGAATT
AGATGGTGATGTTAATGGGCACAAATTTTCTGTCAGAGGAGAGGGTGAAG
GTGATGCTACAATCGGAAAACTCACCCTTAAATTTATTTGCACTACTGGA
AAACTACCTGTTCCtTGGCCAACACTTGTCACTACTCTGACCTATGGTGT
TCAATGCTTTTCCCGTTATCCGGATCACATGAAAAGGCATGACTTTTTCA
AGAGTGCCATGCCCGAAGGTTATGTtCAGGAACGCACTATATCTTTCAAA
GATGACGGGAAATACAAGACGCGTGCTGTAGTCAAGTTTGAAGGTGATAC
CCTTGTTAATCGTATCGAGTTAAAGGGTACTGATTTTAAAGAAGATGGAA
ACATTCTCGGACACAAACTtGAGTACAACTTTAACTCACACAATGTATAC
ATCACGGCAGACAAACAAAAGAATGGAATCAAAGCTAACTTCACAGTTCG
CCACAACGTTGAAGATGGTTCCGTTCAACTAGCAGACCATTATCAACAAA
ATACTCCAATTGGCGATGGCCCTGTCCTTTTACCAGACAACCATTACCTG
TCGACACAAACTGTCCTTTCGAAAGATCCCAACGAAAAGGGTACCTAAGc
tctagaGATCCTGGCCTAGTCTATAGGAGGTTTTGAAAAGAAAGGAGCAA
TAATCATTTTCTTGTTCTATCAAGAGGGTGCTATTGCTCCTTTCTTTTTT
TCTTTTTATTTATTTACTAGTATTTTACTTACATAGACTTTTTTGTTTAC
ATTATAGAAAAAGAAGGAGAGGTTATTTTCTTGCATTTATTCATG

>SEQ ID NO 6 - pAM29. Sulfite-reductase-GFP11 fusion protein, in a T7 P/T cassette.

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCTCGATCCCGCGAAATTAATACGACTCACTATA
GGGGAATTGTGAGCGGATAACAATTCCCCTCTAGAAATAATTTTGTTTAA
CTTTAAGAAGGAGATATACCATGGGCAGCAGCCATCATCATCATCATCAC
AGCAGCGGCCTGGTGCCGCGCGGCAGCCATATGCCAGTGAAATGTCCCGG
CGAGTACCAAGTTGATGGCAAAAAAGTTATACTAGACGAGGACTGTTTTA
TGCAAAACCCAGAGGACTGGGACGAAAAAGTGGCTGAGTGGTTGGCCAGA
GAGCTAGAGGGCATACAAAAAATGACCGAGGAGCATTGGAAGTTGGTAAA
ATACTTAAGGGAGTATTGGGAGACCTTCGGCTCCTGCCCGCCAATTAAAA
TGGTCACTAAAGAGACGGGCTTCTCCTTGGAGAAAATCTACCAGCTATTC
CCCTCGGGGCCTGCGCACGGAGCTTGTAAAGTGGCAGGCGCGCCGAAGCC
CACAGGCTGCGTCGGATCCGGTGGAGGGTCTGGTGGCGGATCAACTAGTC
GTGACCACATGGTCCTTCATGAGTACGTAAATGCTGCTGGGATTACATAA
ttctagaTAACTCGAGCACCACCACCACCACCACTGAGATCCGGCTGCTA
ACAAAGCCCGAAAGGAAGCTGAGTTGGCTGCTGCCACCGCTGAGCAATAA
CTAGCATAACCCCTTGGGGCCTCTAAACGGGTCTTGAGGGGTTTTTTGCT
GAAAGGAGGAACTATATCCGGAT

>SEQ ID NO 7 - pAM30. Pt-VirD2 (without GFP1)1, in a T7 P/T cassette.

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCTCGATCCCGCGAAATTAATACGACTCACTATA
GGGGAATTGTGAGCGGATAACAATTCCCCTCTAGAAATAATTTTGTTTAA
CTTTAAGAAGGAGATATACCATGGGCATGCAGGTGTGGCCAGCGATGGCT
CCCGATCGGGCCCAAGTAATCATTCGCATTGTGCCAGGAGGTGGAACCAA
GACCCTTCAGCAGATAATCAATCAGCTGGAGTACCTGTCCCGAAAGGGAA
AGCTGGAACTCCAGCGTTCAGCCCGGCATCTCGATATTCCCGTTCCGCCG
GATCAAATCCGTGAGCTTGCCCAAAGCTGGGTTACGGAGGCCGGGATTTA
TGACGAAAGTCAGTCAGACGATGACAGGCAACAAGACTTAACAACACACA
TTATTGTGAGCTTCCCCGCAGGTACCGACCAAACCGCAGCTTATGAAGCA
AGCCGGGAATGGGCAGCCGAGATGTTTGGGTCAGGATACGGGGGTGGCCG
CTATAACTATCTGACAGCCTACCACGTGGACCGCGATCATCCACATTTAC
ATGTCGTGGTCAATCGTCGGGAACTTCTGGGGCAGGGGTGGCTGAAAATA
TCCAGGCGCCATCCCCAGCTGAATTATGACGGCTTACGGAAAAAGATGGC
AGAGATTTCACTTCGTCACGGCATAGTCCTGGATGCGACTTCCCGAGCAG
AAAGGGGAATAGCAGAGCGACCAATCACCTATGCTGAATATCGACGTCTT
GAGCGGATGCTCGAAGTTATGGCAGAAGTTCGGCCCATAGCCCGATCCAT
TAAAACGGCTCACGACGATGCGCGAGCGGAATTAATGTCGGCGGACAGAC
CTCGATCAACGCGCGGTCTATGATTCTAGATATACTCGAGCACCACCACC
ACCACCACTGAGATCCGGCTGCTAACAAAGCCCGAAAGGAAGCTGAGTTG
GCTGCTGCCACCGCTGAGCAATAACTAGCATAACCCCTTGGGGCCTCTAA
ACGGGTCTTGAGGGGTTTTTTGCTGAAAGGAGGAACTATATCCGGAT

>SEQ ID NO 8 pAM31

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCTCGATCCCGCGAAATTAATACGACTCACTATA
GGGGAATTGTGAGCGGATAACAATTCCCCTCTAGAAATAATTTTGTTTAA
CTTTAAGAAGGAGATATACCATGGGCATGCAGGTGTGGCCAGCGCGTGAC
CACATGGTCCTTCATGAGTACGTAAATGCTGCTGGGATTACAATGGCTCC
CGATCGGGCCCAAGTAATCATTCGCATTGTGCCAGGAGGTGGAACCAAGA
CCCTTCAGCAGATAATCAATCAGCTGGAGTACCTGTCCCGAAAGGGAAAG
CTGGAACTCCAGCGTTCAGCCCGGCATCTCGATATTCCCGTTCCGCCGGA
TCAAATCCGTGAGCTTGCCCAAAGCTGGGTTACGGAGGCCGGGATTTATG
ACGAAAGTCAGTCAGACGATGACAGGCAACAAGACTTAACAACACACATT
ATTGTGAGCTTCCCCGCAGGTACCGACCAAACCGCAGCTTATGAAGCAAG
CCGGGAATGGGCAGCCGAGATGTTTGGGTCAGGATACGGGGGTGGCCGCT
ATAACTATCTGACAGCCTACCACGTGGACCGCGATCATCCACATTTACAT
GTCGTGGTCAATCGTCGGGAACTTCTGGGGCAGGGGTGGCTGAAAATATC
CAGGCGCCATCCCCAGCTGAATTATGACGGCTTACGGAAAAAGATGGCAG
AGATTTCACTTCGTCACGGCATAGTCCTGGATGCGACTTCCCGAGCAGAA
AGGGGAATAGCAGAGCGACCAATCACCTATGCTGAATATCGACGTCTTGA
GCGGATGCTCGAAGTTATGGCAGAAGTTCGGCCCATAGCCCGATCCATTA
AAACGGCTCACGACGATGCGCGAGCGGAATTAATGTCGGCGGACAGACCT
CGATCAACGCGCGGTCTATGATTCTAGATATACTCGAGCACCACCACCAC
CACCACTGAGATCCGGCTGCTAACAAAGCCCGAAAGGAAGCTGAGTTGGC
TGCTGCCACCGCTGAGCAATAACTAGCATAACCCCTTGGGGCCTCTAAAC
GGGTCTTGAGGGGTTTTTTGCTGAAAGGAGGAACTATATCCGGAT

>SEQ ID NO 9 pAM32

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCTCGATCCCGCGAAATTAATACGACTCACTATA
GGGGAATTGTGAGCGGATAACAATTCCCCTCTAGAAATAATTTTGTTTAA
CTTTAAGAAGGAGATATACCATGGGCATGCAGGTGTGGCCAGCGCGTGAC
CACATGGTCCTTCATGAGTACGTAAATGCTGCTGGGATTACAGGTGGAGG
GTCTGGTGGCGGATCAATGGCTCCCGATCGGGCCCAAGTAATCATTCGCA
TTGTGCCAGGAGGTGGAACCAAGACCCTTCAGCAGATAATCAATCAGCTG
GAGTACCTGTCCCGAAAGGGAAAGCTGGAACTCCAGCGTTCAGCCCGGCA
TCTCGATATTCCCGTTCCGCCGGATCAAATCCGTGAGCTTGCCCAAAGCT
GGGTTACGGAGGCCGGGATTTATGACGAAAGTCAGTCAGACGATGACAGG
CAACAAGACTTAACAACACACATTATTGTGAGCTTCCCCGCAGGTACCGA
CCAAACCGCAGCTTATGAAGCAAGCCGGGAATGGGCAGCCGAGATGTTTG
GGTCAGGATACGGGGGTGGCCGCTATAACTATCTGACAGCCTACCACGTG
GACCGCGATCATCCACATTTACATGTCGTGGTCAATCGTCGGGAACTTCT
GGGGCAGGGGTGGCTGAAAATATCCAGGCGCCATCCCCAGCTGAATTATG
ACGGCTTACGGAAAAAGATGGCAGAGATTTCACTTCGTCACGGCATAGTC
CTGGATGCGACTTCCCGAGCAGAAAGGGGAATAGCAGAGCGACCAATCAC
CTATGCTGAATATCGACGTCTTGAGCGGATGCTCGAAGTTATGGCAGAAG
TTCGGCCCATAGCCCGATCCATTAAAACGGCTCACGACGATGCGCGAGCG
GAATTAATGTCGGCGGACAGACCTCGATCAACGCGCGGTCTATGATTCTA
GATATACTCGAGCACCACCACCACCACCACTGAGATCCGGCTGCTAACAA
AGCCCGAAAGGAAGCTGAGTTGGCTGCTGCCACCGCTGAGCAATAACTAG
CATAACCCCTTGGGGCCTCTAAACGGGTCTTGAGGGGTTTTTTGCTGAAA
GGAGGAACTATATCCGGAT

>SEQ ID NO 10 pAM33

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CGGCCACGGGGCCTGCCACCATACCCACGCCGAAACAAGCGCTCATGAGC
CCGAAGTGGCGAGCCCGATCTTCCCCATCGGTGATGTCGGCGATATAGGC
GCCAGCAACCGCACCTGTGGCGCCGGTGATGCCGGCCACGATGCGTCCGG
CGTAGAGGATCGAGATCTCGATCCCGCGAAATTAATACGACTCACTATAG
GGGAATTGTGAGCGGATAACAATTCCCCTCTAGAAATAATTTTGTTTAAC
TTTAAGAAGGAGATATACCATGGGCATGCAGGTGTGGCCAGCGATGGCTC
CCGATCGGGCCCAAGTAATCATTCGCATTGTGCCAGGAGGTGGAACCAAG
ACCCTTCAGCAGATAATCAATCAGCTGGAGTACCTGTCCCGAAAGGGAAA
GCTGGAACTCCAGCGTTCAGCCCGGCATCTCGATATTCCCGTTCCGCCGG
ATCAAATCCGTGAGCTTGCCCAAAGCTGGGTTACGGAGGCCGGGATTTAT
GACGAAAGTCAGTCAGACGATGACAGGCAACAAGACTTAACAACACACAT
TATTGTGAGCTTCCCCGCAGGTACCGACCAAACCGCAGCTTATGAAGCAA
GCCGGGAATGGGCAGCCGAGATGTTTGGGTCAGGATACGGGGGTGGCCGC
TATAACTATCTGACAGCCTACCACGTGGACCGCGATCATCCACATTTACA
TGTCGTGGTCAATCGTCGGGAACTTCTGGGGCAGGGGTGGCTGAAAATAT
CCAGGCGCCATCCCCAGCTGAATTATGACGGCTTACGGAAAAAGATGGCA
GAGATTTCACTTCGTCACGGCATAGTCCTGGATGCGACTTCCCGAGCAGA
AAGGGGAATAGCAGAGCGACCAATCACCTATGCTGAATATCGACGTCTTG
AGCGGATGCGTGACCACATGGTCCTTCATGAGTACGTAAATGCTGCTGGG
ATTACACTCGAAGTTATGGCAGAAGTTCGGCCCATAGCCCGATCCATTAA
AACGGCTCACGACGATGCGCGAGCGGAATTAATGTCGGCGGACAGACCTC
GATCAACGCGCGGTCTATGATTCTAGATATACTCGAGCACCACCACCACC
ACCACTGAGATCCGGCTGCTAACAAAGCCCGAAAGGAAGCTGAGTTGGCT
GCTGCCACCGCTGAGCAATAACTAGCATAACCCCTTGGGGCCTCTAAACG
GGTCTTGAGGGGTTTTTTGCTGAAAGGAGGAACTATATCCGGAT

>SEQ ID NO 11 pAM34

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCTCGATCCCGCGAAATTAATACGACTCACTATA
GGGGAATTGTGAGCGGATAACAATTCCCCTCTAGAAATAATTTTGTTTAA
CTTTAAGAAGGAGATATACCATGGGCATGCAGGTGTGGCCAGCGATGGCT
CCCGATCGGGCCCAAGTAATCATTCGCATTGTGCCAGGAGGTGGAACCAA
GACCCTTCAGCAGATAATCAATCAGCTGGAGTACCTGTCCCGAAAGGGAA
AGCTGGAACTCCAGCGTTCAGCCCGGCATCTCGATATTCCCGTTCCGCCG
GATCAAATCCGTGAGCTTGCCCAAAGCTGGGTTACGGAGGCCGGGATTTA
TGACGAAAGTCAGTCAGACGATGACAGGCAACAAGACTTAACAACACACA
TTATTGTGAGCTTCCCCGCAGGTACCGACCAAACCGCAGCTTATGAAGCA
AGCCGGGAATGGGCAGCCGAGATGTTTGGGTCAGGATACGGGGGTGGCCG
CTATAACTATCTGACAGCCTACCACGTGGACCGCGATCATCCACATTTAC
ATGTCGTGGTCAATCGTCGGGAACTTCTGGGGCAGGGGTGGCTGAAAATA
TCCAGGCGCCATCCCCAGCTGAATTATGACGGCTTACGGAAAAAGATGGC
AGAGATTTCACTTCGTCACGGCATAGTCCTGGATGCGACTTCCCGAGCAG
AAAGGGGAATAGCAGAGCGACCAATCACCTATGCTGAATATCGACGTCTT
GAGCGGATGGGTGGAGGGTCTGGTGGCGGATCACGTGACCACATGGTCCT
TCATGAGTACGTAAATGCTGCTGGGATTACACTCGAAGTTATGGCAGAAG
TTCGGCCCATAGCCCGATCCATTAAAACGGCTCACGACGATGCGCGAGCG
GAATTAATGTCGGCGGACAGACCTCGATCAACGCGCGGTCTATGATTCTA
GATATACTCGAGCACCACCACCACCACCACTGAGATCCGGCTGCTAACAA
AGCCCGAAAGGAAGCTGAGTTGGCTGCTGCCACCGCTGAGCAATAACTAG
CATAACCCCTTGGGGCCTCTAAACGGGTCTTGAGGGGTTTTTTGCTGAAA
GGAGGAACTATATCCGGAT

>SEQ ID NO 12 pAM35

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCTCGATCCCGCGAAATTAATACGACTCACTATA
GGGGAATTGTGAGCGGATAACAATTCCCCTCTAGAAATAATTTTGTTTAA
CTTTAAGAAGGAGATATACCATGGGCATGCAGGTGTGGCCAGCGATGGCT
CCCGATCGGGCCCAAGTAATCATTCGCATTGTGCCAGGAGGTGGAACCAA
GACCCTTCAGCAGATAATCAATCAGCTGGAGTACCTGTCCCGAAAGGGAA
AGCTGGAACTCCAGCGTTCAGCCCGGCATCTCGATATTCCCGTTCCGCCG
GATCAAATCCGTGAGCTTGCCCAAAGCTGGGTTACGGAGGCCGGGATTTA
TGACGAAAGTCAGTCAGACGATGACAGGCAACAAGACTTAACAACACACA
TTATTGTGAGCTTCCCCGCAGGTACCGACCAAACCGCAGCTTATGAAGCA
AGCCGGGAATGGGCAGCCGAGATGTTTGGGTCAGGATACGGGGGTGGCCG
CTATAACTATCTGACAGCCTACCACGTGGACCGCGATCATCCACATTTAC
ATGTCGTGGTCAATCGTCGGGAACTTCTGGGGCAGGGGTGGCTGAAAATA
TCCAGGCGCCATCCCCAGCTGAATTATGACGGCTTACGGAAAAAGATGGC
AGAGATTTCACTTCGTCACGGCATAGTCCTGGATGCGACTTCCCGAGCAG
AAAGGGGAATAGCAGAGCGACCAATCACCTATGCTGAATATCGACGTCTT
GAGCGGATGCGTGACCACATGGTCCTTCATGAGTACGTAAATGCTGCTGG
GATTACAGGTGGAGGGTCTGGTGGCGGATCACTCGAAGTTATGGCAGAAG
TTCGGCCCATAGCCCGATCCATTAAAACGGCTCACGACGATGCGCGAGCG
GAATTAATGTCGGCGGACAGACCTCGATCAACGCGCGGTCTATGATTCTA
GATATACTCGAGCACCACCACCACCACCACTGAGATCCGGCTGCTAACAA
AGCCCGAAAGGAAGCTGAGTTGGCTGCTGCCACCGCTGAGCAATAACTAG
CATAACCCCTTGGGGCCTCTAAACGGGTCTTGAGGGGTTTTTTGCTGAAA
GGAGGAACTATATCCGGAT

>SEQ ID NO 13 pAM36

TGGCGAATGGGACGCGCCCTGTAGCGGCGCATTAAGCGCGGCGGGTGTGG
TGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGG
CCATCGCCCTGATAGACGGTTTTTCGCCCTTTGACGTTGGAGTCCACGTT
CTTTAATAGTGGACTCTTGTTCCAAACTGGAACAACACTCAACCCTATCT
CGGTCTATTCTTTTGATTTATAAGGGATTTTGCCGATTTCGGCCTATTGG
TTAAAAAATGAGCTGATTTAACAAAAATTTAACGCGAATTTTAACAAAAT
ATTAACGTTTACAATTTCAGGTGGCACTTTTCGGGGAAATGTGCGCGGAA
CCCCTATTTGTTTATTTTTCTAAATACATTCAAATATGTATCCGCTCATG
AATTAATTCTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGACGAAATACGCGATCGCTG
TTAAAAGGACAATTACAAACAGGAATCGAATGCAACCGGCGCAGGAACAC
TGCCAGCGCATCAACAATATTTTCACCTGAATCAGGATATTCTTCTAATA
CCTGGAATGCTGTTTTCCCGGGGATCGCAGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATAACACCCCTTGTATTACTGTTTATG
TAAGCAGACAGTTTTATTGTTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCTGATGCGGTATTTTCTCCTTACGCATCT
GTGCGGTATTTCACACCGCATATATGGTGCACTCTCAGTACAATCTGCTC
TGATGCCGCATAGTTAAGCCAGTATACACTCCGCTATCGCTACGTGACTG
GGTCATGGCTGCGCCCCGACACCCGCCAACACCCGCTGACGCGCCCTGAC
GGGCTTGTCTGCTCCCGGCATCCGCTTACAGACAAGCTGTGACCGTCTCC
GGGAGCTGCATGTGTCAGAGGTTTTCACCGTCATCACCGAAACGCGCGAG
GCAGCTGCGGTAAAGCTCATCAGCGTGGTCGTGAAGCGATTCACAGATGT
CTGCCTGTTCATCCGCGTCCAGCTCGTTGAGTTTCTCCAGAAGCGTTAAT
GTCTGGCTTCTGATAAAGCGGGCCATGTTAAGGGCGGTTTTTTCCTGTTT
GGTCACTGATGCCTCCGTGTAAGGGGGATTTCTGTTCATGGGGGTAATGA
TACCGATGAAACGAGAGAGGATGCTCACGATACGGGTTACTGATGATGAA
CATGCCCGGTTACTGGAACGTTGTGAGGGTAAACAACTGGCGGTATGGAT
GCGGCGGGACCAGAGAAAAATCACTCAGGGTCAATGCCAGCGCTTCGTTA
ATACAGATGTAGGTGTTCCACAGGGTAGCCAGCAGCATCCTGCGATGCAG
ATCCGGAACATAATGGTGCAGGGCGCTGACTTCCGCGTTTCCAGACTTTA
CGAAACACGGAAACCGAAGACCATTCATGTTGTTGCTCAGGTCGCAGACG
TTTTGCAGCAGCAGTCGCTTCACGTTCGCTCGCGTATCGGTGATTCATTC
TGCTAACCAGTAAGGCAACCCCGCCAGCCTAGCCGGGTCCTCAACGACAG
GAGCACGATCATGCGCACCCGTGGGGCCGCCATGCCGGCGATAATGGCCT
GCTTCTCGCCGAAACGTTTGGTGGCGGGACCAGTGACGAAGGCTTGAGCG
AGGGCGTGCAAGATTCCGAATACCGCAAGCGACAGGCCGATCATCGTCGC
GCTCCAGCGAAAGCGGTCCTCGCCGAAAATGACCCAGAGCGCTGCCGGCA
CCTGTCCTACGAGTTGCATGATAAAGAAGACAGTCATAAGTGCGGCGACG
ATAGTCATGCCCCGCGCCCACCGGAAGGAGCTGACTGGGTTGAAGGCTCT
CAAGGGCATCGGTCGAGATCCCGGTGCCTAATGAGTGAGCTAACTTACAT
TAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTAT
TGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCT
GGTTTGCCCCAGCAGGCGAAAATCCTGTTTGATGGTGGTTAACGGCGGGA
TATAACATGAGCTGTCTTCGGTATCGTCGTATCCCACTACCGAGATATCC
GCACCAACGCGCAGCCCGGACTCGGTAATGGCGCGCATTGCGCCCAGCGC
CATCTGATCGTTGGCAACCAGCATCGCAGTGGGAACGATGCCCTCATTCA
GCATTTGCATGGTTTGTTGAAAACCGGACATGGCACTCCAGTCGCCTTCC
CGTTCCGCTATCGGCTGAATTTGATTGCGAGTGAGATATTTATGCCAGCC
AGCCAGACGCAGACGCGCCGAGACAGAACTTAATGGGCCCGCTAACAGCG
CGATTTGCTGGTGACCCAATGCGACCAGATGCTCCACGCCCAGTCGCGTA
CCGTCTTCATGGGAGAAAATAATACTGTTGATGGGTGTCTGGTCAGAGAC
ATCAAGAAATAACGCCGGAACATTAGTGCAGGCAGCTTCCACAGCAATGG
CATCCTGGTCATCCAGCGGATAGTTAATGATCAGCCCACTGACGCGTTGC
GCGAGAAGATTGTGCACCGCCGCTTTACAGGCTTCGACGCCGCTTCGTTC
TACCATCGACACCACCACGCTGGCACCCAGTTGATCGGCGCGAGATTTAA
TCGCCGCGACAATTTGCGACGGCGCGTGCAGGGCCAGACTGGAGGTGGCA
ACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAA
GAGACACCGGCATACTCTGCGACATCGTATAACGTTACTGGTTTCACATT
CACCACCCTGAATTGACTCTCTTCCGGGCGCTATCATGCCATACCGCGAA
AGGTTTTGCGCCATTCGATGGTGTCCGGGATCTCGACGCTCTCCCTTATG
CGACTCCTGCATTAGGAAGCAGCCCAGTAGTAGGTTGAGGCCGTTGAGCA
CCGCCGCCGCAAGGAATGGTGCATGCAAGGAGATGGCGCCCAACAGTCCC
CCGGCCACGGGGCCTGCCACCATACCCACGCCGAAACAAGCGCTCATGAG
CCCGAAGTGGCGAGCCCGATCTTCCCCATCGGTGATGTCGGCGATATAGG
CGCCAGCAACCGCACCTGTGGCGCCGGTGATGCCGGCCACGATGCGTCCG
GCGTAGAGGATCGAGATCTCGATCCCGCGAAATTAATACGACTCACTATA
GGGGAATTGTGAGCGGATAACAATTCCCCTCTAGAAATAATTTTGTTTAA
CTTTAAGAAGGAGATATACCATGGGCATGCAGGTGTGGCCAGCGATGGCT
CCCGATCGGGCCCAAGTAATCATTCGCATTGTGCCAGGAGGTGGAACCAA
GACCCTTCAGCAGATAATCAATCAGCTGGAGTACCTGTCCCGAAAGGGAA
AGCTGGAACTCCAGCGTTCAGCCCGGCATCTCGATATTCCCGTTCCGCCG
GATCAAATCCGTGAGCTTGCCCAAAGCTGGGTTACGGAGGCCGGGATTTA
TGACGAAAGTCAGTCAGACGATGACAGGCAACAAGACTTAACAACACACA
TTATTGTGAGCTTCCCCGCAGGTACCGACCAAACCGCAGCTTATGAAGCA
AGCCGGGAATGGGCAGCCGAGATGTTTGGGTCAGGATACGGGGGTGGCCG
CTATAACTATCTGACAGCCTACCACGTGGACCGCGATCATCCACATTTAC
ATGTCGTGGTCAATCGTCGGGAACTTCTGGGGCAGGGGTGGCTGAAAATA
TCCAGGCGCCATCCCCAGCTGAATTATGACGGCTTACGGAAAAAGATGGC
AGAGATTTCACTTCGTCACGGCATAGTCCTGGATGCGACTTCCCGAGCAG
AAAGGGGAATAGCAGAGCGACCAATCACCTATGCTGAATATCGACGTCTT
GAGCGGATGGGTGGAGGGTCTCGTGACCACATGGTCCTTCATGAGTACGT
AAATGCTGCTGGGATTACAGGTGGCGGATCACTCGAAGTTATGGCAGAAG
TTCGGCCCATAGCCCGATCCATTAAAACGGCTCACGACGATGCGCGAGCG
GAATTAATGTCGGCGGACAGACCTCGATCAACGCGCGGTCTATGATTCTA
GATATACTCGAGCACCACCACCACCACCACTGAGATCCGGCTGCTAACAA
AGCCCGAAAGGAAGCTGAGTTGGCTGCTGCCACCGCTGAGCAATAACTAG
CATAACCCCTTGGGGCCTCTAAACGGGTCTTGAGGGGTTTTTTGCTGAAA
GGAGGAACTATATCCGGAT

>SEQ ID NO 14 pAM407

ccgggctggttgccctcgccgctgggctggcggccgtctatggccctgca
aacgcgccagaaacgccgtcgaagccgtgtgcgagacaccgcggccgccg
gcgttgtggatacctcgcggaaaacttggccctcactgacagatgagggg
cggacgttgacacttgaggggccgactcacccggcgcggcgttgacagat
gaggggcaggctcgatttcggccggcgacgtggagctggccagcctcgca
aatcggcgaaaacgcctgattttacgcgagtttcccacagatgatgtgga
caagcctggggataagtgcctgcggtattgacacttgaggggcgcgacta
ctgacagatgaggggcgcgatccttgacacttgaggggcagagtgctgac
agatgaggggcgcacctattgacatttgaggggctgtccacaggcagaaa
atccagcatttgcaagggtttccgcccgtttttcggccaccgctaacctg
tcttttaacctgcttttaaaccaatatttataaaccttgtttttaaccag
ggctgcgccctgtgcgcgtgaccgcgcacgccgaaggggggtgccccccc
ttctcgaaccctcccggcccgctaacgcgggcctcccatccccccagggg
ctgcgcccctcggccgcgaacggcctcaccccaaaaatggcagcgctggc
agtccttgccattgccgggatcggggcagtaacgggatgggcgatcagcc
cgagcgcgacgcccggaagcattgacgtgccgcaggtgctggcatcgaca
ttcagcgaccaggtgccgggcagtgagggcggcggcctgggtggcggcct
gcccttcacttcggccgtcggggcattcacggacttcatggcggggccgg
caatttttaccttgggcattcttggcatagtggtcgcgggtgccgtgctc
gtgttcgggggtgcgataaacccagcgaaccatttgaggtgataggtaag
attataccgaggtatgaaaacgagaattggacctttacagaattactcta
tgaagcgccatatttaaaaagctaccaagacgaagaggatgaagaggatg
aggaggcagattgccttgaatatattgacaatactgataagataatatat
cttttatatagaagatatcgccgtatgtaaggatttcagggggcaaggca
taggcagcgcgcttatcaatatatctatagaatgggcaaagcataaaaac
ttgcatggactaatgcttgaaacccaggacaataaccttatagcttgtaa
attctatcataattgggtaatgactccaacttattgatagtgttttatgt
tcagataatgcccgatgactttgtcatgcagctccaccgattttgagaac
gacagcgacttccgtcccagccgtgccaggtgctgcctcagattcaggtt
atgccgctcaattcgctgcgtatatcgcttgctgattacgtgcagctttc
ccttcaggcgggattcatacagcggccagccatccgtcatccatatcacc
acgtcaaagggtgacagcaggctcataagacgccccagcgtcgccatagt
gcgttcaccgaatacgtgcgcaacaaccgtcttccggagactgtcatacg
cgtaaaacagccagcgctggcgcgatttagccccgacatagccccactgt
tcgtccatttccgcgcagacgatgacgtcactgcccggctgtatgcgcga
ggttaccgactgcggcctgagttttttaagtgacgtaaaatcgtgttgag
gccaacgcccataatgcgggctgttgcccggcatccaacgccattcatgg
ccatatcaatgattttctggtgcgtaccgggttgagaagcggtgtaagtg
aactgcagttgccatgttttacggcagtgagagcagagatagcgctgatg
tccggcggtgcttttgccgttacgcaccaccccgtcagtagctgaacagg
agggacagctgatagacacagaagccactggagcacctcaaaaacaccat
catacactaaatcagtaagttggcagcatcacccataattgtggtttcaa
aatcggctccgtcgatactatgttatacgccaactttgaaaacaactttg
aaaaagctgttttctggtatttaaggttttagaatgcaaggaacagtgaa
ttggagttcgtcttgttataattagcttcttggggtatctttaaatactg
tagaaaagaggaaggaaataataaatggctaaaatgagaatatcaccgga
attgaaaaaactgatcgaaaaataccgctgcgtaaaagatacggaaggaa
tgtctcctgctaaggtatataagctggtgggagaaaatgaaaacctatat
ttaaaaatgacggacagccggtataaagggaccacctatgatgtggaacg
ggaaaaggacatgatgctatggctggaaggaaagctgcctgttccaaagg
tcctgcactttgaacggcatgatggctggagcaatctgctcatgagtgag
gccgatggcgtcctttgctcggaagagtatgaagatgaacaaagccctga
aaagattatcgagctgtatgcggagtgcatcaggctctttcactccatcg
acatatcggattgtccctatacgaatagcttagacagccgcttagccgaa
ttggattacttactgaataacgatctggccgatgtggattgcgaaaactg
ggaagaagacactccatttaaagatccgcgcgagctgtatgattttttaa
agacggaaaagcccgaagaggaacttgtcttttcccacggcgacctggga
gacagcaacatctttgtgaaagatggcaaagtaagtggctttattgatct
tgggagaagcggcagggcggacaagtggtatgacattgccttctgcgtcc
ggtcgatcagggaggatatcggggaagaacagtatgtcgagctatttttt
gacttactggggatcaagcctgattgggagaaaataaaatattatatttt
actggatgaattgttttagtacctagatgtggcgcaacgatgccggcgac
aagcaggagcgcaccgacttcttccgcatcaagtgttttggctctcaggc
cgaggcccacggcaagtatttgggcaaggggtcgctggtattcgtgcagg
gcaagattcggaataccaagtacgagaaggacggccagacggtctacggg
accgacttcattgccgataaggtggattatctggacaccaaggcaccagg
cgggtcaaatcaggaataagggcacattgccccggcgtgagtcggggcaa
tcccgcaaggagggtgaatgaatcggacgtttgaccggaaggcatacagg
caagaactgatcgacgcggggttttccgccgaggatgccgaaaccatcgc
aagccgcaccgtcatgcgtgcgccccgcgaaaccttccagtccgtcggct
cgatggtccagcaagctacggccaagatcgagcgcgacagcgtgcaactg
gctccccctgccctgcccgcgccatcggccgccgtggagcgttcgcgtcg
tctcgaacaggaggcggcaggtttggcgaagtcgatgaccatcgacacgc
gaggaactatgacgaccaagaagcgaaaaaccgccggcgaggacctggca
aaacaggtcagcgaggccaagcaggccgcgttgctgaaacacacgaagca
gcagatcaaggaaatgcagctttccttgttcgatattgcgccgtggccgg
acacgatgcgagcgatgccaaacgacacggcccgctctgccctgttcacc
acgcgcaacaagaaaatcccgcgcgaggcgctgcaaaacaaggtcatttt
ccacgtcaacaaggacgtgaagatcacctacaccggcgtcgagctgcggg
ccgacgatgacgaactggtgtggcagcaggtgttggagtacgcgaagcgc
acccctatcggcgagccgatcaccttcacgttctacgagctttgccagga
cctgggctggtcgatcaatggccggtattacacgaaggccgaggaatgcc
tgtcgcgcctacaggcgacggcgatgggcttcacgtccgaccgcgttggg
cacctggaatcggtgtcgctgctgcaccgcttccgcgtcctggaccgtgg
caagaaaacgtcccgttgccaggtcctgatcgacgaggaaatcgtcgtgc
tgtttgctggcgaccactacacgaaattcatatgggagaagtaccgcaag
ctgtcgccgacggcccgacggatgttcgactatttcagctcgcaccggga
gccgtacccgctcaagctggaaaccttccgcctcatgtgcggatcggatt
ccacccgcgtgaagaagtggcgcgagcaggtcggcgaagcctgcgaagag
ttgcgaggcagcggcctggtggaacacgcctgggtcaatgatgacctggt
gcattgcaaacgctagggccttgtggggtcagttccggctgggggttcag
cagccagcgctttactggcatttcaggaacaagcgggcactgctcgacgc
acttgcttcgctcagtatcgctcgggacgcacggcgcgctctacgaactg
ccgataaacagaggattaaaattgacaattgtgattaaggctcagattcg
acggcttggagcggccgacgtgcaggatttccgcgagatccgattgtcgg
ccctgaagaaagctccagagatgttcgggtccgtttacgagcacgaggag
aaaaagcccatggaggcgttcgctgaacggttgcgagatgccgtggcatt
cggcgcctacatcgacggcgagatcattgggctgtcggtcttcaaacagg
aggacggccccaaggacgctcacaaggcgcatctgtccggcgttttcgtg
gagcccgaacagcgaggccgaggggtcgccggtatgctgctgcgggcgtt
gccggcgggtttattgctcgtgatgatcgtccgacagattccaacgggaa
tctggtggatgcgcatcttcatcctcggcgcacttaatatttcgctattc
tggagcttgttgtttatttcggtctaccgcctgccgggcggggtcgcggc
gacggtaggcgctgtgcagccgctgatggtcgtgttcatctctgccgctc
tgctaggtagcccgatacgattgatggcggtcctgggggctatttgcgga
actgcgggcgtggcgctgttggtgttgacaccaaacgcagcgctagatcc
tgtcggcgtcgcagcgggcctggcgggggcggtttccatggcgttcggaa
ccgtgctgacccgcaagtggcaacctcccgtgcctctgctcacctttacc
gcctggcaactggcggccggaggacttctgctcgttccagtagctttagt
gtttgatccgccaatcccgatgcctacaggaaccaatgttctcggcctgg
cgtggctcggcctgatcggagcgggtttaacctacttcctttggttccgg
gggatctcgcgactcgaacctacagttgtttccttactgggctttctcag
ccccagatctggggtcgatcagccggggatgcatcaggccgacagtcgga
acttcgggtccccgacctgtaccattcggtgagcaatggataggggagtt
gatatcgtcaacgttcacttctaaagaaatagcgccactcagcttcctca
gcggctttatccagcgatttcctattatgtcggcatagttctcaagatcg
acagcctgtcacggttaagcgagaaatgaataagaaggctgataattcgg
atctctgcgagggagatgatatttgatcacaggcagcaacgctctgtcat
cgttacaatcaacatgctaccctccgcgagatcatccgtgtttcaaaccc
ggcagcttagttgccgttcttccgaatagcatcggtaacatgagcaaagt
ctgccgccttacaacggctctcccgctgacgccgtcccggactgatgggc
tgcctgtatcgagtggtgattttgtgccgagctgccggtcggggagctgt
tggctggctggtggcaggatatattgtggtgtaaacaaattgacgcttag
acaacttaataacacattgcggacgtttttaatgtactggggtggttttt
cttttcaccagtgagacgggcaacagctgattgcccttcaccgcctggcc
ctgagagagttgcagcaagcggtccacgctggtttgccccagcaggcgaa
aatcctgtttgatggtggttccgaaatcggcaaaatcccttataaatcaa
aagaatagcccgagatagggttgagtgttgttccagtttggaacaagagt
ccactattaaagaacgtggactccaacgtcaaagggcgaaaaaccgtcta
tcagggcgatggcccactacgtgaaccatcacccaaatcaagttttttgg
ggtcgaggtgccgtaaagcactaaatcggaaccctaaagggagcccccga
tttagagcttgacggggaaagccggcgaacgtggcgagaaaggaagggaa
gaaagcgaaaggagcgggcgccattcaggctgcgcaactgttgggaaggg
cgatcggtgcgggcctcttcgctattacgccagctggcgaaagggggatg
tgctgcaaggcgattaagttgggtaacgccagggttttcccagtcacgac
gttgtaaaacgacggccagtgAATTCTTAATtaagccaacatggtggagc
acgacactctcgtctactccaagaatatcaaagatacagtctcagaagac
caaagggctattgagacttttcaacaaagggtaatatcgggaaacctcct
cggattccattgcccagctatctgtcacttcatcaaaaggacagtagaaa
aggaaggtggcacctacaaatgccatcattgcgataaaggaaaggctatc
gttcaagatgcctctgccgacagtggtcccaaagatggacccccacccac
gaggagcatcgtggaaaaagaagacgttccaaccacgtcttcaaagcaag
tggattgatgtgaacatggtggagcacgacactctcgtctactccaagaa
tatcaaagatacagtctcagaagaccaaagggctattgagacttttcaac
aaagggtaatatcgggaaacctcctcggattccattgcccagctatctgt
cacttcatcaaaaggacagtagaaaaggaaggtggcacctacaaatgcca
tcattgcgataaaggaaaggctatcgttcaagatgcctctgccgacagtg
gtcccaaagatggacccccacccacgaggagcatcgtggaaaaagaagac
gttccaaccacgtcttcaaagcaagtggattgatgtgatatctccactga
cgtaagggatgacgcacaatcccactatccttcgcaagacccttcctcta
tataaggaagttcatttcatttggagaggacacgctgaaatcaccagtct
ctctctacaaatctatctctcTCGAGgtctcGGTACCATGGCTTCCTCAG
TTCTTTCCTCCGCAGCAGTTGCCACCCGCAGCAATGTTGCTCAAGCTAAC
ATGGTTGCACCTTTCACTGGCCTTAAGTCAGCTGCCTCATTCCCTGTTTC
AAGGAAGCAAAACCTTGACATCACTTCCATTGCCAGCAACGGCGGAAGAG
TGCAATGCATGCAGGTGTGGCCAATGAGCAAAGGAGAAGAACTTTTCACT
GGAGTTGTCCCAATTCTTGTTGAATTAGATGGTGATGTTAATGGGCACAA
ATTTTCTGTCAGAGGAGAGGGTGAAGGTGATGCTACAATCGGAAAACTCA
CCCTTAAATTTATTTGCACTACTGGAAAACTACCTGTTCCTTGGCCAACA
CTTGTCACTACTCTGACCTATGGTGTTCAATGCTTTTCCCGTTATCCGGA
TCACATGAAAAGGCATGACTTTTTCAAGAGTGCCATGCCCGAAGGTTATG
TTCAGGAACGCACTATATCTTTCAAAGATGACGGGAAATACAAGACGCGT
GCTGTAGTCAAGTTTGAAGGTGATACCCTTGTTAATCGTATCGAGTTAAA
GGGTACTGATTTTAAAGAAGATGGAAACATTCTCGGACACAAACTTGAGT
ACAACTTTAACTCACACAATGTATACATCACGGCAGACAAACAAAAGAAT
GGAATCAAAGCTAACTTCACAGTTCGCCACAACGTTGAAGATGGTTCCGT
TCAACTAGCAGACCATTATCAACAAAATACTCCAATTGGCGATGGCCCTG
TCCTTTTACCAGACAACCATTACCTGTCGACACAAACTGTCCTTTCGAAA
GATCCCAACGAAAAGGGAACCTAATTCTAGACTGCTTTAATGAGATATGC
GAGACGCCTATGATCGCATGATATTTGCTTTCAATTCTGTTGTGCACGTT
GTAAAAAACCTGAGCATGTGTAGCTCAGATCCTTACCGCCGGTTTCGGTT
CATTCTAATGAATATATCACCCGTTACTATCGTATTTTTATGAATAATAT
TCTCCGTTCAATTTACTGATTGTCCAagcttggcgtaatcatggtcatag
ctgtttcctgtgtgaaattgttatccgctcacaattccacacaacatacg
agccggaagcataaagtgtaaagcctggggtgcctaatgagtgagctaac
tcacattaattgcgttgcgctcactgcccgctttccagtcgggaaacctg
tcgtgccagctgcattaatgaatcggccaacgcgcggggagaggcggttt
gcgtattgggccaaagacaaaagggcgacattcaaccgattgagggaggg
aaggtaaatattgacggaaattattcattaaaggtgaattatcaccgtca
ccgacttgagccatttgggaattagagccagcaaaatcaccagtagcacc
attaccattagcaaggccggaaacgtcaccaatgaaaccatcgatagcag
caccgtaatcagtagcgacagaatcaagtttgcctttagcgtcagactgt
agcgcgttttcatcggcattttcggtcatagcccccttattagcgtttgc
catcttttcataatcaaaatcaccggaaccagagccaccaccggaaccgc
ctccctcagagccgccaccctcagaaccgccaccctcagagccaccaccc
tcagagccgccaccagaaccaccaccagagccgccgccagcattgacagg
aggcccgatctagtaacatagatgacaccgcgcgcgataatttatcctag
tttgcgcgctatattttgttttctatcgcgtattaaatgtataattgcgg
gactctaatcataaaaacccatctcataaataacgtcatgcattacatgt
taattattacatgcttaacgtaattcaacagaaattatatgataatcatc
gcaagaccggcaacaggattcaatcttaagaaactttattgccaaatgtt
tgaacgatcggggatcatccgggtctgtggcgggaactccacgaaaatat
ccgaacgcagcaagatatcgcggtgcatctcggtcttgcctgggcagtcg
ccgccgacgccgttgatgtggacgccgggcccgatcatattgtcgctcag
gatcgtggcgttgtgcttgtcggccgttgctgtcgtaatgatatcggcac
cttcgaccgcctgttccgcagagatcccgtgggcgaagaactccagcatg
agatccccgcgctggaggatcatccagccggcgtcccggaaaacgattcc
gaagcccaacctttcatagaaggcggcggtggaatcgaaatctcgtgatg
gcaggttgggcgtcgcttggtcggtcatttcgaaccccagagtcccgctc
agaagaactcgtcaagaaggcgatagaaggcgatgcgctgcgaatcggga
gcggcgataccgtaaagcacgaggaagcggtcagcccattcgccgccaag
ctcttcagcaatatcacgggtagccaacgctatgtcctgatagcggtccg
ccacacccagccggccacagtcgatgaatccagaaaagcggccattttcc
accatgatattcggcaagcaggcatcgccatgggtcacgacgagatcatc
gccgtcgggcatgcgcgccttgagcctggcgaacagttcggctggcgcga
gcccctgatgctcttcgtccagatcatcctgatcgacaagaccggcttcc
atccgagtacgtgctcgctcgatgcgatgtttcgcttggtggtcgaatgg
gcaggtagccggatcaagcgtatgcagccgccgcattgcatcagccatga
tggatactttctcggcaggagcaaggtgagatgacaggagatcctgcccc
ggcacttcgcccaatagcagccagtcccttcccgcttcagtgacaacgtc
gagcacagctgcgcaaggaacgcccgtcgtggccagccacgatagccgcg
ctgcctcgtcctgcagttcattcagggcaccggacaggtcggtcttgaca
aaaagaaccgggcgcccctgcgctgacagccggaacacggcggcatcaga
gcagccgattgtctgttgtgcccagtcatagccgaatagcctctccaccc
aagcggccggagaacctgcgtgcaatccatcttgttcaatcatgcgaaac
gatccagatccggtgcagattatttggattgagagtgaatatgagactct
aattggataccgaggggaatttatggaacgtcagtggagcatttttgaca
agaaatatttgctagctgatagtgaccttaggcgacttttgaacgcgcaa
taatggtttctgacgtatgtgcttagctcattaaactccagaaacccgcg
gctgagtggctccttcaacgttgcggttctgtcagttccaaacgtaaaac
ggcttgtcccgcgtcatcggcgggggtcataacgtgactcccttaattct
ccgctcatgatcagattgtcgtttcccgccttcagtttaaactatcagtg
tttgacaggatatattggcgggtaaacctaagagaaaagagcgtttatta
gaataatcggatatttaaaagggcgtgaaaaggtttatccgttcgtccat
ttgtatgtgcatgccaaccacagggttccccagatctggcgccggccagc
gagacgagcaagattggccgccgcccgaaacgatccgacagcgcgcccag
cacaggtgcgcaggcaaattgcaccaacgcatacagcgccagcagaatgc
catagtgggcggtgacgtcgttcgagtgaaccagatcgcgcaggaggccc
ggcagcaccggcataatcaggccgatgccgacagcgtcgagcgcgacagt
gctcagaattacgatcaggggtatgttgggtttcacgtctggcctccgga
ccagcctccgctggtccgattgaacgcgcggattctttatcactgataag
ttggtggacatattatgtttatcagtgataaagtgtcaagcatgacaaag
ttgcagccgaatacagtgatccgtgccgccctggacctgttgaacgaggt
cggcgtagacggtctgacgacacgcaaactggcggaacggttgggggttc
agcagccggcgctttactggcacttcaggaacaagcgggcgctgctcgac
gcactggccgaagccatgctggcggagaatcatacgcattcggtgccgag
agccgacgacgactggcgctcatttctgatcgggaatgcccgcagcttca
ggcaggcgctgctcgcctaccgcgatggcgcgcgcatccatgccggcacg
cgaccgggcgcaccgcagatggaaacggccgacgcgcagcttcgcttcct
ctgcgaggcgggtttttcggccggggacgccgtcaatgcgctgatgacaa
tcagctacttcactgttggggccgtgcttgaggagcaggccggcgacagc
gatgccggcgagcgcggcggcaccgttgaacaggctccgctctcgccgct
gttgcgggccgcgatagacgccttcgacgaagccggtccggacgcagcgt
tcgagcagggactcgcggtgattgtcgatggattggcgaaaaggaggctc
gttgtcaggaacgttgaaggaccgagaaagggtgacgattgatcaggacc
gctgccggagcgcaacccactcactacagcagagccatgtagacaacatc
ccctccccctttccaccgcgtcagacgcccgtagcagcccgctacgggct
ttttcatgccctgccctagcgtccaagcctcacggccgcgctcggcctct
ctggcggccttctggcgctcttccgcttcctcgctcactgactcgctgcg
ctcggtcgttcggctgcggcgagcggtatcagctcactcaaaggcggtaa
tacggttatccacagaatcaggggataacgcaggaaagaacatgtgagca
aaaggccagcaaaaggccaggaaccgtaaaaaggccgcgttgctggcgtt
tttccataggctccgcccccctgacgagcatcacaaaaatcgacgctcaa
gtcagaggtggcgaaacccgacaggactataaagataccaggcgtttccc
cctggaagctccctcgtgcgctctcctgttccgaccctgccgcttaccgg
atacctgtccgcctttctcccttcgggaagcgtggcgcttttccgctgca
taaccctgcttcggggtcattatagcgattttttcggtatatccatcctt
tttcgcacgatatacaggattttgccaaagggttcgtgtagactttcctt
ggtgtatccaacggcgtcagccgggcaggataggtgaagtaggcccaccc
gcgagcgggtgttccttcttcactgtcccttattcgcacctggcggtgct
caacgggaatcctgctctgcgaggctggccggctaccgccggcgtaacag
atgagggcaagcggatggctgatgaaaccaagccaaccaggaagggcagc
ccacctatcaaggtgtactgccttccagacgaacgaagagcgattgagga
aaaggcggcggcggccggcatgagcctgtcggcctacctgctggccgtcg
gccagggctacaaaatcacgggcgtcgtggactatgagcacgtccgcgag
ctggcccgcatcaatggcgacctgggccgcctgggcggcctgctgaaact
ctggctcaccgacgacccgcgcacggcgcggttcggtgatgccacgatcc
tcgccctgctggcgaagatcgaagagaagcaggacgagcttggcaaggtc
atgatgggcgtggtccgcccgagggcagagccatgacttttttagccgct
aaaacggccggggggtgcgcgtgattgccaagcacgtccccatgcgctcc
atcaagaagagcgacttcgcggagctggtgaagtacatcaccgacgagca
aggcaagaccgagcgcctttgcgacgctca

>SEQ ID NO 15 pJF7

AGTACTTTGATCCAACCCCTCCGCTGCTATAGTGCAGTCGGCTTCTGACG
TTCAGTGCAGCCGTCTTCTGAAAACGACATGTCGCACAAGTCCTAAGTTA
CGCGACAGGCTGCCGCCCTGCCCTTTTCCTGGCGTTTTCTTGTCGCGTGT
TTTAGTCGCATAAAGTAGAATACTTGCGACTAGAACCGGAGACATTACGC
CATGAACAAGAGCGCCGCCGCTGGCCTGCTGGGCTATGCCCGCGTCAGCA
CCGACGACCAGGACTTGACCAACCAACGGGCCGAACTGCACGCGGCCGGC
TGCACCAAGCTGTTTTCCGAGAAGATCACCGGCACCAGGCGCGACCGCCC
GGAGCTGGCCAGGATGCTTGACCACCTACGCCCTGGCGACGTTGTGACAG
TGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCC
GTGGGCCGACACCACCACGCCGGCCGGCCGCATGGTGTTGACCGTGTTCG
CCGGCATTGCCGAGTTCGAGCGTTCCCTAATCATCGACCGCACCCGGAGC
GGGCGCGAGGCCGCCAAGGCCCGAGGCGTGAAGTTTGGCCCCCGCCCTAC
CCTCACCCCGGCACAGATCGCGCACGCCCGCGAGCTGATCGACCAGGAAG
GCCGCACCGTGAAAGAGGCGGCTGCACTGCTTGGCGTGCATCGCTCGACC
CTGTACCGCGCACTTGAGCGCAGCGAGGAAGTGACGCCCACCGAGGCCAG
GCGGCGCGGTGCCTTCCGTGAGGACGCATTGACCGAGGCCGACGCCCTGG
CGGCCGCCGAGAATGAACGCCAAGAGGAACAAGCATGAAACCGCACCAGG
ACGGCCAGGACGAACCGTTTTTCATTACCGAAGAGATCGAGGCGGAGATG
ATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGC
CGGCCAGCTTGGCCGCTGAAGAAACCGAGCGCCGCCGTCTAAAAAGGTGA
TGTGTATTTGAGTAAAACAGCTTGCGTCATGCGGTCGCTGCGTATATGAT
GCGATGAGTAAATAAACAAATACGCAAGGGGAACGCATGAAGGTTATCGC
TGTACTTAACCAGAAAGGCGGGTCAGGCAAGACGACCATCGCAACCCATC
TAGCCCGCGCCCTGCAACTCGCCGGGGCCGATGTTCTGTTAGTCGATTCC
GATCCCCAGGGCAGTGCCCGCGATTGGGCGGCCGTGCGGGAAGATCAACC
GCTAACCGTTGTCGGCATCGACCGCCCGACGATTGACCGCGACGTGAAGG
CCATCGGCCGGCGCGACTTCGTAGTGATCGACGGAGCGCCCCAGGCGGCG
GACTTGGCTGTGTCCGCGATCAAGGCAGCCGACTTCGTGCTGATTCCGGT
GCAGCCAAGCCCTTACGACATATGGGCCACCGCCGACCTGGTGGAGCTGG
TTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCG
TGAGCTACCCAGGCACTGCCGCCGCCGGCACAACCGTTCTTGAATCAGAA
CCCGAGGGCGACGCTGCCCGCGAGGTCCAGGCGCTGGCCGCTGAAATTAA
ATCAAAACTCATTTGAGTTAATGAGGTAAAGAGAAAATGAGCAAAAGCAC
AAACACGCTAAGTGCCGGCCGTCCGAGCGCACGCAGCAGCAAGGCTGCAA
CGTTGGCCAGCCTGGCAGACACGCCAGCCATGAAGCGGGTCAACTTTCAG
TTGCCGGCGGAGGATCACACCAAGCTGAAGATGTACGCGGTACGCCAAGG
CAAGACCATTACCGAGCTGCTATCTGAATACATCGCGCAGCTACCAGAGT
AAATGAGCAAATGAATAAATGAGTAGATGAATTTTAGCGGCTAAAGGAGG
CGGCATGGAAAATCAAGAACAACCAGGCACCGACGCCGTGGAATGCCCCA
TGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGAGC
GGTCGCAAACCATCCGGCCCGGTACAAATCGGCGCGGCGCTGGGTGATGA
CCTGGTGGAGAAGTTGAAGGCCGCGCAGGCCGCCCAGCGGCAACGCATCG
AGGCAGAAGCACGCCCCGGTGAATCGTGGCAAGCGGCCGCTGATCGAATC
CGCAAAGAATCCCGGCAACCGCCGGCAGCCGGTGCGCCGTCGATTAGGAA
GCCGCCCAAGGGCGACGAGCAACCAGATTTTTTCGTTCCGATGCTCTATG
ACGTGGGCACCCGCGATAGTCGCAGCATCATGGACGTGGCCGTTTTCCGT
CTGTCGAAGCGTGACCGACGAGCTGGCGAGGTGATCCGCTACGAGCTTCC
AGACGGGCACGTAGAGGTTTCCGCAGGGCCGGCCGGCATGGCCAGTGTGT
GGGATTACGACCTGGTACTGATGGCGGTTTCCCATCTAACCGAATCCATG
AACCGATACCGGGAAGGGAAGGGAGACAAGCCCGGCCGCGTGTTCCGTCC
ACACGTTGCGGACGTACTCAAGTTCTGCCGGCGAGCCGATGGCGGAAAGC
AGAAAGACGACCTGGTAGAAACCTGCATTCGGTTAAACACCACGCACGTT
GCCATGCAGCGTACGAAGAAGGCCAAGAACGGCCGCCTGGTGACGGTATC
CGAGGGTGAAGCCTTGATTAGCCGCTACAAGATCGTAAAGAGCGAAACCG
GGCGGCCGGAGTACATCGAGATCGAGCTAGCTGATTGGATGTACCGCGAG
ATCACAGAAGGCAAGAACCCGGACGTGCTGACGGTTCACCCCGATTACTT
TTTGATCGATCCCGGCATCGGCCGTTTTCTCTACCGCCTGGCACGCCGCG
CCGCAGGCAAGGCAGAAGCCAGATGGTTGTTCAAGACGATCTACGAACGC
AGTGGCAGCGCCGGAGAGTTCAAGAAGTTCTGTTTCACCGTGCGCAAGCT
GATCGGGTCAAATGACCTGCCGGAGTACGATTTGAAGGAGGAGGCGGGGC
AGGCTGGCCCGATCCTAGTCATGCGCTACCGCAACCTGATCGAGGGCGAA
GCATCCGCCGGTTCCTAATGTACGGAGCAGATGCTAGGGCAAATTGCCCT
AGCAGGGGAAAAAGGTCGAAAAGGTCTCTTTCCTGTGGATAGCACGTACA
TTGGGAACCCAAAGCCGTACATTGGGAACCGGAACCCGTACATTGGGAAC
CCAAAGCCGTACATTGGGAACCGGTCACACATGTAAGTGACTGATATAAA
AGAGAAAAAAGGCGATTTTTCCGCCTAAAACTCTTTAAAACTTATTAAAA
CTCTTAAAACCCGCCTGGCCTGTGCATAACTGTCTGGCCAGCGCACAGCC
GAAGAGCTGCAAAAAGCGCCTACCCTTCGGTCGCTGCGCTCCCTACGCCC
CGCCGCTTCGCGTCGGCCTATCGCGGCCGCTGGCCGCTCAAAAATGGCTG
GCCTACGGCCAGGCAATCTACCAGGGCGCGGACAAGCCGCGCCGTCGCCA
CTCGACCGCCGGCGCCCACATCAAGGCACCCTGCCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATA
GCGGAGTGTATACTGGCTTAACTATGCGGCATCAGAGCAGATTGTACTGA
GAGTGCACCATATGCGGTGTGAAATACCGCACAGATGCGTAAGGAGAAAA
TACCGCATCAGGCGCTCTTCCGCTTCCTCGCTCACTGACTCGCTGCGCTC
GGTCGTTCGGCTGCGGCGAGCGGTATCAGCTCACTCAAAGGCGGTAATAC
GGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTA
TCGTCTTGAGTCCAACCCGGTAAGACACGACTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGA
TTTTGGTCATGCATGATATATCTCCCAATTTGTGTAGGGCTTATTATGCA
CGCTTAAAAATAATAAAAGCAGACTTGACCTGATAGTTTGGCTGTGAGCA
ATTATGTGCTTAGTGCATCTAATCGCTTGAGTTAACGCCGGCGAAGCGGC
GTCGGCTTGAACGAATTTCTAGCTAGACATTATTTGCCGACTACCTTGGT
GATCTCGCCTTTCACGTAGTGGACAAATTCTTCCAACTGATCTGCGCGCG
AGGCCAAGCGATCTTCTTCTTGTCCAAGATAAGCCTGTCTAGCTTCAAGT
ATGACGGGCTGATACTGGGCCGGCAGGCGCTCCATTGCCCAGTCGGCAGC
GACATCCTTCGGCGCGATTTTGCCGGTTACTGCGCTGTACCAAATGCGGG
ACAACGTAAGCACTACATTTCGCTCATCGCCAGCCCAGTCGGGCGGCGAG
TTCCATAGCGTTAAGGTTTCATTTAGCGCCTCAAATAGATCCTGTTCAGG
AACCGGATCAAAGAGTTCCTCCGCCGCTGGACCTACCAAGGCAACGCTAT
GTTCTCTTGCTTTTGTCAGCAAGATAGCCAGATCAATGTCGATCGTGGCT
GGCTCGAAGATACCTGCAAGAATGTCATTGCGCTGCCATTCTCCAAATTG
CAGTTCGCGCTTAGCTGGATAACGCCACGGAATGATGTCGTCGTGCACAA
CAATGGTGACTTCTACAGCGCGGAGAATCTCGCTCTCTCCAGGGGAAGCC
GAAGTTTCCAAAAGGTCGTTGATCAAAGCTCGCCGCGTTGTTTCATCAAG
CCTTACGGTCACCGTAACCAGCAAATCAATATCACTGTGTGGCTTCAGGC
CGCCATCCACTGCGGAGCCGTACAAATGTACGGCCAGCAACGTCGGTTCG
AGATGGCGCTCGATGACGCCAACTACCTCTGATAGTTGAGTCGATACTTC
GGCGATCACCGCTTCCCCCATGATGTTTAACTTTGTTTTAGGGCGACTGC
CCTGCTGCGTAACATCGTTGCTGCTCCATAACATCAAACATCGACCCACG
GCGTAACGCGCTTGCTGCTTGGATGCCCGAGGCATAGACTGTACCCCAAA
AAAACATGTCATAACAAGAAGCCATGAAAACCGCCACTGCGCCGTTACCA
CCGCTGCGTTCGGTCAAGGTTCTGGACCAGTTGCGTGACGGCAGTTACGC
TACTTGCATTACAGCTTACGAACCGAACGAGGCTTATGTCCACTGGGTTC
GTGCCCGAATTGATCACAGGCAGCAACGCTCTGTCATCGTTACAATCAAC
ATGCTACCCTCCGCGAGATCATCCGTGTTTCAAACCCGGCAGCTTAGTTG
CCGTTCTTCCGAATAGCATCGGTAACATGAGCAAAGTCTGCCGCCTTACA
ACGGCTCTCCCGCTGACGCCGTCCCGGACTGATGGGCTGCCTGTATCGAG
TGGTGATTTTGTGCCGAGCTGCCGGTCGGGGAGCTGTTGGCTGGCTGGTG
GCAGGATATATTGTGGTGTAAACAAATTGACGCTTAGACAACTTAATAAC
ACATTGCGGACGTTTTTAATGTACTGAATTAACGCCGAATTAATTCGGGG
GATCTATAGGGACTTTAGGTGATCTGGATTTTAGTACTGGATTTTGGTTT
TAGGAATTAGAAATTTTATTGATAGAAGTATTTTACAAATACAAATACAT
ACTAAGGGTTTCTTATATGCTCAACACGTGAGCGAAACCCTATAAGAACC
CTAATTCCCTTATCTGGGAACTACTCACACATTATTATGGAGAAACTCGA
TGTCGATCGACTCTAGCTAGAACGAATTGTTAGGTGGCGGTACTTGGGTC
GATATCAAAGTGCATCACTTCTTCCCGTATGCCCAACTTTGTATAGAGAG
CCACTGCGGGATCGTCACCGTAATCTGCTTGCACGTAGATCACATAAGCA
CCAAGCGCGTTGGCCTCATGCTTGAGGAGATTGATGAGCGCGGTGGCAAT
GCCCTGCCTCCGGTGCTCGCCGGAGACTGCGAGATCATAGATATAGATCT
CACTACGCGGCTGCTCAAACCTGGGCAGAACGTAAGCCGCGAGAGCGCCA
ACAACCGCTTCTTGGTCGAAGGCAGCAAGCGCGATGAATGTCTTACTACG
GAGCAAGTTCCCGAGGTAATCGGAGTCCGGCTGATGTTGGGAGTAGGTGG
CTACGTCTCCGAACTCACGACCGAAAAGATCAAGAGCAGCCCGCATGGAT
TTGACTTGGTCAGGGCCGAGCCTACATGTGCGAATGATGCCCATACTTGA
GCCACCTAACTTTGTTTTAGGGCGACTGCCCTGCTGCGTAACATCGTTGC
TGCTGCGTACCATGGTCGATCGACAGATCTGCGAATCGAGAGAGATAGAT
TTGTAGAGAGAGACTGGTGATTTCAGCGTGTCCTCTCCAAATGAAATGAA
CTTCCTTATATAGAGGAAGGGTCTTGCGAAGGATAGTGGGATTGTGCGTC
ATCCCTTACGTCAGTGGAGATATCACATCAATCCACTTGCTTTGAAGACG
TGGTTGGAACGTCTTCTTTTTCCACGATGCTCCTCGTGGGTGGGGGTCCA
TCTTTGGGACCACTGTCGGCAGAGGCATCTTGAACGATAGCCTTTCCTTT
ATCGCAATGATGGCATTTGTAGGTGCCACCTTCCTTTTCTACTGTCCTTT
TGATGAAGTGACAGATAGCTGGGCAATGGAATCCGAGGAGGTTTCCCGAT
ATTACCCTTTGTTGAAAAGTCTCAATAGCCCTTTGGTCTTCTGAGACTGT
ATCTTTGATATTCTTGGAGTAGACGAGAGTGTCGTGCTCCACCATGTTAT
CACATCAATCCACTTGCTTTGAAGACGTGGTTGGAACGTCTTCTTTTTCC
ACGATGCTCCTCGTGGGTGGGGGTCCATCTTTGGGACCACTGTCGGCAGA
GGCATCTTGAACGATAGCCTTTCCTTTATCGCAATGATGGCATTTGTAGG
TGCCACCTTCCTTTTCTACTGTCCTTTTGATGAAGTGACAGATAGCTGGG
CAATGGAATCCGAGGAGGTTTCCCGATATTACCCTTTGTTGAAAAGTCTC
AATAGCCCTTTGGTCTTCTGAGACTGTATCTTTGATATTCTTGGAGTAGA
CGAGAGTGTCGTGCTCCACCATGTTGGCAAGCTGCTCTAGCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGAATTCGGGATATTTCACAAATTGAACATAGACTA
CAGAATTTTAGAAAACAAACTTTCTCTCTCTTATCTCACCTTTATCTTTT
AGAGAGAAAAAGTTCGATTTCCGGTTGACCGGAATGTATCTTTGTTTTTT
TTGTTTTGTAACATATTTCGTTTTCCGATTTAGATCGGATCTCCTTTTCC
GTTTTGTCGGACCTTCTTCCGGTTTATCCGGATCTAATAATATCCATCTT
AGACTTAGCTAAGTTTGGATCTGTTTTTTGGTTAGCTCTTGTCAATCGCC
TCATCATCAGCAAGAAGGTGAAATTTTTGACAAATAAATCTTAGAATCAT
GTAGTGTCTTTGGACCTTGGGAATGATAGAAACGATTTGTTATAGCTACT
CTATGTATCAGACCCTGACCAAGATCCAACAATCTCATAGGTTTTGTGCA
TATGAAACCTTCGACTAACGAGAAGTGGTCTTTTAATGAGAGAGATATCT
AAAATGTTATCTTAAAAGCCCACTCAAATCTCAAGGCATAAGGTAGAAAT
GCAAATTTGGAAAGTGGGCTGGGCCTTTTGTGGTAAAGGCCTGTAACCTA
GCCCAATATTAGCAAAACCCTAGACGCGTACATTGACATATATAAACCCG
CCTCCTCCTTGTTTAGGGTTTCTACGTGAGAGAGACGAAACACAAACCAT
GGCTTCCTCAGTTCTTTCCTCCGCAGCAGTTGCCACCCGCAGCAATGTTG
CTCAAGCTAACATGGTTGCACCTTTCACTGGCCTTAAGTCAGCTGCCTCA
TTCCCTGTTTCAAGGAAGCAAAACCTTGACATCACTTCCATTGCCAGCAA
CGGCGGAAGAGTGCAATGCATGCAGGTGTGGCCAGCGCGTGACCACATGG
TCCTTCATGAGTACGTAAATGCTGCTGGGATTACAATGGCTCCCGATCGG
GCCCAAGTAATCATTCGCATTGTGCCAGGAGGTGGAACCAAGACCCTTCA
GCAGATAATCAATCAGCTGGAGTACCTGTCCCGAAAGGGAAAGCTGGAAC
TCCAGCGTTCAGCCCGGCATCTCGATATTCCCGTTCCGCCGGATCAAATC
CGTGAGCTTGCCCAAAGCTGGGTTACGGAGGCCGGGATTTATGACGAAAG
TCAGTCAGACGATGACAGGCAACAAGACTTAACAACACACATTATTGTGA
GCTTCCCCGCAGGTACCGACCAAACCGCAGCTTATGAAGCAAGCCGGGAA
TGGGCAGCCGAGATGTTTGGGTCAGGATACGGGGGTGGCCGCTATAACTA
TCTGACAGCCTACCACGTGGACCGCGATCATCCACATTTACATGTCGTGG
TCAATCGTCGGGAACTTCTGGGGCAGGGGTGGCTGAAAATATCCAGGCGC
CATCCCCAGCTGAATTATGACGGCTTACGGAAAAAGATGGCAGAGATTTC
ACTTCGTCACGGCATAGTCCTGGATGCGACTTCCCGAGCAGAAAGGGGAA
TAGCAGAGCGACCAATCACCTATGCTGAATATCGACGTCTTGAGCGGATG
CTCGAAGTTATGGCAGAAGTTCGGCCCATAGCCCGATCCATTAAAACGGC
TCACGACGATGCGCGAGCGGAATTAATGTCGGCGGACAGACCTCGATCAA
CGCGCGGTCTATGATTCTAGAAGAGACTCTTATCAAGAATCCCATCTCTT
GCTTGCTTTTTTTTGTTGCTTCCCTTTGATAGGGTTTGTTTTTCTTGTTT
CAGTGACTTTCTATGTTAAAAGATAATGTCAGTAAAAGGATTTGGTTTTC
TATTATTCTGAATCGATTACGGAAGATTCTTGCTTAATTCCAATCTATAC
AAGTATCGTGAAATAATGACCGTTTATGTAAGCTTGGCACTGGCCGTCGT
TTTACAACGTCGTGACTGGGAAAACCCTGGCGTTACCCAACTTAATCGCC
TTGCAGCACATCCCCCTTTCGCCAGCTGGCGTAATAGCGAAGAGGCCCGC
ACCGATCGCCCTTCCCAACAGTTGCGCAGCCTGAATGGCGAATGCTAGAG
CAGCTTGAGCTTGGATCAGATTGTCGTTTCCCGCCTTCAGTTTAAACTAT
CAGTGTTTGACAGGATATATTGGCGGGTAAACCTAAGAGAAAAGAGCGTT
TATTAGAATAACGGATATTTAAAAGGGCGTGAAAAGGTTTATCCGTTCGT
CCATTTGTATGTGCATGCCAACCACAGGGTTCCCCTCGGGATCAA

>SEQ ID NO 16 pJF8

AGTACTTTGATCCAACCCCTCCGCTGCTATAGTGCAGTCGGCTTCTGACG
TTCAGTGCAGCCGTCTTCTGAAAACGACATGTCGCACAAGTCCTAAGTTA
CGCGACAGGCTGCCGCCCTGCCCTTTTCCTGGCGTTTTCTTGTCGCGTGT
TTTAGTCGCATAAAGTAGAATACTTGCGACTAGAACCGGAGACATTACGC
CATGAACAAGAGCGCCGCCGCTGGCCTGCTGGGCTATGCCCGCGTCAGCA
CCGACGACCAGGACTTGACCAACCAACGGGCCGAACTGCACGCGGCCGGC
TGCACCAAGCTGTTTTCCGAGAAGATCACCGGCACCAGGCGCGACCGCCC
GGAGCTGGCCAGGATGCTTGACCACCTACGCCCTGGCGACGTTGTGACAG
TGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCC
GTGGGCCGACACCACCACGCCGGCCGGCCGCATGGTGTTGACCGTGTTCG
CCGGCATTGCCGAGTTCGAGCGTTCCCTAATCATCGACCGCACCCGGAGC
GGGCGCGAGGCCGCCAAGGCCCGAGGCGTGAAGTTTGGCCCCCGCCCTAC
CCTCACCCCGGCACAGATCGCGCACGCCCGCGAGCTGATCGACCAGGAAG
GCCGCACCGTGAAAGAGGCGGCTGCACTGCTTGGCGTGCATCGCTCGACC
CTGTACCGCGCACTTGAGCGCAGCGAGGAAGTGACGCCCACCGAGGCCAG
GCGGCGCGGTGCCTTCCGTGAGGACGCATTGACCGAGGCCGACGCCCTGG
CGGCCGCCGAGAATGAACGCCAAGAGGAACAAGCATGAAACCGCACCAGG
ACGGCCAGGACGAACCGTTTTTCATTACCGAAGAGATCGAGGCGGAGATG
ATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGC
CGGCCAGCTTGGCCGCTGAAGAAACCGAGCGCCGCCGTCTAAAAAGGTGA
TGTGTATTTGAGTAAAACAGCTTGCGTCATGCGGTCGCTGCGTATATGAT
GCGATGAGTAAATAAACAAATACGCAAGGGGAACGCATGAAGGTTATCGC
TGTACTTAACCAGAAAGGCGGGTCAGGCAAGACGACCATCGCAACCCATC
TAGCCCGCGCCCTGCAACTCGCCGGGGCCGATGTTCTGTTAGTCGATTCC
GATCCCCAGGGCAGTGCCCGCGATTGGGCGGCCGTGCGGGAAGATCAACC
GCTAACCGTTGTCGGCATCGACCGCCCGACGATTGACCGCGACGTGAAGG
CCATCGGCCGGCGCGACTTCGTAGTGATCGACGGAGCGCCCCAGGCGGCG
GACTTGGCTGTGTCCGCGATCAAGGCAGCCGACTTCGTGCTGATTCCGGT
GCAGCCAAGCCCTTACGACATATGGGCCACCGCCGACCTGGTGGAGCTGG
TTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCG
TGAGCTACCCAGGCACTGCCGCCGCCGGCACAACCGTTCTTGAATCAGAA
CCCGAGGGCGACGCTGCCCGCGAGGTCCAGGCGCTGGCCGCTGAAATTAA
ATCAAAACTCATTTGAGTTAATGAGGTAAAGAGAAAATGAGCAAAAGCAC
AAACACGCTAAGTGCCGGCCGTCCGAGCGCACGCAGCAGCAAGGCTGCAA
CGTTGGCCAGCCTGGCAGACACGCCAGCCATGAAGCGGGTCAACTTTCAG
TTGCCGGCGGAGGATCACACCAAGCTGAAGATGTACGCGGTACGCCAAGG
CAAGACCATTACCGAGCTGCTATCTGAATACATCGCGCAGCTACCAGAGT
AAATGAGCAAATGAATAAATGAGTAGATGAATTTTAGCGGCTAAAGGAGG
CGGCATGGAAAATCAAGAACAACCAGGCACCGACGCCGTGGAATGCCCCA
TGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGAGC
GGTCGCAAACCATCCGGCCCGGTACAAATCGGCGCGGCGCTGGGTGATGA
CCTGGTGGAGAAGTTGAAGGCCGCGCAGGCCGCCCAGCGGCAACGCATCG
AGGCAGAAGCACGCCCCGGTGAATCGTGGCAAGCGGCCGCTGATCGAATC
CGCAAAGAATCCCGGCAACCGCCGGCAGCCGGTGCGCCGTCGATTAGGAA
GCCGCCCAAGGGCGACGAGCAACCAGATTTTTTCGTTCCGATGCTCTATG
ACGTGGGCACCCGCGATAGTCGCAGCATCATGGACGTGGCCGTTTTCCGT
CTGTCGAAGCGTGACCGACGAGCTGGCGAGGTGATCCGCTACGAGCTTCC
AGACGGGCACGTAGAGGTTTCCGCAGGGCCGGCCGGCATGGCCAGTGTGT
GGGATTACGACCTGGTACTGATGGCGGTTTCCCATCTAACCGAATCCATG
AACCGATACCGGGAAGGGAAGGGAGACAAGCCCGGCCGCGTGTTCCGTCC
ACACGTTGCGGACGTACTCAAGTTCTGCCGGCGAGCCGATGGCGGAAAGC
AGAAAGACGACCTGGTAGAAACCTGCATTCGGTTAAACACCACGCACGTT
GCCATGCAGCGTACGAAGAAGGCCAAGAACGGCCGCCTGGTGACGGTATC
CGAGGGTGAAGCCTTGATTAGCCGCTACAAGATCGTAAAGAGCGAAACCG
GGCGGCCGGAGTACATCGAGATCGAGCTAGCTGATTGGATGTACCGCGAG
ATCACAGAAGGCAAGAACCCGGACGTGCTGACGGTTCACCCCGATTACTT
TTTGATCGATCCCGGCATCGGCCGTTTTCTCTACCGCCTGGCACGCCGCG
CCGCAGGCAAGGCAGAAGCCAGATGGTTGTTCAAGACGATCTACGAACGC
AGTGGCAGCGCCGGAGAGTTCAAGAAGTTCTGTTTCACCGTGCGCAAGCT
GATCGGGTCAAATGACCTGCCGGAGTACGATTTGAAGGAGGAGGCGGGGC
AGGCTGGCCCGATCCTAGTCATGCGCTACCGCAACCTGATCGAGGGCGAA
GCATCCGCCGGTTCCTAATGTACGGAGCAGATGCTAGGGCAAATTGCCCT
AGCAGGGGAAAAAGGTCGAAAAGGTCTCTTTCCTGTGGATAGCACGTACA
TTGGGAACCCAAAGCCGTACATTGGGAACCGGAACCCGTACATTGGGAAC
CCAAAGCCGTACATTGGGAACCGGTCACACATGTAAGTGACTGATATAAA
AGAGAAAAAAGGCGATTTTTCCGCCTAAAACTCTTTAAAACTTATTAAAA
CTCTTAAAACCCGCCTGGCCTGTGCATAACTGTCTGGCCAGCGCACAGCC
GAAGAGCTGCAAAAAGCGCCTACCCTTCGGTCGCTGCGCTCCCTACGCCC
CGCCGCTTCGCGTCGGCCTATCGCGGCCGCTGGCCGCTCAAAAATGGCTG
GCCTACGGCCAGGCAATCTACCAGGGCGCGGACAAGCCGCGCCGTCGCCA
CTCGACCGCCGGCGCCCACATCAAGGCACCCTGCCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATA
GCGGAGTGTATACTGGCTTAACTATGCGGCATCAGAGCAGATTGTACTGA
GAGTGCACCATATGCGGTGTGAAATACCGCACAGATGCGTAAGGAGAAAA
TACCGCATCAGGCGCTCTTCCGCTTCCTCGCTCACTGACTCGCTGCGCTC
GGTCGTTCGGCTGCGGCGAGCGGTATCAGCTCACTCAAAGGCGGTAATAC
GGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTA
TCGTCTTGAGTCCAACCCGGTAAGACACGACTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGA
TTTTGGTCATGCATGATATATCTCCCAATTTGTGTAGGGCTTATTATGCA
CGCTTAAAAATAATAAAAGCAGACTTGACCTGATAGTTTGGCTGTGAGCA
ATTATGTGCTTAGTGCATCTAATCGCTTGAGTTAACGCCGGCGAAGCGGC
GTCGGCTTGAACGAATTTCTAGCTAGACATTATTTGCCGACTACCTTGGT
GATCTCGCCTTTCACGTAGTGGACAAATTCTTCCAACTGATCTGCGCGCG
AGGCCAAGCGATCTTCTTCTTGTCCAAGATAAGCCTGTCTAGCTTCAAGT
ATGACGGGCTGATACTGGGCCGGCAGGCGCTCCATTGCCCAGTCGGCAGC
GACATCCTTCGGCGCGATTTTGCCGGTTACTGCGCTGTACCAAATGCGGG
ACAACGTAAGCACTACATTTCGCTCATCGCCAGCCCAGTCGGGCGGCGAG
TTCCATAGCGTTAAGGTTTCATTTAGCGCCTCAAATAGATCCTGTTCAGG
AACCGGATCAAAGAGTTCCTCCGCCGCTGGACCTACCAAGGCAACGCTAT
GTTCTCTTGCTTTTGTCAGCAAGATAGCCAGATCAATGTCGATCGTGGCT
GGCTCGAAGATACCTGCAAGAATGTCATTGCGCTGCCATTCTCCAAATTG
CAGTTCGCGCTTAGCTGGATAACGCCACGGAATGATGTCGTCGTGCACAA
CAATGGTGACTTCTACAGCGCGGAGAATCTCGCTCTCTCCAGGGGAAGCC
GAAGTTTCCAAAAGGTCGTTGATCAAAGCTCGCCGCGTTGTTTCATCAAG
CCTTACGGTCACCGTAACCAGCAAATCAATATCACTGTGTGGCTTCAGGC
CGCCATCCACTGCGGAGCCGTACAAATGTACGGCCAGCAACGTCGGTTCG
AGATGGCGCTCGATGACGCCAACTACCTCTGATAGTTGAGTCGATACTTC
GGCGATCACCGCTTCCCCCATGATGTTTAACTTTGTTTTAGGGCGACTGC
CCTGCTGCGTAACATCGTTGCTGCTCCATAACATCAAACATCGACCCACG
GCGTAACGCGCTTGCTGCTTGGATGCCCGAGGCATAGACTGTACCCCAAA
AAAACATGTCATAACAAGAAGCCATGAAAACCGCCACTGCGCCGTTACCA
CCGCTGCGTTCGGTCAAGGTTCTGGACCAGTTGCGTGACGGCAGTTACGC
TACTTGCATTACAGCTTACGAACCGAACGAGGCTTATGTCCACTGGGTTC
GTGCCCGAATTGATCACAGGCAGCAACGCTCTGTCATCGTTACAATCAAC
ATGCTACCCTCCGCGAGATCATCCGTGTTTCAAACCCGGCAGCTTAGTTG
CCGTTCTTCCGAATAGCATCGGTAACATGAGCAAAGTCTGCCGCCTTACA
ACGGCTCTCCCGCTGACGCCGTCCCGGACTGATGGGCTGCCTGTATCGAG
TGGTGATTTTGTGCCGAGCTGCCGGTCGGGGAGCTGTTGGCTGGCTGGTG
GCAGGATATATTGTGGTGTAAACAAATTGACGCTTAGACAACTTAATAAC
ACATTGCGGACGTTTTTAATGTACTGAATTAACGCCGAATTAATTCGGGG
GATCTATAGGGACTTTAGGTGATCTGGATTTTAGTACTGGATTTTGGTTT
TAGGAATTAGAAATTTTATTGATAGAAGTATTTTACAAATACAAATACAT
ACTAAGGGTTTCTTATATGCTCAACACGTGAGCGAAACCCTATAAGAACC
CTAATTCCCTTATCTGGGAACTACTCACACATTATTATGGAGAAACTCGA
TGTCGATCGACTCTAGCTAGAACGAATTGTTAGGTGGCGGTACTTGGGTC
GATATCAAAGTGCATCACTTCTTCCCGTATGCCCAACTTTGTATAGAGAG
CCACTGCGGGATCGTCACCGTAATCTGCTTGCACGTAGATCACATAAGCA
CCAAGCGCGTTGGCCTCATGCTTGAGGAGATTGATGAGCGCGGTGGCAAT
GCCCTGCCTCCGGTGCTCGCCGGAGACTGCGAGATCATAGATATAGATCT
CACTACGCGGCTGCTCAAACCTGGGCAGAACGTAAGCCGCGAGAGCGCCA
ACAACCGCTTCTTGGTCGAAGGCAGCAAGCGCGATGAATGTCTTACTACG
GAGCAAGTTCCCGAGGTAATCGGAGTCCGGCTGATGTTGGGAGTAGGTGG
CTACGTCTCCGAACTCACGACCGAAAAGATCAAGAGCAGCCCGCATGGAT
TTGACTTGGTCAGGGCCGAGCCTACATGTGCGAATGATGCCCATACTTGA
GCCACCTAACTTTGTTTTAGGGCGACTGCCCTGCTGCGTAACATCGTTGC
TGCTGCGTACCATGGTCGATCGACAGATCTGCGAATCGAGAGAGATAGAT
TTGTAGAGAGAGACTGGTGATTTCAGCGTGTCCTCTCCAAATGAAATGAA
CTTCCTTATATAGAGGAAGGGTCTTGCGAAGGATAGTGGGATTGTGCGTC
ATCCCTTACGTCAGTGGAGATATCACATCAATCCACTTGCTTTGAAGACG
TGGTTGGAACGTCTTCTTTTTCCACGATGCTCCTCGTGGGTGGGGGTCCA
TCTTTGGGACCACTGTCGGCAGAGGCATCTTGAACGATAGCCTTTCCTTT
ATCGCAATGATGGCATTTGTAGGTGCCACCTTCCTTTTCTACTGTCCTTT
TGATGAAGTGACAGATAGCTGGGCAATGGAATCCGAGGAGGTTTCCCGAT
ATTACCCTTTGTTGAAAAGTCTCAATAGCCCTTTGGTCTTCTGAGACTGT
ATCTTTGATATTCTTGGAGTAGACGAGAGTGTCGTGCTCCACCATGTTAT
CACATCAATCCACTTGCTTTGAAGACGTGGTTGGAACGTCTTCTTTTTCC
ACGATGCTCCTCGTGGGTGGGGGTCCATCTTTGGGACCACTGTCGGCAGA
GGCATCTTGAACGATAGCCTTTCCTTTATCGCAATGATGGCATTTGTAGG
TGCCACCTTCCTTTTCTACTGTCCTTTTGATGAAGTGACAGATAGCTGGG
CAATGGAATCCGAGGAGGTTTCCCGATATTACCCTTTGTTGAAAAGTCTC
AATAGCCCTTTGGTCTTCTGAGACTGTATCTTTGATATTCTTGGAGTAGA
CGAGAGTGTCGTGCTCCACCATGTTGGCAAGCTGCTCTAGCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGAATTCGGGATATTTCACAAATTGAACATAGACTA
CAGAATTTTAGAAAACAAACTTTCTCTCTCTTATCTCACCTTTATCTTTT
AGAGAGAAAAAGTTCGATTTCCGGTTGACCGGAATGTATCTTTGTTTTTT
TTGTTTTGTAACATATTTCGTTTTCCGATTTAGATCGGATCTCCTTTTCC
GTTTTGTCGGACCTTCTTCCGGTTTATCCGGATCTAATAATATCCATCTT
AGACTTAGCTAAGTTTGGATCTGTTTTTTGGTTAGCTCTTGTCAATCGCC
TCATCATCAGCAAGAAGGTGAAATTTTTGACAAATAAATCTTAGAATCAT
GTAGTGTCTTTGGACCTTGGGAATGATAGAAACGATTTGTTATAGCTACT
CTATGTATCAGACCCTGACCAAGATCCAACAATCTCATAGGTTTTGTGCA
TATGAAACCTTCGACTAACGAGAAGTGGTCTTTTAATGAGAGAGATATCT
AAAATGTTATCTTAAAAGCCCACTCAAATCTCAAGGCATAAGGTAGAAAT
GCAAATTTGGAAAGTGGGCTGGGCCTTTTGTGGTAAAGGCCTGTAACCTA
GCCCAATATTAGCAAAACCCTAGACGCGTACATTGACATATATAAACCCG
CCTCCTCCTTGTTTAGGGTTTCTACGTGAGAGAGACGAAACACAAACCAT
GGCTTCCTCAGTTCTTTCCTCCGCAGCAGTTGCCACCCGCAGCAATGTTG
CTCAAGCTAACATGGTTGCACCTTTCACTGGCCTTAAGTCAGCTGCCTCA
TTCCCTGTTTCAAGGAAGCAAAACCTTGACATCACTTCCATTGCCAGCAA
CGGCGGAAGAGTGCAATGCATGCAGGTGTGGCCAGCGCGTGACCACATGG
TCCTTCATGAGTACGTAAATGCTGCTGGGATTACAGGTGGAGGGTCTGGT
GGCGGATCAATGGCTCCCGATCGGGCCCAAGTAATCATTCGCATTGTGCC
AGGAGGTGGAACCAAGACCCTTCAGCAGATAATCAATCAGCTGGAGTACC
TGTCCCGAAAGGGAAAGCTGGAACTCCAGCGTTCAGCCCGGCATCTCGAT
ATTCCCGTTCCGCCGGATCAAATCCGTGAGCTTGCCCAAAGCTGGGTTAC
GGAGGCCGGGATTTATGACGAAAGTCAGTCAGACGATGACAGGCAACAAG
ACTTAACAACACACATTATTGTGAGCTTCCCCGCAGGTACCGACCAAACC
GCAGCTTATGAAGCAAGCCGGGAATGGGCAGCCGAGATGTTTGGGTCAGG
ATACGGGGGTGGCCGCTATAACTATCTGACAGCCTACCACGTGGACCGCG
ATCATCCACATTTACATGTCGTGGTCAATCGTCGGGAACTTCTGGGGCAG
GGGTGGCTGAAAATATCCAGGCGCCATCCCCAGCTGAATTATGACGGCTT
ACGGAAAAAGATGGCAGAGATTTCACTTCGTCACGGCATAGTCCTGGATG
CGACTTCCCGAGCAGAAAGGGGAATAGCAGAGCGACCAATCACCTATGCT
GAATATCGACGTCTTGAGCGGATGCTCGAAGTTATGGCAGAAGTTCGGCC
CATAGCCCGATCCATTAAAACGGCTCACGACGATGCGCGAGCGGAATTAA
TGTCGGCGGACAGACCTCGATCAACGCGCGGTCTATGATTCTAGAAGAGA
CTCTTATCAAGAATCCCATCTCTTGCTTGCTTTTTTTTGTTGCTTCCCTT
TGATAGGGTTTGTTTTTCTTGTTTCAGTGACTTTCTATGTTAAAAGATAA
TGTCAGTAAAAGGATTTGGTTTTCTATTATTCTGAATCGATTACGGAAGA
TTCTTGCTTAATTCCAATCTATACAAGTATCGTGAAATAATGACCGTTTA
TGTAAGCTTGGCACTGGCCGTCGTTTTACAACGTCGTGACTGGGAAAACC
CTGGCGTTACCCAACTTAATCGCCTTGCAGCACATCCCCCTTTCGCCAGC
TGGCGTAATAGCGAAGAGGCCCGCACCGATCGCCCTTCCCAACAGTTGCG
CAGCCTGAATGGCGAATGCTAGAGCAGCTTGAGCTTGGATCAGATTGTCG
TTTCCCGCCTTCAGTTTAAACTATCAGTGTTTGACAGGATATATTGGCGG
GTAAACCTAAGAGAAAAGAGCGTTTATTAGAATAACGGATATTTAAAAGG
GCGTGAAAAGGTTTATCCGTTCGTCCATTTGTATGTGCATGCCAACCACA
GGGTTCCCCTCGGGATCAA

>SEQ ID NO 17 pJF9

AGTACTTTGATCCAACCCCTCCGCTGCTATAGTGCAGTCGGCTTCTGACG
TTCAGTGCAGCCGTCTTCTGAAAACGACATGTCGCACAAGTCCTAAGTTA
CGCGACAGGCTGCCGCCCTGCCCTTTTCCTGGCGTTTTCTTGTCGCGTGT
TTTAGTCGCATAAAGTAGAATACTTGCGACTAGAACCGGAGACATTACGC
CATGAACAAGAGCGCCGCCGCTGGCCTGCTGGGCTATGCCCGCGTCAGCA
CCGACGACCAGGACTTGACCAACCAACGGGCCGAACTGCACGCGGCCGGC
TGCACCAAGCTGTTTTCCGAGAAGATCACCGGCACCAGGCGCGACCGCCC
GGAGCTGGCCAGGATGCTTGACCACCTACGCCCTGGCGACGTTGTGACAG
TGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCC
GTGGGCCGACACCACCACGCCGGCCGGCCGCATGGTGTTGACCGTGTTCG
CCGGCATTGCCGAGTTCGAGCGTTCCCTAATCATCGACCGCACCCGGAGC
GGGCGCGAGGCCGCCAAGGCCCGAGGCGTGAAGTTTGGCCCCCGCCCTAC
CCTCACCCCGGCACAGATCGCGCACGCCCGCGAGCTGATCGACCAGGAAG
GCCGCACCGTGAAAGAGGCGGCTGCACTGCTTGGCGTGCATCGCTCGACC
CTGTACCGCGCACTTGAGCGCAGCGAGGAAGTGACGCCCACCGAGGCCAG
GCGGCGCGGTGCCTTCCGTGAGGACGCATTGACCGAGGCCGACGCCCTGG
CGGCCGCCGAGAATGAACGCCAAGAGGAACAAGCATGAAACCGCACCAGG
ACGGCCAGGACGAACCGTTTTTCATTACCGAAGAGATCGAGGCGGAGATG
ATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGC
CGGCCAGCTTGGCCGCTGAAGAAACCGAGCGCCGCCGTCTAAAAAGGTGA
TGTGTATTTGAGTAAAACAGCTTGCGTCATGCGGTCGCTGCGTATATGAT
GCGATGAGTAAATAAACAAATACGCAAGGGGAACGCATGAAGGTTATCGC
TGTACTTAACCAGAAAGGCGGGTCAGGCAAGACGACCATCGCAACCCATC
TAGCCCGCGCCCTGCAACTCGCCGGGGCCGATGTTCTGTTAGTCGATTCC
GATCCCCAGGGCAGTGCCCGCGATTGGGCGGCCGTGCGGGAAGATCAACC
GCTAACCGTTGTCGGCATCGACCGCCCGACGATTGACCGCGACGTGAAGG
CCATCGGCCGGCGCGACTTCGTAGTGATCGACGGAGCGCCCCAGGCGGCG
GACTTGGCTGTGTCCGCGATCAAGGCAGCCGACTTCGTGCTGATTCCGGT
GCAGCCAAGCCCTTACGACATATGGGCCACCGCCGACCTGGTGGAGCTGG
TTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCG
TGAGCTACCCAGGCACTGCCGCCGCCGGCACAACCGTTCTTGAATCAGAA
CCCGAGGGCGACGCTGCCCGCGAGGTCCAGGCGCTGGCCGCTGAAATTAA
ATCAAAACTCATTTGAGTTAATGAGGTAAAGAGAAAATGAGCAAAAGCAC
AAACACGCTAAGTGCCGGCCGTCCGAGCGCACGCAGCAGCAAGGCTGCAA
CGTTGGCCAGCCTGGCAGACACGCCAGCCATGAAGCGGGTCAACTTTCAG
TTGCCGGCGGAGGATCACACCAAGCTGAAGATGTACGCGGTACGCCAAGG
CAAGACCATTACCGAGCTGCTATCTGAATACATCGCGCAGCTACCAGAGT
AAATGAGCAAATGAATAAATGAGTAGATGAATTTTAGCGGCTAAAGGAGG
CGGCATGGAAAATCAAGAACAACCAGGCACCGACGCCGTGGAATGCCCCA
TGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGAGC
GGTCGCAAACCATCCGGCCCGGTACAAATCGGCGCGGCGCTGGGTGATGA
CCTGGTGGAGAAGTTGAAGGCCGCGCAGGCCGCCCAGCGGCAACGCATCG
AGGCAGAAGCACGCCCCGGTGAATCGTGGCAAGCGGCCGCTGATCGAATC
CGCAAAGAATCCCGGCAACCGCCGGCAGCCGGTGCGCCGTCGATTAGGAA
GCCGCCCAAGGGCGACGAGCAACCAGATTTTTTCGTTCCGATGCTCTATG
ACGTGGGCACCCGCGATAGTCGCAGCATCATGGACGTGGCCGTTTTCCGT
CTGTCGAAGCGTGACCGACGAGCTGGCGAGGTGATCCGCTACGAGCTTCC
AGACGGGCACGTAGAGGTTTCCGCAGGGCCGGCCGGCATGGCCAGTGTGT
GGGATTACGACCTGGTACTGATGGCGGTTTCCCATCTAACCGAATCCATG
AACCGATACCGGGAAGGGAAGGGAGACAAGCCCGGCCGCGTGTTCCGTCC
ACACGTTGCGGACGTACTCAAGTTCTGCCGGCGAGCCGATGGCGGAAAGC
AGAAAGACGACCTGGTAGAAACCTGCATTCGGTTAAACACCACGCACGTT
GCCATGCAGCGTACGAAGAAGGCCAAGAACGGCCGCCTGGTGACGGTATC
CGAGGGTGAAGCCTTGATTAGCCGCTACAAGATCGTAAAGAGCGAAACCG
GGCGGCCGGAGTACATCGAGATCGAGCTAGCTGATTGGATGTACCGCGAG
ATCACAGAAGGCAAGAACCCGGACGTGCTGACGGTTCACCCCGATTACTT
TTTGATCGATCCCGGCATCGGCCGTTTTCTCTACCGCCTGGCACGCCGCG
CCGCAGGCAAGGCAGAAGCCAGATGGTTGTTCAAGACGATCTACGAACGC
AGTGGCAGCGCCGGAGAGTTCAAGAAGTTCTGTTTCACCGTGCGCAAGCT
GATCGGGTCAAATGACCTGCCGGAGTACGATTTGAAGGAGGAGGCGGGGC
AGGCTGGCCCGATCCTAGTCATGCGCTACCGCAACCTGATCGAGGGCGAA
GCATCCGCCGGTTCCTAATGTACGGAGCAGATGCTAGGGCAAATTGCCCT
AGCAGGGGAAAAAGGTCGAAAAGGTCTCTTTCCTGTGGATAGCACGTACA
TTGGGAACCCAAAGCCGTACATTGGGAACCGGAACCCGTACATTGGGAAC
CCAAAGCCGTACATTGGGAACCGGTCACACATGTAAGTGACTGATATAAA
AGAGAAAAAAGGCGATTTTTCCGCCTAAAACTCTTTAAAACTTATTAAAA
CTCTTAAAACCCGCCTGGCCTGTGCATAACTGTCTGGCCAGCGCACAGCC
GAAGAGCTGCAAAAAGCGCCTACCCTTCGGTCGCTGCGCTCCCTACGCCC
CGCCGCTTCGCGTCGGCCTATCGCGGCCGCTGGCCGCTCAAAAATGGCTG
GCCTACGGCCAGGCAATCTACCAGGGCGCGGACAAGCCGCGCCGTCGCCA
CTCGACCGCCGGCGCCCACATCAAGGCACCCTGCCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATA
GCGGAGTGTATACTGGCTTAACTATGCGGCATCAGAGCAGATTGTACTGA
GAGTGCACCATATGCGGTGTGAAATACCGCACAGATGCGTAAGGAGAAAA
TACCGCATCAGGCGCTCTTCCGCTTCCTCGCTCACTGACTCGCTGCGCTC
GGTCGTTCGGCTGCGGCGAGCGGTATCAGCTCACTCAAAGGCGGTAATAC
GGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTA
TCGTCTTGAGTCCAACCCGGTAAGACACGACTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGA
TTTTGGTCATGCATGATATATCTCCCAATTTGTGTAGGGCTTATTATGCA
CGCTTAAAAATAATAAAAGCAGACTTGACCTGATAGTTTGGCTGTGAGCA
ATTATGTGCTTAGTGCATCTAATCGCTTGAGTTAACGCCGGCGAAGCGGC
GTCGGCTTGAACGAATTTCTAGCTAGACATTATTTGCCGACTACCTTGGT
GATCTCGCCTTTCACGTAGTGGACAAATTCTTCCAACTGATCTGCGCGCG
AGGCCAAGCGATCTTCTTCTTGTCCAAGATAAGCCTGTCTAGCTTCAAGT
ATGACGGGCTGATACTGGGCCGGCAGGCGCTCCATTGCCCAGTCGGCAGC
GACATCCTTCGGCGCGATTTTGCCGGTTACTGCGCTGTACCAAATGCGGG
ACAACGTAAGCACTACATTTCGCTCATCGCCAGCCCAGTCGGGCGGCGAG
TTCCATAGCGTTAAGGTTTCATTTAGCGCCTCAAATAGATCCTGTTCAGG
AACCGGATCAAAGAGTTCCTCCGCCGCTGGACCTACCAAGGCAACGCTAT
GTTCTCTTGCTTTTGTCAGCAAGATAGCCAGATCAATGTCGATCGTGGCT
GGCTCGAAGATACCTGCAAGAATGTCATTGCGCTGCCATTCTCCAAATTG
CAGTTCGCGCTTAGCTGGATAACGCCACGGAATGATGTCGTCGTGCACAA
CAATGGTGACTTCTACAGCGCGGAGAATCTCGCTCTCTCCAGGGGAAGCC
GAAGTTTCCAAAAGGTCGTTGATCAAAGCTCGCCGCGTTGTTTCATCAAG
CCTTACGGTCACCGTAACCAGCAAATCAATATCACTGTGTGGCTTCAGGC
CGCCATCCACTGCGGAGCCGTACAAATGTACGGCCAGCAACGTCGGTTCG
AGATGGCGCTCGATGACGCCAACTACCTCTGATAGTTGAGTCGATACTTC
GGCGATCACCGCTTCCCCCATGATGTTTAACTTTGTTTTAGGGCGACTGC
CCTGCTGCGTAACATCGTTGCTGCTCCATAACATCAAACATCGACCCACG
GCGTAACGCGCTTGCTGCTTGGATGCCCGAGGCATAGACTGTACCCCAAA
AAAACATGTCATAACAAGAAGCCATGAAAACCGCCACTGCGCCGTTACCA
CCGCTGCGTTCGGTCAAGGTTCTGGACCAGTTGCGTGACGGCAGTTACGC
TACTTGCATTACAGCTTACGAACCGAACGAGGCTTATGTCCACTGGGTTC
GTGCCCGAATTGATCACAGGCAGCAACGCTCTGTCATCGTTACAATCAAC
ATGCTACCCTCCGCGAGATCATCCGTGTTTCAAACCCGGCAGCTTAGTTG
CCGTTCTTCCGAATAGCATCGGTAACATGAGCAAAGTCTGCCGCCTTACA
ACGGCTCTCCCGCTGACGCCGTCCCGGACTGATGGGCTGCCTGTATCGAG
TGGTGATTTTGTGCCGAGCTGCCGGTCGGGGAGCTGTTGGCTGGCTGGTG
GCAGGATATATTGTGGTGTAAACAAATTGACGCTTAGACAACTTAATAAC
ACATTGCGGACGTTTTTAATGTACTGAATTAACGCCGAATTAATTCGGGG
GATCTATAGGGACTTTAGGTGATCTGGATTTTAGTACTGGATTTTGGTTT
TAGGAATTAGAAATTTTATTGATAGAAGTATTTTACAAATACAAATACAT
ACTAAGGGTTTCTTATATGCTCAACACGTGAGCGAAACCCTATAAGAACC
CTAATTCCCTTATCTGGGAACTACTCACACATTATTATGGAGAAACTCGA
TGTCGATCGACTCTAGCTAGAACGAATTGTTAGGTGGCGGTACTTGGGTC
GATATCAAAGTGCATCACTTCTTCCCGTATGCCCAACTTTGTATAGAGAG
CCACTGCGGGATCGTCACCGTAATCTGCTTGCACGTAGATCACATAAGCA
CCAAGCGCGTTGGCCTCATGCTTGAGGAGATTGATGAGCGCGGTGGCAAT
GCCCTGCCTCCGGTGCTCGCCGGAGACTGCGAGATCATAGATATAGATCT
CACTACGCGGCTGCTCAAACCTGGGCAGAACGTAAGCCGCGAGAGCGCCA
ACAACCGCTTCTTGGTCGAAGGCAGCAAGCGCGATGAATGTCTTACTACG
GAGCAAGTTCCCGAGGTAATCGGAGTCCGGCTGATGTTGGGAGTAGGTGG
CTACGTCTCCGAACTCACGACCGAAAAGATCAAGAGCAGCCCGCATGGAT
TTGACTTGGTCAGGGCCGAGCCTACATGTGCGAATGATGCCCATACTTGA
GCCACCTAACTTTGTTTTAGGGCGACTGCCCTGCTGCGTAACATCGTTGC
TGCTGCGTACCATGGTCGATCGACAGATCTGCGAATCGAGAGAGATAGAT
TTGTAGAGAGAGACTGGTGATTTCAGCGTGTCCTCTCCAAATGAAATGAA
CTTCCTTATATAGAGGAAGGGTCTTGCGAAGGATAGTGGGATTGTGCGTC
ATCCCTTACGTCAGTGGAGATATCACATCAATCCACTTGCTTTGAAGACG
TGGTTGGAACGTCTTCTTTTTCCACGATGCTCCTCGTGGGTGGGGGTCCA
TCTTTGGGACCACTGTCGGCAGAGGCATCTTGAACGATAGCCTTTCCTTT
ATCGCAATGATGGCATTTGTAGGTGCCACCTTCCTTTTCTACTGTCCTTT
TGATGAAGTGACAGATAGCTGGGCAATGGAATCCGAGGAGGTTTCCCGAT
ATTACCCTTTGTTGAAAAGTCTCAATAGCCCTTTGGTCTTCTGAGACTGT
ATCTTTGATATTCTTGGAGTAGACGAGAGTGTCGTGCTCCACCATGTTAT
CACATCAATCCACTTGCTTTGAAGACGTGGTTGGAACGTCTTCTTTTTCC
ACGATGCTCCTCGTGGGTGGGGGTCCATCTTTGGGACCACTGTCGGCAGA
GGCATCTTGAACGATAGCCTTTCCTTTATCGCAATGATGGCATTTGTAGG
TGCCACCTTCCTTTTCTACTGTCCTTTTGATGAAGTGACAGATAGCTGGG
CAATGGAATCCGAGGAGGTTTCCCGATATTACCCTTTGTTGAAAAGTCTC
AATAGCCCTTTGGTCTTCTGAGACTGTATCTTTGATATTCTTGGAGTAGA
CGAGAGTGTCGTGCTCCACCATGTTGGCAAGCTGCTCTAGCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGAATTCGGGATATTTCACAAATTGAACATAGACTA
CAGAATTTTAGAAAACAAACTTTCTCTCTCTTATCTCACCTTTATCTTTT
AGAGAGAAAAAGTTCGATTTCCGGTTGACCGGAATGTATCTTTGTTTTTT
TTGTTTTGTAACATATTTCGTTTTCCGATTTAGATCGGATCTCCTTTTCC
GTTTTGTCGGACCTTCTTCCGGTTTATCCGGATCTAATAATATCCATCTT
AGACTTAGCTAAGTTTGGATCTGTTTTTTGGTTAGCTCTTGTCAATCGCC
TCATCATCAGCAAGAAGGTGAAATTTTTGACAAATAAATCTTAGAATCAT
GTAGTGTCTTTGGACCTTGGGAATGATAGAAACGATTTGTTATAGCTACT
CTATGTATCAGACCCTGACCAAGATCCAACAATCTCATAGGTTTTGTGCA
TATGAAACCTTCGACTAACGAGAAGTGGTCTTTTAATGAGAGAGATATCT
AAAATGTTATCTTAAAAGCCCACTCAAATCTCAAGGCATAAGGTAGAAAT
GCAAATTTGGAAAGTGGGCTGGGCCTTTTGTGGTAAAGGCCTGTAACCTA
GCCCAATATTAGCAAAACCCTAGACGCGTACATTGACATATATAAACCCG
CCTCCTCCTTGTTTAGGGTTTCTACGTGAGAGAGACGAAACACAAACCAT
GGCTTCCTCAGTTCTTTCCTCCGCAGCAGTTGCCACCCGCAGCAATGTTG
CTCAAGCTAACATGGTTGCACCTTTCACTGGCCTTAAGTCAGCTGCCTCA
TTCCCTGTTTCAAGGAAGCAAAACCTTGACATCACTTCCATTGCCAGCAA
CGGCGGAAGAGTGCAATGCATGCAGGTGTGGCCAGCGATGGCTCCCGATC
GGGCCCAAGTAATCATTCGCATTGTGCCAGGAGGTGGAACCAAGACCCTT
CAGCAGATAATCAATCAGCTGGAGTACCTGTCCCGAAAGGGAAAGCTGGA
ACTCCAGCGTTCAGCCCGGCATCTCGATATTCCCGTTCCGCCGGATCAAA
TCCGTGAGCTTGCCCAAAGCTGGGTTACGGAGGCCGGGATTTATGACGAA
AGTCAGTCAGACGATGACAGGCAACAAGACTTAACAACACACATTATTGT
GAGCTTCCCCGCAGGTACCGACCAAACCGCAGCTTATGAAGCAAGCCGGG
AATGGGCAGCCGAGATGTTTGGGTCAGGATACGGGGGTGGCCGCTATAAC
TATCTGACAGCCTACCACGTGGACCGCGATCATCCACATTTACATGTCGT
GGTCAATCGTCGGGAACTTCTGGGGCAGGGGTGGCTGAAAATATCCAGGC
GCCATCCCCAGCTGAATTATGACGGCTTACGGAAAAAGATGGCAGAGATT
TCACTTCGTCACGGCATAGTCCTGGATGCGACTTCCCGAGCAGAAAGGGG
AATAGCAGAGCGACCAATCACCTATGCTGAATATCGACGTCTTGAGCGGA
TGCGTGACCACATGGTCCTTCATGAGTACGTAAATGCTGCTGGGATTACA
CTCGAAGTTATGGCAGAAGTTCGGCCCATAGCCCGATCCATTAAAACGGC
TCACGACGATGCGCGAGCGGAATTAATGTCGGCGGACAGACCTCGATCAA
CGCGCGGTCTATGATTCTAGAAGAGACTCTTATCAAGAATCCCATCTCTT
GCTTGCTTTTTTTTGTTGCTTCCCTTTGATAGGGTTTGTTTTTCTTGTTT
CAGTGACTTTCTATGTTAAAAGATAATGTCAGTAAAAGGATTTGGTTTTC
TATTATTCTGAATCGATTACGGAAGATTCTTGCTTAATTCCAATCTATAC
AAGTATCGTGAAATAATGACCGTTTATGTAAGCTTGGCACTGGCCGTCGT
TTTACAACGTCGTGACTGGGAAAACCCTGGCGTTACCCAACTTAATCGCC
TTGCAGCACATCCCCCTTTCGCCAGCTGGCGTAATAGCGAAGAGGCCCGC
ACCGATCGCCCTTCCCAACAGTTGCGCAGCCTGAATGGCGAATGCTAGAG
CAGCTTGAGCTTGGATCAGATTGTCGTTTCCCGCCTTCAGTTTAAACTAT
CAGTGTTTGACAGGATATATTGGCGGGTAAACCTAAGAGAAAAGAGCGTT
TATTAGAATAACGGATATTTAAAAGGGCGTGAAAAGGTTTATCCGTTCGT
CCATTTGTATGTGCATGCCAACCACAGGGTTCCCCTCGGGATCAA

>SEQ ID NO 18 pJF10

AGTACTTTGATCCAACCCCTCCGCTGCTATAGTGCAGTCGGCTTCTGACG
TTCAGTGCAGCCGTCTTCTGAAAACGACATGTCGCACAAGTCCTAAGTTA
CGCGACAGGCTGCCGCCCTGCCCTTTTCCTGGCGTTTTCTTGTCGCGTGT
TTTAGTCGCATAAAGTAGAATACTTGCGACTAGAACCGGAGACATTACGC
CATGAACAAGAGCGCCGCCGCTGGCCTGCTGGGCTATGCCCGCGTCAGCA
CCGACGACCAGGACTTGACCAACCAACGGGCCGAACTGCACGCGGCCGGC
TGCACCAAGCTGTTTTCCGAGAAGATCACCGGCACCAGGCGCGACCGCCC
GGAGCTGGCCAGGATGCTTGACCACCTACGCCCTGGCGACGTTGTGACAG
TGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCC
GTGGGCCGACACCACCACGCCGGCCGGCCGCATGGTGTTGACCGTGTTCG
CCGGCATTGCCGAGTTCGAGCGTTCCCTAATCATCGACCGCACCCGGAGC
GGGCGCGAGGCCGCCAAGGCCCGAGGCGTGAAGTTTGGCCCCCGCCCTAC
CCTCACCCCGGCACAGATCGCGCACGCCCGCGAGCTGATCGACCAGGAAG
GCCGCACCGTGAAAGAGGCGGCTGCACTGCTTGGCGTGCATCGCTCGACC
CTGTACCGCGCACTTGAGCGCAGCGAGGAAGTGACGCCCACCGAGGCCAG
GCGGCGCGGTGCCTTCCGTGAGGACGCATTGACCGAGGCCGACGCCCTGG
CGGCCGCCGAGAATGAACGCCAAGAGGAACAAGCATGAAACCGCACCAGG
ACGGCCAGGACGAACCGTTTTTCATTACCGAAGAGATCGAGGCGGAGATG
ATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGC
CGGCCAGCTTGGCCGCTGAAGAAACCGAGCGCCGCCGTCTAAAAAGGTGA
TGTGTATTTGAGTAAAACAGCTTGCGTCATGCGGTCGCTGCGTATATGAT
GCGATGAGTAAATAAACAAATACGCAAGGGGAACGCATGAAGGTTATCGC
TGTACTTAACCAGAAAGGCGGGTCAGGCAAGACGACCATCGCAACCCATC
TAGCCCGCGCCCTGCAACTCGCCGGGGCCGATGTTCTGTTAGTCGATTCC
GATCCCCAGGGCAGTGCCCGCGATTGGGCGGCCGTGCGGGAAGATCAACC
GCTAACCGTTGTCGGCATCGACCGCCCGACGATTGACCGCGACGTGAAGG
CCATCGGCCGGCGCGACTTCGTAGTGATCGACGGAGCGCCCCAGGCGGCG
GACTTGGCTGTGTCCGCGATCAAGGCAGCCGACTTCGTGCTGATTCCGGT
GCAGCCAAGCCCTTACGACATATGGGCCACCGCCGACCTGGTGGAGCTGG
TTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCG
TGAGCTACCCAGGCACTGCCGCCGCCGGCACAACCGTTCTTGAATCAGAA
CCCGAGGGCGACGCTGCCCGCGAGGTCCAGGCGCTGGCCGCTGAAATTAA
ATCAAAACTCATTTGAGTTAATGAGGTAAAGAGAAAATGAGCAAAAGCAC
AAACACGCTAAGTGCCGGCCGTCCGAGCGCACGCAGCAGCAAGGCTGCAA
CGTTGGCCAGCCTGGCAGACACGCCAGCCATGAAGCGGGTCAACTTTCAG
TTGCCGGCGGAGGATCACACCAAGCTGAAGATGTACGCGGTACGCCAAGG
CAAGACCATTACCGAGCTGCTATCTGAATACATCGCGCAGCTACCAGAGT
AAATGAGCAAATGAATAAATGAGTAGATGAATTTTAGCGGCTAAAGGAGG
CGGCATGGAAAATCAAGAACAACCAGGCACCGACGCCGTGGAATGCCCCA
TGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGAGC
GGTCGCAAACCATCCGGCCCGGTACAAATCGGCGCGGCGCTGGGTGATGA
CCTGGTGGAGAAGTTGAAGGCCGCGCAGGCCGCCCAGCGGCAACGCATCG
AGGCAGAAGCACGCCCCGGTGAATCGTGGCAAGCGGCCGCTGATCGAATC
CGCAAAGAATCCCGGCAACCGCCGGCAGCCGGTGCGCCGTCGATTAGGAA
GCCGCCCAAGGGCGACGAGCAACCAGATTTTTTCGTTCCGATGCTCTATG
ACGTGGGCACCCGCGATAGTCGCAGCATCATGGACGTGGCCGTTTTCCGT
CTGTCGAAGCGTGACCGACGAGCTGGCGAGGTGATCCGCTACGAGCTTCC
AGACGGGCACGTAGAGGTTTCCGCAGGGCCGGCCGGCATGGCCAGTGTGT
GGGATTACGACCTGGTACTGATGGCGGTTTCCCATCTAACCGAATCCATG
AACCGATACCGGGAAGGGAAGGGAGACAAGCCCGGCCGCGTGTTCCGTCC
ACACGTTGCGGACGTACTCAAGTTCTGCCGGCGAGCCGATGGCGGAAAGC
AGAAAGACGACCTGGTAGAAACCTGCATTCGGTTAAACACCACGCACGTT
GCCATGCAGCGTACGAAGAAGGCCAAGAACGGCCGCCTGGTGACGGTATC
CGAGGGTGAAGCCTTGATTAGCCGCTACAAGATCGTAAAGAGCGAAACCG
GGCGGCCGGAGTACATCGAGATCGAGCTAGCTGATTGGATGTACCGCGAG
ATCACAGAAGGCAAGAACCCGGACGTGCTGACGGTTCACCCCGATTACTT
TTTGATCGATCCCGGCATCGGCCGTTTTCTCTACCGCCTGGCACGCCGCG
CCGCAGGCAAGGCAGAAGCCAGATGGTTGTTCAAGACGATCTACGAACGC
AGTGGCAGCGCCGGAGAGTTCAAGAAGTTCTGTTTCACCGTGCGCAAGCT
GATCGGGTCAAATGACCTGCCGGAGTACGATTTGAAGGAGGAGGCGGGGC
AGGCTGGCCCGATCCTAGTCATGCGCTACCGCAACCTGATCGAGGGCGAA
GCATCCGCCGGTTCCTAATGTACGGAGCAGATGCTAGGGCAAATTGCCCT
AGCAGGGGAAAAAGGTCGAAAAGGTCTCTTTCCTGTGGATAGCACGTACA
TTGGGAACCCAAAGCCGTACATTGGGAACCGGAACCCGTACATTGGGAAC
CCAAAGCCGTACATTGGGAACCGGTCACACATGTAAGTGACTGATATAAA
AGAGAAAAAAGGCGATTTTTCCGCCTAAAACTCTTTAAAACTTATTAAAA
CTCTTAAAACCCGCCTGGCCTGTGCATAACTGTCTGGCCAGCGCACAGCC
GAAGAGCTGCAAAAAGCGCCTACCCTTCGGTCGCTGCGCTCCCTACGCCC
CGCCGCTTCGCGTCGGCCTATCGCGGCCGCTGGCCGCTCAAAAATGGCTG
GCCTACGGCCAGGCAATCTACCAGGGCGCGGACAAGCCGCGCCGTCGCCA
CTCGACCGCCGGCGCCCACATCAAGGCACCCTGCCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATA
GCGGAGTGTATACTGGCTTAACTATGCGGCATCAGAGCAGATTGTACTGA
GAGTGCACCATATGCGGTGTGAAATACCGCACAGATGCGTAAGGAGAAAA
TACCGCATCAGGCGCTCTTCCGCTTCCTCGCTCACTGACTCGCTGCGCTC
GGTCGTTCGGCTGCGGCGAGCGGTATCAGCTCACTCAAAGGCGGTAATAC
GGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTA
TCGTCTTGAGTCCAACCCGGTAAGACACGACTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGA
TTTTGGTCATGCATGATATATCTCCCAATTTGTGTAGGGCTTATTATGCA
CGCTTAAAAATAATAAAAGCAGACTTGACCTGATAGTTTGGCTGTGAGCA
ATTATGTGCTTAGTGCATCTAATCGCTTGAGTTAACGCCGGCGAAGCGGC
GTCGGCTTGAACGAATTTCTAGCTAGACATTATTTGCCGACTACCTTGGT
GATCTCGCCTTTCACGTAGTGGACAAATTCTTCCAACTGATCTGCGCGCG
AGGCCAAGCGATCTTCTTCTTGTCCAAGATAAGCCTGTCTAGCTTCAAGT
ATGACGGGCTGATACTGGGCCGGCAGGCGCTCCATTGCCCAGTCGGCAGC
GACATCCTTCGGCGCGATTTTGCCGGTTACTGCGCTGTACCAAATGCGGG
ACAACGTAAGCACTACATTTCGCTCATCGCCAGCCCAGTCGGGCGGCGAG
TTCCATAGCGTTAAGGTTTCATTTAGCGCCTCAAATAGATCCTGTTCAGG
AACCGGATCAAAGAGTTCCTCCGCCGCTGGACCTACCAAGGCAACGCTAT
GTTCTCTTGCTTTTGTCAGCAAGATAGCCAGATCAATGTCGATCGTGGCT
GGCTCGAAGATACCTGCAAGAATGTCATTGCGCTGCCATTCTCCAAATTG
CAGTTCGCGCTTAGCTGGATAACGCCACGGAATGATGTCGTCGTGCACAA
CAATGGTGACTTCTACAGCGCGGAGAATCTCGCTCTCTCCAGGGGAAGCC
GAAGTTTCCAAAAGGTCGTTGATCAAAGCTCGCCGCGTTGTTTCATCAAG
CCTTACGGTCACCGTAACCAGCAAATCAATATCACTGTGTGGCTTCAGGC
CGCCATCCACTGCGGAGCCGTACAAATGTACGGCCAGCAACGTCGGTTCG
AGATGGCGCTCGATGACGCCAACTACCTCTGATAGTTGAGTCGATACTTC
GGCGATCACCGCTTCCCCCATGATGTTTAACTTTGTTTTAGGGCGACTGC
CCTGCTGCGTAACATCGTTGCTGCTCCATAACATCAAACATCGACCCACG
GCGTAACGCGCTTGCTGCTTGGATGCCCGAGGCATAGACTGTACCCCAAA
AAAACATGTCATAACAAGAAGCCATGAAAACCGCCACTGCGCCGTTACCA
CCGCTGCGTTCGGTCAAGGTTCTGGACCAGTTGCGTGACGGCAGTTACGC
TACTTGCATTACAGCTTACGAACCGAACGAGGCTTATGTCCACTGGGTTC
GTGCCCGAATTGATCACAGGCAGCAACGCTCTGTCATCGTTACAATCAAC
ATGCTACCCTCCGCGAGATCATCCGTGTTTCAAACCCGGCAGCTTAGTTG
CCGTTCTTCCGAATAGCATCGGTAACATGAGCAAAGTCTGCCGCCTTACA
ACGGCTCTCCCGCTGACGCCGTCCCGGACTGATGGGCTGCCTGTATCGAG
TGGTGATTTTGTGCCGAGCTGCCGGTCGGGGAGCTGTTGGCTGGCTGGTG
GCAGGATATATTGTGGTGTAAACAAATTGACGCTTAGACAACTTAATAAC
ACATTGCGGACGTTTTTAATGTACTGAATTAACGCCGAATTAATTCGGGG
GATCTATAGGGACTTTAGGTGATCTGGATTTTAGTACTGGATTTTGGTTT
TAGGAATTAGAAATTTTATTGATAGAAGTATTTTACAAATACAAATACAT
ACTAAGGGTTTCTTATATGCTCAACACGTGAGCGAAACCCTATAAGAACC
CTAATTCCCTTATCTGGGAACTACTCACACATTATTATGGAGAAACTCGA
TGTCGATCGACTCTAGCTAGAACGAATTGTTAGGTGGCGGTACTTGGGTC
GATATCAAAGTGCATCACTTCTTCCCGTATGCCCAACTTTGTATAGAGAG
CCACTGCGGGATCGTCACCGTAATCTGCTTGCACGTAGATCACATAAGCA
CCAAGCGCGTTGGCCTCATGCTTGAGGAGATTGATGAGCGCGGTGGCAAT
GCCCTGCCTCCGGTGCTCGCCGGAGACTGCGAGATCATAGATATAGATCT
CACTACGCGGCTGCTCAAACCTGGGCAGAACGTAAGCCGCGAGAGCGCCA
ACAACCGCTTCTTGGTCGAAGGCAGCAAGCGCGATGAATGTCTTACTACG
GAGCAAGTTCCCGAGGTAATCGGAGTCCGGCTGATGTTGGGAGTAGGTGG
CTACGTCTCCGAACTCACGACCGAAAAGATCAAGAGCAGCCCGCATGGAT
TTGACTTGGTCAGGGCCGAGCCTACATGTGCGAATGATGCCCATACTTGA
GCCACCTAACTTTGTTTTAGGGCGACTGCCCTGCTGCGTAACATCGTTGC
TGCTGCGTACCATGGTCGATCGACAGATCTGCGAATCGAGAGAGATAGAT
TTGTAGAGAGAGACTGGTGATTTCAGCGTGTCCTCTCCAAATGAAATGAA
CTTCCTTATATAGAGGAAGGGTCTTGCGAAGGATAGTGGGATTGTGCGTC
ATCCCTTACGTCAGTGGAGATATCACATCAATCCACTTGCTTTGAAGACG
TGGTTGGAACGTCTTCTTTTTCCACGATGCTCCTCGTGGGTGGGGGTCCA
TCTTTGGGACCACTGTCGGCAGAGGCATCTTGAACGATAGCCTTTCCTTT
ATCGCAATGATGGCATTTGTAGGTGCCACCTTCCTTTTCTACTGTCCTTT
TGATGAAGTGACAGATAGCTGGGCAATGGAATCCGAGGAGGTTTCCCGAT
ATTACCCTTTGTTGAAAAGTCTCAATAGCCCTTTGGTCTTCTGAGACTGT
ATCTTTGATATTCTTGGAGTAGACGAGAGTGTCGTGCTCCACCATGTTAT
CACATCAATCCACTTGCTTTGAAGACGTGGTTGGAACGTCTTCTTTTTCC
ACGATGCTCCTCGTGGGTGGGGGTCCATCTTTGGGACCACTGTCGGCAGA
GGCATCTTGAACGATAGCCTTTCCTTTATCGCAATGATGGCATTTGTAGG
TGCCACCTTCCTTTTCTACTGTCCTTTTGATGAAGTGACAGATAGCTGGG
CAATGGAATCCGAGGAGGTTTCCCGATATTACCCTTTGTTGAAAAGTCTC
AATAGCCCTTTGGTCTTCTGAGACTGTATCTTTGATATTCTTGGAGTAGA
CGAGAGTGTCGTGCTCCACCATGTTGGCAAGCTGCTCTAGCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGAATTCGGGATATTTCACAAATTGAACATAGACTA
CAGAATTTTAGAAAACAAACTTTCTCTCTCTTATCTCACCTTTATCTTTT
AGAGAGAAAAAGTTCGATTTCCGGTTGACCGGAATGTATCTTTGTTTTTT
TTGTTTTGTAACATATTTCGTTTTCCGATTTAGATCGGATCTCCTTTTCC
GTTTTGTCGGACCTTCTTCCGGTTTATCCGGATCTAATAATATCCATCTT
AGACTTAGCTAAGTTTGGATCTGTTTTTTGGTTAGCTCTTGTCAATCGCC
TCATCATCAGCAAGAAGGTGAAATTTTTGACAAATAAATCTTAGAATCAT
GTAGTGTCTTTGGACCTTGGGAATGATAGAAACGATTTGTTATAGCTACT
CTATGTATCAGACCCTGACCAAGATCCAACAATCTCATAGGTTTTGTGCA
TATGAAACCTTCGACTAACGAGAAGTGGTCTTTTAATGAGAGAGATATCT
AAAATGTTATCTTAAAAGCCCACTCAAATCTCAAGGCATAAGGTAGAAAT
GCAAATTTGGAAAGTGGGCTGGGCCTTTTGTGGTAAAGGCCTGTAACCTA
GCCCAATATTAGCAAAACCCTAGACGCGTACATTGACATATATAAACCCG
CCTCCTCCTTGTTTAGGGTTTCTACGTGAGAGAGACGAAACACAAACCAT
GGCTTCCTCAGTTCTTTCCTCCGCAGCAGTTGCCACCCGCAGCAATGTTG
CTCAAGCTAACATGGTTGCACCTTTCACTGGCCTTAAGTCAGCTGCCTCA
TTCCCTGTTTCAAGGAAGCAAAACCTTGACATCACTTCCATTGCCAGCAA
CGGCGGAAGAGTGCAATGCATGCAGGTGTGGCCAGCGATGGCTCCCGATC
GGGCCCAAGTAATCATTCGCATTGTGCCAGGAGGTGGAACCAAGACCCTT
CAGCAGATAATCAATCAGCTGGAGTACCTGTCCCGAAAGGGAAAGCTGGA
ACTCCAGCGTTCAGCCCGGCATCTCGATATTCCCGTTCCGCCGGATCAAA
TCCGTGAGCTTGCCCAAAGCTGGGTTACGGAGGCCGGGATTTATGACGAA
AGTCAGTCAGACGATGACAGGCAACAAGACTTAACAACACACATTATTGT
GAGCTTCCCCGCAGGTACCGACCAAACCGCAGCTTATGAAGCAAGCCGGG
AATGGGCAGCCGAGATGTTTGGGTCAGGATACGGGGGTGGCCGCTATAAC
TATCTGACAGCCTACCACGTGGACCGCGATCATCCACATTTACATGTCGT
GGTCAATCGTCGGGAACTTCTGGGGCAGGGGTGGCTGAAAATATCCAGGC
GCCATCCCCAGCTGAATTATGACGGCTTACGGAAAAAGATGGCAGAGATT
TCACTTCGTCACGGCATAGTCCTGGATGCGACTTCCCGAGCAGAAAGGGG
AATAGCAGAGCGACCAATCACCTATGCTGAATATCGACGTCTTGAGCGGA
TGGGTGGAGGGTCTGGTGGCGGATCACGTGACCACATGGTCCTTCATGAG
TACGTAAATGCTGCTGGGATTACACTCGAAGTTATGGCAGAAGTTCGGCC
CATAGCCCGATCCATTAAAACGGCTCACGACGATGCGCGAGCGGAATTAA
TGTCGGCGGACAGACCTCGATCAACGCGCGGTCTATGATTCTAGAAGAGA
CTCTTATCAAGAATCCCATCTCTTGCTTGCTTTTTTTTGTTGCTTCCCTT
TGATAGGGTTTGTTTTTCTTGTTTCAGTGACTTTCTATGTTAAAAGATAA
TGTCAGTAAAAGGATTTGGTTTTCTATTATTCTGAATCGATTACGGAAGA
TTCTTGCTTAATTCCAATCTATACAAGTATCGTGAAATAATGACCGTTTA
TGTAAGCTTGGCACTGGCCGTCGTTTTACAACGTCGTGACTGGGAAAACC
CTGGCGTTACCCAACTTAATCGCCTTGCAGCACATCCCCCTTTCGCCAGC
TGGCGTAATAGCGAAGAGGCCCGCACCGATCGCCCTTCCCAACAGTTGCG
CAGCCTGAATGGCGAATGCTAGAGCAGCTTGAGCTTGGATCAGATTGTCG
TTTCCCGCCTTCAGTTTAAACTATCAGTGTTTGACAGGATATATTGGCGG
GTAAACCTAAGAGAAAAGAGCGTTTATTAGAATAACGGATATTTAAAAGG
GCGTGAAAAGGTTTATCCGTTCGTCCATTTGTATGTGCATGCCAACCACA
GGGTTCCCCTCGGGATCAA

>SEQ ID NO 19 pJF1 1

AGTACTTTGATCCAACCCCTCCGCTGCTATAGTGCAGTCGGCTTCTGACG
TTCAGTGCAGCCGTCTTCTGAAAACGACATGTCGCACAAGTCCTAAGTTA
CGCGACAGGCTGCCGCCCTGCCCTTTTCCTGGCGTTTTCTTGTCGCGTGT
TTTAGTCGCATAAAGTAGAATACTTGCGACTAGAACCGGAGACATTACGC
CATGAACAAGAGCGCCGCCGCTGGCCTGCTGGGCTATGCCCGCGTCAGCA
CCGACGACCAGGACTTGACCAACCAACGGGCCGAACTGCACGCGGCCGGC
TGCACCAAGCTGTTTTCCGAGAAGATCACCGGCACCAGGCGCGACCGCCC
GGAGCTGGCCAGGATGCTTGACCACCTACGCCCTGGCGACGTTGTGACAG
TGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCC
GTGGGCCGACACCACCACGCCGGCCGGCCGCATGGTGTTGACCGTGTTCG
CCGGCATTGCCGAGTTCGAGCGTTCCCTAATCATCGACCGCACCCGGAGC
GGGCGCGAGGCCGCCAAGGCCCGAGGCGTGAAGTTTGGCCCCCGCCCTAC
CCTCACCCCGGCACAGATCGCGCACGCCCGCGAGCTGATCGACCAGGAAG
GCCGCACCGTGAAAGAGGCGGCTGCACTGCTTGGCGTGCATCGCTCGACC
CTGTACCGCGCACTTGAGCGCAGCGAGGAAGTGACGCCCACCGAGGCCAG
GCGGCGCGGTGCCTTCCGTGAGGACGCATTGACCGAGGCCGACGCCCTGG
CGGCCGCCGAGAATGAACGCCAAGAGGAACAAGCATGAAACCGCACCAGG
ACGGCCAGGACGAACCGTTTTTCATTACCGAAGAGATCGAGGCGGAGATG
ATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGC
CGGCCAGCTTGGCCGCTGAAGAAACCGAGCGCCGCCGTCTAAAAAGGTGA
TGTGTATTTGAGTAAAACAGCTTGCGTCATGCGGTCGCTGCGTATATGAT
GCGATGAGTAAATAAACAAATACGCAAGGGGAACGCATGAAGGTTATCGC
TGTACTTAACCAGAAAGGCGGGTCAGGCAAGACGACCATCGCAACCCATC
TAGCCCGCGCCCTGCAACTCGCCGGGGCCGATGTTCTGTTAGTCGATTCC
GATCCCCAGGGCAGTGCCCGCGATTGGGCGGCCGTGCGGGAAGATCAACC
GCTAACCGTTGTCGGCATCGACCGCCCGACGATTGACCGCGACGTGAAGG
CCATCGGCCGGCGCGACTTCGTAGTGATCGACGGAGCGCCCCAGGCGGCG
GACTTGGCTGTGTCCGCGATCAAGGCAGCCGACTTCGTGCTGATTCCGGT
GCAGCCAAGCCCTTACGACATATGGGCCACCGCCGACCTGGTGGAGCTGG
TTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCG
TGAGCTACCCAGGCACTGCCGCCGCCGGCACAACCGTTCTTGAATCAGAA
CCCGAGGGCGACGCTGCCCGCGAGGTCCAGGCGCTGGCCGCTGAAATTAA
ATCAAAACTCATTTGAGTTAATGAGGTAAAGAGAAAATGAGCAAAAGCAC
AAACACGCTAAGTGCCGGCCGTCCGAGCGCACGCAGCAGCAAGGCTGCAA
CGTTGGCCAGCCTGGCAGACACGCCAGCCATGAAGCGGGTCAACTTTCAG
TTGCCGGCGGAGGATCACACCAAGCTGAAGATGTACGCGGTACGCCAAGG
CAAGACCATTACCGAGCTGCTATCTGAATACATCGCGCAGCTACCAGAGT
AAATGAGCAAATGAATAAATGAGTAGATGAATTTTAGCGGCTAAAGGAGG
CGGCATGGAAAATCAAGAACAACCAGGCACCGACGCCGTGGAATGCCCCA
TGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGAGC
GGTCGCAAACCATCCGGCCCGGTACAAATCGGCGCGGCGCTGGGTGATGA
CCTGGTGGAGAAGTTGAAGGCCGCGCAGGCCGCCCAGCGGCAACGCATCG
AGGCAGAAGCACGCCCCGGTGAATCGTGGCAAGCGGCCGCTGATCGAATC
CGCAAAGAATCCCGGCAACCGCCGGCAGCCGGTGCGCCGTCGATTAGGAA
GCCGCCCAAGGGCGACGAGCAACCAGATTTTTTCGTTCCGATGCTCTATG
ACGTGGGCACCCGCGATAGTCGCAGCATCATGGACGTGGCCGTTTTCCGT
CTGTCGAAGCGTGACCGACGAGCTGGCGAGGTGATCCGCTACGAGCTTCC
AGACGGGCACGTAGAGGTTTCCGCAGGGCCGGCCGGCATGGCCAGTGTGT
GGGATTACGACCTGGTACTGATGGCGGTTTCCCATCTAACCGAATCCATG
AACCGATACCGGGAAGGGAAGGGAGACAAGCCCGGCCGCGTGTTCCGTCC
ACACGTTGCGGACGTACTCAAGTTCTGCCGGCGAGCCGATGGCGGAAAGC
AGAAAGACGACCTGGTAGAAACCTGCATTCGGTTAAACACCACGCACGTT
GCCATGCAGCGTACGAAGAAGGCCAAGAACGGCCGCCTGGTGACGGTATC
CGAGGGTGAAGCCTTGATTAGCCGCTACAAGATCGTAAAGAGCGAAACCG
GGCGGCCGGAGTACATCGAGATCGAGCTAGCTGATTGGATGTACCGCGAG
ATCACAGAAGGCAAGAACCCGGACGTGCTGACGGTTCACCCCGATTACTT
TTTGATCGATCCCGGCATCGGCCGTTTTCTCTACCGCCTGGCACGCCGCG
CCGCAGGCAAGGCAGAAGCCAGATGGTTGTTCAAGACGATCTACGAACGC
AGTGGCAGCGCCGGAGAGTTCAAGAAGTTCTGTTTCACCGTGCGCAAGCT
GATCGGGTCAAATGACCTGCCGGAGTACGATTTGAAGGAGGAGGCGGGGC
AGGCTGGCCCGATCCTAGTCATGCGCTACCGCAACCTGATCGAGGGCGAA
GCATCCGCCGGTTCCTAATGTACGGAGCAGATGCTAGGGCAAATTGCCCT
AGCAGGGGAAAAAGGTCGAAAAGGTCTCTTTCCTGTGGATAGCACGTACA
TTGGGAACCCAAAGCCGTACATTGGGAACCGGAACCCGTACATTGGGAAC
CCAAAGCCGTACATTGGGAACCGGTCACACATGTAAGTGACTGATATAAA
AGAGAAAAAAGGCGATTTTTCCGCCTAAAACTCTTTAAAACTTATTAAAA
CTCTTAAAACCCGCCTGGCCTGTGCATAACTGTCTGGCCAGCGCACAGCC
GAAGAGCTGCAAAAAGCGCCTACCCTTCGGTCGCTGCGCTCCCTACGCCC
CGCCGCTTCGCGTCGGCCTATCGCGGCCGCTGGCCGCTCAAAAATGGCTG
GCCTACGGCCAGGCAATCTACCAGGGCGCGGACAAGCCGCGCCGTCGCCA
CTCGACCGCCGGCGCCCACATCAAGGCACCCTGCCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATA
GCGGAGTGTATACTGGCTTAACTATGCGGCATCAGAGCAGATTGTACTGA
GAGTGCACCATATGCGGTGTGAAATACCGCACAGATGCGTAAGGAGAAAA
TACCGCATCAGGCGCTCTTCCGCTTCCTCGCTCACTGACTCGCTGCGCTC
GGTCGTTCGGCTGCGGCGAGCGGTATCAGCTCACTCAAAGGCGGTAATAC
GGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTA
TCGTCTTGAGTCCAACCCGGTAAGACACGACTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGA
TTTTGGTCATGCATGATATATCTCCCAATTTGTGTAGGGCTTATTATGCA
CGCTTAAAAATAATAAAAGCAGACTTGACCTGATAGTTTGGCTGTGAGCA
ATTATGTGCTTAGTGCATCTAATCGCTTGAGTTAACGCCGGCGAAGCGGC
GTCGGCTTGAACGAATTTCTAGCTAGACATTATTTGCCGACTACCTTGGT
GATCTCGCCTTTCACGTAGTGGACAAATTCTTCCAACTGATCTGCGCGCG
AGGCCAAGCGATCTTCTTCTTGTCCAAGATAAGCCTGTCTAGCTTCAAGT
ATGACGGGCTGATACTGGGCCGGCAGGCGCTCCATTGCCCAGTCGGCAGC
GACATCCTTCGGCGCGATTTTGCCGGTTACTGCGCTGTACCAAATGCGGG
ACAACGTAAGCACTACATTTCGCTCATCGCCAGCCCAGTCGGGCGGCGAG
TTCCATAGCGTTAAGGTTTCATTTAGCGCCTCAAATAGATCCTGTTCAGG
AACCGGATCAAAGAGTTCCTCCGCCGCTGGACCTACCAAGGCAACGCTAT
GTTCTCTTGCTTTTGTCAGCAAGATAGCCAGATCAATGTCGATCGTGGCT
GGCTCGAAGATACCTGCAAGAATGTCATTGCGCTGCCATTCTCCAAATTG
CAGTTCGCGCTTAGCTGGATAACGCCACGGAATGATGTCGTCGTGCACAA
CAATGGTGACTTCTACAGCGCGGAGAATCTCGCTCTCTCCAGGGGAAGCC
GAAGTTTCCAAAAGGTCGTTGATCAAAGCTCGCCGCGTTGTTTCATCAAG
CCTTACGGTCACCGTAACCAGCAAATCAATATCACTGTGTGGCTTCAGGC
CGCCATCCACTGCGGAGCCGTACAAATGTACGGCCAGCAACGTCGGTTCG
AGATGGCGCTCGATGACGCCAACTACCTCTGATAGTTGAGTCGATACTTC
GGCGATCACCGCTTCCCCCATGATGTTTAACTTTGTTTTAGGGCGACTGC
CCTGCTGCGTAACATCGTTGCTGCTCCATAACATCAAACATCGACCCACG
GCGTAACGCGCTTGCTGCTTGGATGCCCGAGGCATAGACTGTACCCCAAA
AAAACATGTCATAACAAGAAGCCATGAAAACCGCCACTGCGCCGTTACCA
CCGCTGCGTTCGGTCAAGGTTCTGGACCAGTTGCGTGACGGCAGTTACGC
TACTTGCATTACAGCTTACGAACCGAACGAGGCTTATGTCCACTGGGTTC
GTGCCCGAATTGATCACAGGCAGCAACGCTCTGTCATCGTTACAATCAAC
ATGCTACCCTCCGCGAGATCATCCGTGTTTCAAACCCGGCAGCTTAGTTG
CCGTTCTTCCGAATAGCATCGGTAACATGAGCAAAGTCTGCCGCCTTACA
ACGGCTCTCCCGCTGACGCCGTCCCGGACTGATGGGCTGCCTGTATCGAG
TGGTGATTTTGTGCCGAGCTGCCGGTCGGGGAGCTGTTGGCTGGCTGGTG
GCAGGATATATTGTGGTGTAAACAAATTGACGCTTAGACAACTTAATAAC
ACATTGCGGACGTTTTTAATGTACTGAATTAACGCCGAATTAATTCGGGG
GATCTATAGGGACTTTAGGTGATCTGGATTTTAGTACTGGATTTTGGTTT
TAGGAATTAGAAATTTTATTGATAGAAGTATTTTACAAATACAAATACAT
ACTAAGGGTTTCTTATATGCTCAACACGTGAGCGAAACCCTATAAGAACC
CTAATTCCCTTATCTGGGAACTACTCACACATTATTATGGAGAAACTCGA
TGTCGATCGACTCTAGCTAGAACGAATTGTTAGGTGGCGGTACTTGGGTC
GATATCAAAGTGCATCACTTCTTCCCGTATGCCCAACTTTGTATAGAGAG
CCACTGCGGGATCGTCACCGTAATCTGCTTGCACGTAGATCACATAAGCA
CCAAGCGCGTTGGCCTCATGCTTGAGGAGATTGATGAGCGCGGTGGCAAT
GCCCTGCCTCCGGTGCTCGCCGGAGACTGCGAGATCATAGATATAGATCT
CACTACGCGGCTGCTCAAACCTGGGCAGAACGTAAGCCGCGAGAGCGCCA
ACAACCGCTTCTTGGTCGAAGGCAGCAAGCGCGATGAATGTCTTACTACG
GAGCAAGTTCCCGAGGTAATCGGAGTCCGGCTGATGTTGGGAGTAGGTGG
CTACGTCTCCGAACTCACGACCGAAAAGATCAAGAGCAGCCCGCATGGAT
TTGACTTGGTCAGGGCCGAGCCTACATGTGCGAATGATGCCCATACTTGA
GCCACCTAACTTTGTTTTAGGGCGACTGCCCTGCTGCGTAACATCGTTGC
TGCTGCGTACCATGGTCGATCGACAGATCTGCGAATCGAGAGAGATAGAT
TTGTAGAGAGAGACTGGTGATTTCAGCGTGTCCTCTCCAAATGAAATGAA
CTTCCTTATATAGAGGAAGGGTCTTGCGAAGGATAGTGGGATTGTGCGTC
ATCCCTTACGTCAGTGGAGATATCACATCAATCCACTTGCTTTGAAGACG
TGGTTGGAACGTCTTCTTTTTCCACGATGCTCCTCGTGGGTGGGGGTCCA
TCTTTGGGACCACTGTCGGCAGAGGCATCTTGAACGATAGCCTTTCCTTT
ATCGCAATGATGGCATTTGTAGGTGCCACCTTCCTTTTCTACTGTCCTTT
TGATGAAGTGACAGATAGCTGGGCAATGGAATCCGAGGAGGTTTCCCGAT
ATTACCCTTTGTTGAAAAGTCTCAATAGCCCTTTGGTCTTCTGAGACTGT
ATCTTTGATATTCTTGGAGTAGACGAGAGTGTCGTGCTCCACCATGTTAT
CACATCAATCCACTTGCTTTGAAGACGTGGTTGGAACGTCTTCTTTTTCC
ACGATGCTCCTCGTGGGTGGGGGTCCATCTTTGGGACCACTGTCGGCAGA
GGCATCTTGAACGATAGCCTTTCCTTTATCGCAATGATGGCATTTGTAGG
TGCCACCTTCCTTTTCTACTGTCCTTTTGATGAAGTGACAGATAGCTGGG
CAATGGAATCCGAGGAGGTTTCCCGATATTACCCTTTGTTGAAAAGTCTC
AATAGCCCTTTGGTCTTCTGAGACTGTATCTTTGATATTCTTGGAGTAGA
CGAGAGTGTCGTGCTCCACCATGTTGGCAAGCTGCTCTAGCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGAATTCGGGATATTTCACAAATTGAACATAGACTA
CAGAATTTTAGAAAACAAACTTTCTCTCTCTTATCTCACCTTTATCTTTT
AGAGAGAAAAAGTTCGATTTCCGGTTGACCGGAATGTATCTTTGTTTTTT
TTGTTTTGTAACATATTTCGTTTTCCGATTTAGATCGGATCTCCTTTTCC
GTTTTGTCGGACCTTCTTCCGGTTTATCCGGATCTAATAATATCCATCTT
AGACTTAGCTAAGTTTGGATCTGTTTTTTGGTTAGCTCTTGTCAATCGCC
TCATCATCAGCAAGAAGGTGAAATTTTTGACAAATAAATCTTAGAATCAT
GTAGTGTCTTTGGACCTTGGGAATGATAGAAACGATTTGTTATAGCTACT
CTATGTATCAGACCCTGACCAAGATCCAACAATCTCATAGGTTTTGTGCA
TATGAAACCTTCGACTAACGAGAAGTGGTCTTTTAATGAGAGAGATATCT
AAAATGTTATCTTAAAAGCCCACTCAAATCTCAAGGCATAAGGTAGAAAT
GCAAATTTGGAAAGTGGGCTGGGCCTTTTGTGGTAAAGGCCTGTAACCTA
GCCCAATATTAGCAAAACCCTAGACGCGTACATTGACATATATAAACCCG
CCTCCTCCTTGTTTAGGGTTTCTACGTGAGAGAGACGAAACACAAACCAT
GGCTTCCTCAGTTCTTTCCTCCGCAGCAGTTGCCACCCGCAGCAATGTTG
CTCAAGCTAACATGGTTGCACCTTTCACTGGCCTTAAGTCAGCTGCCTCA
TTCCCTGTTTCAAGGAAGCAAAACCTTGACATCACTTCCATTGCCAGCAA
CGGCGGAAGAGTGCAATGCATGCAGGTGTGGCCAGCGATGGCTCCCGATC
GGGCCCAAGTAATCATTCGCATTGTGCCAGGAGGTGGAACCAAGACCCTT
CAGCAGATAATCAATCAGCTGGAGTACCTGTCCCGAAAGGGAAAGCTGGA
ACTCCAGCGTTCAGCCCGGCATCTCGATATTCCCGTTCCGCCGGATCAAA
TCCGTGAGCTTGCCCAAAGCTGGGTTACGGAGGCCGGGATTTATGACGAA
AGTCAGTCAGACGATGACAGGCAACAAGACTTAACAACACACATTATTGT
GAGCTTCCCCGCAGGTACCGACCAAACCGCAGCTTATGAAGCAAGCCGGG
AATGGGCAGCCGAGATGTTTGGGTCAGGATACGGGGGTGGCCGCTATAAC
TATCTGACAGCCTACCACGTGGACCGCGATCATCCACATTTACATGTCGT
GGTCAATCGTCGGGAACTTCTGGGGCAGGGGTGGCTGAAAATATCCAGGC
GCCATCCCCAGCTGAATTATGACGGCTTACGGAAAAAGATGGCAGAGATT
TCACTTCGTCACGGCATAGTCCTGGATGCGACTTCCCGAGCAGAAAGGGG
AATAGCAGAGCGACCAATCACCTATGCTGAATATCGACGTCTTGAGCGGA
TGCGTGACCACATGGTCCTTCATGAGTACGTAAATGCTGCTGGGATTACA
GGTGGAGGGTCTGGTGGCGGATCACTCGAAGTTATGGCAGAAGTTCGGCC
CATAGCCCGATCCATTAAAACGGCTCACGACGATGCGCGAGCGGAATTAA
TGTCGGCGGACAGACCTCGATCAACGCGCGGTCTATGATTCTAGAAGAGA
CTCTTATCAAGAATCCCATCTCTTGCTTGCTTTTTTTTGTTGCTTCCCTT
TGATAGGGTTTGTTTTTCTTGTTTCAGTGACTTTCTATGTTAAAAGATAA
TGTCAGTAAAAGGATTTGGTTTTCTATTATTCTGAATCGATTACGGAAGA
TTCTTGCTTAATTCCAATCTATACAAGTATCGTGAAATAATGACCGTTTA
TGTAAGCTTGGCACTGGCCGTCGTTTTACAACGTCGTGACTGGGAAAACC
CTGGCGTTACCCAACTTAATCGCCTTGCAGCACATCCCCCTTTCGCCAGC
TGGCGTAATAGCGAAGAGGCCCGCACCGATCGCCCTTCCCAACAGTTGCG
CAGCCTGAATGGCGAATGCTAGAGCAGCTTGAGCTTGGATCAGATTGTCG
TTTCCCGCCTTCAGTTTAAACTATCAGTGTTTGACAGGATATATTGGCGG
GTAAACCTAAGAGAAAAGAGCGTTTATTAGAATAACGGATATTTAAAAGG
GCGTGAAAAGGTTTATCCGTTCGTCCATTTGTATGTGCATGCCAACCACA
GGGTTCCCCTCGGGATCAA

> SEQ ID NO 20 pJF12

AGTACTTTGATCCAACCCCTCCGCTGCTATAGTGCAGTCGGCTTCTGACG
TTCAGTGCAGCCGTCTTCTGAAAACGACATGTCGCACAAGTCCTAAGTTA
CGCGACAGGCTGCCGCCCTGCCCTTTTCCTGGCGTTTTCTTGTCGCGTGT
TTTAGTCGCATAAAGTAGAATACTTGCGACTAGAACCGGAGACATTACGC
CATGAACAAGAGCGCCGCCGCTGGCCTGCTGGGCTATGCCCGCGTCAGCA
CCGACGACCAGGACTTGACCAACCAACGGGCCGAACTGCACGCGGCCGGC
TGCACCAAGCTGTTTTCCGAGAAGATCACCGGCACCAGGCGCGACCGCCC
GGAGCTGGCCAGGATGCTTGACCACCTACGCCCTGGCGACGTTGTGACAG
TGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCC
GTGGGCCGACACCACCACGCCGGCCGGCCGCATGGTGTTGACCGTGTTCG
CCGGCATTGCCGAGTTCGAGCGTTCCCTAATCATCGACCGCACCCGGAGC
GGGCGCGAGGCCGCCAAGGCCCGAGGCGTGAAGTTTGGCCCCCGCCCTAC
CCTCACCCCGGCACAGATCGCGCACGCCCGCGAGCTGATCGACCAGGAAG
GCCGCACCGTGAAAGAGGCGGCTGCACTGCTTGGCGTGCATCGCTCGACC
CTGTACCGCGCACTTGAGCGCAGCGAGGAAGTGACGCCCACCGAGGCCAG
GCGGCGCGGTGCCTTCCGTGAGGACGCATTGACCGAGGCCGACGCCCTGG
CGGCCGCCGAGAATGAACGCCAAGAGGAACAAGCATGAAACCGCACCAGG
ACGGCCAGGACGAACCGTTTTTCATTACCGAAGAGATCGAGGCGGAGATG
ATCGCGGCCGGGTACGTGTTCGAGCCGCCCGCGCACGTCTCAACCGTGCG
GCTGCATGAAATCCTGGCCGGTTTGTCTGATGCCAAGCTGGCGGCCTGGC
CGGCCAGCTTGGCCGCTGAAGAAACCGAGCGCCGCCGTCTAAAAAGGTGA
TGTGTATTTGAGTAAAACAGCTTGCGTCATGCGGTCGCTGCGTATATGAT
GCGATGAGTAAATAAACAAATACGCAAGGGGAACGCATGAAGGTTATCGC
TGTACTTAACCAGAAAGGCGGGTCAGGCAAGACGACCATCGCAACCCATC
TAGCCCGCGCCCTGCAACTCGCCGGGGCCGATGTTCTGTTAGTCGATTCC
GATCCCCAGGGCAGTGCCCGCGATTGGGCGGCCGTGCGGGAAGATCAACC
GCTAACCGTTGTCGGCATCGACCGCCCGACGATTGACCGCGACGTGAAGG
CCATCGGCCGGCGCGACTTCGTAGTGATCGACGGAGCGCCCCAGGCGGCG
GACTTGGCTGTGTCCGCGATCAAGGCAGCCGACTTCGTGCTGATTCCGGT
GCAGCCAAGCCCTTACGACATATGGGCCACCGCCGACCTGGTGGAGCTGG
TTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGC
GCTGGCCGGGTACGAGCTGCCCATTCTTGAGTCCCGTATCACGCAGCGCG
TGAGCTACCCAGGCACTGCCGCCGCCGGCACAACCGTTCTTGAATCAGAA
CCCGAGGGCGACGCTGCCCGCGAGGTCCAGGCGCTGGCCGCTGAAATTAA
ATCAAAACTCATTTGAGTTAATGAGGTAAAGAGAAAATGAGCAAAAGCAC
AAACACGCTAAGTGCCGGCCGTCCGAGCGCACGCAGCAGCAAGGCTGCAA
CGTTGGCCAGCCTGGCAGACACGCCAGCCATGAAGCGGGTCAACTTTCAG
TTGCCGGCGGAGGATCACACCAAGCTGAAGATGTACGCGGTACGCCAAGG
CAAGACCATTACCGAGCTGCTATCTGAATACATCGCGCAGCTACCAGAGT
AAATGAGCAAATGAATAAATGAGTAGATGAATTTTAGCGGCTAAAGGAGG
CGGCATGGAAAATCAAGAACAACCAGGCACCGACGCCGTGGAATGCCCCA
TGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGAGC
GGTCGCAAACCATCCGGCCCGGTACAAATCGGCGCGGCGCTGGGTGATGA
CCTGGTGGAGAAGTTGAAGGCCGCGCAGGCCGCCCAGCGGCAACGCATCG
AGGCAGAAGCACGCCCCGGTGAATCGTGGCAAGCGGCCGCTGATCGAATC
CGCAAAGAATCCCGGCAACCGCCGGCAGCCGGTGCGCCGTCGATTAGGAA
GCCGCCCAAGGGCGACGAGCAACCAGATTTTTTCGTTCCGATGCTCTATG
ACGTGGGCACCCGCGATAGTCGCAGCATCATGGACGTGGCCGTTTTCCGT
CTGTCGAAGCGTGACCGACGAGCTGGCGAGGTGATCCGCTACGAGCTTCC
AGACGGGCACGTAGAGGTTTCCGCAGGGCCGGCCGGCATGGCCAGTGTGT
GGGATTACGACCTGGTACTGATGGCGGTTTCCCATCTAACCGAATCCATG
AACCGATACCGGGAAGGGAAGGGAGACAAGCCCGGCCGCGTGTTCCGTCC
ACACGTTGCGGACGTACTCAAGTTCTGCCGGCGAGCCGATGGCGGAAAGC
AGAAAGACGACCTGGTAGAAACCTGCATTCGGTTAAACACCACGCACGTT
GCCATGCAGCGTACGAAGAAGGCCAAGAACGGCCGCCTGGTGACGGTATC
CGAGGGTGAAGCCTTGATTAGCCGCTACAAGATCGTAAAGAGCGAAACCG
GGCGGCCGGAGTACATCGAGATCGAGCTAGCTGATTGGATGTACCGCGAG
ATCACAGAAGGCAAGAACCCGGACGTGCTGACGGTTCACCCCGATTACTT
TTTGATCGATCCCGGCATCGGCCGTTTTCTCTACCGCCTGGCACGCCGCG
CCGCAGGCAAGGCAGAAGCCAGATGGTTGTTCAAGACGATCTACGAACGC
AGTGGCAGCGCCGGAGAGTTCAAGAAGTTCTGTTTCACCGTGCGCAAGCT
GATCGGGTCAAATGACCTGCCGGAGTACGATTTGAAGGAGGAGGCGGGGC
AGGCTGGCCCGATCCTAGTCATGCGCTACCGCAACCTGATCGAGGGCGAA
GCATCCGCCGGTTCCTAATGTACGGAGCAGATGCTAGGGCAAATTGCCCT
AGCAGGGGAAAAAGGTCGAAAAGGTCTCTTTCCTGTGGATAGCACGTACA
TTGGGAACCCAAAGCCGTACATTGGGAACCGGAACCCGTACATTGGGAAC
CCAAAGCCGTACATTGGGAACCGGTCACACATGTAAGTGACTGATATAAA
AGAGAAAAAAGGCGATTTTTCCGCCTAAAACTCTTTAAAACTTATTAAAA
CTCTTAAAACCCGCCTGGCCTGTGCATAACTGTCTGGCCAGCGCACAGCC
GAAGAGCTGCAAAAAGCGCCTACCCTTCGGTCGCTGCGCTCCCTACGCCC
CGCCGCTTCGCGTCGGCCTATCGCGGCCGCTGGCCGCTCAAAAATGGCTG
GCCTACGGCCAGGCAATCTACCAGGGCGCGGACAAGCCGCGCCGTCGCCA
CTCGACCGCCGGCGCCCACATCAAGGCACCCTGCCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATA
GCGGAGTGTATACTGGCTTAACTATGCGGCATCAGAGCAGATTGTACTGA
GAGTGCACCATATGCGGTGTGAAATACCGCACAGATGCGTAAGGAGAAAA
TACCGCATCAGGCGCTCTTCCGCTTCCTCGCTCACTGACTCGCTGCGCTC
GGTCGTTCGGCTGCGGCGAGCGGTATCAGCTCACTCAAAGGCGGTAATAC
GGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTA
TCGTCTTGAGTCCAACCCGGTAAGACACGACTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGA
TTTTGGTCATGCATGATATATCTCCCAATTTGTGTAGGGCTTATTATGCA
CGCTTAAAAATAATAAAAGCAGACTTGACCTGATAGTTTGGCTGTGAGCA
ATTATGTGCTTAGTGCATCTAATCGCTTGAGTTAACGCCGGCGAAGCGGC
GTCGGCTTGAACGAATTTCTAGCTAGACATTATTTGCCGACTACCTTGGT
GATCTCGCCTTTCACGTAGTGGACAAATTCTTCCAACTGATCTGCGCGCG
AGGCCAAGCGATCTTCTTCTTGTCCAAGATAAGCCTGTCTAGCTTCAAGT
ATGACGGGCTGATACTGGGCCGGCAGGCGCTCCATTGCCCAGTCGGCAGC
GACATCCTTCGGCGCGATTTTGCCGGTTACTGCGCTGTACCAAATGCGGG
ACAACGTAAGCACTACATTTCGCTCATCGCCAGCCCAGTCGGGCGGCGAG
TTCCATAGCGTTAAGGTTTCATTTAGCGCCTCAAATAGATCCTGTTCAGG
AACCGGATCAAAGAGTTCCTCCGCCGCTGGACCTACCAAGGCAACGCTAT
GTTCTCTTGCTTTTGTCAGCAAGATAGCCAGATCAATGTCGATCGTGGCT
GGCTCGAAGATACCTGCAAGAATGTCATTGCGCTGCCATTCTCCAAATTG
CAGTTCGCGCTTAGCTGGATAACGCCACGGAATGATGTCGTCGTGCACAA
CAATGGTGACTTCTACAGCGCGGAGAATCTCGCTCTCTCCAGGGGAAGCC
GAAGTTTCCAAAAGGTCGTTGATCAAAGCTCGCCGCGTTGTTTCATCAAG
CCTTACGGTCACCGTAACCAGCAAATCAATATCACTGTGTGGCTTCAGGC
CGCCATCCACTGCGGAGCCGTACAAATGTACGGCCAGCAACGTCGGTTCG
AGATGGCGCTCGATGACGCCAACTACCTCTGATAGTTGAGTCGATACTTC
GGCGATCACCGCTTCCCCCATGATGTTTAACTTTGTTTTAGGGCGACTGC
CCTGCTGCGTAACATCGTTGCTGCTCCATAACATCAAACATCGACCCACG
GCGTAACGCGCTTGCTGCTTGGATGCCCGAGGCATAGACTGTACCCCAAA
AAAACATGTCATAACAAGAAGCCATGAAAACCGCCACTGCGCCGTTACCA
CCGCTGCGTTCGGTCAAGGTTCTGGACCAGTTGCGTGACGGCAGTTACGC
TACTTGCATTACAGCTTACGAACCGAACGAGGCTTATGTCCACTGGGTTC
GTGCCCGAATTGATCACAGGCAGCAACGCTCTGTCATCGTTACAATCAAC
ATGCTACCCTCCGCGAGATCATCCGTGTTTCAAACCCGGCAGCTTAGTTG
CCGTTCTTCCGAATAGCATCGGTAACATGAGCAAAGTCTGCCGCCTTACA
ACGGCTCTCCCGCTGACGCCGTCCCGGACTGATGGGCTGCCTGTATCGAG
TGGTGATTTTGTGCCGAGCTGCCGGTCGGGGAGCTGTTGGCTGGCTGGTG
GCAGGATATATTGTGGTGTAAACAAATTGACGCTTAGACAACTTAATAAC
ACATTGCGGACGTTTTTAATGTACTGAATTAACGCCGAATTAATTCGGGG
GATCTATAGGGACTTTAGGTGATCTGGATTTTAGTACTGGATTTTGGTTT
TAGGAATTAGAAATTTTATTGATAGAAGTATTTTACAAATACAAATACAT
ACTAAGGGTTTCTTATATGCTCAACACGTGAGCGAAACCCTATAAGAACC
CTAATTCCCTTATCTGGGAACTACTCACACATTATTATGGAGAAACTCGA
TGTCGATCGACTCTAGCTAGAACGAATTGTTAGGTGGCGGTACTTGGGTC
GATATCAAAGTGCATCACTTCTTCCCGTATGCCCAACTTTGTATAGAGAG
CCACTGCGGGATCGTCACCGTAATCTGCTTGCACGTAGATCACATAAGCA
CCAAGCGCGTTGGCCTCATGCTTGAGGAGATTGATGAGCGCGGTGGCAAT
GCCCTGCCTCCGGTGCTCGCCGGAGACTGCGAGATCATAGATATAGATCT
CACTACGCGGCTGCTCAAACCTGGGCAGAACGTAAGCCGCGAGAGCGCCA
ACAACCGCTTCTTGGTCGAAGGCAGCAAGCGCGATGAATGTCTTACTACG
GAGCAAGTTCCCGAGGTAATCGGAGTCCGGCTGATGTTGGGAGTAGGTGG
CTACGTCTCCGAACTCACGACCGAAAAGATCAAGAGCAGCCCGCATGGAT
TTGACTTGGTCAGGGCCGAGCCTACATGTGCGAATGATGCCCATACTTGA
GCCACCTAACTTTGTTTTAGGGCGACTGCCCTGCTGCGTAACATCGTTGC
TGCTGCGTACCATGGTCGATCGACAGATCTGCGAATCGAGAGAGATAGAT
TTGTAGAGAGAGACTGGTGATTTCAGCGTGTCCTCTCCAAATGAAATGAA
CTTCCTTATATAGAGGAAGGGTCTTGCGAAGGATAGTGGGATTGTGCGTC
ATCCCTTACGTCAGTGGAGATATCACATCAATCCACTTGCTTTGAAGACG
TGGTTGGAACGTCTTCTTTTTCCACGATGCTCCTCGTGGGTGGGGGTCCA
TCTTTGGGACCACTGTCGGCAGAGGCATCTTGAACGATAGCCTTTCCTTT
ATCGCAATGATGGCATTTGTAGGTGCCACCTTCCTTTTCTACTGTCCTTT
TGATGAAGTGACAGATAGCTGGGCAATGGAATCCGAGGAGGTTTCCCGAT
ATTACCCTTTGTTGAAAAGTCTCAATAGCCCTTTGGTCTTCTGAGACTGT
ATCTTTGATATTCTTGGAGTAGACGAGAGTGTCGTGCTCCACCATGTTAT
CACATCAATCCACTTGCTTTGAAGACGTGGTTGGAACGTCTTCTTTTTCC
ACGATGCTCCTCGTGGGTGGGGGTCCATCTTTGGGACCACTGTCGGCAGA
GGCATCTTGAACGATAGCCTTTCCTTTATCGCAATGATGGCATTTGTAGG
TGCCACCTTCCTTTTCTACTGTCCTTTTGATGAAGTGACAGATAGCTGGG
CAATGGAATCCGAGGAGGTTTCCCGATATTACCCTTTGTTGAAAAGTCTC
AATAGCCCTTTGGTCTTCTGAGACTGTATCTTTGATATTCTTGGAGTAGA
CGAGAGTGTCGTGCTCCACCATGTTGGCAAGCTGCTCTAGCCAATACGCA
AACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAG
TTAGCTCACTCATTAGGCACCCCAGGCTTTACACTTTATGCTTCCGGCTC
GTATGTTGTGTGGAATTGTGAGCGGATAACAATTTCACACAGGAAACAGC
TATGACCATGATTACGAATTCGGGATATTTCACAAATTGAACATAGACTA
CAGAATTTTAGAAAACAAACTTTCTCTCTCTTATCTCACCTTTATCTTTT
AGAGAGAAAAAGTTCGATTTCCGGTTGACCGGAATGTATCTTTGTTTTTT
TTGTTTTGTAACATATTTCGTTTTCCGATTTAGATCGGATCTCCTTTTCC
GTTTTGTCGGACCTTCTTCCGGTTTATCCGGATCTAATAATATCCATCTT
AGACTTAGCTAAGTTTGGATCTGTTTTTTGGTTAGCTCTTGTCAATCGCC
TCATCATCAGCAAGAAGGTGAAATTTTTGACAAATAAATCTTAGAATCAT
GTAGTGTCTTTGGACCTTGGGAATGATAGAAACGATTTGTTATAGCTACT
CTATGTATCAGACCCTGACCAAGATCCAACAATCTCATAGGTTTTGTGCA
TATGAAACCTTCGACTAACGAGAAGTGGTCTTTTAATGAGAGAGATATCT
AAAATGTTATCTTAAAAGCCCACTCAAATCTCAAGGCATAAGGTAGAAAT
GCAAATTTGGAAAGTGGGCTGGGCCTTTTGTGGTAAAGGCCTGTAACCTA
GCCCAATATTAGCAAAACCCTAGACGCGTACATTGACATATATAAACCCG
CCTCCTCCTTGTTTAGGGTTTCTACGTGAGAGAGACGAAACACAAACCAT
GGCTTCCTCAGTTCTTTCCTCCGCAGCAGTTGCCACCCGCAGCAATGTTG
CTCAAGCTAACATGGTTGCACCTTTCACTGGCCTTAAGTCAGCTGCCTCA
TTCCCTGTTTCAAGGAAGCAAAACCTTGACATCACTTCCATTGCCAGCAA
CGGCGGAAGAGTGCAATGCATGCAGGTGTGGCCAGCGATGGCTCCCGATC
GGGCCCAAGTAATCATTCGCATTGTGCCAGGAGGTGGAACCAAGACCCTT
CAGCAGATAATCAATCAGCTGGAGTACCTGTCCCGAAAGGGAAAGCTGGA
ACTCCAGCGTTCAGCCCGGCATCTCGATATTCCCGTTCCGCCGGATCAAA
TCCGTGAGCTTGCCCAAAGCTGGGTTACGGAGGCCGGGATTTATGACGAA
AGTCAGTCAGACGATGACAGGCAACAAGACTTAACAACACACATTATTGT
GAGCTTCCCCGCAGGTACCGACCAAACCGCAGCTTATGAAGCAAGCCGGG
AATGGGCAGCCGAGATGTTTGGGTCAGGATACGGGGGTGGCCGCTATAAC
TATCTGACAGCCTACCACGTGGACCGCGATCATCCACATTTACATGTCGT
GGTCAATCGTCGGGAACTTCTGGGGCAGGGGTGGCTGAAAATATCCAGGC
GCCATCCCCAGCTGAATTATGACGGCTTACGGAAAAAGATGGCAGAGATT
TCACTTCGTCACGGCATAGTCCTGGATGCGACTTCCCGAGCAGAAAGGGG
AATAGCAGAGCGACCAATCACCTATGCTGAATATCGACGTCTTGAGCGGA
TGGGTGGAGGGTCTCGTGACCACATGGTCCTTCATGAGTACGTAAATGCT
GCTGGGATTACAGGTGGCGGATCACTCGAAGTTATGGCAGAAGTTCGGCC
CATAGCCCGATCCATTAAAACGGCTCACGACGATGCGCGAGCGGAATTAA
TGTCGGCGGACAGACCTCGATCAACGCGCGGTCTATGATTCTAGAAGAGA
CTCTTATCAAGAATCCCATCTCTTGCTTGCTTTTTTTTGTTGCTTCCCTT
TGATAGGGTTTGTTTTTCTTGTTTCAGTGACTTTCTATGTTAAAAGATAA
TGTCAGTAAAAGGATTTGGTTTTCTATTATTCTGAATCGATTACGGAAGA
TTCTTGCTTAATTCCAATCTATACAAGTATCGTGAAATAATGACCGTTTA
TGTAAGCTTGGCACTGGCCGTCGTTTTACAACGTCGTGACTGGGAAAACC
CTGGCGTTACCCAACTTAATCGCCTTGCAGCACATCCCCCTTTCGCCAGC
TGGCGTAATAGCGAAGAGGCCCGCACCGATCGCCCTTCCCAACAGTTGCG
CAGCCTGAATGGCGAATGCTAGAGCAGCTTGAGCTTGGATCAGATTGTCG
TTTCCCGCCTTCAGTTTAAACTATCAGTGTTTGACAGGATATATTGGCGG
GTAAACCTAAGAGAAAAGAGCGTTTATTAGAATAACGGATATTTAAAAGG
GCGTGAAAAGGTTTATCCGTTCGTCCATTTGTATGTGCATGCCAACCACA
GGGTTCCCCTCGGGATCAA

> SEQ ID NO 21 Deleted virD2 sequence in XYA105DvirD2

atgcccgatcgcgctcaagtaatcattcgcattgtgccaggaggtggaac
caagacccttcagcagataatcaatcagctggagtacctgtcccgaaagg
gaaagctggaactgcagcgttcagcccggcatctcgatattcccgttccg
ccggatcaaatccgtgagcttgcccaaagctgggttacggaggccgggat
ttatgacgaaagtcagtcagacgatgacaggcaacaagacttaacaacac
acattattgtaagcttccccgcaggtaccgaccaaaccgcagcttatgaa
gcaagccgggaatgggcagccgagatgtttgggtcaggatacgggggtgg
ccgctataactatctgacagcctaccacgtcgaccgcgatcatccacatt
tacatgtcgtggtcaatcgtcgggaacttctggggcaggggtggctgaaa
atatccaggcgccatccccagctgaattatgacggcttacggaaaaagat
ggcagagatttcacttcgtcacggcatagtcctggatgcgacttcgcgag
cagaaaggggaatagcagagcgaccaatcacatatgctgaatatcgacgc
cttgagcggatgcaggctcaaaagattcaattcgaagatacagattttga
tgagacctcgcctgaggaagatcgtcgggacctcagtcaatcgttcgatc
catttcgatcggacgcatctgccggcgaaccggaccgtgcaacccgacat
gacaaacaaccgcttgaaccgcacgcccgtttccaggagcccgccggctc
cagcatcaaagccgacgcacggatccgcgtaccattggagagcgagcggg
gtgcccaaccatccgcgtccaaaatccctgtaactgggcatttcgggatt
gagacttcgtatgtcgctgaagccagcgtgcccaaacaaagcggcaattc
cgatacttctcgcccggtgactgacgttgccatgcacacagtcgagcgcc
agcagcgatcaaaacgacgtcatgacgaggaggcaggtccgagcggagca
aaccgtaaaagattgaaggccgcgcaagttgattccgaggcaaatgtcgg
tgagcccgacggtcgcgatgacagcaacaaggcggctgatccggtgtctg
cttccatccgtaccgagcaaccggaagcttctccaacgtgtccgcgtgac
cgtcacgatggagaattgggagaacgcaaacgtgcaagaggtaatcgtcg
cgacgatgggcgcggggggacctag

> SEQ ID NO 22 DNA sequence of backbone vector pAGa

actttgatccaacccctccgctgctatagtgcagtcggcttctgacgttc
agtgcagccgtcttctgaaaacgacatgtcgcacaagtcctaagttacgc
gacaggctgccgccctgcccttttcctggcgttttcttgtcgcgtgtttt
agtcgcataaagtagaatacttgcgactagaaccggagacattacgccat
gaacaagagcgccgccgctggcctgctgggctatgcccgcgtcagcaccg
acgaccaggacttgaccaaccaacgggccgaactgcacgcggccggctgc
accaagctgttttccgagaagatcaccggcaccaggcgcgaccgcccgga
gctggccaggatgcttgaccacctacgccctggcgacgttgtgacagtga
ccaggctagaccgcctggcccgcagcacccgcgacctactggacattgcc
gagcgcatccaggaggccggcgcgggcctgcgtagcctggcagagccgtg
ggccgacaccaccacgccggccggccgcatggtgttgaccgtgttcgccg
gcattgccgagttcgagcgttccctaatcatcgaccgcacccggagcggg
cgcgaggccgccaaggcccgaggcgtgaagtttggcccccgccctaccct
caccccggcacagatcgcgcacgcccgcgagctgatcgaccaggaaggcc
gcaccgtgaaagaggcggctgcactgcttggcgtgcatcgctcgaccctg
taccgcgcacttgagcgcagcgaggaagtgacgcccaccgaggccaggcg
gcgcggtgccttccgtgaggacgcattgaccgaggccgacgccctggcgg
ccgccgagaatgaacgccaagaggaacaagcatgaaaccgcaccaggacg
gccaggacgaaccgtttttcattaccgaagagatcgaggcggagatgatc
gcggccgggtacgtgttcgagccgcccgcgcacgtctcaaccgtgcggct
gcatgaaatcctggccggtttgtctgatgccaagctggcggcctggccgg
ccagcttggccgctgaagaaaccgagcgccgccgtctaaaaaggtgatgt
gtatttgagtaaaacagcttgcgtcatgcggtcgctgcgtatatgatgcg
atgagtaaataaacaaatacgcaaggggaacgcatgaaggttatcgctgt
acttaaccagaaaggcgggtcaggcaagacgaccatcgcaacccatctag
cccgcgccctgcaactcgccggggccgatgttctgttagtcgattccgat
ccccagggcagtgcccgcgattgggcggccgtgcgggaagatcaaccgct
aaccgttgtcggcatcgaccgcccgacgattgaccgcgacgtgaaggcca
tcggccggcgcgacttcgtagtgatcgacggagcgccccaggcggcggac
ttggctgtgtccgcgatcaaggcagccgacttcgtgctgattccggtgca
gccaagcccttacgacatatgggccaccgccgacctggtggagctggtta
agcagcgcattgaggtcacggatggaaggctacaagcggcctttgtcgtg
tcgcgggcgatcaaaggcacgcgcatcggcggtgaggttgccgaggcgct
ggccgggtacgagctgcccattcttgagtcccgtatcacgcagcgcgtga
gctacccaggcactgccgccgccggcacaaccgttcttgaatcagaaccc
gagggcgacgctgcccgcgaggtccaggcgctggccgctgaaattaaatc
aaaactcatttgagttaatgaggtaaagagaaaatgagcaaaagcacaaa
cacgctaagtgccggccgtccgagcgcacgcagcagcaaggctgcaacgt
tggccagcctggcagacacgccagccatgaagcgggtcaactttcagttg
ccggcggaggatcacaccaagctgaagatgtacgcggtacgccaaggcaa
gaccattaccgagctgctatctgaatacatcgcgcagctaccagagtaaa
tgagcaaatgaataaatgagtagatgaattttagcggctaaaggaggcgg
catggaaaatcaagaacaaccaggcaccgacgccgtggaatgccccatgt
gtggaggaacgggcggttggccaggcgtaagcggctgggttgtctgccgg
ccctgcaatggcactggaacccccaagcccgaggaatcggcgtgagcggt
cgcaaaccatccggcccggtacaaatcggcgcggcgctgggtgatgacct
ggtggagaagttgaaggccgcgcaggccgcccagcggcaacgcatcgagg
cagaagcacgccccggtgaatcgtggcaagcggccgctgatcgaatccgc
aaagaatcccggcaaccgccggcagccggtgcgccgtcgattaggaagcc
gcccaagggcgacgagcaaccagattttttcgttccgatgctctatgacg
tgggcacccgcgatagtcgcagcatcatggacgtggccgttttccgtctg
tcgaagcgtgaccgacgagctggcgaggtgatccgctacgagcttccaga
cgggcacgtagaggtttccgcagggccggccggcatggccagtgtgtggg
attacgacctggtactgatggcggtttcccatctaaccgaatccatgaac
cgataccgggaagggaagggagacaagcccggccgcgtgttccgtccaca
cgttgcggacgtactcaagttctgccggcgagccgatggcggaaagcaga
aagacgacctggtagaaacctgcattcggttaaacaccacgcacgttgcc
atgcagcgtacgaagaaggccaagaacggccgcctggtgacggtatccga
gggtgaagccttgattagccgctacaagatcgtaaagagcgaaaccgggc
ggccggagtacatcgagatcgagctagctgattggatgtaccgcgagatc
acagaaggcaagaacccggacgtgctgacggttcaccccgattacttttt
gatcgatcccggcatcggccgttttctctaccgcctggcacgccgcgccg
caggcaaggcagaagccagatggttgttcaagacgatctacgaacgcagt
ggcagcgccggagagttcaagaagttctgtttcaccgtgcgcaagctgat
cgggtcaaatgacctgccggagtacgatttgaaggaggaggcggggcagg
ctggcccgatcctagtcatgcgctaccgcaacctgatcgagggcgaagca
tccgccggttcctaatgtacggagcagatgctagggcaaattgccctagc
aggggaaaaaggtcgaaaaggtctctttcctgtggatagcacgtacattg
ggaacccaaagccgtacattgggaaccggaacccgtacattgggaaccca
aagccgtacattgggaaccggtcacacatgtaagtgactgatataaaaga
gaaaaaaggcgatttttccgcctaaaactctttaaaacttattaaaactc
ttaaaacccgcctggcctgtgcataactgtctggccagcgcacagccgaa
gagctgcaaaaagcgcctacccttcggtcgctgcgctccctacgccccgc
cgcttcgcgtcggcctatcgcggccgctggccgctcaaaaatggctggcc
tacggccaggcaatctaccagggcgcggacaagccgcgccgtcgccactc
gaccgccggcgcccacatcaaggcaccctgcctcgcgcgtttcggtgatg
acggtgaaaacctctgacacatgcagctcccggagacggtcacagcttgt
ctgtaagcggatgccgggagcagacaagcccgtcagggcgcgtcagcggg
tgttggcgggtgtcggggcgcagccatgacccagtcacgtagcgatagcg
gagtgtatactggcttaactatgcggcatcagagcagattgtactgagag
tgcaccatatgcggtgtgaaataccgcacagatgcgtaaggagaaaatac
cgcatcaggcgctcttccgcttcctcgctcactgactcgctgcgctcggt
cgttcggctgcggcgagcggtatcagctcactcaaaggcggtaatacggt
tatccacagaatcaggggataacgcaggaaagaacatgtgagcaaaaggc
cagcaaaaggccaggaaccgtaaaaaggccgcgttgctggcgtttttcca
taggctccgcccccctgacgagcatcacaaaaatcgacgctcaagtcaga
ggtggcgaaacccgacaggactataaagataccaggcgtttccccctgga
agctccctcgtgcgctctcctgttccgaccctgccgcttaccggatacct
gtccgcctttctcccttcgggaagcgtggcgctttctcatagctcacgct
gtaggtatctcagttcggtgtaggtcgttcgctccaagctgggctgtgtg
cacgaaccccccgttcagcccgaccgctgcgccttatccggtaactatcg
tcttgagtccaacccggtaagacacgacttatcgccactggcagcagcca
ctggtaacaggattagcagagcgaggtatgtaggcggtgctacagagttc
ttgaagtggtggcctaactacggctacactagaaggacagtatttggtat
ctgcgctctgctgaagccagttaccttcggaaaaagagttggtagctctt
gatccggcaaacaaaccaccgctggtagcggtggtttttttgtttgcaag
cagcagattacgcgcagaaaaaaaggatctcaagaagatcctttgatctt
ttctacggggtctgacgctcagtggaacgaaaactcacgttaagggattt
tggtcatgcatGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGCTTGCGCCGTCCCGTCAAGTCAGCGTAATGCTCTGCCA
GTGTTACAACCAATTAACCAATTCTGATTAGAAAAACTCATCGAGCATCA
AATGAAACTGCAATTTATTCACATCAGGATTATCAATACCATATTTTTGA
AAAAGCCGTTTCTGTAATGAAGGAGAAAACTCACCGAGGCAGTTCCATAG
GATGGCAAGATCCTGGTATCGGTCTGCGATTCCGACTCGTCCAACATCAA
TACAACCTATTAATTTCCCCTCGTCAAAAATAAGGTTATCAAGTGAGAAA
TCACCATGAGTGACGACTGAATCCGGTGAGAATGGCAAAAGTTTgTGCAT
TTCTTTCCAGACTTGTTCAACAGGCCAGCCATTACGCTCGTCATCAAAAT
CACTCGCATCAACCAAACCGTTATTCATTCGTGATTGCGCCTGAGCAAGA
CGAAATACGCGATCGCTGTTAAAAGGACAATTACAAACAGGAATCGAATG
CAACCGGCGCAGGAACACTGCCAGCGCATCAACAATATTTTCACCTGAAT
CAGGATATTCTTCTAATACCTGGAATGCTGTTTTTCCGGGGATCGCAGTG
GTGAGTAACCAcGCATCATCAGGAGTACGGATAAAATGCTTGATGGTCGG
AAGAGGCATAAATTCCGTCAGCCAGTTTAGTCTGACCATCTCATCTGTAA
CATCATTGGCAACGCTACCTTTGCCATGTTTCAGAAACAACTCTGGCGCA
TCGGGCTTCCCATACAAGCGATAGATTGTCGCACCTGATTGCCCGACATT
ATCGCGtGCCCATTTATACCCATATAAATCAGCATCCATGTTGGAATTTA
ATCGCGGCCTCGACGTTTCCCGTTGAATATGGCTCATAACACCCCTTGTA
TTACTGTTTATGTAAGCAGACAGTTTTATTGTTCATGATGATATATTTTT
ATCTTGTGCAATGTAACATCAGAGATTTTGAGACACAACGTGGCTTTCCC
CCCCCCCCCCATGACATTAACCTATAAAAATAGGCGTATCACGAGGCCag
gcctcTGGCAGGATATATTGTGGTGTAAACCGAGCTGTaTaGGCTGGCTG
GTGGCAGGATATATTGTGGTGTAAACttaattaaTAGGGATAACAGGGTA
ATGGCGCGCCtgacaggatatattggcgggtaaactaagtcgctgtatgt
gtttgtttggaattcgagctcggtacccggggatcctctagagtcgacct
gcaggcatgcaagcttagt

> SEQ ID NO 23 DNA sequence of backbone vector pAGb

actttgatccaacccctccgctgctatagtgcagtcggcttctgacgttc
agtgcagccgtcttctgaaaacgacatgtcgcacaagtcctaagttacgc
gacaggctgccgccctgcccttttcctggcgttttcttgtcgcgtgtttt
agtcgcataaagtagaatacttgcgactagaaccggagacattacgccat
gaacaagagcgccgccgctggcctgctgggctatgcccgcgtcagcaccg
acgaccaggacttgaccaaccaacgggccgaactgcacgcggccggctgc
accaagctgttttccgagaagatcaccggcaccaggcgcgaccgcccgga
gctggccaggatgcttgaccacctacgccctggcgacgttgtgacagtga
ccaggctagaccgcctggcccgcagcacccgcgacctactggacattgcc
gagcgcatccaggaggccggcgcgggcctgcgtagcctggcagagccgtg
ggccgacaccaccacgccggccggccgcatggtgttgaccgtgttcgccg
gcattgccgagttcgagcgttccctaatcatcgaccgcacccggagcggg
cgcgaggccgccaaggcccgaggcgtgaagtttggcccccgccctaccct
caccccggcacagatcgcgcacgcccgcgagctgatcgaccaggaaggcc
gcaccgtgaaagaggcggctgcactgcttggcgtgcatcgctcgaccctg
taccgcgcacttgagcgcagcgaggaagtgacgcccaccgaggccaggcg
gcgcggtgccttccgtgaggacgcattgaccgaggccgacgccctggcgg
ccgccgagaatgaacgccaagaggaacaagcatgaaaccgcaccaggacg
gccaggacgaaccgtttttcattaccgaagagatcgaggcggagatgatc
gcggccgggtacgtgttcgagccgcccgcgcacgtctcaaccgtgcggct
gcatgaaatcctggccggtttgtctgatgccaagctggcggcctggccgg
ccagcttggccgctgaagaaaccgagcgccgccgtctaaaaaggtgatgt
gtatttgagtaaaacagcttgcgtcatgcggtcgctgcgtatatgatgcg
atgagtaaataaacaaatacgcaaggggaacgcatgaaggttatcgctgt
acttaaccagaaaggcgggtcaggcaagacgaccatcgcaacccatctag
cccgcgccctgcaactcgccggggccgatgttctgttagtcgattccgat
ccccagggcagtgcccgcgattgggcggccgtgcgggaagatcaaccgct
aaccgttgtcggcatcgaccgcccgacgattgaccgcgacgtgaaggcca
tcggccggcgcgacttcgtagtgatcgacggagcgccccaggcggcggac
ttggctgtgtccgcgatcaaggcagccgacttcgtgctgattccggtgca
gccaagcccttacgacatatgggccaccgccgacctggtggagctggtta
agcagcgcattgaggtcacggatggaaggctacaagcggcctttgtcgtg
tcgcgggcgatcaaaggcacgcgcatcggcggtgaggttgccgaggcgct
ggccgggtacgagctgcccattcttgagtcccgtatcacgcagcgcgtga
gctacccaggcactgccgccgccggcacaaccgttcttgaatcagaaccc
gagggcgacgctgcccgcgaggtccaggcgctggccgctgaaattaaatc
aaaactcatttgagttaatgaggtaaagagaaaatgagcaaaagcacaaa
cacgctaagtgccggccgtccgagcgcacgcagcagcaaggctgcaacgt
tggccagcctggcagacacgccagccatgaagcgggtcaactttcagttg
ccggcggaggatcacaccaagctgaagatgtacgcggtacgccaaggcaa
gaccattaccgagctgctatctgaatacatcgcgcagctaccagagtaaa
tgagcaaatgaataaatgagtagatgaattttagcggctaaaggaggcgg
catggaaaatcaagaacaaccaggcaccgacgccgtggaatgccccatgt
gtggaggaacgggcggttggccaggcgtaagcggctgggttgtctgccgg
ccctgcaatggcactggaacccccaagcccgaggaatcggcgtgagcggt
cgcaaaccatccggcccggtacaaatcggcgcggcgctgggtgatgacct
ggtggagaagttgaaggccgcgcaggccgcccagcggcaacgcatcgagg
cagaagcacgccccggtgaatcgtggcaagcggccgctgatcgaatccgc
aaagaatcccggcaaccgccggcagccggtgcgccgtcgattaggaagcc
gcccaagggcgacgagcaaccagattttttcgttccgatgctctatgacg
tgggcacccgcgatagtcgcagcatcatggacgtggccgttttccgtctg
tcgaagcgtgaccgacgagctggcgaggtgatccgctacgagcttccaga
cgggcacgtagaggtttccgcagggccggccggcatggccagtgtgtggg
attacgacctggtactgatggcggtttcccatctaaccgaatccatgaac
cgataccgggaagggaagggagacaagcccggccgcgtgttccgtccaca
cgttgcggacgtactcaagttctgccggcgagccgatggcggaaagcaga
aagacgacctggtagaaacctgcattcggttaaacaccacgcacgttgcc
atgcagcgtacgaagaaggccaagaacggccgcctggtgacggtatccga
gggtgaagccttgattagccgctacaagatcgtaaagagcgaaaccgggc
ggccggagtacatcgagatcgagctagctgattggatgtaccgcgagatc
acagaaggcaagaacccggacgtgctgacggttcaccccgattacttttt
gatcgatcccggcatcggccgttttctctaccgcctggcacgccgcgccg
caggcaaggcagaagccagatggttgttcaagacgatctacgaacgcagt
ggcagcgccggagagttcaagaagttctgtttcaccgtgcgcaagctgat
cgggtcaaatgacctgccggagtacgatttgaaggaggaggcggggcagg
ctggcccgatcctagtcatgcgctaccgcaacctgatcgagggcgaagca
tccgccggttcctaatgtacggagcagatgctagggcaaattgccctagc
aggggaaaaaggtcgaaaaggtctctttcctgtggatagcacgtacattg
ggaacccaaagccgtacattgggaaccggaacccgtacattgggaaccca
aagccgtacattgggaaccggtcacacatgtaagtgactgatataaaaga
gaaaaaaggcgatttttccgcctaaaactctttaaaacttattaaaactc
ttaaaacccgcctggcctgtgcataactgtctggccagcgcacagccgaa
gagctgcaaaaagcgcctacccttcggtcgctgcgctccctacgccccgc
cgcttcgcgtcggcctatcgcggccgctggccgctcaaaaatggctggcc
tacggccaggcaatctaccagggcgcggacaagccgcgccgtcgccactc
gaccgccggcgcccacatcaaggcaccctgcctcgcgcgtttcggtgatg
acggtgaaaacctctgacacatgcagctcccggagacggtcacagcttgt
ctgtaagcggatgccgggagcagacaagcccgtcagggcgcgtcagcggg
tgttggcgggtgtcggggcgcagccatgacccagtcacgtagcgatagcg
gagtgtatactggcttaactatgcggcatcagagcagattgtactgagag
tgcaccatatgcggtgtgaaataccgcacagatgcgtaaggagaaaatac
cgcatcaggcgctcttccgcttcctcgctcactgactcgctgcgctcggt
cgttcggctgcggcgagcggtatcagctcactcaaaggcggtaatacggt
tatccacagaatcaggggataacgcaggaaagaacatgtgagcaaaaggc
cagcaaaaggccaggaaccgtaaaaaggccgcgttgctggcgtttttcca
taggctccgcccccctgacgagcatcacaaaaatcgacgctcaagtcaga
ggtggcgaaacccgacaggactataaagataccaggcgtttccccctgga
agctccctcgtgcgctctcctgttccgaccctgccgcttaccggatacct
gtccgcctttctcccttcgggaagcgtggcgctttctcatagctcacgct
gtaggtatctcagttcggtgtaggtcgttcgctccaagctgggctgtgtg
cacgaaccccccgttcagcccgaccgctgcgccttatccggtaactatcg
tcttgagtccaacccggtaagacacgacttatcgccactggcagcagcca
ctggtaacaggattagcagagcgaggtatgtaggcggtgctacagagttc
ttgaagtggtggcctaactacggctacactagaaggacagtatttggtat
ctgcgctctgctgaagccagttaccttcggaaaaagagttggtagctctt
gatccggcaaacaaaccaccgctggtagcggtggtttttttgtttgcaag
cagcagattacgcgcagaaaaaaaggatctcaagaagatcctttgatctt
ttctacggggtctgacgctcagtggaacgaaaactcacgttaagggattt
tggtcatgcatGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGCTTGCGCCGTCCCGTCAAGTCAGCGTAATGCTCTGCCA
GTGTTACAACCAATTAACCAATTCTGATTAGAAAAACTCATCGAGCATCA
AATGAAACTGCAATTTATTCACATCAGGATTATCAATACCATATTTTTGA
AAAAGCCGTTTCTGTAATGAAGGAGAAAACTCACCGAGGCAGTTCCATAG
GATGGCAAGATCCTGGTATCGGTCTGCGATTCCGACTCGTCCAACATCAA
TACAACCTATTAATTTCCCCTCGTCAAAAATAAGGTTATCAAGTGAGAAA
TCACCATGAGTGACGACTGAATCCGGTGAGAATGGCAAAAGTTTgTGCAT
TTCTTTCCAGACTTGTTCAACAGGCCAGCCATTACGCTCGTCATCAAAAT
CACTCGCATCAACCAAACCGTTATTCATTCGTGATTGCGCCTGAGCAAGA
CGAAATACGCGATCGCTGTTAAAAGGACAATTACAAACAGGAATCGAATG
CAACCGGCGCAGGAACACTGCCAGCGCATCAACAATATTTTCACCTGAAT
CAGGATATTCTTCTAATACCTGGAATGCTGTTTTTCCGGGGATCGCAGTG
GTGAGTAACCAcGCATCATCAGGAGTACGGATAAAATGCTTGATGGTCGG
AAGAGGCATAAATTCCGTCAGCCAGTTTAGTCTGACCATCTCATCTGTAA
CATCATTGGCAACGCTACCTTTGCCATGTTTCAGAAACAACTCTGGCGCA
TCGGGCTTCCCATACAAGCGATAGATTGTCGCACCTGATTGCCCGACATT
ATCGCGtGCCCATTTATACCCATATAAATCAGCATCCATGTTGGAATTTA
ATCGCGGCCTCGACGTTTCCCGTTGAATATGGCTCATAACACCCCTTGTA
TTACTGTTTATGTAAGCAGACAGTTTTATTGTTCATGATGATATATTTTT
ATCTTGTGCAATGTAACATCAGAGATTTTGAGACACAACGTGGCTTTCCC
CCCCCCCCCCATGACATTAACCTATAAAAATAGGCGTATCACGAGGCCag
gcctcTGGCAGGATATATTGTGGTGTAAACCGAGCTGTaTaGGCTGGCTG
GTGGCAGGATATATTGTGGTGTAAACttaattaaTAGGGATAACAGGGTA
ATGGCGCGCCtgacaggatatattggcgggtaaactaagtcgctgtatgt
gtttgtttgaagcttgcatgcctgcaggtcgactctagaggatccccggg
taccgagctcgaattcagt

> SEQ ID NO 24 DNA sequence of backbone vector pAGc

actttgatccaacccctccgctgctatagtgcagtcggcttctgacgttc
agtgcagccgtcttctgaaaacgacatgtcgcacaagtcctaagttacgc
gacaggctgccgccctgcccttttcctggcgttttcttgtcgcgtgtttt
agtcgcataaagtagaatacttgcgactagaaccggagacattacgccat
gaacaagagcgccgccgctggcctgctgggctatgcccgcgtcagcaccg
acgaccaggacttgaccaaccaacgggccgaactgcacgcggccggctgc
accaagctgttttccgagaagatcaccggcaccaggcgcgaccgcccgga
gctggccaggatgcttgaccacctacgccctggcgacgttgtgacagtga
ccaggctagaccgcctggcccgcagcacccgcgacctactggacattgcc
gagcgcatccaggaggccggcgcgggcctgcgtagcctggcagagccgtg
ggccgacaccaccacgccggccggccgcatggtgttgaccgtgttcgccg
gcattgccgagttcgagcgttccctaatcatcgaccgcacccggagcggg
cgcgaggccgccaaggcccgaggcgtgaagtttggcccccgccctaccct
caccccggcacagatcgcgcacgcccgcgagctgatcgaccaggaaggcc
gcaccgtgaaagaggcggctgcactgcttggcgtgcatcgctcgaccctg
taccgcgcacttgagcgcagcgaggaagtgacgcccaccgaggccaggcg
gcgcggtgccttccgtgaggacgcattgaccgaggccgacgccctggcgg
ccgccgagaatgaacgccaagaggaacaagcatgaaaccgcaccaggacg
gccaggacgaaccgtttttcattaccgaagagatcgaggcggagatgatc
gcggccgggtacgtgttcgagccgcccgcgcacgtctcaaccgtgcggct
gcatgaaatcctggccggtttgtctgatgccaagctggcggcctggccgg
ccagcttggccgctgaagaaaccgagcgccgccgtctaaaaaggtgatgt
gtatttgagtaaaacagcttgcgtcatgcggtcgctgcgtatatgatgcg
atgagtaaataaacaaatacgcaaggggaacgcatgaaggttatcgctgt
acttaaccagaaaggcgggtcaggcaagacgaccatcgcaacccatctag
cccgcgccctgcaactcgccggggccgatgttctgttagtcgattccgat
ccccagggcagtgcccgcgattgggcggccgtgcgggaagatcaaccgct
aaccgttgtcggcatcgaccgcccgacgattgaccgcgacgtgaaggcca
tcggccggcgcgacttcgtagtgatcgacggagcgccccaggcggcggac
ttggctgtgtccgcgatcaaggcagccgacttcgtgctgattccggtgca
gccaagcccttacgacatatgggccaccgccgacctggtggagctggtta
agcagcgcattgaggtcacggatggaaggctacaagcggcctttgtcgtg
tcgcgggcgatcaaaggcacgcgcatcggcggtgaggttgccgaggcgct
ggccgggtacgagctgcccattcttgagtcccgtatcacgcagcgcgtga
gctacccaggcactgccgccgccggcacaaccgttcttgaatcagaaccc
gagggcgacgctgcccgcgaggtccaggcgctggccgctgaaattaaatc
aaaactcatttgagttaatgaggtaaagagaaaatgagcaaaagcacaaa
cacgctaagtgccggccgtccgagcgcacgcagcagcaaggctgcaacgt
tggccagcctggcagacacgccagccatgaagcgggtcaactttcagttg
ccggcggaggatcacaccaagctgaagatgtacgcggtacgccaaggcaa
gaccattaccgagctgctatctgaatacatcgcgcagctaccagagtaaa
tgagcaaatgaataaatgagtagatgaattttagcggctaaaggaggcgg
catggaaaatcaagaacaaccaggcaccgacgccgtggaatgccccatgt
gtggaggaacgggcggttggccaggcgtaagcggctgggttgtctgccgg
ccctgcaatggcactggaacccccaagcccgaggaatcggcgtgagcggt
cgcaaaccatccggcccggtacaaatcggcgcggcgctgggtgatgacct
ggtggagaagttgaaggccgcgcaggccgcccagcggcaacgcatcgagg
cagaagcacgccccggtgaatcgtggcaagcggccgctgatcgaatccgc
aaagaatcccggcaaccgccggcagccggtgcgccgtcgattaggaagcc
gcccaagggcgacgagcaaccagattttttcgttccgatgctctatgacg
tgggcacccgcgatagtcgcagcatcatggacgtggccgttttccgtctg
tcgaagcgtgaccgacgagctggcgaggtgatccgctacgagcttccaga
cgggcacgtagaggtttccgcagggccggccggcatggccagtgtgtggg
attacgacctggtactgatggcggtttcccatctaaccgaatccatgaac
cgataccgggaagggaagggagacaagcccggccgcgtgttccgtccaca
cgttgcggacgtactcaagttctgccggcgagccgatggcggaaagcaga
aagacgacctggtagaaacctgcattcggttaaacaccacgcacgttgcc
atgcagcgtacgaagaaggccaagaacggccgcctggtgacggtatccga
gggtgaagccttgattagccgctacaagatcgtaaagagcgaaaccgggc
ggccggagtacatcgagatcgagctagctgattggatgtaccgcgagatc
acagaaggcaagaacccggacgtgctgacggttcaccccgattacttttt
gatcgatcccggcatcggccgttttctctaccgcctggcacgccgcgccg
caggcaaggcagaagccagatggttgttcaagacgatctacgaacgcagt
ggcagcgccggagagttcaagaagttctgtttcaccgtgcgcaagctgat
cgggtcaaatgacctgccggagtacgatttgaaggaggaggcggggcagg
ctggcccgatcctagtcatgcgctaccgcaacctgatcgagggcgaagca
tccgccggttcctaatgtacggagcagatgctagggcaaattgccctagc
aggggaaaaaggtcgaaaaggtctctttcctgtggatagcacgtacattg
ggaacccaaagccgtacattgggaaccggaacccgtacattgggaaccca
aagccgtacattgggaaccggtcacacatgtaagtgactgatataaaaga
gaaaaaaggcgatttttccgcctaaaactctttaaaacttattaaaactc
ttaaaacccgcctggcctgtgcataactgtctggccagcgcacagccgaa
gagctgcaaaaagcgcctacccttcggtcgctgcgctccctacgccccgc
cgcttcgcgtcggcctatcgcggccgctggccgctcaaaaatggctggcc
tacggccaggcaatctaccagggcgcggacaagccgcgccgtcgccactc
gaccgccggcgcccacatcaaggcaccctgcctcgcgcgtttcggtgatg
acggtgaaaacctctgacacatgcagctcccggagacggtcacagcttgt
ctgtaagcggatgccgggagcagacaagcccgtcagggcgcgtcagcggg
tgttggcgggtgtcggggcgcagccatgacccagtcacgtagcgatagcg
gagtgtatactggcttaactatgcggcatcagagcagattgtactgagag
tgcaccatatgcggtgtgaaataccgcacagatgcgtaaggagaaaatac
cgcatcaggcgctcttccgcttcctcgctcactgactcgctgcgctcggt
cgttcggctgcggcgagcggtatcagctcactcaaaggcggtaatacggt
tatccacagaatcaggggataacgcaggaaagaacatgtgagcaaaaggc
cagcaaaaggccaggaaccgtaaaaaggccgcgttgctggcgtttttcca
taggctccgcccccctgacgagcatcacaaaaatcgacgctcaagtcaga
ggtggcgaaacccgacaggactataaagataccaggcgtttccccctgga
agctccctcgtgcgctctcctgttccgaccctgccgcttaccggatacct
gtccgcctttctcccttcgggaagcgtggcgctttctcatagctcacgct
gtaggtatctcagttcggtgtaggtcgttcgctccaagctgggctgtgtg
cacgaaccccccgttcagcccgaccgctgcgccttatccggtaactatcg
tcttgagtccaacccggtaagacacgacttatcgccactggcagcagcca
ctggtaacaggattagcagagcgaggtatgtaggcggtgctacagagttc
ttgaagtggtggcctaactacggctacactagaaggacagtatttggtat
ctgcgctctgctgaagccagttaccttcggaaaaagagttggtagctctt
gatccggcaaacaaaccaccgctggtagcggtggtttttttgtttgcaag
cagcagattacgcgcagaaaaaaaggatctcaagaagatcctttgatctt
ttctacggggtctgacgctcagtggaacgaaaactcacgttaagggattt
tggtcatgcatGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGCTTGCGCCGTCCCGTCAAGTCAGCGTAATGCTCTGCCA
GTGTTACAACCAATTAACCAATTCTGATTAGAAAAACTCATCGAGCATCA
AATGAAACTGCAATTTATTCACATCAGGATTATCAATACCATATTTTTGA
AAAAGCCGTTTCTGTAATGAAGGAGAAAACTCACCGAGGCAGTTCCATAG
GATGGCAAGATCCTGGTATCGGTCTGCGATTCCGACTCGTCCAACATCAA
TACAACCTATTAATTTCCCCTCGTCAAAAATAAGGTTATCAAGTGAGAAA
TCACCATGAGTGACGACTGAATCCGGTGAGAATGGCAAAAGTTTgTGCAT
TTCTTTCCAGACTTGTTCAACAGGCCAGCCATTACGCTCGTCATCAAAAT
CACTCGCATCAACCAAACCGTTATTCATTCGTGATTGCGCCTGAGCAAGA
CGAAATACGCGATCGCTGTTAAAAGGACAATTACAAACAGGAATCGAATG
CAACCGGCGCAGGAACACTGCCAGCGCATCAACAATATTTTCACCTGAAT
CAGGATATTCTTCTAATACCTGGAATGCTGTTTTTCCGGGGATCGCAGTG
GTGAGTAACCAcGCATCATCAGGAGTACGGATAAAATGCTTGATGGTCGG
AAGAGGCATAAATTCCGTCAGCCAGTTTAGTCTGACCATCTCATCTGTAA
CATCATTGGCAACGCTACCTTTGCCATGTTTCAGAAACAACTCTGGCGCA
TCGGGCTTCCCATACAAGCGATAGATTGTCGCACCTGATTGCCCGACATT
ATCGCGtGCCCATTTATACCCATATAAATCAGCATCCATGTTGGAATTTA
ATCGCGGCCTCGACGTTTCCCGTTGAATATGGCTCATAACACCCCTTGTA
TTACTGTTTATGTAAGCAGACAGTTTTATTGTTCATGATGATATATTTTT
ATCTTGTGCAATGTAACATCAGAGATTTTGAGACACAACGTGGCTTTCCC
CCCCCCCCCCATGACATTAACCTATAAAAATAGGCGTATCACGAGGCCag
gcctgaattcgagctcggtacccggggatcctctagagtcgacctgcagg
catgcaagcttcTGGCAGGATATATTGTGGTGTAAACCGAGCTGTaTaGG
CTGGCTGGTGGCAGGATATATTGTGGTGTAAACttaattaaTAGGGATAA
CAGGGTAATGGCGCGCCtgacaggatatattggcgggtaaactaagtcgc
tgtatgtgtttgtttgagtact

> SEQ ID NO 25 DNA sequence of backbone vector pAGd

actttgatccaacccctccgctgctatagtgcagtcggcttctgacgttc
agtgcagccgtcttctgaaaacgacatgtcgcacaagtcctaagttacgc
gacaggctgccgccctgcccttttcctggcgttttcttgtcgcgtgtttt
agtcgcataaagtagaatacttgcgactagaaccggagacattacgccat
gaacaagagcgccgccgctggcctgctgggctatgcccgcgtcagcaccg
acgaccaggacttgaccaaccaacgggccgaactgcacgcggccggctgc
accaagctgttttccgagaagatcaccggcaccaggcgcgaccgcccgga
gctggccaggatgcttgaccacctacgccctggcgacgttgtgacagtga
ccaggctagaccgcctggcccgcagcacccgcgacctactggacattgcc
gagcgcatccaggaggccggcgcgggcctgcgtagcctggcagagccgtg
ggccgacaccaccacgccggccggccgcatggtgttgaccgtgttcgccg
gcattgccgagttcgagcgttccctaatcatcgaccgcacccggagcggg
cgcgaggccgccaaggcccgaggcgtgaagtttggcccccgccctaccct
caccccggcacagatcgcgcacgcccgcgagctgatcgaccaggaaggcc
gcaccgtgaaagaggcggctgcactgcttggcgtgcatcgctcgaccctg
taccgcgcacttgagcgcagcgaggaagtgacgcccaccgaggccaggcg
gcgcggtgccttccgtgaggacgcattgaccgaggccgacgccctggcgg
ccgccgagaatgaacgccaagaggaacaagcatgaaaccgcaccaggacg
gccaggacgaaccgtttttcattaccgaagagatcgaggcggagatgatc
gcggccgggtacgtgttcgagccgcccgcgcacgtctcaaccgtgcggct
gcatgaaatcctggccggtttgtctgatgccaagctggcggcctggccgg
ccagcttggccgctgaagaaaccgagcgccgccgtctaaaaaggtgatgt
gtatttgagtaaaacagcttgcgtcatgcggtcgctgcgtatatgatgcg
atgagtaaataaacaaatacgcaaggggaacgcatgaaggttatcgctgt
acttaaccagaaaggcgggtcaggcaagacgaccatcgcaacccatctag
cccgcgccctgcaactcgccggggccgatgttctgttagtcgattccgat
ccccagggcagtgcccgcgattgggcggccgtgcgggaagatcaaccgct
aaccgttgtcggcatcgaccgcccgacgattgaccgcgacgtgaaggcca
tcggccggcgcgacttcgtagtgatcgacggagcgccccaggcggcggac
ttggctgtgtccgcgatcaaggcagccgacttcgtgctgattccggtgca
gccaagcccttacgacatatgggccaccgccgacctggtggagctggtta
agcagcgcattgaggtcacggatggaaggctacaagcggcctttgtcgtg
tcgcgggcgatcaaaggcacgcgcatcggcggtgaggttgccgaggcgct
ggccgggtacgagctgcccattcttgagtcccgtatcacgcagcgcgtga
gctacccaggcactgccgccgccggcacaaccgttcttgaatcagaaccc
gagggcgacgctgcccgcgaggtccaggcgctggccgctgaaattaaatc
aaaactcatttgagttaatgaggtaaagagaaaatgagcaaaagcacaaa
cacgctaagtgccggccgtccgagcgcacgcagcagcaaggctgcaacgt
tggccagcctggcagacacgccagccatgaagcgggtcaactttcagttg
ccggcggaggatcacaccaagctgaagatgtacgcggtacgccaaggcaa
gaccattaccgagctgctatctgaatacatcgcgcagctaccagagtaaa
tgagcaaatgaataaatgagtagatgaattttagcggctaaaggaggcgg
catggaaaatcaagaacaaccaggcaccgacgccgtggaatgccccatgt
gtggaggaacgggcggttggccaggcgtaagcggctgggttgtctgccgg
ccctgcaatggcactggaacccccaagcccgaggaatcggcgtgagcggt
cgcaaaccatccggcccggtacaaatcggcgcggcgctgggtgatgacct
ggtggagaagttgaaggccgcgcaggccgcccagcggcaacgcatcgagg
cagaagcacgccccggtgaatcgtggcaagcggccgctgatcgaatccgc
aaagaatcccggcaaccgccggcagccggtgcgccgtcgattaggaagcc
gcccaagggcgacgagcaaccagattttttcgttccgatgctctatgacg
tgggcacccgcgatagtcgcagcatcatggacgtggccgttttccgtctg
tcgaagcgtgaccgacgagctggcgaggtgatccgctacgagcttccaga
cgggcacgtagaggtttccgcagggccggccggcatggccagtgtgtggg
attacgacctggtactgatggcggtttcccatctaaccgaatccatgaac
cgataccgggaagggaagggagacaagcccggccgcgtgttccgtccaca
cgttgcggacgtactcaagttctgccggcgagccgatggcggaaagcaga
aagacgacctggtagaaacctgcattcggttaaacaccacgcacgttgcc
atgcagcgtacgaagaaggccaagaacggccgcctggtgacggtatccga
gggtgaagccttgattagccgctacaagatcgtaaagagcgaaaccgggc
ggccggagtacatcgagatcgagctagctgattggatgtaccgcgagatc
acagaaggcaagaacccggacgtgctgacggttcaccccgattacttttt
gatcgatcccggcatcggccgttttctctaccgcctggcacgccgcgccg
caggcaaggcagaagccagatggttgttcaagacgatctacgaacgcagt
ggcagcgccggagagttcaagaagttctgtttcaccgtgcgcaagctgat
cgggtcaaatgacctgccggagtacgatttgaaggaggaggcggggcagg
ctggcccgatcctagtcatgcgctaccgcaacctgatcgagggcgaagca
tccgccggttcctaatgtacggagcagatgctagggcaaattgccctagc
aggggaaaaaggtcgaaaaggtctctttcctgtggatagcacgtacattg
ggaacccaaagccgtacattgggaaccggaacccgtacattgggaaccca
aagccgtacattgggaaccggtcacacatgtaagtgactgatataaaaga
gaaaaaaggcgatttttccgcctaaaactctttaaaacttattaaaactc
ttaaaacccgcctggcctgtgcataactgtctggccagcgcacagccgaa
gagctgcaaaaagcgcctacccttcggtcgctgcgctccctacgccccgc
cgcttcgcgtcggcctatcgcggccgctggccgctcaaaaatggctggcc
tacggccaggcaatctaccagggcgcggacaagccgcgccgtcgccactc
gaccgccggcgcccacatcaaggcaccctgcctcgcgcgtttcggtgatg
acggtgaaaacctctgacacatgcagctcccggagacggtcacagcttgt
ctgtaagcggatgccgggagcagacaagcccgtcagggcgcgtcagcggg
tgttggcgggtgtcggggcgcagccatgacccagtcacgtagcgatagcg
gagtgtatactggcttaactatgcggcatcagagcagattgtactgagag
tgcaccatatgcggtgtgaaataccgcacagatgcgtaaggagaaaatac
cgcatcaggcgctcttccgcttcctcgctcactgactcgctgcgctcggt
cgttcggctgcggcgagcggtatcagctcactcaaaggcggtaatacggt
tatccacagaatcaggggataacgcaggaaagaacatgtgagcaaaaggc
cagcaaaaggccaggaaccgtaaaaaggccgcgttgctggcgtttttcca
taggctccgcccccctgacgagcatcacaaaaatcgacgctcaagtcaga
ggtggcgaaacccgacaggactataaagataccaggcgtttccccctgga
agctccctcgtgcgctctcctgttccgaccctgccgcttaccggatacct
gtccgcctttctcccttcgggaagcgtggcgctttctcatagctcacgct
gtaggtatctcagttcggtgtaggtcgttcgctccaagctgggctgtgtg
cacgaaccccccgttcagcccgaccgctgcgccttatccggtaactatcg
tcttgagtccaacccggtaagacacgacttatcgccactggcagcagcca
ctggtaacaggattagcagagcgaggtatgtaggcggtgctacagagttc
ttgaagtggtggcctaactacggctacactagaaggacagtatttggtat
ctgcgctctgctgaagccagttaccttcggaaaaagagttggtagctctt
gatccggcaaacaaaccaccgctggtagcggtggtttttttgtttgcaag
cagcagattacgcgcagaaaaaaaggatctcaagaagatcctttgatctt
ttctacggggtctgacgctcagtggaacgaaaactcacgttaagggattt
tggtcatgcatGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGCTTGCGCCGTCCCGTCAAGTCAGCGTAATGCTCTGCCA
GTGTTACAACCAATTAACCAATTCTGATTAGAAAAACTCATCGAGCATCA
AATGAAACTGCAATTTATTCACATCAGGATTATCAATACCATATTTTTGA
AAAAGCCGTTTCTGTAATGAAGGAGAAAACTCACCGAGGCAGTTCCATAG
GATGGCAAGATCCTGGTATCGGTCTGCGATTCCGACTCGTCCAACATCAA
TACAACCTATTAATTTCCCCTCGTCAAAAATAAGGTTATCAAGTGAGAAA
TCACCATGAGTGACGACTGAATCCGGTGAGAATGGCAAAAGTTTgTGCAT
TTCTTTCCAGACTTGTTCAACAGGCCAGCCATTACGCTCGTCATCAAAAT
CACTCGCATCAACCAAACCGTTATTCATTCGTGATTGCGCCTGAGCAAGA
CGAAATACGCGATCGCTGTTAAAAGGACAATTACAAACAGGAATCGAATG
CAACCGGCGCAGGAACACTGCCAGCGCATCAACAATATTTTCACCTGAAT
CAGGATATTCTTCTAATACCTGGAATGCTGTTTTTCCGGGGATCGCAGTG
GTGAGTAACCAcGCATCATCAGGAGTACGGATAAAATGCTTGATGGTCGG
AAGAGGCATAAATTCCGTCAGCCAGTTTAGTCTGACCATCTCATCTGTAA
CATCATTGGCAACGCTACCTTTGCCATGTTTCAGAAACAACTCTGGCGCA
TCGGGCTTCCCATACAAGCGATAGATTGTCGCACCTGATTGCCCGACATT
ATCGCGtGCCCATTTATACCCATATAAATCAGCATCCATGTTGGAATTTA
ATCGCGGCCTCGACGTTTCCCGTTGAATATGGCTCATAACACCCCTTGTA
TTACTGTTTATGTAAGCAGACAGTTTTATTGTTCATGATGATATATTTTT
ATCTTGTGCAATGTAACATCAGAGATTTTGAGACACAACGTGGCTTTCCC
CCCCCCCCCCATGACATTAACCTATAAAAATAGGCGTATCACGAGGCCag
gcctaagcttgcatgcctgcaggtcgactctagaggatccccgggtaccg
agctcgaattccTGGCAGGATATATTGTGGTGTAAACCGAGCTGTaTaGG
CTGGCTGGTGGCAGGATATATTGTGGTGTAAACttaattaaTAGGGATAA
CAGGGTAATGGCGCGCCtgacaggatatattggcgggtaaactaagtcgc
tgtatgtgtttgtttgagtact

>SEQ ID NO 26 DNA sequence of binary chloroplast transformation vector pAG-CpTEST_ Note dicistronic aadA-gfp marker genes

actttgatccaacccctccgctgctatagtgcagtcggcttctgacgttc
agtgcagccgtcttctgaaaacgacatgtcgcacaagtcctaagttacgc
gacaggctgccgccctgcccttttcctggcgttttcttgtcgcgtgtttt
agtcgcataaagtagaatacttgcgactagaaccggagacattacgccat
gaacaagagcgccgccgctggcctgctgggctatgcccgcgtcagcaccg
acgaccaggacttgaccaaccaacgggccgaactgcacgcggccggctgc
accaagctgttttccgagaagatcaccggcaccaggcgcgaccgcccgga
gctggccaggatgcttgaccacctacgccctggcgacgttgtgacagtga
ccaggctagaccgcctggcccgcagcacccgcgacctactggacattgcc
gagcgcatccaggaggccggcgcgggcctgcgtagcctggcagagccgtg
ggccgacaccaccacgccggccggccgcatggtgttgaccgtgttcgccg
gcattgccgagttcgagcgttccctaatcatcgaccgcacccggagcggg
cgcgaggccgccaaggcccgaggcgtgaagtttggcccccgccctaccct
caccccggcacagatcgcgcacgcccgcgagctgatcgaccaggaaggcc
gcaccgtgaaagaggcggctgcactgcttggcgtgcatcgctcgaccctg
taccgcgcacttgagcgcagcgaggaagtgacgcccaccgaggccaggcg
gcgcggtgccttccgtgaggacgcattgaccgaggccgacgccctggcgg
ccgccgagaatgaacgccaagaggaacaagcatgaaaccgcaccaggacg
gccaggacgaaccgtttttcattaccgaagagatcgaggcggagatgatc
gcggccgggtacgtgttcgagccgcccgcgcacgtctcaaccgtgcggct
gcatgaaatcctggccggtttgtctgatgccaagctggcggcctggccgg
ccagcttggccgctgaagaaaccgagcgccgccgtctaaaaaggtgatgt
gtatttgagtaaaacagcttgcgtcatgcggtcgctgcgtatatgatgcg
atgagtaaataaacaaatacgcaaggggaacgcatgaaggttatcgctgt
acttaaccagaaaggcgggtcaggcaagacgaccatcgcaacccatctag
cccgcgccctgcaactcgccggggccgatgttctgttagtcgattccgat
ccccagggcagtgcccgcgattgggcggccgtgcgggaagatcaaccgct
aaccgttgtcggcatcgaccgcccgacgattgaccgcgacgtgaaggcca
tcggccggcgcgacttcgtagtgatcgacggagcgccccaggcggcggac
ttggctgtgtccgcgatcaaggcagccgacttcgtgctgattccggtgca
gccaagcccttacgacatatgggccaccgccgacctggtggagctggtta
agcagcgcattgaggtcacggatggaaggctacaagcggcctttgtcgtg
tcgcgggcgatcaaaggcacgcgcatcggcggtgaggttgccgaggcgct
ggccgggtacgagctgcccattcttgagtcccgtatcacgcagcgcgtga
gctacccaggcactgccgccgccggcacaaccgttcttgaatcagaaccc
gagggcgacgctgcccgcgaggtccaggcgctggccgctgaaattaaatc
aaaactcatttgagttaatgaggtaaagagaaaatgagcaaaagcacaaa
cacgctaagtgccggccgtccgagcgcacgcagcagcaaggctgcaacgt
tggccagcctggcagacacgccagccatgaagcgggtcaactttcagttg
ccggcggaggatcacaccaagctgaagatgtacgcggtacgccaaggcaa
gaccattaccgagctgctatctgaatacatcgcgcagctaccagagtaaa
tgagcaaatgaataaatgagtagatgaattttagcggctaaaggaggcgg
catggaaaatcaagaacaaccaggcaccgacgccgtggaatgccccatgt
gtggaggaacgggcggttggccaggcgtaagcggctgggttgtctgccgg
ccctgcaatggcactggaacccccaagcccgaggaatcggcgtgagcggt
cgcaaaccatccggcccggtacaaatcggcgcggcgctgggtgatgacct
ggtggagaagttgaaggccgcgcaggccgcccagcggcaacgcatcgagg
cagaagcacgccccggtgaatcgtggcaagcggccgctgatcgaatccgc
aaagaatcccggcaaccgccggcagccggtgcgccgtcgattaggaagcc
gcccaagggcgacgagcaaccagattttttcgttccgatgctctatgacg
tgggcacccgcgatagtcgcagcatcatggacgtggccgttttccgtctg
tcgaagcgtgaccgacgagctggcgaggtgatccgctacgagcttccaga
cgggcacgtagaggtttccgcagggccggccggcatggccagtgtgtggg
attacgacctggtactgatggcggtttcccatctaaccgaatccatgaac
cgataccgggaagggaagggagacaagcccggccgcgtgttccgtccaca
cgttgcggacgtactcaagttctgccggcgagccgatggcggaaagcaga
aagacgacctggtagaaacctgcattcggttaaacaccacgcacgttgcc
atgcagcgtacgaagaaggccaagaacggccgcctggtgacggtatccga
gggtgaagccttgattagccgctacaagatcgtaaagagcgaaaccgggc
ggccggagtacatcgagatcgagctagctgattggatgtaccgcgagatc
acagaaggcaagaacccggacgtgctgacggttcaccccgattacttttt
gatcgatcccggcatcggccgttttctctaccgcctggcacgccgcgccg
caggcaaggcagaagccagatggttgttcaagacgatctacgaacgcagt
ggcagcgccggagagttcaagaagttctgtttcaccgtgcgcaagctgat
cgggtcaaatgacctgccggagtacgatttgaaggaggaggcggggcagg
ctggcccgatcctagtcatgcgctaccgcaacctgatcgagggcgaagca
tccgccggttcctaatgtacggagcagatgctagggcaaattgccctagc
aggggaaaaaggtcgaaaaggtctctttcctgtggatagcacgtacattg
ggaacccaaagccgtacattgggaaccggaacccgtacattgggaaccca
aagccgtacattgggaaccggtcacacatgtaagtgactgatataaaaga
gaaaaaaggcgatttttccgcctaaaactctttaaaacttattaaaactc
ttaaaacccgcctggcctgtgcataactgtctggccagcgcacagccgaa
gagctgcaaaaagcgcctacccttcggtcgctgcgctccctacgccccgc
cgcttcgcgtcggcctatcgcggccgctggccgctcaaaaatggctggcc
tacggccaggcaatctaccagggcgcggacaagccgcgccgtcgccactc
gaccgccggcgcccacatcaaggcaccctgcctcgcgcgtttcggtgatg
acggtgaaaacctctgacacatgcagctcccggagacggtcacagcttgt
ctgtaagcggatgccgggagcagacaagcccgtcagggcgcgtcagcggg
tgttggcgggtgtcggggcgcagccatgacccagtcacgtagcgatagcg
gagtgtatactggcttaactatgcggcatcagagcagattgtactgagag
tgcaccatatgcggtgtgaaataccgcacagatgcgtaaggagaaaatac
cgcatcaggcgctcttccgcttcctcgctcactgactcgctgcgctcggt
cgttcggctgcggcgagcggtatcagctcactcaaaggcggtaatacggt
tatccacagaatcaggggataacgcaggaaagaacatgtgagcaaaaggc
cagcaaaaggccaggaaccgtaaaaaggccgcgttgctggcgtttttcca
taggctccgcccccctgacgagcatcacaaaaatcgacgctcaagtcaga
ggtggcgaaacccgacaggactataaagataccaggcgtttccccctgga
agctccctcgtgcgctctcctgttccgaccctgccgcttaccggatacct
gtccgcctttctcccttcgggaagcgtggcgctttctcatagctcacgct
gtaggtatctcagttcggtgtaggtcgttcgctccaagctgggctgtgtg
cacgaaccccccgttcagcccgaccgctgcgccttatccggtaactatcg
tcttgagtccaacccggtaagacacgacttatcgccactggcagcagcca
ctggtaacaggattagcagagcgaggtatgtaggcggtgctacagagttc
ttgaagtggtggcctaactacggctacactagaaggacagtatttggtat
ctgcgctctgctgaagccagttaccttcggaaaaagagttggtagctctt
gatccggcaaacaaaccaccgctggtagcggtggtttttttgtttgcaag
cagcagattacgcgcagaaaaaaaggatctcaagaagatcctttgatctt
ttctacggggtctgacgctcagtggaacgaaaactcacgttaagggattt
tggtcatgcatGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGCTTGCGCCGTCCCGTCAAGTCAGCGTAATGCTCTGCCA
GTGTTACAACCAATTAACCAATTCTGATTAGAAAAACTCATCGAGCATCA
AATGAAACTGCAATTTATTCACATCAGGATTATCAATACCATATTTTTGA
AAAAGCCGTTTCTGTAATGAAGGAGAAAACTCACCGAGGCAGTTCCATAG
GATGGCAAGATCCTGGTATCGGTCTGCGATTCCGACTCGTCCAACATCAA
TACAACCTATTAATTTCCCCTCGTCAAAAATAAGGTTATCAAGTGAGAAA
TCACCATGAGTGACGACTGAATCCGGTGAGAATGGCAAAAGTTTgTGCAT
TTCTTTCCAGACTTGTTCAACAGGCCAGCCATTACGCTCGTCATCAAAAT
CACTCGCATCAACCAAACCGTTATTCATTCGTGATTGCGCCTGAGCAAGA
CGAAATACGCGATCGCTGTTAAAAGGACAATTACAAACAGGAATCGAATG
CAACCGGCGCAGGAACACTGCCAGCGCATCAACAATATTTTCACCTGAAT
CAGGATATTCTTCTAATACCTGGAATGCTGTTTTTCCGGGGATCGCAGTG
GTGAGTAACCAcGCATCATCAGGAGTACGGATAAAATGCTTGATGGTCGG
AAGAGGCATAAATTCCGTCAGCCAGTTTAGTCTGACCATCTCATCTGTAA
CATCATTGGCAACGCTACCTTTGCCATGTTTCAGAAACAACTCTGGCGCA
TCGGGCTTCCCATACAAGCGATAGATTGTCGCACCTGATTGCCCGACATT
ATCGCGtGCCCATTTATACCCATATAAATCAGCATCCATGTTGGAATTTA
ATCGCGGCCTCGACGTTTCCCGTTGAATATGGCTCATAACACCCCTTGTA
TTACTGTTTATGTAAGCAGACAGTTTTATTGTTCATGATGATATATTTTT
ATCTTGTGCAATGTAACATCAGAGATTTTGAGACACAACGTGGCTTTCCC
CCCCCCCCCCATGACATTAACCTATAAAAATAGGCGTATCACGAGGCCag
gcctcTGGCAGGATATATTGTGGTGTAAACCGAGCTGTaTaGGCTGGCTG
GTGGCAGGATATATTGTGGTGTAAACttaattaaAATTCACCGCCGTATG
GCTGACCGGCGATTACTAGCGATTCCGGCTTCATGCAGGCGAGTTGCAGC
CTGCAATCCGAACTGAGGACGGGTTTTTGGGGTTAGCTCACCCTCGCGGG
ATCGCGACCCTTTGTCCCGGCCATTGTAGCACGTGTGTCGCCCAGGGCAT
AAGGGGCATGATGACTTGACGTCATCCTCACCTTCCTCCGGCTTATCACC
GGCAGTCTGTTCAGGGTTCCAAACTCAACGATGGCAACTAAACACGAGGG
TTGCGCTCGTTGCGGGACTTAACCCAACACCTTACGGCACGAGCTGACGA
CAGCCATGCACCACCTGTGTCCGCGTTCCCGAAGGCACCCCTCTCTTTCA
AGAGGATTCGCGGCATGTCAAGCCCTGGTAAGGTTCTTCGCTTTGCATCG
AATTAAACCACATGCTCCACCGCTTGTGCGGGCCCCCGTCAATTCCTTTG
AGTTTCATTCTTGCGAACGTACTCCCCAGGCGGGATACTTAACGCGTTAG
CTACAGCACTGCACGGGTCGATACGCACAGCGCCTAGTATCCATCGTTTA
CGGCTAGGACTACTGGGGTATCTAATCCCATTCGCTCCCCTAGCTTTCGT
CTCTCAGTGTCAGTGTCGGCCCAGCAGAGTGCTTTCGCCGTTGGTGTTCT
TTCCGATCTCTACGCATTTCACCGCTCCACCGGAAATTCCCTCTGCCCCT
ACCGTACTCCAGCTTGGTAGTTTCCACCGCCTGTCCAGGGTTGAGCCCTG
GGATTTGACGGCGGACTTAAAAAGCCACCTACAGACGCTTTACGCCCAAT
CATTCCGGATAACGCTTGCATCCTCTGTATTACCGCGGCTGCTGGCACAG
AGTTAGCCGATGCTTATTCCCCAGATACCGTCATTGCTTCTTCTCCGGGA
AAAGAAGTTCACGACCCGTGGGCCTTCTACCTCCACGCGGCATTGCTCCG
TCAGGCTTTCGCCCATTGCGGAAAATTCCCCACTGCTGCCTCCCGTAGGA
GTCTGGGCCGTGTCTCAGTCCCAGTGTGGCTGATCATCCTCTCGGACCAG
CTACTGATCATCGCCTTGGTAAGCTATTGCCTCACCAACTAGCTAATCAG
ACGCGAGCCCCTCCTCGGGCGGATTCCTCCTTTTGCTCCTCAGCCTACGG
GGTATTAGCAGCCGTTTCCAGCTGTTGTTCCCCTCCCAAGGGCAGGTTCT
TACGCGTTACTCACCCGTCCGCCACTGGAAACACCACTTCCCGTCCGACT
TGCATGTGTTAAGCATGCCGCCAGCGTTCATCCTGAGCCAGGATCGAACT
CTCCATGAGATTCATAGTTGCATTACTTATAGCTTCCTTGTTCGTAGACA
AAGCGGATTCGGAATTGTCTTTCATTCCAAGGCATAACTTGTATCCATGC
GCTTCATATTCGCCCGGAGTTCGCTCCCAGAAATATAGCCATCCCTGCCC
CCTCACGTCAATCCCACGAGCCTCTTATCCATTCTCATTGAACGACGGCG
GGGGAGCAAATCCAACTAGAAAAACTCACATTGGGCTTAGGGATAATCAG
GCTCGAACTGATGACTTCCACCACGTCAAGGTGACACTCTACCGCTGAGT
TATATCCCTTCCCCGCCCCATCGAGAAATAGAACTGACTAATCCTAAGTC
AAAGGGTCGAGAAACTCAACGCCACTATTCTTGAACAACTTGGAGCCGGG
CCTTCTTTTCGCACTATTACGGATATGAAAATAATGGTCAAAATCGGATT
CAATTGTCTACcGCTCCCCCGCCGTCGTTCAATGAGAATGGATAAGAGGC
TCGTGGGATTGACGTGAGGGGGCAGGGATGGCTATATTTCTGGGAGAATT
AACCGATCGACGTGCaAGCGGACATTTATTTTaAATTCGATAATTTTTGC
AAAAACATTTCGACATATTTATTTATTTTATTATTATGAGAATCAATCCT
ACTACTTCTGGTTCTGGGGTTTCCACGgctactagcGAAGCGGTGATCGC
CGAAGTATCGACTCAACTATCAGAGGTAGTTGGCGTCATCGAGCGCCATC
TCGAACCGACGTTGCTGGCCGTACATTTGTACGGCTCCGCAGTGGATGGC
GGCCTGAAGCCACACAGTGATATTGATTTGCTGGTTACGGTGACCGTAAG
GCTTGATGAAACAACGCGGCGAGCTTTGATCAACGACCTTTTGGAAACTT
CGGCTTCCCCTGGAGAGAGCGAGATTCTCCGCGCTGTAGAAGTCACCATT
GTTGTGCACGACGACATCATTCCGTGGCGTTATCCAGCTAAGCGCGAACT
GCAATTTGGAGAATGGCAGCGCAATGACATTCTTGCAGGTATCTTCGAGC
CAGCCACGATCGACATTGATCTGGCTATCTTGCTGACAAAAGCAAGAGAA
CATAGCGTTGCCTTGGTAGGTCCAGCGGCGGAGGAACTCTTTGATCCGGT
TCCTGAACAGGATCTATTTGAGGCGCTAAATGAAACCTTAACGCTATGGA
ACTCGCCGCCCGACTGGGCTGGCGATGAGCGAAATGTAGTGCTTACGTTG
TCCCGCATTTGGTACAGCGCAGTAACCGGCAAAATCGCGCCGAAGGATGT
CGCTGCCGACTGGGCAATGGAGCGCCTGCCGGCCCAGTATCAGCCCGTCA
TACTTGAAGCTAGACAGGCTTATCTTGGACAAGAAGAAGATCGCTTGGCC
TCGCGCGCAGATCAGTTGGAAGAATTTGTCCACTACGTGAAAGGCGAGAT
CACCAAGGTAGTgGGCAAAgaaCAAAAACTCATTTCTGAAGAAGACTTGT
AACTGCAGATAACCCAAATAATGTTTTAAAATTTTAAAAATAATGTAGGA
GGAAAAATTATGgctagcAGTAAAGGAGAAGAACTTTTCACTGGAGTTGT
CCCAATTCTTGTTGAATTAGATGGTGATGTTAATGGGCACAAATTTTCTG
TCAGTGGAGAGGGTGAAGGTGATGCAACATACGGAAAACTTACCCTTAAA
TTTATTTGCACTACTGGAAAACTACCTGTTCCtTGGCCAACACTTGTCAC
TACTTTCTCTTATGGTGTTCAATGCTTTTCAAGATACCCAGATCATATGA
AGCGGCACGACTTCTTCAAGAGCGCCATGCCTGAGGGATACGTGCAGGAG
AGGACCATCTCTTTCAAGGACGACGGGAACTACAAGACACGTGCTGAAGT
CAAGTTTGAGGGAGACACCCTCGTCAACAGGATCGAGCTTAAGGGAATCG
ATTTCAAGGAGGACGGAAACATCCTCGGCCACAAGTTGGAATACAACTAC
AACTCCCACAACGTATACATCACGGCAGACAAACAAAAGAATGGAATCAA
AGCTAACTTCAAAATTAGACACAACATTGAAGATGGAAGCGTTCAACTAG
CAGACCATTATCAACAAAATACTCCAATTGGCGATGGCCCTGTCCTTTTA
CCAGACAACCATTACCTGTCCACACAATCTGCCCTTTCGAAAGATCCCAA
CGAAAAGAGAGACCACATGGTCCTTCTTGAGTTTGTAACAGCTGCTGGGA
TTACACATGGCATGGATGAACTATACAAATAAGctctagCTAGAGCgatc
ctggcctagtctataggaggttttgaaaagaaaggagcaataatcatttt
cttgttctatcaagagggtgctattgctcctttctttttttctttttatt
tatttactagtattttacttacatagacttttttgtttacattatagaaa
aagaaggagaggttattttcttgcatttattcatgGGGGATCAAAGCTAA
CAACTGCCCCTATCGGAAATAGGATTGACTACCGATTCCGAAGGAACTGG
AGTTACATCTCTTTTCCATTCAAGAGTTCTTATGCGTTTCCACGCCCCTT
TGAGACCCCGAAAAATGGACAAATTCCTTTTCTTAGGAACACATACAAGA
TTCGTCACTACAAAAAGGATAATGGTAACCCTACCATTAACTACTTCATT
TATGAATTTCATAGTAATAGAAATACATGTCCTACCGAGACAGAATTTGG
AACTTGCTATCCTCTTGCCTAGCAGGCAAAGATTTACCTCCGTGGAAAGG
ATGATTCATTCGGATCGACATGAGAGTCCAACTACATTGCCAGAATCCAT
GTTGTATATTTGAAAGAGGTTGACCTCCTTGCTTCTCTCATGGTACACTC
CTCTTCCCGCCGAGCCCCTTTTCTCCTCGGTCCACAGAGACAAAATGTAG
GACTGGTGCCAACAATTCATCAGACTCACTAAGTCGGGATCACTAACTAA
TACTAATCTAATATAATAGTCTAATATATCTAATATAATAGAAAATACTA
ATATAATAGAAAAGAACTGTCTTTTCTGTATACTTTCCCCGGTTCCGTTG
CTACCGCGGGCTTTACGCAATCGATCGGATTAGATAGATATCCCTTCAAC
ATAGGTCATCGAAAGGATCTCGGAGACCCACCAAAGTACGAAAGCCAGGA
TCTTTCAGAAAACGGATTCCTATTCAAAGAGTGCATAACCGCATGGATAA
GCTCACACTAACCCGTCAATTTGGGATCCAAATTCGAGATTTTCCTTGGG
AGGTATCGGGAAGGATTTGGAATGGAATAATATCGATTCATACAGAAGAA
AAGGTTCTCTATTGATTCAAACACTGTACCTAACCTATGGGATAGGGATC
GAGGAAGGGGAAAAACCGAAGATTTCACATGGTACTTTTATCAATCTGAT
TTATTTCGTACCTTTCGTTCAATGAGAAAATGGGTCAAATTCTACAGGAT
CAAACCTATGGGACTTAAGGAATGATATAAAAAAAAGAGAGGGAAAATAT
TCATATTAAATAAATATGAAGTAGAAGAACCCAGATTCCAAATGAACAAA
TTCAAACTTGAAAAGGATCTTCCTTATTCTTGAAGAATGAGGGGCAAAGG
GATTGATCAAGAAAGATCGGCGCGCCtgacaggatatattggcgggtaaa
ctaagtcgctgtatgtgtttgtttggaattcgagctcggtacccggggat
cctctagagtcgacctgcaggcatgcaagcttagt

> SEQ ID NO 27 The DNA sequence of Pt-VirD2 in a VirD promoter/VirF terminator cassette. The Pt-VirD2 has the tobacco SSU Transit peptide+5aa, the mutant minimal VirD2-T4SS signal.

ttatcgatgagtaaaagcgttgttacactatttttatttcacattcgtta
taagacaattgcaaatgtagcaagtatattcagtattgactgtaaatgta
ctgttgatttcatattgagcagggctagacttccatccgtctacccgggc
acatttcgtgctggagtatccagaccttccgctttctttggaggaagcta
tggcttcctcagttctttcctccgcagcagttgccacccgcagcaatgtt
gctcaagctaacatggttgcacctttcactggccttaagtcagctgcctc
attccctgtttcaaggaagcaaaaccttgacatcacttccattgccagca
acggcggaagagtgcaatgcatgCAGGTGTGGCCAGCGATGGCTCCCGAT
CGGGCCcaagtaatcattcgcattgtgccaggaggtggaaccaagaccct
tcagcagataatcaatcagctggagtacctgtccacgaagggaaagctgg
aactccagcgttcagcccggcatctcgatattcccgttccgccggatcaa
atccgtgagcttgcccaaagctgggttacggaggccgggatttatgacga
aagtcagtcagacgatgacaggcaacaagacttaacaacacacattattg
tgagcttccccgcaggtacggaccaaaccgcagcttatgaagcaagccgg
gaatgggcagccgagatgtttgggtcaggatacgggggtggccgctataa
ctatctgacagcctaccacgtggaccgcgatcatccacatttacatgtcg
tggtcaatcgtcgggaacttctggggcaggggtggctgaaaatatccagg
cgccatccccagctgaattatgacggcttacggaaaaagatggcagagat
ttcacttcgtcacggcatagtcctggatgcgacttcccgagcagaaaggg
gaatagcagagcgaccaatcacctatgctgaatatcgacgtCTTGAGCGG
ATGCTCGAAGTTATGGCAGAAGTTCGGCCCATAGCCCGATCCATTAAAAC
GGCTCACGACGATGCGCGAGCGGAATTAATGTCGGCGGACAGACCTCGAT
CAACGCGCGGTCTATGATTctagaatccgtgctgttcgtcacccgcttat
agttgccatcacaatcggatttcaactgccccggtctttaggtgcggccg
aggacaatgtagcacgcacttacggtgcgtctcgtctctgacgctttcta
tgttccgca

> SEQ ID NO 28 The DNA sequence of Pt-VirD2 in a VirE promoter/VirF terminator cassette. The Pt-VirD2 has the tobacco SSU Transit peptide+5aa, the mutant minimal VirD2-T4SS signal.

cggctgctcgtcaccaacaatgctcacagggatacgatggcctcgacagt
caggccgcgtgcgtccagtctttccagttcctccctttcaggtcgattgt
ggcatcatttattgcctgctcattgcagttgaaacgcgatatccgtttca
agacccgagtatggatggtactttggagatatgagcacttgccagacttc
ccggtcggagacttaaaaattccgataagaggttactgacgacggacctt
ataagtgggtcgtctagtggtcgcactgatcaaaacagttccgcccccgg
ctttgctcaaagtagcaaagcagctttatctcgggttgcagaggattttc
tggaaaaccgcaattttgcaggagagaacatatggcttcctcagttcttt
cctccgcagcagttgccacccgcagcaatgttgctcaagctaacatggtt
gcacctttcactggccttaagtcagctgcctcattccctgtttcaaggaa
gcaaaaccttgacatcacttccattgccagcaacggcggaagagtgcaat
gcatgCAGGTGTGGCCAGCGATGGCTCCCGATCGGGCCcaagtaatcatt
cgcattgtgccaggaggtggaaccaagacccttcagcagataatcaatca
gctggagtacctgtccacgaagggaaagctggaactccagcgttcagccc
ggcatctcgatattcccgttccgccggatcaaatccgtgagcttgcccaa
agctgggttacggaggccgggatttatgacgaaagtcagtcagacgatga
caggcaacaagacttaacaacacacattattgtgagcttccccgcaggta
cggaccaaaccgcagcttatgaagcaagccgggaatgggcagccgagatg
tttgggtcaggatacgggggtggccgctataactatctgacagcctacca
cgtggaccgcgatcatccacatttacatgtcgtggtcaatcgtcgggaac
ttctggggcaggggtggctgaaaatatccaggcgccatccccagctgaat
tatgacggcttacggaaaaagatggcagagatttcacttcgtcacggcat
agtcctggatgcgacttcccgagcagaaaggggaatagcagagcgaccaa
tcacctatgctgaatatcgacgtCTTGAGCGGATGCTCGAAGTTATGGCA
GAAGTTCGGCCCATAGCCCGATCCATTAAAACGGCTCACGACGATGCGCG
AGCGGAATTAATGTCGGCGGACAGACCTCGATCAACGCGCGGTCTATGAT
Tctagaatccgtgctgttcgtcacccgcttatagttgccatcacaatcgg
atttcaactgccccggtctttaggtgcggccgaggacaatgtagcacgca
cttacggtgcgtctcgtctctgacgctttctatgttccgca

> SEQ ID NO 29 DNA sequence of pAG-CHL-1 chloroplast binary transformation vector for introduction of transgenes (goi) into the plastid genome. The binary vector carries a Pt-VirD2 gene outside the T-DNA listed as SEQ ID No 27.

actttgatccaacccctccgctgctatagtgcagtcggcttctgacgttc
agtgcagccgtcttctgaaaacgacatgtcgcacaagtcctaagttacgc
gacaggctgccgccctgcccttttcctggcgttttcttgtcgcgtgtttt
agtcgcataaagtagaatacttgcgactagaaccggagacattacgccat
gaacaagagcgccgccgctggcctgctgggctatgcccgcgtcagcaccg
acgaccaggacttgaccaaccaacgggccgaactgcacgcggccggctgc
accaagctgttttccgagaagatcaccggcaccaggcgcgaccgcccgga
gctggccaggatgcttgaccacctacgccctggcgacgttgtgacagtga
ccaggctagaccgcctggcccgcagcacccgcgacctactggacattgcc
gagcgcatccaggaggccggcgcgggcctgcgtagcctggcagagccgtg
ggccgacaccaccacgccggccggccgcatggtgttgaccgtgttcgccg
gcattgccgagttcgagcgttccctaatcatcgaccgcacccggagcggg
cgcgaggccgccaaggcccgaggcgtgaagtttggcccccgccctaccct
caccccggcacagatcgcgcacgcccgcgagctgatcgaccaggaaggcc
gcaccgtgaaagaggcggctgcactgcttggcgtgcatcgctcgaccctg
taccgcgcacttgagcgcagcgaggaagtgacgcccaccgaggccaggcg
gcgcggtgccttccgtgaggacgcattgaccgaggccgacgccctggcgg
ccgccgagaatgaacgccaagaggaacaagcatgaaaccgcaccaggacg
gccaggacgaaccgtttttcattaccgaagagatcgaggcggagatgatc
gcggccgggtacgtgttcgagccgcccgcgcacgtctcaaccgtgcggct
gcatgaaatcctggccggtttgtctgatgccaagctggcggcctggccgg
ccagcttggccgctgaagaaaccgagcgccgccgtctaaaaaggtgatgt
gtatttgagtaaaacagcttgcgtcatgcggtcgctgcgtatatgatgcg
atgagtaaataaacaaatacgcaaggggaacgcatgaaggttatcgctgt
acttaaccagaaaggcgggtcaggcaagacgaccatcgcaacccatctag
cccgcgccctgcaactcgccggggccgatgttctgttagtcgattccgat
ccccagggcagtgcccgcgattgggcggccgtgcgggaagatcaaccgct
aaccgttgtcggcatcgaccgcccgacgattgaccgcgacgtgaaggcca
tcggccggcgcgacttcgtagtgatcgacggagcgccccaggcggcggac
ttggctgtgtccgcgatcaaggcagccgacttcgtgctgattccggtgca
gccaagcccttacgacatatgggccaccgccgacctggtggagctggtta
agcagcgcattgaggtcacggatggaaggctacaagcggcctttgtcgtg
tcgcgggcgatcaaaggcacgcgcatcggcggtgaggttgccgaggcgct
ggccgggtacgagctgcccattcttgagtcccgtatcacgcagcgcgtga
gctacccaggcactgccgccgccggcacaaccgttcttgaatcagaaccc
gagggcgacgctgcccgcgaggtccaggcgctggccgctgaaattaaatc
aaaactcatttgagttaatgaggtaaagagaaaatgagcaaaagcacaaa
cacgctaagtgccggccgtccgagcgcacgcagcagcaaggctgcaacgt
tggccagcctggcagacacgccagccatgaagcgggtcaactttcagttg
ccggcggaggatcacaccaagctgaagatgtacgcggtacgccaaggcaa
gaccattaccgagctgctatctgaatacatcgcgcagctaccagagtaaa
tgagcaaatgaataaatgagtagatgaattttagcggctaaaggaggcgg
catggaaaatcaagaacaaccaggcaccgacgccgtggaatgccccatgt
gtggaggaacgggcggttggccaggcgtaagcggctgggttgtctgccgg
ccctgcaatggcactggaacccccaagcccgaggaatcggcgtgagcggt
cgcaaaccatccggcccggtacaaatcggcgcggcgctgggtgatgacct
ggtggagaagttgaaggccgcgcaggccgcccagcggcaacgcatcgagg
cagaagcacgccccggtgaatcgtggcaagcggccgctgatcgaatccgc
aaagaatcccggcaaccgccggcagccggtgcgccgtcgattaggaagcc
gcccaagggcgacgagcaaccagattttttcgttccgatgctctatgacg
tgggcacccgcgatagtcgcagcatcatggacgtggccgttttccgtctg
tcgaagcgtgaccgacgagctggcgaggtgatccgctacgagcttccaga
cgggcacgtagaggtttccgcagggccggccggcatggccagtgtgtggg
attacgacctggtactgatggcggtttcccatctaaccgaatccatgaac
cgataccgggaagggaagggagacaagcccggccgcgtgttccgtccaca
cgttgcggacgtactcaagttctgccggcgagccgatggcggaaagcaga
aagacgacctggtagaaacctgcattcggttaaacaccacgcacgttgcc
atgcagcgtacgaagaaggccaagaacggccgcctggtgacggtatccga
gggtgaagccttgattagccgctacaagatcgtaaagagcgaaaccgggc
ggccggagtacatcgagatcgagctagctgattggatgtaccgcgagatc
acagaaggcaagaacccggacgtgctgacggttcaccccgattacttttt
gatcgatcccggcatcggccgttttctctaccgcctggcacgccgcgccg
caggcaaggcagaagccagatggttgttcaagacgatctacgaacgcagt
ggcagcgccggagagtcaagaagttctgtttcaccgtgcgcaagctgatc
gggtcaaatgacctgccggagtacgatttgaaggaggaggcggggcaggc
tggcccgatcctagtcatgcgctaccgcaacctgatcgagggcgaagcat
ccgccggttcctaatgtacggagcagatgctagggcaaattgccctagca
ggggaaaaaggtcgaaaaggtctctttcctgtggatagcacgtacattgg
gaacccaaagccgtacattgggaaccggaacccgtacattgggaacccaa
agccgtacattgggaaccggtcacacatgtaagtgactgatataaaagag
aaaaaaggcgatttttccgcctaaaactctttaaaacttattaaaactct
taaaacccgcctggcctgtgcataactgtctggccagcgcacagccgaag
agctgcaaaaagcgcctacccttcggtcgctgcgctccctacgccccgcc
gcttcgcgtcggcctatcgcggccgctggccgctcaaaaatggctggcct
acggccaggcaatctaccagggcgcggacaagccgcgccgtcgccactcg
accgccggcgcccacatcaaggcaccctgcctcgcgcgtttcggtgatga
cggtgaaaacctctgacacatgcagctcccggagacggtcacagcttgtc
tgtaagcggatgccgggagcagacaagcccgtcagggcgcgtcagcgggt
gttggcgggtgtcggggcgcagccatgacccagtcacgtagcgatagcgg
agtgtatactggcttaactatgcggcatcagagcagattgtactgagagt
gcaccatatgcggtgtgaaataccgcacagatgcgtaaggagaaaatacc
gcatcaggcgctcttccgcttcctcgctcactgactcgctgcgctcggtc
gttcggctgcggcgagcggtatcagctcactcaaaggcggtaatacggtt
atccacagaatcaggggataacgcaggaaagaacatgtgagcaaaaggcc
agcaaaaggccaggaaccgtaaaaaggccgcgttgctggcgtttttccat
aggctccgcccccctgacgagcatcacaaaaatcgacgctcaagtcagag
gtggcgaaacccgacaggactataaagataccaggcgtttccccctggaa
gctccctcgtgcgctctcctgttccgaccctgccgcttaccggatacctg
tccgcctttctcccttcgggaagcgtggcgctttctcatagctcacgctg
taggtatctcagttcggtgtaggtcgttcgctccaagctgggctgtgtgc
acgaaccccccgttcagcccgaccgctgcgccttatccggtaactatcgt
cttgagtccaacccggtaagacacgacttatcgccactggcagcagccac
tggtaacaggattagcagagcgaggtatgtaggcggtgctacagagttct
tgaagtggtggcctaactacggctacactagaaggacagtatttggtatc
tgcgctctgctgaagccagttaccttcggaaaaagagttggtagctcttg
atccggcaaacaaaccaccgctggtagcggtggtttttttgtttgcaagc
agcagattacgcgcagaaaaaaaggatctcaagaagatcctttgatcttt
tctacggggtctgacgctcagtggaacgaaaactcacgttaagggatttt
ggtcatgcatGCTGAAGCCAGTTACCTTCGGAAAAAGAGTTGGTAGCTCT
TGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCT
TTTCTACGGGGTCTGACGCTCAGTGGAACGAAAACTCACGTTAAGGGATT
TTGGTCATGAGCTTGCGCCGTCCCGTCAAGTCAGCGTAATGCTCTGCCAG
TGTTACAACCAATTAACCAATTCTGATTAGAAAAACTCATCGAGCATCAA
ATGAAACTGCAATTTATTCACATCAGGATTATCAATACCATATTTTTGAA
AAAGCCGTTTCTGTAATGAAGGAGAAAACTCACCGAGGCAGTTCCATAGG
ATGGCAAGATCCTGGTATCGGTCTGCGATTCCGACTCGTCCAACATCAAT
ACAACCTATTAATTTCCCCTCGTCAAAAATAAGGTTATCAAGTGAGAAAT
CACCATGAGTGACGACTGAATCCGGTGAGAATGGCAAAAGTTTgTGCATT
TCTTTCCAGACTTGTTCAACAGGCCAGCCATTACGCTCGTCATCAAAATC
ACTCGCATCAACCAAACCGTTATTCATTCGTGATTGCGCCTGAGCAAGAC
GAAATACGCGATCGCTGTTAAAAGGACAATTACAAACAGGAATCGAATGC
AACCGGCGCAGGAACACTGCCAGCGCATCAACAATATTTTCACCTGAATC
AGGATATTCTTCTAATACCTGGAATGCTGTTTTTCCGGGGATCGCAGTGG
TGAGTAACCAcGCATCATCAGGAGTACGGATAAAATGCTTGATGGTCGGA
AGAGGCATAAATTCCGTCAGCCAGTTTAGTCTGACCATCTCATCTGTAAC
ATCATTGGCAACGCTACCTTTGCCATGTTTCAGAAACAACTCTGGCGCAT
CGGGCTTCCCATACAAGCGATAGATTGTCGCACCTGATTGCCCGACATTA
TCGCGtGCCCATTTATACCCATATAAATCAGCATCCATGTTGGAATTTAA
TCGCGGCCTCGACGTTTCCCGTTGAATATGGCTCATAACACCCCTTGTAT
TACTGTTTATGTAAGCAGACAGTTTTATTGTTCATGATGATATATTTTTA
TCTTGTGCAATGTAACATCAGAGATTTTGAGACACAACGTGGCTTTCCCC
CCCCCCCCCATGACATTAACCTATAAAAATAGGCGTATCACGAGGCCagg
cctcTGGCAGGATATATTGTGGTGTAAACCGAGCTGTaTaGGCTGGCTGG
TGGCAGGATATATTGTGGTGTAAACttaattaaAATTCACCGCCGTATGG
CTGACCGGCGATTACTAGCGATTCCGGCTTCATGCAGGCGAGTTGCAGCC
TGCAATCCGAACTGAGGACGGGTTTTTGGGGTTAGCTCACCCTCGCGGGA
TCGCGACCCTTTGTCCCGGCCATTGTAGCACGTGTGTCGCCCAGGGCATA
AGGGGCATGATGACTTGACGTCATCCTCACCTTCCTCCGGCTTATCACCG
GCAGTCTGTTCAGGGTTCCAAACTCAACGATGGCAACTAAACACGAGGGT
TGCGCTCGTTGCGGGACTTAACCCAACACCTTACGGCACGAGCTGACGAC
AGCCATGCACCACCTGTGTCCGCGTTCCCGAAGGCACCCCTCTCTTTCAA
GAGGATTCGCGGCATGTCAAGCCCTGGTAAGGTTCTTCGCTTTGCATCGA
ATTAAACCACATGCTCCACCGCTTGTGCGGGCCCCCGTCAATTCCTTTGA
GTTTCATTCTTGCGAACGTACTCCCCAGGCGGGATACTTAACGCGTTAGC
TACAGCACTGCACGGGTCGATACGCACAGCGCCTAGTATCCATCGTTTAC
GGCTAGGACTACTGGGGTATCTAATCCCATTCGCTCCCCTAGCTTTCGTC
TCTCAGTGTCAGTGTCGGCCCAGCAGAGTGCTTTCGCCGTTGGTGTTCTT
TCCGATCTCTACGCATTTCACCGCTCCACCGGAAATTCCCTCTGCCCCTA
CCGTACTCCAGCTTGGTAGTTTCCACCGCCTGTCCAGGGTTGAGCCCTGG
GATTTGACGGCGGACTTAAAAAGCCACCTACAGACGCTTTACGCCCAATC
ATTCCGGATAACGCTTGCATCCTCTGTATTACCGCGGCTGCTGGCACAGA
GTTAGCCGATGCTTATTCCCCAGATACCGTCATTGCTTCTTCTCCGGGAA
AAGAAGTTCACGACCCGTGGGCCTTCTACCTCCACGCGGCATTGCTCCGT
CAGGCTTTCGCCCATTGCGGAAAATTCCCCACTGCTGCCTCCCGTAGGAG
TCTGGGCCGTGTCTCAGTCCCAGTGTGGCTGATCATCCTCTCGGACCAGC
TACTGATCATCGCCTTGGTAAGCTATTGCCTCACCAACTAGCTAATCAGA
CGCGAGCCCCTCCTCGGGCGGATTCCTCCTTTTGCTCCTCAGCCTACGGG
GTATTAGCAGCCGTTTCCAGCTGTTGTTCCCCTCCCAAGGGCAGGTTCTT
ACGCGTTACTCACCCGTCCGCCACTGGAAACACCACTTCCCGTCCGACTT
GCATGTGTTAAGCATGCCGCCAGCGTTCATCCTGAGCCAGGATCGAACTC
TCCATGAGATTCATAGTTGCATTACTTATAGCTTCCTTGTTCGTAGACAA
AGCGGATTCGGAATTGTCTTTCATTCCAAGGCATAACTTGTATCCATGCG
CTTCATATTCGCCCGGAGTTCGCTCCCAGAAATATAGCCATCCCTGCCCC
CTCACGTCAATCCCACGAGCCTCTTATCCATTCTCATTGAACGACGGCGG
GGGAGCAAATCCAACTAGAAAAACTCACATTGGGCTTAGGGATAATCAGG
CTCGAACTGATGACTTCCACCACGTCAAGGTGACACTCTACCGCTGAGTT
ATATCCCTTCCCCGCCCCATCGAGAAATAGAACTGACTAATCCTAAGTCA
AAGGGTCGAGAAACTCAACGCCACTATTCTTGAACAACTTGGAGCCGGGC
CTTCTTTTCGCACTATTACGGATATGAAAATAATGGTCAAAATCGGATTC
AATTGTCTACcatgaataaatgcaagaaaataacctctccttctttttct
ataatgtaaacaaaaaagtctatgtaagtaaaatactagtaaataaataa
aaagaaaaaaagaaaggagcaatagcaccctcttgatagaacaagaaaat
gattattgctcctttcttttcaaaacctcctatagactaggccaggatcT
TACAAGTCTTCTTCAGAAATGAGTTTTTGttcTTTGCCcACTACCTTGGT
GATCTCGCCTTTCACGTAGTGGACAAATTCTTCCAACTGATCTGCGCGCG
AGGCCAAGCGATCTTCTTCTTGTCCAAGATAAGCCTGTCTAGCTTCAAGT
ATGACGGGCTGATACTGGGCCGGCAGGCGCTCCATTGCCCAGTCGGCAGC
GACATCCTTCGGCGCGATTTTGCCGGTTACTGCGCTGTACCAAATGCGGG
ACAACGTAAGCACTACATTTCGCTCATCGCCAGCCCAGTCGGGCGGCGAG
TTCCATAGCGTTAAGGTTTCATTTAGCGCCTCAAATAGATCCTGTTCAGG
AACCGGATCAAAGAGTTCCTCCGCCGCTGGACCTACCAAGGCAACGCTAT
GTTCTCTTGCTTTTGTCAGCAAGATAGCCAGATCAATGTCGATCGTGGCT
GGCTCGAAGATACCTGCAAGAATGTCATTGCGCTGCCATTCTCCAAATTG
CAGTTCGCGCTTAGCTGGATAACGCCACGGAATGATGTCGTCGTGCACAA
CAATGGTGACTTCTACAGCGCGGAGAATCTCGCTCTCTCCAGGGGAAGCC
GAAGTTTCCAAAAGGTCGTTGATCAAAGCTCGCCGCGTTGTTTCATCAAG
CCTTACGGTCACCGTAACCAGCAAATCAATATCACTGTGTGGCTTCAGGC
CGCCATCCACTGCGGAGCCGTACAAATGTACGGCCAGCAACGTCGGTTCG
AGATGGCGCTCGATGACGCCAACTACCTCTGATAGTTGAGTCGATACTTC
GGCGATCACCGCTTCgctagtagcCGTGGAAACCCCAGAACCAGAAGTAG
TAGGATTGATTCTCATAATAATAAAATAAATAAATATGTCGAAATGTTTT
TGCAAAAATTATCGAATTtAAAATAAATGTCCGCTtGCACGTCGATCGGT
TAATTCTCCCAGAAATATAGCCATCCCTGCCCCCTCACGTCAATCCCACG
AGCCTCTTATCCATTCTCATTGAACGACGGCGGGGGAGCGTAgaattcga
gctcggtacccggggatcctctagagtcgacctgcaggcatgcaagcttG
TTAACAACTGCCCCTATCGGAAATAGGATTGACTACCGATTCCGAAGGAA
CTGGAGTTACATCTCTTTTCCATTCAAGAGTTCTTATGCGTTTCCACGCC
CCTTTGAGACCCCGAAAAATGGACAAATTCCTTTTCTTAGGAACACATAC
AAGATTCGTCACTACAAAAAGGATAATGGTAACCCTACCATTAACTACTT
CATTTATGAATTTCATAGTAATAGAAATACATGTCCTACCGAGACAGAAT
TTGGAACTTGCTATCCTCTTGCCTAGCAGGCAAAGATTTACCTCCGTGGA
AAGGATGATTCATTCGGATCGACATGAGAGTCCAACTACATTGCCAGAAT
CCATGTTGTATATTTGAAAGAGGTTGACCTCCTTGCTTCTCTCATGGTAC
ACTCCTCTTCCCGCCGAGCCCCTTTTCTCCTCGGTCCACAGAGACAAAAT
GTAGGACTGGTGCCAACAATTCATCAGACTCACTAAGTCGGGATCACTAA
CTAATACTAATCTAATATAATAGTCTAATATATCTAATATAATAGAAAAT
ACTAATATAATAGAAAAGAACTGTCTTTTCTGTATACTTTCCCCGGTTCC
GTTGCTACCGCGGGCTTTACGCAATCGATCGGATTAGATAGATATCCCTT
CAACATAGGTCATCGAAAGGATCTCGGAGACCCACCAAAGTACGAAAGCC
AGGATCTTTCAGAAAACGGATTCCTATTCAAAGAGTGCATAACCGCATGG
ATAAGCTCACACTAACCCGTCAATTTGGGATCCAAATTCGAGATTTTCCT
TGGGAGGTATCGGGAAGGATTTGGAATGGAATAATATCGATTCATACAGA
AGAAAAGGTTCTCTATTGATTCAAACACTGTACCTAACCTATGGGATAGG
GATCGAGGAAGGGGAAAAACCGAAGATTTCACATGGTACTTTTATCAATC
TGATTTATTTCGTACCTTTCGTTCAATGAGAAAATGGGTCAAATTCTACA
GGATCAAACCTATGGGACTTAAGGAATGATATAAAAAAAAGAGAGGGAAA
ATATTCATATTAAATAAATATGAAGTAGAAGAACCCAGATTCCAAATGAA
CAAATTCAAACTTGAAAAGGATCTTCCTTATTCTTGAAGAATGAGGGGCA
AAGGGATTGATCAAGAAAGATCGGCGCGCCtgacaggatatattggcggg
taaactaagtcgctgtatgtgtttgtttggaattttatcgatgagtaaaa
gcgttgttacactatttttatttcacattcgttataagacaattgcaaat
gtagcaagtatattcagtattgactgtaaatgtactgttgatttcatatt
gagcagggctagacttccatccgtctacccgggcacatttcgtgctggag
tatccagaccttccgctttctttggaggaagctatggcttcctcagttct
ttcctccgcagcagttgccacccgcagcaatgttgctcaagctaacatgg
ttgcacctttcactggccttaagtcagctgcctcattccctgtttcaagg
aagcaaaaccttgacatcacttccattgccagcaacggcggaagagtgca
atgcatgCAGGTGTGGCCAGCGATGGCTCCCGATCGGGCCcaagtaatca
ttcgcattgtgccaggaggtggaaccaagacccttcagcagataatcaat
cagctggagtacctgtccacgaagggaaagctggaactccagcgttcagc
ccggcatctcgatattcccgttccgccggatcaaatccgtgagcttgccc
aaagctgggttacggaggccgggatttatgacgaaagtcagtcagacgat
gacaggcaacaagacttaacaacacacattattgtgagcttccccgcagg
tacggaccaaaccgcagcttatgaagcaagccgggaatgggcagccgaga
tgtttgggtcaggatacgggggtggccgctataactatctgacagcctac
cacgtggaccgcgatcatccacatttacatgtcgtggtcaatcgtcggga
acttctggggcaggggtggctgaaaatatccaggcgccatccccagctga
attatgacggcttacggaaaaagatggcagagatttcacttcgtcacggc
atagtcctggatgcgacttcccgagcagaaaggggaatagcagagcgacc
aatcacctatgctgaatatcgacgtCTTGAGCGGATGCTCGAAGTTATGG
CAGAAGTTCGGCCCATAGCCCGATCCATTAAAACGGCTCACGACGATGCG
CGAGCGGAATTAATGTCGGCGGACAGACCTCGATCAACGCGCGGTCTATG
ATTctagaatccgtgctgttcgtcacccgcttatagttgccatcacaatc
ggatttcaactgccccggtctttaggtgcggccgaggacaatgtagcacg
cacttacggtgcgtctcgtctctgacgctttctatgttccgcagcttagt

> SEQ ID NO 30 DNA sequence of kanamycin resistance gene in StuI-NsiI fragment

aggcctGGCCTCGTGATACGCCTATTTTTATAGGTTAATGTCATGGGGGG
GGGGGGGAAAGCCACGTTGTGTCTCAAAATCTCTGATGTTACATTGCACA
AGATAAAAATATATCATCATGAACAATAAAACTGTCTGCTTACATAAACA
GTAATACAAGGGGTGTTATGAGCCATATTCAACGGGAAACGTCGAGGCCG
CGATTAAATTCCAACATGGATGCTGATTTATATGGGTATAAATGGGCaCG
CGATAATGTCGGGCAATCAGGTGCGACAATCTATCGCTTGTATGGGAAGC
CCGATGCGCCAGAGTTGTTTCTGAAACATGGCAAAGGTAGCGTTGCCAAT
GATGTTACAGATGAGATGGTCAGACTAAACTGGCTGACGGAATTTATGCC
TCTTCCGACCATCAAGCATTTTATCCGTACTCCTGATGATGCgTGGTTAC
TCACCACTGCGATCCCCGGAAAAACAGCATTCCAGGTATTAGAAGAATAT
CCTGATTCAGGTGAAAATATTGTTGATGCGCTGGCAGTGTTCCTGCGCCG
GTTGCATTCGATTCCTGTTTGTAATTGTCCTTTTAACAGCGATCGCGTAT
TTCGTCTTGCTCAGGCGCAATCACGAATGAATAACGGTTTGGTTGATGCG
AGTGATTTTGATGACGAGCGTAATGGCTGGCCTGTTGAACAAGTCTGGAA
AGAAATGCAcAAACTTTTGCCATTCTCACCGGATTCAGTCGTCACTCATG
GTGATTTCTCACTTGATAACCTTATTTTTGACGAGGGGAAATTAATAGGT
TGTATTGATGTTGGACGAGTCGGAATCGCAGACCGATACCAGGATCTTGC
CATCCTATGGAACTGCCTCGGTGAGTTTTCTCCTTCATTACAGAAACGGC
TTTTTCAAAAATATGGTATTGATAATCCTGATGTGAATAAATTGCAGTTT
CATTTGATGCTCGATGAGTTTTTCTAATCAGAATTGGTTAATTGGTTGTA
ACACTGGCAGAGCATTACGCTGACTTGACGGGACGGCGCAAGCTCATGAC
CAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGA
TCAAGAGCTACCAACTCTTTTTCCGAAGGTAACTGGCTTCAGCatgcat

> SEQ ID NO 31 The DNA sequence of Pt-VirD2-3 in a VirD promoter/VirF terminator cassette where Pt-VirD2 has the wild type NLS1. The Pt-VirD2-3 has the tobacco SSU Transit peptide+5aa, the wt minimal VirD2-T4SS signal.

ttatcgatgagtaaaagcgttgttacactatttttatttcacattcgtta
taagacaattgcaaatgtagcaagtatattcagtattgactgtaaatgta
ctgttgatttcatattgagcagggctagacttccatccgtctacccgggc
acatttcgtgctggagtatccagaccttccgctttctttggaggaagcta
tggcttcctcagttctttcctccgcagcagttgccacccgcagcaatgtt
gctcaagctaacatggttgcacctttcactggccttaagtcagctgcctc
attccctgtttcaaggaagcaaaaccttgacatcacttccattgccagca
acggcggaagagtgcaatgcatgCAGGTGTGGCCAGCGATGGCTCCCGAT
CGGGCCCAAGTAATCATTCGCATTGTGCCAGGAGGTGGAACCAAGACCCT
TCAGCAGATAATCAATCAGCTGGAGTACCTGTCCCGAAAGGGAAAGCTGG
AACTCCAGCGTTCAGCCCGGCATCTCGATATTCCCGTTCCGCCGGATCAA
ATCCGTGAGCTTGCCCAAAGCTGGGTTACGGAGGCCGGGATTTATGACGA
AAGTCAGTCAGACGATGACAGGCAACAAGACTTAACAACACACATTATTG
TGAGCTTCCCCGCAGGTACCGACCAAACCGCAGCTTATGAAGCAAGCCGG
GAATGGGCAGCCGAGATGTTTGGGTCAGGATACGGGGGTGGCCGCTATAA
CTATCTGACAGCCTACCACGTGGACCGCGATCATCCACATTTACATGTCG
TGGTCAATCGTCGGGAACTTCTGGGGCAGGGGTGGCTGAAAATATCCAGG
CGCCATCCCCAGCTGAATTATGACGGCTTACGGAAAAAGATGGCAGAGAT
TTCACTTCGTCACGGCATAGTCCTGGATGCGACTTCCCGAGCAGAAAGGG
GAATAGCAGAGCGACCAATCACCTATGCTGAATATCGACGTCTTGAGCGG
ATGCTCGAAGTTATGGCAGAAGTTCGGCCCATAGCCCGATCCATTAAAAC
GGCTCACGACGATGCGCGAGCGGAATTAATGTCGGCGGACAGACCTCGAT
CAACGCGCGGTCTATGATTctagaatccgtgctgttcgtcacccgcttat
agttgccatcacaatcggatttcaactgccccggtctttaggtgcggccg
aggacaatgtagcacgcacttacggtgcgtctcgtctctgacgctttcta
tgttccgca

> SEQ ID NO 32 The DNA sequence of Pt-VirD2-4 in a VirE promoter/VirF terminator cassette where Pt-VirD2 has the wild type NLS1. The Pt-VirD2-4 has the tobacco SSU Transit peptide+5aa, the wt minimal VirD2-T4SS signal.

cggctgctcgtcaccaacaatgctcacagggatacgatggcctcgacagt
caggccgcgtgcgtccagtctttccagttcctccctttcaggtcgattgt
ggcatcatttattgcctgctcattgcagttgaaacgcgatatccgtttca
agacccgagtatggatggtactttggagatatgagcacttgccagacttc
ccggtcggagacttaaaaattccgataagaggttactgacgacggacctt
ataagtgggtcgtctagtggtcgcactgatcaaaacagttccgcccccgg
ctttgctcaaagtagcaaagcagctttatctcgggttgcagaggattttc
tggaaaaccgcaattttgcaggagagaacatatggcttcctcagttcttt
cctccgcagcagttgccacccgcagcaatgttgctcaagctaacatggtt
gcacctttcactggccttaagtcagctgcctcattccctgtttcaaggaa
gcaaaaccttgacatcacttccattgccagcaacggcggaagagtgcaat
gcatgCAGGTGTGGCCAGCGATGGCTCCCGATCGGGCCCAAGTAATCATT
CGCATTGTGCCAGGAGGTGGAACCAAGACCCTTCAGCAGATAATCAATCA
GCTGGAGTACCTGTCCCGAAAGGGAAAGCTGGAACTCCAGCGTTCAGCCC
GGCATCTCGATATTCCCGTTCCGCCGGATCAAATCCGTGAGCTTGCCCAA
AGCTGGGTTACGGAGGCCGGGATTTATGACGAAAGTCAGTCAGACGATGA
CAGGCAACAAGACTTAACAACACACATTATTGTGAGCTTCCCCGCAGGTA
CCGACCAAACCGCAGCTTATGAAGCAAGCCGGGAATGGGCAGCCGAGATG
TTTGGGTCAGGATACGGGGGTGGCCGCTATAACTATCTGACAGCCTACCA
CGTGGACCGCGATCATCCACATTTACATGTCGTGGTCAATCGTCGGGAAC
TTCTGGGGCAGGGGTGGCTGAAAATATCCAGGCGCCATCCCCAGCTGAAT
TATGACGGCTTACGGAAAAAGATGGCAGAGATTTCACTTCGTCACGGCAT
AGTCCTGGATGCGACTTCCCGAGCAGAAAGGGGAATAGCAGAGCGACCAA
TCACCTATGCTGAATATCGACGTCTTGAGCGGATGCTCGAAGTTATGGCA
GAAGTTCGGCCCATAGCCCGATCCATTAAAACGGCTCACGACGATGCGCG
AGCGGAATTAATGTCGGCGGACAGACCTCGATCAACGCGCGGTCTATGAT
Tctagaatccgtgctgttcgtcacccgcttatagttgccatcacaatcgg
atttcaactgccccggtctttaggtgcggccgaggacaatgtagcacgca
cttacggtgcgtctcgtctctgacgctttctatgttccgca

> SEQ ID NO 33 DNA sequence introducing a C to T point mutation at nucleotide 473 in tobacco plastid 16S RNA coding sequence flanked by PacI and AscI conferring streptomycin resistance

TTAATTAAAATAAGCATCGGCTAACTCTGTGCCAGCAGICGCGGTAATAC
AGAGGATGCAAGCGTTATCGGCGCGCC

> SEQ ID NO 34 DNA sequence introducing a C to A point mutation at nucleotide 861 in tobacco plastid 16S RNA coding sequence flanked by PacI and AscI conferring streptomycin resistance

TTAATTAACCTGGGGAGTACGTTCGCAAGAATGAAACTAAAAGGAATTGA
CGGGGGCCCGCACAAGCGGGGCGCGCC

> SEQ ID NO 35 DNA sequence introducing a C to T point mutation at nucleotide 861 in tobacco plastid 16S RNA coding sequence flanked by PacI and AscI conferring streptomycin resistance

TTAATTAACCTGGGGAGTACGTTCGCAAGAATGAAACTIAAAGGAATTGA
CGGGGGCCCGCACAAGCGGGGCGCGCC

> SEQ ID NO 36 DNA sequence introducing a G to A point mutation at nucleotide 1013 in tobacco plastid 16S RNA coding sequence flanked by PacI and AscI conferring spectinomycin resistance

TTAATTAAGAACGCGGACACAGGTGGTGCATGGCTGTCATCAGCTCGTGC
CGTAAGGTGTTGGGTTAAGGGCGCGCC

> SEQ ID NO 37 DNA sequence introducing a A to C point mutation at nucleotide 1139 in tobacco plastid 16S RNA coding sequence flanked by PacI and AscI conferring spectinomycin resistance

TTAATTAACCGGTGATAAGCCGGAGGAAGGTGAGGATGCCGTCAAGTCAT
CATGCCCCTTATGCCCTGGGGCGCGCC

> SEQ ID NO 38 DNA sequence introducing a C to T point mutation at nucleotide 1140 in tobacco plastid 16S RNA coding sequence flanked by PacI and AscI conferring spectinomycin resistance

TTAATTAACGGTGATAAGCCGGAGGAAGGTGAGGATGATGTCAAGTCATC
ATGCCCCTTATGCCCTGGGGGCGCGCC

> SEQ ID NO 39 DNA sequence introducing a G to A point mutation at nucleotide 1141 in tobacco plastid 16S RNA coding sequence flanked by PacI and AscI conferring spectinomycin resistance

TTAATTAAGGTGATAAGCCGGAGGAAGGTGAGGATGACATCAAGTCATCA
TGCCCCTTATGCCCTGGGCGGCGCGCC

> SEQ ID NO 40 DNA sequence introducing a G to A point mutation at nucleotide 1335 in tobacco plastid 16S RNA coding sequence flanked by PacI and AscI conferring spectinomycin resistance

TTAATTAAGTCAGCCATACGGCGGTGAATTCGTTCCCGAGCCTTGTACAC
ACCGCCCGTCACACTATGGGGCGCGCC

> SEQ ID NO 41 DNA sequence introducing streptomycin resistance and a silent mutation in the tobacco rps12 coding sequence. Flanked by PacI and AscI restriction sites.. C271T changes Pro to Ser conferring streptomycin resistance. A270G is a silent mutation.

TTAATTAAGTCTTAGTAAGAGGGGGAAGGGTTAAGGATTTGTCCGGTGTG
AGATATCACATTGTTCGAGGAAGGCGCGCC

> SEQ ID NO 42 DNA sequence of Pt-ACC2-virD2 in a VirE promoter/VirF terminator cassette (P/T are in lower case) where Pt-ACC2-virD2 has A. thaliana ACC2 transit peptide+6aa fused with wt minimal VirD2 and T4SS signal.

ttatcgatgagtaaaagcgttgttacactatttttatttcacattcgtta
taagacaattgcaaatgtagcaagtatattcagtattgactgtaaatgta
ctgttgatttcatattgagcagggctagacttccatccgtctacccgggc
acatttcgtgctggagtatccagaccttccgctttctttggaggaagcta
tggagatgagagctttgggttcttcgtgttctactggtaatggaggttct
gctccgattaccctcacgaatatatctccttggatcacaacagtttttcc
atctacagtgaagctgagaagtagtttgagaaccttcaaaggagtttcgt
caagagtgagaacctttaaaggagtttcttcgacaagagttttgtctcgg
accaaacaacagtttcctctgttttgtttcctaaaccctgatccgATGGC
TCCCGATCGGGCCCAAGTAATCATTCGCATTGTGCCAGGAGGTGGAACCA
AGACCCTTCAGCAGATAATCAATCAGCTGGAGTACCTGTCCCGAAAGGGA
AAGCTGGAACTCCAGCGTTCAGCCCGGCATCTCGATATTCCCGTTCCGCC
GGATCAAATCCGTGAGCTTGCCCAAAGCTGGGTTACGGAGGCCGGGATTT
ATGACGAAAGTCAGTCAGACGATGACAGGCAACAAGACTTAACAACACAC
ATTATTGTGAGCTTCCCCGCAGGTACCGACCAAACCGCAGCTTATGAAGC
AAGCCGGGAATGGGCAGCCGAGATGTTTGGGTCAGGATACGGGGGTGGCC
GCTATAACTATCTGACAGCCTACCACGTGGACCGCGATCATCCACATTTA
CATGTCGTGGTCAATCGTCGGGAACTTCTGGGGCAGGGGTGGCTGAAAAT
ATCCAGGCGCCATCCCCAGCTGAATTATGACGGCTTACGGAAAAAGATGG
CAGAGATTTCACTTCGTCACGGCATAGTCCTGGATGCGACTTCCCGAGCA
GAAAGGGGAATAGCAGAGCGACCAATCACCTATGCTGAATATCGACGTCT
TGAGCGGATGCTCGAAGTTATGGCAGAAGTTCGGCCCATAGCCCGATCCA
TTAAAACGGCTCACGACGATGCGCGAGCGGAATTAATGTCGGCGGACAGA
CCTCGATCAACGCGCGGTCTATGAttctagaatccgtgctgttcgtcacc
cgcttatagttgccatcacaatcggatttcaactgccccggtctttaggt
gcggccgaggacaatgtagcacgcacttacggtgcgtctcgtctctgacg
ctttctatgttccgca

> SEQ ID NO 43 DNA sequence of PtMKK4-virD2 in a VirE promoter/VirF terminator cassette (P/T are in lower case) where PtMKK4-virD2 has A. thaliana MKK4 transit peptide fused with wt minimal VirD2 and T4SS signal.

ttatcgatgagtaaaagcgttgttacactatttttatttcacattcgtta
taagacaattgcaaatgtagcaagtatattcagtattgactgtaaatgta
ctgttgatttcatattgagcagggctagacttccatccgtctacccgggc
acatttcgtgctggagtatccagaccttccgctttctttggaggaagctA
TGAGACCGATTCAATCGCCTCCAGGAGTTTCCGTTCCGGTGAAAAGCCGT
CCCCGTCGCCGTCCTGATCTTACCTTACCGCTTCCTCAACGCGATGTTTC
TCTCGCTGTACCTCTTCCTCTCCCACCTACTTCCGGTGGTTCCGGTGGCT
CTAGTGGATCTGCGCCGTCTTCTGGTGGTTCGGCGTCTTCAACGATGGCT
CCCGATCGGGCCCAAGTAATCATTCGCATTGTGCCAGGAGGTGGAACCAA
GACCCTTCAGCAGATAATCAATCAGCTGGAGTACCTGTCCCGAAAGGGAA
AGCTGGAACTCCAGCGTTCAGCCCGGCATCTCGATATTCCCGTTCCGCCG
GATCAAATCCGTGAGCTTGCCCAAAGCTGGGTTACGGAGGCCGGGATTTA
TGACGAAAGTCAGTCAGACGATGACAGGCAACAAGACTTAACAACACACA
TTATTGTGAGCTTCCCCGCAGGTACCGACCAAACCGCAGCTTATGAAGCA
AGCCGGGAATGGGCAGCCGAGATGTTTGGGTCAGGATACGGGGGTGGCCG
CTATAACTATCTGACAGCCTACCACGTGGACCGCGATCATCCACATTTAC
ATGTCGTGGTCAATCGTCGGGAACTTCTGGGGCAGGGGTGGCTGAAAATA
TCCAGGCGCCATCCCCAGCTGAATTATGACGGCTTACGGAAAAAGATGGC
AGAGATTTCACTTCGTCACGGCATAGTCCTGGATGCGACTTCCCGAGCAG
AAAGGGGAATAGCAGAGCGACCAATCACCTATGCTGAATATCGACGTCTT
GAGCGGATGCTCGAAGTTATGGCAGAAGTTCGGCCCATAGCCCGATCCAT
TAAAACGGCTCACGACGATGCGCGAGCGGAATTAATGTCGGCGGACAGAC
CTCGATCAACGCGCGGTCTATGAttctagaatccgtgctgttcgtcaccc
gcttatagttgccatcacaatcggatttcaactgccccggtctttaggtg
cggccgaggacaatgtagcacgcacttacggtgcgtctcgtctctgacgc
tttctatgttccgca

> SEQ ID NO 44 DNA sequence of pEX18Kan. pEX18Tc (Accession no. AF047519) derivative with kanamycin resistance as selectable marker.

tcgcgcgtttcggtgatgacggtgaaaacctctgacacatgcagctcccg
gagacggtcacagcttgtctgtaagcggatgccgggagcagacaagcccg
tcagggcgcgtcagcgggtgttggcgggtgtcggggctggcttaactatg
cggcatcagagcagattgtactgagagtgcaccataatcggcattttctt
ttgcgtttttatttgttaactgttaattgtccttgttcaaggatgctgtc
tttgacaacagatgttttcttgcctttgatgttcagcaggaagctaggcg
caaacgttgattgtttgtctgcgtagaatcctctgtttgtcatatagctt
gtaatcacgacattgtttcctttcgcttgaggtacagcgaagtgtgagta
agtaaaggttacatcgttaggatcaagatccatttttaacacaaggccag
ttttgttcagcggcttgtatgggccagttaaagaattagaaacataacca
agcatgtaaatatcgttagacgtaatgccgtcaatcgtcatttttgatcc
gcgggagtcagtgaacagataccatttgccgttcattttaaagacgttcg
cgcgttcaatttcatctgttactgtgttagatgcaatcagcggtttcatc
acttttttcagtgtgtaatcatcgtttagctcaatcataccgagagcgcc
gtttgctaactcagccgtgcgttttttatcgctttgcagaagtttttgac
tttcttgacggaagaatgatgtgcttttgccatagtatgctttgttaaat
aaagattcttcgccttggtagccatcttcagttccagtgtttgcttcaaa
tactaagtatttgtggcctttatcttctacgtagtgaggatctctcagcg
tatggttgtcgcctgagctgtagttgccttcatcgatgaactgctgtaca
ttttgatacgtttttccgtcaccgtcaaagattgatttataatcctctac
accgttgatgttcaaagagctgtctgatgctgatacgttaacttgtgcag
ttgtcagtgtttgtttgccgtaatgtttaccggagaaatcagtgtagaat
aaacggatttttccgtcagatgtaaatgtggctgaacctgaccattcttg
tgtttggtcttttaggatagaatcatttgcatcgaatttgtcgctgtctt
taaagacgcggccagcgtttttccagctgtcaatagaagtttcgccgact
ttttgatagaacatgtaaatcgatgtgtcatccgcatttttaggatctcc
ggctaatgcaaagacgatgtggtagccgtgatagtttgcgacagtgccgt
cagcgttttgtaatggccagctgtcccaaacgtccaggccttttgcagaa
gagatatttttaattgtggacgaatcgaactcaggaacttgatatttttc
atttttttgctgttcagggatttgcagcatatcatggcgtgtaatatggg
aaatgccgtatgtttccttatatggcttttggttcgtttctttcgcaaac
gcttgagttgcgcctcctgccagcagtgcggtagtaaaggttaatactgt
tgcttgttttgcaaactttttgatgttcatcgttcatgtctcctttttta
tgtactgtgttagcggtctgcttcttccagccctcctgtttgaagatggc
aagttagttacgcacaataaaaaaagacctaaaatatgtaaggggtgacg
ccaaagtatacactttgccctttacacattttaggtcttgcctgctttat
cagtaacaaacccgcgcgatttacttttcgacctcattctattagactct
cgtttggattgcaactggtctattttcctcttttgtttgatagaaaatca
taaaaggatttgcagactacgggcctaaagaactaaaaaatctatctgtt
tcttttcattctctgtattttttatagtttctgttgcatgggcataaagt
tgcctttttaatcacaattcagaaaatatcataatatctcatttcactaa
ataatagtgaacggcaggtatatgtgatgggttaaaaaggatcgatcctc
tagctagagtcgatcttcgccagcagggcgaggatcgtggcatcaccgaa
ccgcgccgtgcgcgggtcgtcggtgagccagagtttcagcaggccgccca
ggcggcccaggtcgccattgatgcgggccagctcgcggacgtgctcatag
tccacgacgcccgtgattttgtagccctggccgacggccagcaggtaggc
cgacaggctcatgccggccgccgccgccttttcctcaatcgctcttcgtt
cgtctggaaggcagtacaccttgataggtgggctgcccttcctggttggc
ttggtttcatcagccatccgcttgccctcatctgttacgccggcggtagc
cggccagcctcgcagagcaggattcccgttgagcaccgccaggtgcgaat
aagggacagtgaagaaggaacacccgctcgcgggtgggcctacttcacct
atcctgcccggctgacgccgttggatacaccaaggaaagtctacacgaac
cctttggcaaaatcctgtatatcgtgcgaaaaaggatggatataccgaaa
aaatcgctataatgaccccgaagcagggttatgcagcggaaaagcgctgc
ttccctgctgttttgtggaatatctaccgactggaaacaggcaaatgcag
gaaattactgaactgaggggacaggcgagagacgatgccaaagagctaca
ccgacgagctggccgagtgggttgaatcccgcgcggccaagaagcgccgg
cgtgatgaggctgcggttgcgttcctggcggtgagggcggatgtcgatat
gcgtaaggagaaaataccgcatcaggcgcatatttgaatgtatttagaaa
aataaacaaaaagagtttgtagaaacgcaaaaaggccatccgtcaggatg
gccttctgcttaatttgatgcctggcagtttatggcgggcgtcctgcccg
ccaccctccgggccgttgcttcgcaacgttcaaatccgctcccggcggat
ttgtcctactcaggagagcgttcaccgacaaacaacagataaaacgaaag
gcccagtctttcgactgagcctttcgttttatttgatgcctggcagttcc
ctactctcgcatggggagaccccacactaccatcggcgctacggcgtttc
acttctgagttcggcatggggtcaggtgggaccaccgcgctactgccgcc
aggcaaattctgttttatcagaccgcttctgcgttctgatttaatctgta
tcaggctgaaaatcttctctcatccgccaaaacagccaagctcgccattc
gccattcaggctgcgcaactgttgggaagggcgatcggtgcgggcctctt
cgctattacgccagctggcgaaagggggatgtgctgcaaggcgattaagt
tgggtaacgccagggttttcccagtcacgacgttgtaaaacgacggccag
tgccaagcttgcatgcctgcaggtcgactctagaggatccccgggtaccg
agctcgaattcgtaatcatggtcatagctgtttcctgtgtgaaattgtta
tccgctcacaattccacacaacatacgagccggaagcataaagtgtaaag
cctggggtgcctaatgagtgagctaactcacattaattgcgttgcgctca
ctgcccgctttccagtcgggaaacctgtcgtgccagctgcattaatgaat
cggccaacgcgcggggagaggcggtttgcgtattgggcgctcttccgctt
cctcgctcactgactcgctgcgctcggtcgttcggctgcggcgagcggta
tcagctcactcaaaggcggtaatacggttatccacagaatcaggggataa
cgcaggaaagaacatgtgagcaaaaggccagcaaaaggccaggaaccgta
aaaaggccgcgttgctggcgtttttccataggctccgcccccctgacgag
catcacaaaaatcgacgctcaagtcagaggtggcgaaacccgacaggact
ataaagataccaggcgtttccccctggaagctccctcgtgcgctctcctg
ttccgaccctgccgcttaccggatacctgtccgcctttctcccttcggga
agcgtggcgctttctcatagctcacgctgtaggtatctcagttcggtgta
ggtcgttcgctccaagctgggctgtgtgcacgaaccccccgttcagcccg
accgctgcgccttatccggtaactatcgtcttgagtccaacccggtaaga
cacgacttatcgccactggcagcagccactggtaacaggattagcagagc
gaggtatgtaggcggtgctacagagttcttgaagtggtggcctaactacg
gctacactagaaggacagtatttggtatctgcgctctgctgaagccagtt
accttcggaaaaagagttggtagctcttgatccggcaaacaaaccaccgc
tggtagcggtggtttttttgtttgcaagcagcagattacgcgcagaaaaa
aaggatctcaagaagatcctttgatcttttctacggggtctgacgctcag
tggaacgaaaactcacgttaagggattttggtcatgagattatcaaaaag
gatcttcacctagatccttttaaattaaaaatgaagttttaaatcaatct
aaagtatatatgagtaaacttggtctgacaatcgatgcaagggttggttt
gTGCTGAAGCCAGTTACCTTCGGAAAAAGAGTTGGTAGCTCTTGATCCGG
CAAACAAACCACCGCTGGTAGCGGTGGTTTTTTTGTTTGCAAGCAGCAGA
TTACGCGCAGAAAAAAAGGATCTCAAGAAGATCCTTTGATCTTTTCTACG
GGGTCTGACGCTCAGTGGAACGAAAACTCACGTTAAGGGATTTTGGTCAT
GAGCTTGCGCCGTCCCGTCAAGTCAGCGTAATGCTCTGCCAGTGTTACAA
CCAATTAACCAATTCTGATTAGAAAAACTCATCGAGCATCAAATGAAACT
GCAATTTATTCACATCAGGATTATCAATACCATATTTTTGAAAAAGCCGT
TTCTGTAATGAAGGAGAAAACTCACCGAGGCAGTTCCATAGGATGGCAAG
ATCCTGGTATCGGTCTGCGATTCCGACTCGTCCAACATCAATACAACCTA
TTAATTTCCCCTCGTCAAAAATAAGGTTATCAAGTGAGAAATCACCATGA
GTGACGACTGAATCCGGTGAGAATGGCAAAAGTTTgTGCATTTCTTTCCA
GACTTGTTCAACAGGCCAGCCATTACGCTCGTCATCAAAATCACTCGCAT
CAACCAAACCGTTATTCATTCGTGATTGCGCCTGAGCAAGACGAAATACG
CGATCGCTGTTAAAAGGACAATTACAAACAGGAATCGAATGCAACCGGCG
CAGGAACACTGCCAGCGCATCAACAATATTTTCACCTGAATCAGGATATT
CTTCTAATACCTGGAATGCTGTTTTTCCGGGGATCGCAGTGGTGAGTAAC
CAcGCATCATCAGGAGTACGGATAAAATGCTTGATGGTCGGAAGAGGCAT
AAATTCCGTCAGCCAGTTTAGTCTGACCATCTCATCTGTAACATCATTGG
CAACGCTACCTTTGCCATGTTTCAGAAACAACTCTGGCGCATCGGGCTTC
CCATACAAGCGATAGATTGTCGCACCTGATTGCCCGACATTATCGCGTGC
CCATTTATACCCATATAAATCAGCATCCATGTTGGAATTTAATCGCGGCC
TCGACGTTTCCCGTTGAATATGGCTCATAACACCCCTTGTATTACTGTTT
ATGTAAGCAGACAGTTTTATTGTTCATGATGATATATTTTTATCTTGTGC
AATGTAACATCAGAGATTTTGAGACACAACGTGGCTTTCCCCCCCCCCCC
CATGACATTAACCTATAAAAATAGGCGTATCACGAGGCCAGGctaagaaa
ccattattatcatgacattaacctataaaaataggcgtatcacgaggccc
tttcgtc

> SEQ ID NO 45. EcoRI-HindIII fragment of Pt-virD2 flanked with 500 bp of virD1 and 500 bp of virD3. The PtVirD2 has the tobacco SSU Transit peptide, the wt minimal VirD2-T4SS signal listed as SEQ ID NO 31.

gaattcttccgctttctttggaggaagctatgtcaaaacacaccagagcc
acgtcgagtgagactaccatcaaccagcatcgatccctgaaagttgaagg
gttcaaggtcgtgagtgcccgtctgcgatcggccgagtatgaaacctttt
cctatcaagcgcgcctgctgggactttcggatagtatggcaattcgcgtt
gcggtgcgccgcatcgggggctttctcgaaatagatgcagacacacgaga
aaagatggaagccatacttcagtccatcggaatactctcaagcaatgtat
ccatgcttctatctgcctacgccgaagaccctcgatcggatctggaggct
gtgcgagatgaacgtattgcttttggcgaggctttcgccgccctcgatgg
actactgcgctccattttgtccgtatcccggcgacggatcgacggtcgct
cgttactgaaaggtgccttgtagcacttgaccacgcacctgacgggagaa
aattggATGGCTTCCTCAGTTCTTTCCTCCGCAGCAGTTGCCACCCGCAG
CAATGTTGCTCAAGCTAACATGGTTGCACCTTTCACTGGCCTTAAGTCAG
CTGCCTCATTCCCTGTTTCAAGGAAGCAAAACCTTGACATCACTTCCATT
GCCAGCAACGGCGGAAGAGTGCAATGCATGCAGGTGTGGCCAGCGATGGC
TCCCGATCGGGCCCAAGTAATCATTCGCATTGTGCCAGGAGGTGGAACCA
AGACCCTTCAGCAGATAATCAATCAGCTGGAGTACCTGTCCCGAAAGGGA
AAGCTGGAACTCCAGCGTTCAGCCCGGCATCTCGATATTCCCGTTCCGCC
GGATCAAATCCGTGAGCTTGCCCAAAGCTGGGTTACGGAGGCCGGGATTT
ATGACGAAAGTCAGTCAGACGATGACAGGCAACAAGACTTAACAACACAC
ATTATTGTGAGCTTCCCCGCAGGTACCGACCAAACCGCAGCTTATGAAGC
AAGCCGGGAATGGGCAGCCGAGATGTTTGGGTCAGGATACGGGGGTGGCC
GCTATAACTATCTGACAGCCTACCACGTGGACCGCGATCATCCACATTTA
CATGTCGTGGTCAATCGTCGGGAACTTCTGGGGCAGGGGTGGCTGAAAAT
ATCCAGGCGCCATCCCCAGCTGAATTATGACGGCTTACGGAAAAAGATGG
CAGAGATTTCACTTCGTCACGGCATAGTCCTGGATGCGACTTCCCGAGCA
GAAAGGGGAATAGCAGAGCGACCAATCACCTATGCTGAATATCGACGTCT
TGAGCGGATGCTCGAAGTTATGGCAGAAGTTCGGCCCATAGCCCGATCCA
TTAAAACGGCTCACGACGATGCGCGAGCGGAATTAATGTCGGCGGACAGA
CCTCGATCAACGCGCGGTCTATGAagacaggaaggaccgaataatggcaa
atggtcagttcacgatacgctctgctcgcccggcctccgtcggactgaca
ggcgaacggcgtggagccgcatccgcctctagctctgcactgtccaatgt
tcaaagagatgttagggataggctgattccaactagctcaccaagattac
caaatgcagccatattgcgtgattcctcgggaagagcgtcgactggtctg
cggtacatggcggctactcttcattggtctgcgatcgcgccattatcgct
aataaacagcaacgacctggctccggccgcttatgactttgagacgcgaa
ataacgcaagaaatgtgactgccaaagtcggcagggcagtccctgttccc
aagcaaggcgggctcggcaaaacgctcgcacccgtaccccttagtacacg
tatatcaagggtcaattccgaccgaagactgcccgctgacgcagaagacc
gccctgaaacgcgcgacccccagaaagctt

> SEQ ID NO 46. EcoRI-HindIII fragment of PtACC2-virD2 flanked with 500 bp of virD1 and 500 bp of virD3. The PtACC2-VirD2 has the Arabidopsis ACC2 Transit peptide, the wt minimal VirD2-T4SS signal listed as SEQ ID NO 42.

gaattcttccgctttctttggaggaagctatgtcaaaacacaccagagcc
acgtcgagtgagactaccatcaaccagcatcgatccctgaaagttgaagg
gttcaaggtcgtgagtgcccgtctgcgatcggccgagtatgaaacctttt
cctatcaagcgcgcctgctgggactttcggatagtatggcaattcgcgtt
gcggtgcgccgcatcgggggctttctcgaaatagatgcagacacacgaga
aaagatggaagccatacttcagtccatcggaatactctcaagcaatgtat
ccatgcttctatctgcctacgccgaagaccctcgatcggatctggaggct
gtgcgagatgaacgtattgcttttggcgaggctttcgccgccctcgatgg
actactgcgctccattttgtccgtatcccggcgacggatcgacggtcgct
cgttactgaaaggtgccttgtagcacttgaccacgcacctgacgggagaa
aattggATGGAGATGAGAGCTTTGGGTTCTTCGTGTTCTACTGGTAATGG
AGGTTCTGCTCCGATTACCCTCACGAATATATCTCCTTGGATCACAACAG
TTTTTCCATCTACAGTGAAGCTGAGAAGTAGTTTGAGAACCTTCAAAGGA
GTTTCGTCAAGAGTGAGAACCTTTAAAGGAGTTTCTTCGACAAGAGTTTT
GTCTCGGACCAAACAACAGTTTCCTCTGTTTTGTTTCCTAAACCCTGATC
CGATGGCTCCCGATCGGGCCCAAGTAATCATTCGCATTGTGCCAGGAGGT
GGAACCAAGACCCTTCAGCAGATAATCAATCAGCTGGAGTACCTGTCCCG
AAAGGGAAAGCTGGAACTCCAGCGTTCAGCCCGGCATCTCGATATTCCCG
TTCCGCCGGATCAAATCCGTGAGCTTGCCCAAAGCTGGGTTACGGAGGCC
GGGATTTATGACGAAAGTCAGTCAGACGATGACAGGCAACAAGACTTAAC
AACACACATTATTGTGAGCTTCCCCGCAGGTACCGACCAAACCGCAGCTT
ATGAAGCAAGCCGGGAATGGGCAGCCGAGATGTTTGGGTCAGGATACGGG
GGTGGCCGCTATAACTATCTGACAGCCTACCACGTGGACCGCGATCATCC
ACATTTACATGTCGTGGTCAATCGTCGGGAACTTCTGGGGCAGGGGTGGC
TGAAAATATCCAGGCGCCATCCCCAGCTGAATTATGACGGCTTACGGAAA
AAGATGGCAGAGATTTCACTTCGTCACGGCATAGTCCTGGATGCGACTTC
CCGAGCAGAAAGGGGAATAGCAGAGCGACCAATCACCTATGCTGAATATC
GACGTCTTGAGCGGATGCTCGAAGTTATGGCAGAAGTTCGGCCCATAGCC
CGATCCATTAAAACGGCTCACGACGATGCGCGAGCGGAATTAATGTCGGC
GGACAGACCTCGATCAACGCGCGGTCTATGAagacaggaaggaccgaata
atggcaaatggtcagttcacgatacgctctgctcgcccggcctccgtcgg
actgacaggcgaacggcgtggagccgcatccgcctctagctctgcactgt
ccaatgttcaaagagatgttagggataggctgattccaactagctcacca
agattaccaaatgcagccatattgcgtgattcctcgggaagagcgtcgac
tggtctgcggtacatggcggctactcttcattggtctgcgatcgcgccat
tatcgctaataaacagcaacgacctggctccggccgcttatgactttgag
acgcgaaataacgcaagaaatgtgactgccaaagtcggcagggcagtccc
tgttcccaagcaaggcgggctcggcaaaacgctcgcacccgtacccctta
gtacacgtatatcaagggtcaattccgaccgaagactgcccgctgacgca
gaagaccgccctgaaacgcgcgacccccagaaagctt

> SEQ ID NO 47. EcoRI-HindIII fragment of PtMKK4-virD2 flanked with 500 bp of virD1 and 500 bp of virD3. The PtMKK4-VirD2 has the Arabidopsis MKK4 Transit peptide, the wt minimal VirD2-T4SS signal listed as SEQ ID NO 43.

gaattcttccgctttctttggaggaagctatgtcaaaacacaccagagcc
acgtcgagtgagactaccatcaaccagcatcgatccctgaaagttgaagg
gttcaaggtcgtgagtgcccgtctgcgatcggccgagtatgaaacctttt
cctatcaagcgcgcctgctgggactttcggatagtatggcaattcgcgtt
gcggtgcgccgcatcgggggctttctcgaaatagatgcagacacacgaga
aaagatggaagccatacttcagtccatcggaatactctcaagcaatgtat
ccatgcttctatctgcctacgccgaagaccctcgatcggatctggaggct
gtgcgagatgaacgtattgcttttggcgaggctttcgccgccctcgatgg
actactgcgctccattttgtccgtatcccggcgacggatcgacggtcgct
cgttactgaaaggtgccttgtagcacttgaccacgcacctgacgggagaa
aattggATGAGACCGATTCAATCGCCTCCAGGAGTTTCCGTTCCGGTGAA
AAGCCGTCCCCGTCGCCGTCCTGATCTTACCTTACCGCTTCCTCAACGCG
ATGTTTCTCTCGCTGTACCTCTTCCTCTCCCACCTACTTCCGGTGGTTCC
GGTGGCTCTAGTGGATCTGCGCCGTCTTCTGGTGGTTCGGCGTCTTCAAC
GATGGCTCCCGATCGGGCCCAAGTAATCATTCGCATTGTGCCAGGAGGTG
GAACCAAGACCCTTCAGCAGATAATCAATCAGCTGGAGTACCTGTCCCGA
AAGGGAAAGCTGGAACTCCAGCGTTCAGCCCGGCATCTCGATATTCCCGT
TCCGCCGGATCAAATCCGTGAGCTTGCCCAAAGCTGGGTTACGGAGGCCG
GGATTTATGACGAAAGTCAGTCAGACGATGACAGGCAACAAGACTTAACA
ACACACATTATTGTGAGCTTCCCCGCAGGTACCGACCAAACCGCAGCTTA
TGAAGCAAGCCGGGAATGGGCAGCCGAGATGTTTGGGTCAGGATACGGGG
GTGGCCGCTATAACTATCTGACAGCCTACCACGTGGACCGCGATCATCCA
CATTTACATGTCGTGGTCAATCGTCGGGAACTTCTGGGGCAGGGGTGGCT
GAAAATATCCAGGCGCCATCCCCAGCTGAATTATGACGGCTTACGGAAAA
AGATGGCAGAGATTTCACTTCGTCACGGCATAGTCCTGGATGCGACTTCC
CGAGCAGAAAGGGGAATAGCAGAGCGACCAATCACCTATGCTGAATATCG
ACGTCTTGAGCGGATGCTCGAAGTTATGGCAGAAGTTCGGCCCATAGCCC
GATCCATTAAAACGGCTCACGACGATGCGCGAGCGGAATTAATGTCGGCG
GACAGACCTCGATCAACGCGCGGTCTATGAagacaggaaggaccgaataa
tggcaaatggtcagttcacgatacgctctgctcgcccggcctccgtcgga
ctgacaggcgaacggcgtggagccgcatccgcctctagctctgcactgtc
caatgttcaaagagatgttagggataggctgattccaactagctcaccaa
gattaccaaatgcagccatattgcgtgattcctcgggaagagcgtcgact
ggtctgcggtacatggcggctactcttcattggtctgcgatcgcgccatt
atcgctaataaacagcaacgacctggctccggccgcttatgactttgaga
cgcgaaataacgcaagaaatgtgactgccaaagtcggcagggcagtccct
gttcccaagcaaggcgggctcggcaaaacgctcgcacccgtaccccttag
tacacgtatatcaagggtcaattccgaccgaagactgcccgctgacgcag
aagaccgccctgaaacgcgcgacccccagaaagctt

>SEQ ID NO 48 - AM102 Primer

GTTGAGTCGATACTTCGGCG

>SEQ ID NO 49 - AM103

GGCACCAGTCCTACATTTTG

>SEQ ID NO 50 - GFP11

RDHMBLHEYBNAAGIT

>SEQ ID NO 51 - Left Border sequence

TGGCAGGATATATTGTGGTGTAAAC

>SEQ ID NO 52 - Spacer between Left Border repeats

CGAGCTGTATAGGCTGGCTGG

>SEQ ID NO 53 - Right Border sequence

TGACAGGATATATTGGCGGGTAAAC

>SEQ ID NO 54 - Overdrive sequence

TAAGTCGCTGTATGTGTTTGTTTG

SEQ ID NO 55 DNA sequence introducing streptomycin resistance and a silent mutation in the tobacco rps12 coding sequence. Flanked by PacI and AscI restriction sites.. C271T changes Pro to Ser conferring streptomycin resistance. T276A is a silent mutation.

TTAATTAAGTCTTAGTAAGAGGGGGAAGGGTTAAGGATTTATCCGGAGTG
AGATATCACATTGTTCGAGGAAGGCGCGCC

>SEQ ID NO 56. A variant of SEQ ID NO 29 (FIG. 11), with shorter plastid targeting sequences. Note that the PacI and AscI sites have been blunted.

CCGCCAGCGTTCATCCTGAGCCAGGATCGAACTCTCCATGAGATTCATAG
TTGCATTACTTATAGCTTCCTTGTTCGTAGACAAAGCGGATTCGGAATTG
TCTTTCATTCCAAGGCATAACTTGTATCCATGCGCTTCATATTCGCCCGG
AGTTCGCTCCCAGAAATATAGCCATCCCTGCCCCCTCACGTCAATCCCAC
GAGCCTCTTATCCATTCTCATTGAACGACGGCGGGGGAGCAAATCCAACT
AGAAAAACTCACATTGGGCTTAGGGATAATCAGGCTCGAACTGATGACTT
CCACCACGTCAAGGTGACACTCTACCGCTGAGTTATATCCCTTCCCCGCC
CCATCGAGAAATAGAACTGACTAATCCTAAGTCAAAGGGTCGAGAAACTC
AACGCCACTATTCTTGAACAACTTGGAGCCGGGCCTTCTTTTCGCACTAT
TACGGATATGAAAATAATGGTCAAAATCGGATTCAATTCCCgggTTGGGT
TGCGCTATATATATGAAAGAGTATACAATAATGATGTATTTGagttgtag
ggagggatttATGgGGGAAGCGGTGATCGCCGAAGTATCGACTCAACTAT
CAGAGGTAGTTGGCGTCATCGAGCGCCATCTCGAACCGACGTTGCTGGCC
GTACATTTGTACGGCTCCGCAGTGGATGGCGGCCTGAAGCCACACAGTGA
TATTGATTTGCTGGTTACGGTGACCGTAAGGCTTGATGAAACAACGCGGC
GAGCTTTGATCAACGACCTTTTGGAAACTTCGGCTTCCCCTGGAGAGAGC
GAGATTCTCCGCGCTGTAGAAGTCACCATTGTTGTGCACGACGACATCAT
TCCGTGGCGTTATCCAGCTAAGCGCGAACTGCAATTTGGAGAATGGCAGC
GCAATGACATTCTTGCAGGTATCTTCGAGCCAGCCACGATCGACATTGAT
CTGGCTATCTTGCTGACAAAAGCAAGAGAACATAGCGTTGCCTTGGTAGG
TCCAGCGGCGGAGGAACTCTTTGATCCGGTTCCTGAACAGGATCTATTTG
AGGCGCTAAATGAAACCTTAACGCTATGGAACTCGCCGCCCGACTGGGCT
GGCGATGAGCGAAATGTAGTGCTTACGTTGTCCCGCATTTGGTACAGCGC
AGTAACCGGCAAAATCGCGCCGAAGGATGTCGCTGCCGACTGGGCAATGG
AGCGCCTGCCGGCCCAGTATCAGCCCGTCATACTTGAAGCTAGACAGGCT
TATCTTGGACAAGAAGAAGATCGCTTGGCCTCGCGCGCAGATCAGTTGGA
AGAATTTGTCCACTACGTGAAAGGCGAGATCACCAAGGTAGTgGGCAAAg
aacaaaaactcatttctgaagaagacttgtgaGATCCTGGCCTAGTCTAT
AGGAGGTTTTGAAAAGAAAGGAGCAATAATCATTTTCTTGTTCTATCAAG
AGGGTGCTATTGCTCCTTTCTTTTTTTCTTTTTATTTATTTACTAGTATT
TTACTTACATAGACTTTTTTGTTTACATTATAGAAAAAGAAGGAGAGGTT
ATTTTCTTGCATTTATTCATGcccGGGCAACTGCCCCTATCGGAAATAGG
ATTGACTACCGATTCCGAAGGAACTGGAGTTACATCTCTTTTCCATTCAA
GAGTTCTTATGCGTTTCCACGCCCCTTTGAGACCCCGAAAAATGGACAAA
TTCCTTTTCTTAGGAACACATACAAGATTCGTCACTACAAAAAGGATAAT
GGTAACCCTACCATTAACTACTTCATTTATGAATTTCATAGTAATAGAAA
TACATGTCCTACCGAGACAGAATTTGGAACTTGCTATCCTCTTGCCTAGC
AGGCAAAGATTTACCTCCGTGGAAAGGATGATTCATTCGGATCGACATGA
GAGTCCAACTACATTGCCAGAATCCATGTTGTATATTTGAAAGAGGTTGA
CCTCCTTGCTTCTCTCATGGTACACTCCTCTTCCCGCCGAGCCCCTTTTC
TCCTCGGTCCACAGAGACAAAATGTAGGACTGGTGCCAACAATTCATCAG
ACTCACTAAGTCGGGATCACTAACTAATACTAATCTAATATAATAGTCTA
ATATATCTAATATAATAGAAAATACTAATATAATAGAAAAGAACTGTCTT
TTCTGTATACTTTCCCCGGTTCCGTTGCTACCGCGGGCTTTACGCAATCG
ATCGGATTAGATAGAT

>SEQ ID NO 57. attB sequence.

CCAGGGCGTGCCCTTGGGCTCCCCGGGCG

>SEQ ID NO 58. attP sequence.

ACTGGGGTAACCTTTGAGTTCTCTCAGTT

>SEQ ID NO 59. attL sequence.

CCAGGGCGTGCCCTTGAGTTCTCTCAGTT

>SEQ ID NO 60. attR sequence.

ACTGGGGTAACCTTTGGGCTCCCCGGGCG

>SEQ ID NO 61 Oligonucleotide confers streptomycin resistance and the silent mutations create a KpnI restriction site in the rps12 plastid gene.

GTCTTAGTAAGAGGGGGAAGGGTTAAGGATTTATCCGGTGTGAGGTACCA
CATTGTTCGAGGAACCCTAGATGCTGTC

### References

Armbruster, U., Hertle, A., Makarenko, E., Zuhlke, J., Pribil, M., Dietzmann, A., Schliebner, I., Aseeva, E., Fenino, E., Scharfenberg, M., Voigt, C. and Leister, D. (2009) Chloroplast proteins without cleavable transit peptides: rare exceptions or a major constituent of the chloroplast proteome? Mol Plant, 2, 1325-1335.

Bevan, M. (1984) Binary Agrobacterium vectors for plant transformation. Nucleic Acids Res., 12, 8711-8721. Brunaud, V., Balzergue, S., Dubreucq, B., Aubourg, S., Samson, F., Chauvin, S., Bechtold, N., Cruaud, C., DeRose, R., Pelletier, G., Lepiniec, L., Caboche, M. and Lecharny, A. (2002) T-DNA integration into the Arabidopsis genome depends on sequences of pre-insertion sites. EMBO Rep, 3, 1152-1157.

Cabantous, S., Terwilliger, T.C. and Waldo, G.S. (2005) Protein tagging and detection with engineered self-assembling fragments of green fluorescent protein. Nat. Biotechnol., 23, 102-107.

Chinault, A.C., Blakesley, V.A., Roessler, E., Willis, D.G., Smith, C.A., Cook, R., G, and Fenwick, R.G. (1986) Characterization of transferable plasmids for Shigella flexneri 2a that confer resistance to trimethoprim, stfreptomycin and sulfonamides. Plasmid, 15, 119-131.

Clough, S.J. and Bent, A.F. (1998) Floral dip: a simplified method for Agrobacterium-mediated transformation of Arabidopsis thaliana. Plant J., 16, 735-743.

Gelvin, S.B. (2003) Agrobacterium-mediated plant transformation: the biology behind the “gene-jockeying” tool. Microbiol Mol Biol Rev, 67, 16-37.

Gelvin, S.B. (2010) Plant Proteins Involved in Agrobacterium-Mediated Genetic Transformation. Annu Rev Phytopathol, 48, 45-68.

Gelvin, S.B. (2012) Traversing the cell: Agrobacterium T-DNA’s journey to the host genome. Front Plant Sci, 3, 52.

Gelvin, S.B. (2017) Integration of Agrobacterium T-DNA into the Plant Genome. Annu Rev Genet, 51, 195-217. Gnanasambandam, A., Polkinghome, I.G. and Birch, R.G. (2007) Heterologous signals allow efficient targeting of a nuclear-encoded fusion protein to plastids and endoplasmic reticulum in diverse plant species. Plant Biotechnol. J., 5, 290-296.

Groth, A.C., Olivares, E.C., Thyagarajan, B. and Calos, M.P. (2000) A phage integrase directs efficient site-specific integration in human cells. PNAS, 97, 5995-6000.

Hajdukiewicz, P., Svab, Z. and Maliga, P. (1994) The small, versatile pPZP family of Agrobacterium binary vectors for plant transformation. Plant Mol. Biol., 25, 989-994.

Heeb, S., Itoh, Y., Nishijyo, T., Schnider, U., Keel, C., Wade, J., Walsh, U., O’Gara, F. and Haas, D. (2000) Small, stable shuttle vectors based on the minimal pVS1 replicon for use in gram-negative, plant-associated bacteria. Mol Plant Microbe In, 13, 232-237.

Herrera-Estrella, A., Van Montagu, M. and Wang, K. (1990) A Bacterial Peptide Acting as a Plant Nuclear Targeting Signal - the Amino-Terminal Portion of Vird2 Protein Directs a Beta-Galactosidase Fusion Protein into Tobacco Nuclei. Proc. Natl. Acad. Sci. USA, 87, 9534-9537.

Hoekema, A., Hirsch, P.R., Hooykaas, P.J.J. and Schilperoort, R.A. (1983) A Binary Plant Vector Strategy Based on Separation of Vir-Region and T-Region of the Agrobacterium-Tumefaciens Ti-Plasmid. Nature, 303, 179-180. Hood, E.E., Gelvin, S.B., Melchers, L.S. and Hoekema, A. (1993) New Agrobacterium helper plasmids for gene transfer to plants. Transgenic Res., 2, 208-218.

Hood, E.E., Helmer, G.L., Fraley, R.T. and Chilton, M.D. (1986) The hypervirulance of Agrobacterium tumefaciens A281 is encoded in a region of pTiBo542 outside of T-DNA. J Bacteriol, 168, 1291-1301.

Howard, E.A., Zupan, J.R., Citovsky, V. and Zambryski, P.C. (1992) The VirD2 protein of A. tumefaciens contain a C-terminalbipartite nuclear localization signal: implications for nuclear uptake of DNA in plant cells. Cell, 68, 109-118.

Jen, G.C. and Chilton, M.D. (1986) Activity of T-DNA Borders in Plant-Cell Transformation by Mini-T Plasmids. J Bacteriol, 166, 491-499.

Kapila, J., De Rycke, R., Van Montagu, M. and Angenon, G. (1997) An Agrobacterium-mediated transient gene expression system for intact leaves. Plant Sci., 122, 101-108.

Komori, T., Imayama, T., Kato, N., Ishida, Y., Ueki, J. and Komari, T. (2007) Current status of binary vectors and superbinary vectors. Plant Physiol., 145, 1155-1160.

Kosugi, S., Hasebe, M., Tomita, M. and Yanagawa, H. (2009) Systematic identification of cell cycle-dependent yeast nucleocytoplasmic shuttling proteins by prediction of composite motifs (vol 106, pg 10171, 2009). Proc. Natl. Acad. Sci. USA, 106, 13142-13142.

Krieg, A.M., Wu, T., Weeratna, R., Efler, S.M., Love-Homan, L., Yang, L., Yi, A.K., Short, D. and Davis, H.L. (1998) Sequence motifs in adenoviral DNA block immune activation by stimulatory CpG motifs. Proc. Natl. Acad. Sci. USA, 95, 12631-12636.

Kuraya, Y., Ohta, S., Fukuda, M., Hiei, Y., Murai, N., Hamada, K., Ueki, J., Imaseki, H. and Komari, T. (2004) Suppression of transfer of non-T-DNA ‘vector backbone’ sequences by multiple left border repeats in vectors for transformation of higher plants mediated by Agrobacterium tumefaciens. Molecular Breeding, 14, 309-320. Lee, L.Y. and Gelvin, S.B. (2008) T-DNA binary vectors and systems. Plant Physiol., 146, 325-332.

Li, X.Y., Yang, Q.H., Peng, L., Tu, H.T., Lee, L.Y., Gelvin, S.B. and Pan, S.Q. (2020) Agrobacterium-delivered VirE2 interacts with host nucleoporin CG1 to facilitate the nuclear import of VirE2-coated T complex. Proc. Natl. Acad. Sci. USA, 117, 26389-26397.

Liu, W.S., Zhu, X.H., Lei, M.G., Xia, Q.Y., Botella, J.R., Zhu, J.K. and Mao, Y.F. (2015) A detailed procedure for CRISPR/Cas9-mediated gene editing in Arabidopsis thaliana. Sci Bull, 60, 1332-1347.

Liu, Y.G., Liu, H.M., Chen, L.T., Qiu, W.H., Zhang, Q.Y., Wu, H., Yang, C.Y., Su, J., Wang, Z.H., Tian, D.S. and Mei, M.T. (2002) Development of new transformation-competent artificial chromosome vectors and rice genomic libraries for efficient gene cloning. Gene, 282, 247-255.

Lutz, K., Corneille, S., Azhagiri, A.K., Svab, Z. and Maliga, P. (2004) A novel approach to plastid transformation utilizes the phiC31 phage integrase. Plant J., 37, 906-913.

Lutz, K.A., Bosacchi, M.H. and Maliga, P. (2006) Plastid marker gene excision by transiently expressed CRE recombinase. Plant J., 45, 447-456.

Matsuoka, A. (2015) PhD Thesis: Genetic engineering of Agrobcterium tumefaciens to target chloroplasts and identification of a novel nuclear localization signal of VirD2 protein: University of Lethbridge, Alberta, Canada. Matsuoka, A. and Maliga, P. (2021) Progress in re-engineering Agrobacterium T-DNA delivery to chloroplasts. Plant Physiol., 186, 215-220.

McCullen, C.A. and Binns, A.N. (2006) Agrobacterium tumefaciens and plant cell interactions and activities required for interkingdom macromolecular transfer. Annu Rev Cell Dev Biol, 22, 101-127.

Melchers, L.S., Maroney, M.J., den Dulk-Ras, A., Thompson, D.V., van Vuuren, H.A., Schilperoort, R.A. and Hooykaas, P.J. (1990) Octopine and nopaline strains of Agrobacterium tumefaciens differ in virulence; molecular characterization of the virF locus. Plant Mol. Biol., 14, 249-259.

Peralta, E.G., Hellmiss, R. and Ream, W. (1986) Overdrive, a T-DNA Transmission Enhancer on the a-Tumefaciens Tumor-Inducing Plasmid. EMBO J., 5, 1137-1142.

Peralta, E.G. and Ream, L.W. (1985) T-DNA Border Sequences Required for Crown Gall Tumorigenesis. Proc. Natl. Acad. Sci. USA, 82, 5112-5116.

Podevin, N., De Buck, S., De Wilde, C. and Depicker, A. (2006) Insights into recognition of the T-DNA border repeats as termination sites for T-strand synthesis by Agrobacterium tumefaciens. Transgenic Res., 15, 557-571. Rossi, L., Hohn, B. and Tinland, B. (1993) The Vird2 Protein of Agrobacterium-Tumefaciens Carries Nuclear-Localization Signals Important for Transfer of T-DNA to Plants. Mol. Gen. Genet., 239, 345-353.

Ruf, S., Forner, J., Hasse, C., Kroop, X., Seeger, S., Schollbach, L., Schadach, A. and Bock, R. (2019) High-efficiency generation of fertile transplastomic Arabidopsis plants. Nat Plants, 5, 282-289.

Shibata, D. and Liu, Y.G. (2000) Agrobacterium-mediated plant transformation with large DNA fragments. Trends Plant Sci, 5, 354-357.

Sjuts, I., Soll, J. and Bolter, B. (2017) Import of Soluble Proteins into Chloroplasts and Potential Regulatory Mechanisms. Front Plant Sci, 8, 168.

Sparkes, I.A., Runions, J., Kearns, A. and Hawes, C. (2006) Rapid, transient expression of fluorescent fusion proteins in tobacco plants and generation of stably transformed plants. Nat. Protocols, 1, 2019-2025.

Svab, Z. and Maliga, P. (1993) High-frequency plastid transformation in tobacco by selection for a chimeric aadA gene. Proc. Natl. Acad. Sci. USA, 90, 913-917.

Thole, V., Worland, B., Snape, J.W. and Vain, P. (2007) The pCLEAN dual binary vector system for Agrobacterium-Mediated plant transformation. Plant Physiol., 145, 1211-1219.

Tinland, B., Koukolikova-Nicola, Z., Hall, M.N. and Hohn, B. (1992) The T-DNA-Linked Vird2 Protein Contains 2 Distinct Functional Nuclear-Localization Signals. Proc. Natl. Acad. Sci. USA, 89, 7442-7446.

Toro, N., Datta, A., Carmi, O.A., Young, C., Prusti, R.K. and Nester, E.W. (1989) The Agrobacterium-Tumefaciens Virc1 Gene-Product Binds to Overdrive, a T-DNA Transfer Enhancer. J Bacteriol, 171, 6845-6849. Tungsuchat-Huang, T., Sinagawa-Garcia, S.R., Paredes-Lopez, O. and Maliga, P. (2010) Study of plastid genome stability in tobacco reveals that the loss of marker genes is more likely by gene conversion than by recombination between 34-bp loxP repeats. Plant Physiol., 153, 252-259.

Tzfira, T., Li, J.X., Lacroix, B. and Citovsky, V. (2004) Agrobacterium T-DNA integration: molecules and models. Trends Genet., 20, 375-383.

van Kregten, M., Lindhout, B.I., Hooykaas, P.J.J. and van der Zaal, B.J. (2009) Agrobacterium-Mediated T-DNA Transfer and Integration by Minimal VirD2 Consisting of the Relaxase Domain and a Type IV Secretion System Translocation Signal. Mol Plant Microbe In, 22, 1356-1365.

Vergunst, A.C., Schrammeijer, B., den Dulk-Ras, A., de Vlaam, C.M.T., Regensburg-Tuink, T.J.G. and Hoojkaas, P.J.J. (2000) VirB/D4-dependent protein translocation from Agrobacterium into plant cells. Science, 290, 979-982. Vergunst, A.C., van Lier, M.C.M., den Dulk-Ras, A., Stuve, T.A., Ouwehand, A. and Hooykaas, P.J.J. (2005) Positive charge is an important feature of the C-terminal transport signal of the VirB/VirD4-translocated proteins of Agrobacterium. Proc Natl Acad Sci U.S.A., 102, 823-837.

Winans, S.C., Allenza, P., Stachel, S.E., Mcbride, K.E. and Nester, E.W. (1987) Characterization of the virE Operon of the Agrobacterium Ti-Plasmid PtiA6. Nucleic Acids Res., 15, 825-837.

Wohlleben, W., Arnold, W., Bissonnette, L., Pelletier, A., Tanguay, A., Roy, P.H., Gamboa, G.C., Barry, G.F., Aubert, E., Davies, J. and et al. (1989) On the evolution of Tn21-like multiresistance transposons: sequence analysis of the gene (aacC1) for gentamicin acetyltransferase-3-I(AAC(3)-I), another member of the Tn21-based expression cassette. Mol Gen Genet, 217, 202-208.

Wolterink-van Loo, S., Ayala, A.A.E., Hooykaas, P.J.J. and van Heusden, G.P.H. (2015) Interaction of the Agrobacterium tumefaciens virulence protein VirD2 with histones. Microbiol-Sgm, 161, 401-410.

Wood, D.W., Setubal, J.C., Kaul, R., Monks, D.E., Kitajima, J.P., Okura, V.K., Zhou, Y., Chen, L., Wood, G.E., Almeida, N.F., Jr., Woo, L., Chen, Y., Paulsen, I.T., Eisen, J.A., Karp, P.D., Bovee, D., Sr., Chapman, P., Clendenning, J., Deatherage, G., Gillet, W., Grant, C., Kutyavin, T., Levy, R., Li, M.J., McClelland, E., Palmieri, A., Raymond, C., Rouse, G., Saenphimmachak, C., Wu, Z., Romero, P., Gordon, D., Zhang, S., Yoo, H., Tao, Y., Biddle, P., Jung, M., Krespan, W., Perry, M., Gordon-Kamm, B., Liao, L., Kim, S., Hendrick, C., Zhao, Z.Y., Dolan, M., Chumley, F., Tingey, S.V., Tomb, J.F., Gordon, M.P., Olson, M.V. and Nester, E.W. (2001) The genome of the natural genetic engineer Agrobacterium tumefaciens C58. Science (New York, N Y ), 294, 2317-2323.

Yu, Q., LaManna, L., Kelly, M.E., Lutz, K.A. and Maliga, P. (2019) New Tools for Engineering the Arabidopsis Plastid Genome. Plant Physiol., 181, 394-398.

Yu, Q., Lutz, K.A. and Maliga, P. (2017) Efficient plastid transformation in Arabidopsis. Plant Physiol., 175, 186-193.

Yu, Q., Tungsuchat-Huang, T., Verma, K., Radler, M.R. and Maliga, P. (2020) Independent translation of ORFs in dicistronic operons, synthetic buiding blocks for polycistronic chloroplast gene expression. Plant J., 103, 2318-2329. Ziemienowicz, A., Merkle, T., Schoumacher, F., Hohn, B. and Rossi, L. (2001) Import of agrobacterium T-DNA into plant nuclei: Two distinct functions of VirD2 and VirE2 proteins. Plant Cell, 13, 369-383.

Zoubenko, O.V., Allison, L.A., Svab, Z. and Maliga, P. (1994) Efficient targeting of foreign genes into the tobacco plastid genome. Nucleic Acids Res., 22, 3819-3824.

Galili S, Fromm H, Aviv D, Edelman M, Galun E (1989) Ribosomal protein S12 as a site for streptomycin resistance in Nicotiana chloroplasts. Mol Gen Genet 218: 289-292

Hoang TT, Karkhoff-Schweizer RR, Kutchma AJ, Schweizer HP (1998) A broad-host-range Flp-FRT recombination system for site-specific excision of chromosomally-located DNA sequences: application for isolation of unmarked Pseudomonas aeruginosa mutants. Gene 212: 77-86

Hsu CM, Yang WP, Chen CC, Lai YK, Lin TY (1993) A point mutation in the chloroplast rps12 gene from Nicotiana plumbaginifolia confers streptomycin resistance. Plant Mol Biol 23: 179-183

Huang W, Wilks A (2017) A rapid seamless method for gene knockout in Pseudomonas aeruginosa. BMC Microbiol 17: 199

Li XY, Yang QH, Peng L, Tu HT, Lee LY, Gelvin SB, Pan SQ (2020) Agrobacterium-delivered VirE2 interacts with host nucleoporin CG1 to facilitate the nuclear import of VirE2-coated T complex. Proceedings of the National Academy of Sciences of the United States of America 117: 26389-26397

Samuel MA, Chaal BK, Lampard G, Green BR, Ellis BE (2008) Surviving the passage: Non-canonical stromal targeting of an Arabidopsis mitogen-activated protein kinase kinase. Plant Signal Behav 3: 6-12 Sjuts I, Soll J, Bolter B (2017) Import of Soluble Proteins into Chloroplasts and Potential Regulatory Mechanisms. Frontiers in Plant Science 8

Stachel SE, Timmerman B, Zambryski P (1986) Generation of Single-Stranded T-DNA Molecules during the Initial-Stages of T-DNA Transfer from Agrobacterium-Tumefaciens to Plant-Cells. Nature 322: 706-712

Staub JM, Maliga P (1992) Long regions of homologous DNA are incorporated into the tobacco plastid genome by transformation. Plant Cell 4: 39-45

Svab Z, Maliga P (1991) Mutation proximal to the tRNA binding region of the Nicotiana plastid 16S rRNA confers resistance to spectinomycin. Molecular And General Genetics 228: 316-319

Rathore DS, Mullins E (2018) Alternative Non-Agrobacterium Based Methods for Plant Transformation. Annual Plant Reviews Online 1: 891-907

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for delivering a polynucleotide of interest to plant cell chloroplasts, said method comprising
a) providing an Agrobacterium variant strain lacking VirD2 in the Vir operon,
b) introducing a binary vector plasmid encoding a polynucleotide fusion construct encoding a VirD2 variant protein with a chloroplast transit peptide operably linked to a promoter and terminator which drives expression in said Agrobacterium variant strain and a T DNA comprising a chloroplast sequence mediating homologous recombination into the chloroplast genome, a nucleic acid encoding a polypeptide of interest, and a nucleic acid encoding a selectable marker, wherein said VirD2 variant protein forms a complex with said T-DNA and delivers said complex to the chloroplast,
c) selecting infected plant cells which survive in the presence of a selection agent, thereby identifying cells expressing said polynucleotide of interest.

2. The method of claim 1, wherein said VirD2 variant protein is expressed from a binary plasmid comprising a sequence selected from SEQ ID NO: 27, SEQ ID NO: 28, SEQ ID NO: 31, SEQ ID NO: 32, SEQ ID NO: 42 and SEQ ID NO: 43.

3. The method of claim 1, wherein the selectable marker in the binary vector is encoded by a sequence selected from SEQ ID NO: 26, SEQ ID NO: 29, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO 40, SEQ ID NO: 41 and SEQ ID NO: 55.

4. The method of claim 1, wherein said polynucleotide fusion construct is present in a binary vector backbone selected from a pAGa vector of SEQ ID NO: 22, a pAGb vector of SEQ ID NO: 23, a pAGc vector of SEQ ID NO: 24, and a pAGd vector of SEQ ID NO: 25.

5. The method of claim 4, wherein said pAG binary vector encoding any PtVirD2 variant is introduced into a XYA105ΔvirD2 strain for co-cultivation and chloroplast transformation in a plant and said ptVirD2 variant is encoded by a sequence selected from SEQ ID NO: 27, SEQ ID NO: 28, SEQ ID NO: 31, SEQ ID NO: 32, SEQ ID NO: 42 and SEQ ID NO: 43.

6. The method of claim 1, wherein said binary vector of , wherein said binary is a pAG vector comprising a PtVirD2 variant encoded by SEQ ID NO: 29.

7. An isolated Agrobacterium strain XYA105Pt variant selected from XYA105Pt45, XYA105Pt46, and XYA105Pt47, comprising a reconstituted virD operon encoding a VirD2 variant selected from SEQ ID NO: 45, SEQ ID NO: 46 and SEQ ID NO: 47, said variant Agrobacterium strain optionally comprising a binary vector harboring a T-DNA encoding a nucleic acid of interest and a selectable marker, said T-DNA comprising a sequence which mediates homologous recombination in a chloroplast genome in said plant cell.

8. The method of claim 1, wherein said plant is infected with a XYA105ΔvirD2 Agrobacterium strain carrying said binary vector comprising said nucleic acids encoding said T-DNA and said Pt-VirD2 variant.

9. A method for delivering a polynucleotide of interest to plant cell chloroplasts, said method comprising
a) infiltrating a plant with XYA105Pt variant Agrobacterium strain as claimed in claim 7, said strain harboring said T-DNA,
b) culturing treated leaf sections in selection media comprising a selective agent; and
c) isolating transplastomic plant cells resistant to said selection agent.

10. The method of claim 9, wherein saidVirD2 variant is encoded by SEQ ID NO: 27, SEQ ID NO: 28, SEQ ID NO: 31, SEQ ID NO: 32, SEQ ID NO: 42 and SEQ ID NO: 43.

11. The method of claim 1, when the VirD2 variant is incorporated in the virD operon and has a sequence selected from SEQ ID NO: 45, SEQ ID NO: 46 or SEQ ID NO: 47 for reconstitution of said operon.

12. The method of claim 1 wherein said plant cell is a tobacco, Arabidopsis, corn, soybean, sorghum, wheat, rice, alfalfa, sunflower, or Brassica sp. cell.

13. The method of claim 1 wherein selectable marker is a protein.

14. The method of claim 13, wherein said selectable marker protein is a visible or chemical selectable marker protein.

15. The method of claim 14, wherein said selectable marker confers spectinomycin resistance, streptomycin resistance.

16. The method of claim 14, wherein said selectable marker is Green Fluorescent Protein, m-cherry, m-scarlett, luciferase or GUS encoding the beta-glucuronidase enzyme.

17. The method of claim 14, wherein said visual-selectable marker is selected from aadA^{au,} encoded by the sequence provided in GENBANK ID NO. HQ023426 and bar^{au} encoded by the sequence provided in GENBANK ID NO. GH980699.

18. The method of claim 1, wherein said plant cell is a monocot.

19. The method of claim 1, wherein said plant cell is a dicot.

20. The method of claim 1, wherein said plant cell is the plant cell is a Brassica sp., tobacco, Arabidopsis, corn, soybean, sorghum, wheat, rice, alfalfa, or sunflower cell.

21. The method of claim 1, further comprising growing the plant cell to produce a plant.

22. The method of claim 1, further comprising exposing a flower from said plant to said Agrobacterium, harvesting seed from said flower and optionally germinating said seed on selective media.

23. The method of claims 1, further comprising exposing a flower from said plant to said Agrobacterium, harvesting seed from said flower and identifying transplastomic events by leaf phenotypic change.

24. A binary vector for use in the method of claim 5 comprising a transgene expressing a protein or RNA of interest.

25. The vector of claim 24, present in a transplastomic plant.

26. A method for delivering a polynucleotide of interest to plant cell chloroplasts, said method comprising
a) provide an Agrobacterium variant strain with a wild type Vir operon,
b) introducing a binary vector plasmid encoding a polynucleotide fusion construct encoding a VirD2 variant protein operably linked to a promoter and terminator which drives expression in said Agrobacterium variant strain, and
a T DNA comprising a chloroplast sequence mediating homologous recombination into the chloroplast genome, a nucleic acid encoding a polypeptide of interest, and a nucleic acid encoding a selectable marker, wherein said VirD2 variant protein forms a complex with said T-DNA and delivers said complex to the chloroplast,
c) selecting infected plant cells which survive in the presence of a selection agent, thereby identifying cells expressing said polynucleotide of interest.

27. The method of claim 26, wherein said VirD2 variant protein is expressed from a binary plasmid comprising a sequence selected from SEQ ID NO: 27, SEQ ID NO: 28, SEQ ID NO: 31, SEQ ID NO: 32, SEQ ID NO: 42 and SEQ ID NO: 43.

28. The method of claim 8, wherein said plant cell is the plant cell is a Brassica sp., tobacco, Arabidopsis, corn, soybean, sorghum, wheat, rice, alfalfa, or sunflower cell.

29. The method of claim 8, further comprising growing the plant cell to produce a plant.

30. The method of claim 8, further comprising exposing a flower from said plant to said Agrobacterium, harvesting seed from said flower and optionally germinating said seed on selective media.

31. The method of claim 9, wherein said plant cell is the plant cell is a Brassica sp., tobacco, Arabidopsis, corn, soybean, sorghum, wheat, rice, alfalfa, or sunflower cell.

32. The method of claim 9, further comprising growing the plant cell to produce a plant.

33. The method of claim 9, further comprising exposing a flower from said plant to said Agrobacterium, harvesting seed from said flower and optionally germinating said seed on selective media.

34. A kit for practicing the method of claim 1.


SEQUENCE LISTING (61 sequences)

SEQ ID NO: 1 - DNA, 918 bp, synthetic construct
  source 1..918: mol_type = other DNA
  misc_feature 1..918: note = DNA sequence of Pt-VirD2. Coding sequence, tobacco SSU-TP+5aa fused with mutant minimal VirD2+T4SS signal
atggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtccacgaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtacggaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatga

SEQ ID NO: 2 - DNA, 2656 bp, synthetic construct
  source 1..2656: mol_type = other DNA
  misc_feature 1..2656: note = DNA sequence of integrase gene in binary vector pJCD6B
tgcggaacatagaaagcgtcagagacgagacgcaccgtaagtgcgtgctacattgtcctcggccgcacctaaagaccggggcagttgaaatccgattgtgatggcaactataagcgggtgacgaacagcacggattctagaatcatagaccgcgcgttgatcgaggtctgtccgccgacattaattccgctcgcgcatcgtcgtgagccgttttaatggatcgggctatgggccgaacttctgccataacttcgagggcgcccgccgcgacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggcagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcttgccgaagcgtcgggcggcttcccacagaagcgccaacgtctcttcgtcgccttcggcgtgcctgatcttgttgaagatgcgttccgcaacgaacttgtcgagtgccgccatgctgacgttgcacgtgccttcgtgctgcccaggtgcggacgggtcgaccaccttccggcgacggcagcggtaagagtccttgatcgattcttccccgcgcttcgaagtcatgacggcgccacactcgcagtacagcttgtccatcgcggacagaatggcttgcccccgggaaagccccttgccgcgccccctgccgtccaaccacgcctgaagctcataccactcagcgggctcgatgatcggtccgcaatcaagctcgaccggccggagcgtgatcgggtcgcgctgaatgcggtaaccctcaatcttcgtggtcggcgtgccgtccggcttcttcttgtagatcacctcagcggcgaagcccgcaatacgcgggtcccgaaggattcgcataacggttgccgggtcccaggcgcttgaagcggtcttcttcccaatcgtctcgccccgggtcggcacggcgtcagcgtccatgcgcttacaaagccccgtgatgctgcccgggtgaatggcggcttgactgcccggcttgaagggaaggtgtttgtgcgtcttgatctcacgccaccaccaccggattacgtcgggctcgaactcgaagggtccggtaaggggagtggtcgagtgcgccagcttgttgatgacgacattgaccattcggccgttgcgcgtgatctccttcgtctccgaaacaagctcgaagccgtaaggcgccttcccgccgacgtacccgcccaattcgcgctgaaggttcttcgtgtcgagaatcttcgccgacttcagcgaagattctttgtgcgacgcgtcgagccgcataatcaggtgaatcaggtccatgacgtttccctgccggaagacgccttcctgagtggaaacaatcgtcacgcccagggcgagcaattccgagacaatcggaatcgcgtccatgaccttcaggcgcgagaagcgcgacacgtcatagacaatgatcatgttgagccgcccggcgcggcattcgttcaggatgcgttcgaactccgggcgctccgccgtcccgaacgccgacgtgcccggcgcttcgctgaaatgcccgacgaacctgaaccggcccccgtcgcgctcgacttcgcgctgaaggtcggccgccttgtcttcgttggcgctacgctgtgtcgctgggcttgctgcgctcgaattttcgcgctcgcgcgactgacggtcgtaagcacccgcgtacgtgtccatgctagccatggctggccacacctgcatgcattgcactcttccgccgttgctggcaatggaagtgatgtcaaggttttgcttccttgaaacagggaatgaggcagctgacttaaggccagtgaaaggtgcaaccatgttagcttgagcaacattgctgcgggtggcaactgctgcggaggaaagaactgaggaagccatatgttctctcctgcaaaattgcggttttccagaaaatcctctgcaacccgagataaagctgctttgctactttgagcaaagccgggggcggaactgttttgatcagtgcgaccactagacgacccacttataaggtccgtcgtcagtaacctcttatcggaatttttaagtctccgaccgggaagtctggcaagtgctcatatctccaaagtaccatccatactcgggtcttgaaacggatatcgcgtttcaactgcaatgagcaggcaataaatgatgccacaatcgacctgaaagggaggaactggaaagactggacgcacgcggcctgactgtcgaggccatcgtatccctgtgagcattgttggtgacgagcagccg

SEQ ID NO: 3 - protein, 710 aa, synthetic construct
  REGION 1..710: note = Amino acid sequence of the phiC31 integrase in plasmid pJCD6
MASSVLSSAAVATRSNVAQANMVAPFTGLKSAASFPVSRKQNLDITSIASNGGRVQCMQVWPAMASMDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAAGALEVMAEVRPIARSIKTAHDDARAELMSADRPRSTRGL

SEQ ID NO: 4 - DNA, 8832 bp, synthetic construct
  source 1..8832: mol_type = other DNA
  misc_feature 1..8832: note = DNA sequence of binary plasmid pJCD6B
agctttgcggaacatagaaagcgtcagagacgagacgcaccgtaagtgcgtgctacattgtcctcggccgcacctaaagaccggggcagttgaaatccgattgtgatggcaactataagcgggtgacgaacagcacggattctagaatcatagaccgcgcgttgatcgaggtctgtccgccgacattaattccgctcgcgcatcgtcgtgagccgttttaatggatcgggctatgggccgaacttctgccataacttcgagggcgcccgccgcgacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggcagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcttgccgaagcgtcgggcggcttcccacagaagcgccaacgtctcttcgtcgccttcggcgtgcctgatcttgttgaagatgcgttccgcaacgaacttgtcgagtgccgccatgctgacgttgcacgtgccttcgtgctgcccaggtgcggacgggtcgaccaccttccggcgacggcagcggtaagagtccttgatcgattcttccccgcgcttcgaagtcatgacggcgccacactcgcagtacagcttgtccatcgcggacagaatggcttgcccccgggaaagccccttgccgcgccccctgccgtccaaccacgcctgaagctcataccactcagcgggctcgatgatcggtccgcaatcaagctcgaccggccggagcgtgatcgggtcgcgctgaatgcggtaaccctcaatcttcgtggtcggcgtgccgtccggcttcttcttgtagatcacctcagcggcgaagcccgcaatacgcgggtcccgaaggattcgcataacggttgccgggtcccaggcgcttgaagcggtcttcttcccaatcgtctcgccccgggtcggcacggcgtcagcgtccatgcgcttacaaagccccgtgatgctgcccgggtgaatggcggcttgactgcccggcttgaagggaaggtgtttgtgcgtcttgatctcacgccaccaccaccggattacgtcgggctcgaactcgaagggtccggtaaggggagtggtcgagtgcgccagcttgttgatgacgacattgaccattcggccgttgcgcgtgatctccttcgtctccgaaacaagctcgaagccgtaaggcgccttcccgccgacgtacccgcccaattcgcgctgaaggttcttcgtgtcgagaatcttcgccgacttcagcgaagattctttgtgcgacgcgtcgagccgcataatcaggtgaatcaggtccatgacgtttccctgccggaagacgccttcctgagtggaaacaatcgtcacgcccagggcgagcaattccgagacaatcggaatcgcgtccatgaccttcaggcgcgagaagcgcgacacgtcatagacaatgatcatgttgagccgcccggcgcggcattcgttcaggatgcgttcgaactccgggcgctccgccgtcccgaacgccgacgtgcccggcgcttcgctgaaatgcccgacgaacctgaaccggcccccgtcgcgctcgacttcgcgctgaaggtcggccgccttgtcttcgttggcgctacgctgtgtcgctgggcttgctgcgctcgaattttcgcgctcgcgcgactgacggtcgtaagcacccgcgtacgtgtccatgctagccatggctggccacacctgcatgcattgcactcttccgccgttgctggcaatggaagtgatgtcaaggttttgcttccttgaaacagggaatgaggcagctgacttaaggccagtgaaaggtgcaaccatgttagcttgagcaacattgctgcgggtggcaactgctgcggaggaaagaactgaggaagccatatgttctctcctgcaaaattgcggttttccagaaaatcctctgcaacccgagataaagctgctttgctactttgagcaaagccgggggcggaactgttttgatcagtgcgaccactagacgacccacttataaggtccgtcgtcagtaacctcttatcggaatttttaagtctccgaccgggaagtctggcaagtgctcatatctccaaagtaccatccatactcgggtcttgaaacggatatcgcgtttcaactgcaatgagcaggcaataaatgatgccacaatcgacctgaaagggaggaactggaaagactggacgcacgcggcctgactgtcgaggccatcgtatccctgtgagcattgttggtgacgagcagccggaattactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatccagg

SEQ ID NO: 5 - DNA, 1945 bp, synthetic construct
  misc_feature 1..1945: note = pAM305, with dicistronic aadA-gfp1-10 gene, for expression inchloroplasts
  source 1..1945: mol_type = other DNA
gctcccccgccgtcgttcaatgagaatggataagaggctcgtgggattgacgtgagggggcagggatggctatatttctgggagaattaaccgatcgacgtgcaagcggacatttattttaaattcgataatttttgcaaaaacatttcgacatatttatttattttattattatgagaatcaatcctactacttctggttctggggtttccacggctactagcgaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtgggcaaagaacaaaaactcatttctgaagaagacttgtaactgcagataacccaaataatgttttaaaattttaaaaataatgtaggaggaaaaattatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccttggccaacacttgtcactactctgacctatggtgttcaatgcttttcccgttatccggatcacatgaaaaggcatgactttttcaagagtgccatgcccgaaggttatgttcaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaacttgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcgacacaaactgtcctttcgaaagatcccaacgaaaagggtacctaagctctagagatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatg

SEQ ID NO: 6 - DNA, 5723 bp, synthetic construct
  misc_feature 1..5723: note = pAM29
  source 1..5723: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgccagtgaaatgtcccggcgagtaccaagttgatggcaaaaaagttatactagacgaggactgttttatgcaaaacccagaggactgggacgaaaaagtggctgagtggttggccagagagctagagggcatacaaaaaatgaccgaggagcattggaagttggtaaaatacttaagggagtattgggagaccttcggctcctgcccgccaattaaaatggtcactaaagagacgggcttctccttggagaaaatctaccagctattcccctcggggcctgcgcacggagcttgtaaagtggcaggcgcgccgaagcccacaggctgcgtcggatccggtggagggtctggtggcggatcaactagtcgtgaccacatggtccttcatgagtacgtaaatgctgctgggattacataattctagataactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 7 - DNA, 5997 bp, synthetic construct
  misc_feature 1..5997: note = pAM30
  source 1..5997: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagatatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 8 - DNA, 6045 bp, synthetic construct
  misc_feature 1..6045: note = pAM31
  source 1..6045: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgcaggtgtggccagcgcgtgaccacatggtccttcatgagtacgtaaatgctgctgggattacaatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagatatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 9 - DNA, 6069 bp, synthetic construct
  misc_feature 1..6069: note = pAM32
  source 1..6069: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgcaggtgtggccagcgcgtgaccacatggtccttcatgagtacgtaaatgctgctgggattacaggtggagggtctggtggcggatcaatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagatatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 10 - DNA, 6045 bp, synthetic construct
  misc_feature 1..6045: note = pAM33
  source 1..6045: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgcgtgaccacatggtccttcatgagtacgtaaatgctgctgggattacactcgaagttatggcagaagttcggcccatagcccgatccattaaaacggctcacgacgatgcgcgagcggaattaatgtcggcggacagacctcgatcaacgcgcggtctatgattctagatatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - DNA, 6069 bp, synthetic construct
  misc_feature 1..6069: note = pAM34
  source 1..6069: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgggtggagggtctggtggcggatcacgtgaccacatggtccttcatgagtacgtaaatgctgctgggattacactcgaagttatggcagaagttcggcccatagcccgatccattaaaacggctcacgacgatgcgcgagcggaattaatgtcggcggacagacctcgatcaacgcgcggtctatgattctagatatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 12 - DNA, 6069 bp, synthetic construct
  misc_feature 1..6069: note = pAM35
  source 1..6069: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgcgtgaccacatggtccttcatgagtacgtaaatgctgctgggattacaggtggagggtctggtggcggatcactcgaagttatggcagaagttcggcccatagcccgatccattaaaacggctcacgacgatgcgcgagcggaattaatgtcggcggacagacctcgatcaacgcgcggtctatgattctagatatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 13 - DNA, 6069 bp, synthetic construct
  misc_feature 1..6069: note = pAM36
  source 1..6069: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgggtggagggtctcgtgaccacatggtccttcatgagtacgtaaatgctgctgggattacaggtggcggatcactcgaagttatggcagaagttcggcccatagcccgatccattaaaacggctcacgacgatgcgcgagcggaattaatgtcggcggacagacctcgatcaacgcgcggtctatgattctagatatactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 14 - DNA, 13581 bp, synthetic construct
  misc_feature 1..13581: note = pAM407
  source 1..13581: mol_type = other DNA
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttaattaagccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgaggtctcggtaccatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccaatgagcaaaggagaagaacttttcactggagttgtcccaattcttgttgaattagatggtgatgttaatgggcacaaattttctgtcagaggagagggtgaaggtgatgctacaatcggaaaactcacccttaaatttatttgcactactggaaaactacctgttccttggccaacacttgtcactactctgacctatggtgttcaatgcttttcccgttatccggatcacatgaaaaggcatgactttttcaagagtgccatgcccgaaggttatgttcaggaacgcactatatctttcaaagatgacgggaaatacaagacgcgtgctgtagtcaagtttgaaggtgatacccttgttaatcgtatcgagttaaagggtactgattttaaagaagatggaaacattctcggacacaaacttgagtacaactttaactcacacaatgtatacatcacggcagacaaacaaaagaatggaatcaaagctaacttcacagttcgccacaacgttgaagatggttccgttcaactagcagaccattatcaacaaaatactccaattggcgatggccctgtccttttaccagacaaccattacctgtcgacacaaactgtcctttcgaaagatcccaacgaaaagggaacctaattctagactgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 15 - DNA, 10545 bp, synthetic construct
  misc_feature 1..10545: note = pJF7
  source 1..10545: mol_type = other DNA
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatcttagaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagagacgaaacacaaaccatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgcgtgaccacatggtccttcatgagtacgtaaatgctgctgggattacaatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagaagagactcttatcaagaatcccatctcttgcttgcttttttttgttgcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 16 - DNA, 10569 bp, synthetic construct
  misc_feature 1..10569: note = pJF8
  source 1..10569: mol_type = other DNA
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatcttagaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagagacgaaacacaaaccatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgcgtgaccacatggtccttcatgagtacgtaaatgctgctgggattacaggtggagggtctggtggcggatcaatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagaagagactcttatcaagaatcccatctcttgcttgcttttttttgttgcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 17 - DNA, 10545 bp, synthetic construct
  misc_feature 1..10545: note = pJF9
  source 1..10545: mol_type = other DNA
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatcttagaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagagacgaaacacaaaccatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgcgtgaccacatggtccttcatgagtacgtaaatgctgctgggattacactcgaagttatggcagaagttcggcccatagcccgatccattaaaacggctcacgacgatgcgcgagcggaattaatgtcggcggacagacctcgatcaacgcgcggtctatgattctagaagagactcttatcaagaatcccatctcttgcttgcttttttttgttgcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 18 - DNA, 10569 bp, synthetic construct
  misc_feature 1..10569: note = pJF10
  source 1..10569: mol_type = other DNA
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatcttagaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagagacgaaacacaaaccatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgggtggagggtctggtggcggatcacgtgaccacatggtccttcatgagtacgtaaatgctgctgggattacactcgaagttatggcagaagttcggcccatagcccgatccattaaaacggctcacgacgatgcgcgagcggaattaatgtcggcggacagacctcgatcaacgcgcggtctatgattctagaagagactcttatcaagaatcccatctcttgcttgcttttttttgttgcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 19 - DNA, 10569 bp, synthetic construct
  misc_feature 1..10569: note = pJF11
  source 1..10569: mol_type = other DNA
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatcttagaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagagacgaaacacaaaccatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgcgtgaccacatggtccttcatgagtacgtaaatgctgctgggattacaggtggagggtctggtggcggatcactcgaagttatggcagaagttcggcccatagcccgatccattaaaacggctcacgacgatgcgcgagcggaattaatgtcggcggacagacctcgatcaacgcgcggtctatgattctagaagagactcttatcaagaatcccatctcttgcttgcttttttttgttgcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 20 - DNA, 10569 bp, synthetic construct
  misc_feature 1..10569: note = pJF12
  source 1..10569: mol_type = other DNA
agtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaactcgatgtcgatcgactctagctagaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaccatggtcgatcgacagatctgcgaatcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgggatatttcacaaattgaacatagactacagaattttagaaaacaaactttctctctcttatctcacctttatcttttagagagaaaaagttcgatttccggttgaccggaatgtatctttgttttttttgttttgtaacatatttcgttttccgatttagatcggatctccttttccgttttgtcggaccttcttccggtttatccggatctaataatatccatcttagacttagctaagtttggatctgttttttggttagctcttgtcaatcgcctcatcatcagcaagaaggtgaaatttttgacaaataaatcttagaatcatgtagtgtctttggaccttgggaatgatagaaacgatttgttatagctactctatgtatcagaccctgaccaagatccaacaatctcataggttttgtgcatatgaaaccttcgactaacgagaagtggtcttttaatgagagagatatctaaaatgttatcttaaaagcccactcaaatctcaaggcataaggtagaaatgcaaatttggaaagtgggctgggccttttgtggtaaaggcctgtaacctagcccaatattagcaaaaccctagacgcgtacattgacatatataaacccgcctcctccttgtttagggtttctacgtgagagagacgaaacacaaaccatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgggtggagggtctcgtgaccacatggtccttcatgagtacgtaaatgctgctgggattacaggtggcggatcactcgaagttatggcagaagttcggcccatagcccgatccattaaaacggctcacgacgatgcgcgagcggaattaatgtcggcggacagacctcgatcaacgcgcggtctatgattctagaagagactcttatcaagaatcccatctcttgcttgcttttttttgttgcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaa

SEQ ID NO: 21 - DNA, 1275 bp, synthetic construct
  misc_feature 1..1275: note = Deleted virD2 sequence in XYA105 virD2
  source 1..1275: mol_type = other DNA
atgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctag

SEQ ID NO: 22 - DNA, 6369 bp, synthetic construct
  misc_feature 1..6369: note = DNA sequence of backbone vector pAGa
  source 1..6369: mol_type = other DNA
actttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttgtgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccacgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgtgcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccaggcctctggcaggatatattgtggtgtaaaccgagctgtataggctggctggtggcaggatatattgtggtgtaaacttaattaatagggataacagggtaatggcgcgcctgacaggatatattggcgggtaaactaagtcgctgtatgtgtttgtttggaattcgagctcggtacccggggatcctctagagtcgacctgcaggcatgcaagcttagt

SEQ ID NO: 23 - DNA, 6369 bp, synthetic construct
  misc_feature 1..6369: note = DNA sequence of backbone vector pAGb
  source 1..6369: mol_type = other DNA
actttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttgtgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccacgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgtgcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccaggcctctggcaggatatattgtggtgtaaaccgagctgtataggctggctggtggcaggatatattgtggtgtaaacttaattaatagggataacagggtaatggcgcgcctgacaggatatattggcgggtaaactaagtcgctgtatgtgtttgtttgaagcttgcatgcctgcaggtcgactctagaggatccccgggtaccgagctcgaattcagt

SEQ ID NO: 24 - DNA, 6372 bp, synthetic construct
  misc_feature 1..6372: note = DNA sequence of backbone vector pAGc
  source 1..6372: mol_type = other DNA
actttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttgtgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccacgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgtgcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccaggcctgaattcgagctcggtacccggggatcctctagagtcgacctgcaggcatgcaagcttctggcaggatatattgtggtgtaaaccgagctgtataggctggctggtggcaggatatattgtggtgtaaacttaattaatagggataacagggtaatggcgcgcctgacaggatatattggcgggtaaactaagtcgctgtatgtgtttgtttgagtact

SEQ ID NO: 25 - DNA, 6372 bp, synthetic construct
  misc_feature 1..6372: note = DNA sequence of backbone vector pAGd
  source 1..6372: mol_type = other DNA
actttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttgtgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccacgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgtgcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccaggcctaagcttgcatgcctgcaggtcgactctagaggatccccgggtaccgagctcgaattcctggcaggatatattgtggtgtaaaccgagctgtataggctggctggtggcaggatatattgtggtgtaaacttaattaatagggataacagggtaatggcgcgcctgacaggatatattggcgggtaaactaagtcgctgtatgtgtttgtttgagtact

SEQ ID NO: 26 - DNA, 11335 bp, synthetic construct
  misc_feature 1..11335: note = DNA sequence of binary chloroplast transformation vectorpAG-CpTEST_ Note dicistronic aadA-gfp marker genes
  source 1..11335: mol_type = other DNA
actttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttgtgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccacgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgtgcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccaggcctctggcaggatatattgtggtgtaaaccgagctgtataggctggctggtggcaggatatattgtggtgtaaacttaattaaaattcaccgccgtatggctgaccggcgattactagcgattccggcttcatgcaggcgagttgcagcctgcaatccgaactgaggacgggtttttggggttagctcaccctcgcgggatcgcgaccctttgtcccggccattgtagcacgtgtgtcgcccagggcataaggggcatgatgacttgacgtcatcctcaccttcctccggcttatcaccggcagtctgttcagggttccaaactcaacgatggcaactaaacacgagggttgcgctcgttgcgggacttaacccaacaccttacggcacgagctgacgacagccatgcaccacctgtgtccgcgttcccgaaggcacccctctctttcaagaggattcgcggcatgtcaagccctggtaaggttcttcgctttgcatcgaattaaaccacatgctccaccgcttgtgcgggcccccgtcaattcctttgagtttcattcttgcgaacgtactccccaggcgggatacttaacgcgttagctacagcactgcacgggtcgatacgcacagcgcctagtatccatcgtttacggctaggactactggggtatctaatcccattcgctcccctagctttcgtctctcagtgtcagtgtcggcccagcagagtgctttcgccgttggtgttctttccgatctctacgcatttcaccgctccaccggaaattccctctgcccctaccgtactccagcttggtagtttccaccgcctgtccagggttgagccctgggatttgacggcggacttaaaaagccacctacagacgctttacgcccaatcattccggataacgcttgcatcctctgtattaccgcggctgctggcacagagttagccgatgcttattccccagataccgtcattgcttcttctccgggaaaagaagttcacgacccgtgggccttctacctccacgcggcattgctccgtcaggctttcgcccattgcggaaaattccccactgctgcctcccgtaggagtctgggccgtgtctcagtcccagtgtggctgatcatcctctcggaccagctactgatcatcgccttggtaagctattgcctcaccaactagctaatcagacgcgagcccctcctcgggcggattcctccttttgctcctcagcctacggggtattagcagccgtttccagctgttgttcccctcccaagggcaggttcttacgcgttactcacccgtccgccactggaaacaccacttcccgtccgacttgcatgtgttaagcatgccgccagcgttcatcctgagccaggatcgaactctccatgagattcatagttgcattacttatagcttccttgttcgtagacaaagcggattcggaattgtctttcattccaaggcataacttgtatccatgcgcttcatattcgcccggagttcgctcccagaaatatagccatccctgccccctcacgtcaatcccacgagcctcttatccattctcattgaacgacggcgggggagcaaatccaactagaaaaactcacattgggcttagggataatcaggctcgaactgatgacttccaccacgtcaaggtgacactctaccgctgagttatatcccttccccgccccatcgagaaatagaactgactaatcctaagtcaaagggtcgagaaactcaacgccactattcttgaacaacttggagccgggccttcttttcgcactattacggatatgaaaataatggtcaaaatcggattcaattgtctaccgctcccccgccgtcgttcaatgagaatggataagaggctcgtgggattgacgtgagggggcagggatggctatatttctgggagaattaaccgatcgacgtgcaagcggacatttattttaaattcgataatttttgcaaaaacatttcgacatatttatttattttattattatgagaatcaatcctactacttctggttctggggtttccacggctactagcgaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtgggcaaagaacaaaaactcatttctgaagaagacttgtaactgcagataacccaaataatgttttaaaattttaaaaataatgtaggaggaaaaattatggctagcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagctctagctagagcgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgggggatcaaagctaacaactgcccctatcggaaataggattgactaccgattccgaaggaactggagttacatctcttttccattcaagagttcttatgcgtttccacgcccctttgagaccccgaaaaatggacaaattccttttcttaggaacacatacaagattcgtcactacaaaaaggataatggtaaccctaccattaactacttcatttatgaatttcatagtaatagaaatacatgtcctaccgagacagaatttggaacttgctatcctcttgcctagcaggcaaagatttacctccgtggaaaggatgattcattcggatcgacatgagagtccaactacattgccagaatccatgttgtatatttgaaagaggttgacctccttgcttctctcatggtacactcctcttcccgccgagccccttttctcctcggtccacagagacaaaatgtaggactggtgccaacaattcatcagactcactaagtcgggatcactaactaatactaatctaatataatagtctaatatatctaatataatagaaaatactaatataatagaaaagaactgtcttttctgtatactttccccggttccgttgctaccgcgggctttacgcaatcgatcggattagatagatatcccttcaacataggtcatcgaaaggatctcggagacccaccaaagtacgaaagccaggatctttcagaaaacggattcctattcaaagagtgcataaccgcatggataagctcacactaacccgtcaatttgggatccaaattcgagattttccttgggaggtatcgggaaggatttggaatggaataatatcgattcatacagaagaaaaggttctctattgattcaaacactgtacctaacctatgggatagggatcgaggaaggggaaaaaccgaagatttcacatggtacttttatcaatctgatttatttcgtacctttcgttcaatgagaaaatgggtcaaattctacaggatcaaacctatgggacttaaggaatgatataaaaaaaagagagggaaaatattcatattaaataaatatgaagtagaagaacccagattccaaatgaacaaattcaaacttgaaaaggatcttccttattcttgaagaatgaggggcaaagggattgatcaagaaagatcggcgcgcctgacaggatatattggcgggtaaactaagtcgctgtatgtgtttgtttggaattcgagctcggtacccggggatcctctagagtcgacctgcaggcatgcaagcttagt

SEQ ID NO: 27 - DNA, 1259 bp, synthetic construct
  misc_feature 1..1259: note = The DNA sequence of Pt-VirD2 in a VirD promoter/VirF terminatorcassette. The Pt-VirD2 has the tobacco SSU Transit peptide+5aa,the mutant minimal VirD2-T4SS signal.
  source 1..1259: mol_type = other DNA
ttatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtccacgaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtacggaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagaatccgtgctgttcgtcacccgcttatagttgccatcacaatcggatttcaactgccccggtctttaggtgcggccgaggacaatgtagcacgcacttacggtgcgtctcgtctctgacgctttctatgttccgca

SEQ ID NO: 28 - DNA, 1441 bp, synthetic construct
  misc_feature 1..1441: note = The DNA sequence of Pt-VirD2 in a VirE promoter/VirF terminatorcassette. The Pt-VirD2 has the tobacco SSU Transit peptide+5aa,the mutant minimal VirD2-T4SS signal
  source 1..1441: mol_type = other DNA
cggctgctcgtcaccaacaatgctcacagggatacgatggcctcgacagtcaggccgcgtgcgtccagtctttccagttcctccctttcaggtcgattgtggcatcatttattgcctgctcattgcagttgaaacgcgatatccgtttcaagacccgagtatggatggtactttggagatatgagcacttgccagacttcccggtcggagacttaaaaattccgataagaggttactgacgacggaccttataagtgggtcgtctagtggtcgcactgatcaaaacagttccgcccccggctttgctcaaagtagcaaagcagctttatctcgggttgcagaggattttctggaaaaccgcaattttgcaggagagaacatatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtccacgaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtacggaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagaatccgtgctgttcgtcacccgcttatagttgccatcacaatcggatttcaactgccccggtctttaggtgcggccgaggacaatgtagcacgcacttacggtgcgtctcgtctctgacgctttctatgttccgca

SEQ ID NO: 29 - DNA, 11801 bp, synthetic construct
  misc_feature 1..11801: note = DNA sequence of pAG-CHL-1 chloroplast binary transformationvector for introduction of transgenes (goi) into the plastidgenome.The binary vector carries a Pt-VirD2 gene outside the T-DNA listed as SEQ ID No 27.
  source 1..11801: mol_type = other DNA
actttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttgtgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccacgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgtgcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccaggcctctggcaggatatattgtggtgtaaaccgagctgtataggctggctggtggcaggatatattgtggtgtaaacttaattaaaattcaccgccgtatggctgaccggcgattactagcgattccggcttcatgcaggcgagttgcagcctgcaatccgaactgaggacgggtttttggggttagctcaccctcgcgggatcgcgaccctttgtcccggccattgtagcacgtgtgtcgcccagggcataaggggcatgatgacttgacgtcatcctcaccttcctccggcttatcaccggcagtctgttcagggttccaaactcaacgatggcaactaaacacgagggttgcgctcgttgcgggacttaacccaacaccttacggcacgagctgacgacagccatgcaccacctgtgtccgcgttcccgaaggcacccctctctttcaagaggattcgcggcatgtcaagccctggtaaggttcttcgctttgcatcgaattaaaccacatgctccaccgcttgtgcgggcccccgtcaattcctttgagtttcattcttgcgaacgtactccccaggcgggatacttaacgcgttagctacagcactgcacgggtcgatacgcacagcgcctagtatccatcgtttacggctaggactactggggtatctaatcccattcgctcccctagctttcgtctctcagtgtcagtgtcggcccagcagagtgctttcgccgttggtgttctttccgatctctacgcatttcaccgctccaccggaaattccctctgcccctaccgtactccagcttggtagtttccaccgcctgtccagggttgagccctgggatttgacggcggacttaaaaagccacctacagacgctttacgcccaatcattccggataacgcttgcatcctctgtattaccgcggctgctggcacagagttagccgatgcttattccccagataccgtcattgcttcttctccgggaaaagaagttcacgacccgtgggccttctacctccacgcggcattgctccgtcaggctttcgcccattgcggaaaattccccactgctgcctcccgtaggagtctgggccgtgtctcagtcccagtgtggctgatcatcctctcggaccagctactgatcatcgccttggtaagctattgcctcaccaactagctaatcagacgcgagcccctcctcgggcggattcctccttttgctcctcagcctacggggtattagcagccgtttccagctgttgttcccctcccaagggcaggttcttacgcgttactcacccgtccgccactggaaacaccacttcccgtccgacttgcatgtgttaagcatgccgccagcgttcatcctgagccaggatcgaactctccatgagattcatagttgcattacttatagcttccttgttcgtagacaaagcggattcggaattgtctttcattccaaggcataacttgtatccatgcgcttcatattcgcccggagttcgctcccagaaatatagccatccctgccccctcacgtcaatcccacgagcctcttatccattctcattgaacgacggcgggggagcaaatccaactagaaaaactcacattgggcttagggataatcaggctcgaactgatgacttccaccacgtcaaggtgacactctaccgctgagttatatcccttccccgccccatcgagaaatagaactgactaatcctaagtcaaagggtcgagaaactcaacgccactattcttgaacaacttggagccgggccttcttttcgcactattacggatatgaaaataatggtcaaaatcggattcaattgtctaccatgaataaatgcaagaaaataacctctccttctttttctataatgtaaacaaaaaagtctatgtaagtaaaatactagtaaataaataaaaagaaaaaaagaaaggagcaatagcaccctcttgatagaacaagaaaatgattattgctcctttcttttcaaaacctcctatagactaggccaggatcttacaagtcttcttcagaaatgagtttttgttctttgcccactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcgctagtagccgtggaaaccccagaaccagaagtagtaggattgattctcataataataaaataaataaatatgtcgaaatgtttttgcaaaaattatcgaatttaaaataaatgtccgcttgcacgtcgatcggttaattctcccagaaatatagccatccctgccccctcacgtcaatcccacgagcctcttatccattctcattgaacgacggcgggggagcgtagaattcgagctcggtacccggggatcctctagagtcgacctgcaggcatgcaagcttgttaacaactgcccctatcggaaataggattgactaccgattccgaaggaactggagttacatctcttttccattcaagagttcttatgcgtttccacgcccctttgagaccccgaaaaatggacaaattccttttcttaggaacacatacaagattcgtcactacaaaaaggataatggtaaccctaccattaactacttcatttatgaatttcatagtaatagaaatacatgtcctaccgagacagaatttggaacttgctatcctcttgcctagcaggcaaagatttacctccgtggaaaggatgattcattcggatcgacatgagagtccaactacattgccagaatccatgttgtatatttgaaagaggttgacctccttgcttctctcatggtacactcctcttcccgccgagccccttttctcctcggtccacagagacaaaatgtaggactggtgccaacaattcatcagactcactaagtcgggatcactaactaatactaatctaatataatagtctaatatatctaatataatagaaaatactaatataatagaaaagaactgtcttttctgtatactttccccggttccgttgctaccgcgggctttacgcaatcgatcggattagatagatatcccttcaacataggtcatcgaaaggatctcggagacccaccaaagtacgaaagccaggatctttcagaaaacggattcctattcaaagagtgcataaccgcatggataagctcacactaacccgtcaatttgggatccaaattcgagattttccttgggaggtatcgggaaggatttggaatggaataatatcgattcatacagaagaaaaggttctctattgattcaaacactgtacctaacctatgggatagggatcgaggaaggggaaaaaccgaagatttcacatggtacttttatcaatctgatttatttcgtacctttcgttcaatgagaaaatgggtcaaattctacaggatcaaacctatgggacttaaggaatgatataaaaaaaagagagggaaaatattcatattaaataaatatgaagtagaagaacccagattccaaatgaacaaattcaaacttgaaaaggatcttccttattcttgaagaatgaggggcaaagggattgatcaagaaagatcggcgcgcctgacaggatatattggcgggtaaactaagtcgctgtatgtgtttgtttggaattttatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtccacgaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtacggaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagaatccgtgctgttcgtcacccgcttatagttgccatcacaatcggatttcaactgccccggtctttaggtgcggccgaggacaatgtagcacgcacttacggtgcgtctcgtctctgacgctttctatgttccgcagcttagt

SEQ ID NO: 30 - DNA, 1249 bp, synthetic construct
  misc_feature 1..1249: note = DNA sequence of kanamycin resistance gene in StuI-NsiI fragment
  source 1..1249: mol_type = other DNA
aggcctggcctcgtgatacgcctatttttataggttaatgtcatgggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggcacgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcgtggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcacaaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatgtgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcatgcat

SEQ ID NO: 31 - DNA, 1259 bp, synthetic construct
  misc_feature 1..1259: note = 31 The DNA sequence of Pt-VirD2-3 in a VirD promoter/VirFterminator cassette where Pt-VirD2 has the wild type NLS1. ThePt-VirD2-3 has the tobacco SSU Transit peptide+5aa, the wtminimal VirD2-T4SS signal
  source 1..1259: mol_type = other DNA
ttatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagaatccgtgctgttcgtcacccgcttatagttgccatcacaatcggatttcaactgccccggtctttaggtgcggccgaggacaatgtagcacgcacttacggtgcgtctcgtctctgacgctttctatgttccgca

SEQ ID NO: 32 - DNA, 1441 bp, synthetic construct
  misc_feature 1..1441: note = The DNA sequence of Pt-VirD2-4 in a VirE promoter/VirF terminatorcassette where Pt-VirD2 has the wild type NLS1. The Pt-VirD2-4has the tobacco SSU Transit peptide+5aa, the wt minimalVirD2-T4SS signal
  source 1..1441: mol_type = other DNA
cggctgctcgtcaccaacaatgctcacagggatacgatggcctcgacagtcaggccgcgtgcgtccagtctttccagttcctccctttcaggtcgattgtggcatcatttattgcctgctcattgcagttgaaacgcgatatccgtttcaagacccgagtatggatggtactttggagatatgagcacttgccagacttcccggtcggagacttaaaaattccgataagaggttactgacgacggaccttataagtgggtcgtctagtggtcgcactgatcaaaacagttccgcccccggctttgctcaaagtagcaaagcagctttatctcgggttgcagaggattttctggaaaaccgcaattttgcaggagagaacatatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgattctagaatccgtgctgttcgtcacccgcttatagttgccatcacaatcggatttcaactgccccggtctttaggtgcggccgaggacaatgtagcacgcacttacggtgcgtctcgtctctgacgctttctatgttccgca

SEQ ID NO: 33 - DNA, 77 bp, synthetic construct
  misc_feature 1..77: note = DNA sequence introducing a C to T point mutation at nucleotide473 in tobacco plastid 16S RNA coding sequence flanked by PacIand AscI conferring streptomycin resistance
  source 1..77: mol_type = other DNA
ttaattaaaataagcatcggctaactctgtgccagcagtcgcggtaatacagaggatgcaagcgttatcggcgcgcc

SEQ ID NO: 34 - DNA, 77 bp, synthetic construct
  misc_feature 1..77: note = DNA sequence introducing a C to A point mutation at nucleotide861 in tobacco plastid 16S RNA coding sequence flanked by PacIand AscI conferring streptomycin resistance
  source 1..77: mol_type = other DNA
ttaattaacctggggagtacgttcgcaagaatgaaactaaaaggaattgacgggggcccgcacaagcggggcgcgcc

SEQ ID NO: 35 - DNA, 77 bp, synthetic construct
  misc_feature 1..77: note = DNA sequence introducing a C to T point mutation at nucleotide861 in tobacco plastid 16S RNA coding sequence flanked by PacIand AscI conferring streptomycin resistance
  source 1..77: mol_type = other DNA
ttaattaacctggggagtacgttcgcaagaatgaaacttaaaggaattgacgggggcccgcacaagcggggcgcgcc

SEQ ID NO: 36 - DNA, 77 bp, synthetic construct
  misc_feature 1..77: note = DNA sequence introducing a G to A point mutation at nucleotide1013 in tobacco plastid 16S RNA coding sequence flanked by PacIand AscI conferring spectinomycin resistance
  source 1..77: mol_type = other DNA
ttaattaagaacgcggacacaggtggtgcatggctgtcatcagctcgtgccgtaaggtgttgggttaagggcgcgcc

SEQ ID NO: 37 - DNA, 77 bp, synthetic construct
  misc_feature 1..77: note = DNA sequence introducing a A to C point mutation at nucleotide1139 in tobacco plastid 16S RNA coding sequence flanked by PacIand AscI conferring spectinomycin resistance
  source 1..77: mol_type = other DNA
ttaattaaccggtgataagccggaggaaggtgaggatgccgtcaagtcatcatgccccttatgccctggggcgcgcc

SEQ ID NO: 38 - DNA, 77 bp, synthetic construct
  misc_feature 1..77: note = DNA sequence introducing a C to T point mutation at nucleotide1140 in tobacco plastid 16S RNA coding sequence flanked by PacIand AscI conferring spectinomycin resistance
  source 1..77: mol_type = other DNA
ttaattaacggtgataagccggaggaaggtgaggatgatgtcaagtcatcatgccccttatgccctgggggcgcgcc

SEQ ID NO: 39 - DNA, 77 bp, synthetic construct
  misc_feature 1..77: note = DNA sequence introducing a G to A point mutation at nucleotide1141 in tobacco plastid 16S RNA coding sequence flanked by PacIand AscI conferring spectinomycin resistance
  source 1..77: mol_type = other DNA
ttaattaaggtgataagccggaggaaggtgaggatgacatcaagtcatcatgccccttatgccctgggcggcgcgcc

SEQ ID NO: 40 - DNA, 77 bp, synthetic construct
  misc_feature 1..77: note = DNA sequence introducing a G to A point mutation at nucleotide1335 in tobacco plastid 16S RNA coding sequence flanked by PacIand AscI conferring spectinomycin resistance
  source 1..77: mol_type = other DNA
ttaattaagtcagccatacggcggtgaattcgttcccgagccttgtacacaccgcccgtcacactatggggcgcgcc

SEQ ID NO: 41 - DNA, 80 bp, synthetic construct
  misc_feature 1..80: note = DNA sequence introducing streptomycin resistance and a silent mutation in the tobacco rps12 coding sequence. Flanked by PacI and AscI restriction sites.. C271T changes Pro to Ser conferring streptomycin resistance. A270G is a silent mutation.
  source 1..80: mol_type = other DNA
ttaattaagtcttagtaagagggggaagggttaaggatttgtccggtgtgagatatcacattgttcgaggaaggcgcgcc

SEQ ID NO: 42 - DNA, 975 bp, synthetic construct
  misc_feature 1..975: note = DNA sequence of PtACC2-virD2. Coding sequence of A. thaliana ACC2transit peptide+6aa (lower case) fused with wt minimal VirD2 andT4SS
  source 1..975: mol_type = other DNA
atggagatgagagctttgggttcttcgtgttctactggtaatggaggttctgctccgattaccctcacgaatatatctccttggatcacaacagtttttccatctacagtgaagctgagaagtagtttgagaaccttcaaaggagtttcgtcaagagtgagaacctttaaaggagtttcttcgacaagagttttgtctcggaccaaacaacagtttcctctgttttgtttcctaaaccctgatccgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatga

SEQ ID NO: 43 - DNA, 924 bp, synthetic construct
  misc_feature 1..924: note = DNA sequence of PtMKK4-virD2. Coding sequence of A. thaliana MKK4transit peptide (lower case) fused with wt minimal VirD2 andT4SS.
  source 1..924: mol_type = other DNA
atgagaccgattcaatcgcctccaggagtttccgttccggtgaaaagccgtccccgtcgccgtcctgatcttaccttaccgcttcctcaacgcgatgtttctctcgctgtacctcttcctctcccacctacttccggtggttccggtggctctagtggatctgcgccgtcttctggtggttcggcgtcttcaacgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatga

SEQ ID NO: 44 - DNA, 6107 bp, synthetic construct
  misc_feature 1..6107: note = DNA sequence of pEX18Kan. pEX18Tc (Accession no. AF047519)derivative with kanamycin resistance as selectable marker
  source 1..6107: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataatcggcattttcttttgcgtttttatttgttaactgttaattgtccttgttcaaggatgctgtctttgacaacagatgttttcttgcctttgatgttcagcaggaagctaggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcgaactcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacaatcgatgcaagggttggtttgtgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttgtgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccacgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgtgcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccaggctaagaaaccattattatcatgacattaacctataaaaataggcgtatcacgaggccctttcgtc

SEQ ID NO: 45 - DNA, 1930 bp, synthetic construct
  misc_feature 1..1930: note = EcoRI-HindIII fragment of Pt-virD2 flanked with 500 bp of virD1and 500 bp of virD3. The PtVirD2 has the tobacco SSU Transitpeptide, the wt minimal VirD2-T4SS signal listed as SEQ ID NO 31
  source 1..1930: mol_type = other DNA
gaattcttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatggcttcctcagttctttcctccgcagcagttgccacccgcagcaatgttgctcaagctaacatggttgcacctttcactggccttaagtcagctgcctcattccctgtttcaaggaagcaaaaccttgacatcacttccattgccagcaacggcggaagagtgcaatgcatgcaggtgtggccagcgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgaagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaagctt

SEQ ID NO: 46 - DNA, 1987 bp, synthetic construct
  misc_feature 1..1987: note = EcoRI-HindIII fragment of PtACC2-virD2 flanked with 500 bp ofvirD1 and 500 bp of virD3. The PtACC2-VirD2 has the ArabidopsisACC2 Transit peptide, the wt minimal VirD2-T4SS signal listed as SEQ ID NO 42
  source 1..1987: mol_type = other DNA
gaattcttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatggagatgagagctttgggttcttcgtgttctactggtaatggaggttctgctccgattaccctcacgaatatatctccttggatcacaacagtttttccatctacagtgaagctgagaagtagtttgagaaccttcaaaggagtttcgtcaagagtgagaacctttaaaggagtttcttcgacaagagttttgtctcggaccaaacaacagtttcctctgttttgtttcctaaaccctgatccgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgaagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaagctt

SEQ ID NO: 47 - DNA, 1936 bp, synthetic construct
  misc_feature 1..1936: note = EcoRI-HindIII fragment of PtMKK4-virD2 flanked with 500 bp ofvirD1 and 500 bp of virD3. The PtMKK4-VirD2 has the ArabidopsisMKK4 Transit peptide, the wt minimal VirD2-T4SS signal listed as SEQ ID NO 43
  source 1..1936: mol_type = other DNA
gaattcttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgagaccgattcaatcgcctccaggagtttccgttccggtgaaaagccgtccccgtcgccgtcctgatcttaccttaccgcttcctcaacgcgatgtttctctcgctgtacctcttcctctcccacctacttccggtggttccggtggctctagtggatctgcgccgtcttctggtggttcggcgtcttcaacgatggctcccgatcgggcccaagtaatcattcgcattgtgccaggaggtggaaccaagacccttcagcagataatcaatcagctggagtacctgtcccgaaagggaaagctggaactccagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtgagcttccccgcaggtaccgaccaaaccgcagcttatgaagcaagccgggaatgggcagccgagatgtttgggtcaggatacgggggtggccgctataactatctgacagcctaccacgtggaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcccgagcagaaaggggaatagcagagcgaccaatcacctatgctgaatatcgacgtcttgagcggatgctcgaagttatggcagaagttcggcccatagcccgatccattaaaacggctcacgacgatgcgcgagcggaattaatgtcggcggacagacctcgatcaacgcgcggtctatgaagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaagctt

SEQ ID NO: 48 - DNA, 20 bp, synthetic construct
  misc_feature 1..20: note = AM102
  source 1..20: mol_type = other DNA
gttgagtcgatacttcggcg

SEQ ID NO: 49 - DNA, 20 bp, synthetic construct
  misc_feature 1..20: note = AM103
  source 1..20: mol_type = other DNA
ggcaccagtcctacattttg

SEQ ID NO: 50 - protein, 16 aa, synthetic construct
  REGION 1..16: note = GFP11
RDHMBLHEYBNAAGIT

SEQ ID NO: 51 - DNA, 25 bp, synthetic construct
  source 1..25: mol_type = other DNA
  misc_feature 1..25: note = Left Border Sequence
tggcaggatatattgtggtgtaaac

SEQ ID NO: 52 - DNA, 21 bp, synthetic construct
  source 1..21: mol_type = other DNA
  misc_feature 1..21: note = Spacer between Left Border repeats
cgagctgtataggctggctgg

SEQ ID NO: 53 - DNA, 25 bp, synthetic construct
  source 1..25: mol_type = other DNA
  misc_feature 1..25: note = Right Border Sequence
tgacaggatatattggcgggtaaac

SEQ ID NO: 54 - DNA, 24 bp, synthetic construct
  source 1..24: mol_type = other DNA
  misc_feature 1..24: note = Overdrive Sequence
taagtcgctgtatgtgtttgtttg

SEQ ID NO: 55 - DNA, 80 bp, synthetic construct
  source 1..80: mol_type = other DNA
  misc_feature 1..80: note = DNA sequence introducing streptomycin resistance and a silent mutation in the tobacco rps12 coding sequence. Flanked by PacI and AscI restriction sites. C271T changes Pro to Ser conferring streptomycin resistance. T276A is a silent mutation.
ttaattaagtcttagtaagagggggaagggttaaggatttatccggagtgagatatcacattgttcgaggaaggcgcgcc

SEQ ID NO: 56 - DNA, 2216 bp, synthetic construct
  source 1..2216: mol_type = other DNA
  misc_feature 1..2216: note = pAG-CHL-1 chloroplast binary transformation vector variant with shorter plastid targeting sequences for introduction of transgenes (goi) into plastid.PacI and AscI sites have been blunted
ccgccagcgttcatcctgagccaggatcgaactctccatgagattcatagttgcattacttatagcttccttgttcgtagacaaagcggattcggaattgtctttcattccaaggcataacttgtatccatgcgcttcatattcgcccggagttcgctcccagaaatatagccatccctgccccctcacgtcaatcccacgagcctcttatccattctcattgaacgacggcgggggagcaaatccaactagaaaaactcacattgggcttagggataatcaggctcgaactgatgacttccaccacgtcaaggtgacactctaccgctgagttatatcccttccccgccccatcgagaaatagaactgactaatcctaagtcaaagggtcgagaaactcaacgccactattcttgaacaacttggagccgggccttcttttcgcactattacggatatgaaaataatggtcaaaatcggattcaattcccgggttgggttgcgctatatatatgaaagagtatacaataatgatgtatttgagttgtagggagggatttatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtgggcaaagaacaaaaactcatttctgaagaagacttgtgagatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgcccgggcaactgcccctatcggaaataggattgactaccgattccgaaggaactggagttacatctcttttccattcaagagttcttatgcgtttccacgcccctttgagaccccgaaaaatggacaaattccttttcttaggaacacatacaagattcgtcactacaaaaaggataatggtaaccctaccattaactacttcatttatgaatttcatagtaatagaaatacatgtcctaccgagacagaatttggaacttgctatcctcttgcctagcaggcaaagatttacctccgtggaaaggatgattcattcggatcgacatgagagtccaactacattgccagaatccatgttgtatatttgaaagaggttgacctccttgcttctctcatggtacactcctcttcccgccgagccccttttctcctcggtccacagagacaaaatgtaggactggtgccaacaattcatcagactcactaagtcgggatcactaactaatactaatctaatataatagtctaatatatctaatataatagaaaatactaatataatagaaaagaactgtcttttctgtatactttccccggttccgttgctaccgcgggctttacgcaatcgatcggattagatagat

SEQ ID NO: 57 - DNA, 29 bp, synthetic construct
  source 1..29: mol_type = other DNA
  misc_feature 1..29: note = attB
ccagggcgtgcccttgggctccccgggcg

SEQ ID NO: 58 - DNA, 29 bp, synthetic construct
  source 1..29: mol_type = other DNA
  misc_feature 1..29: note = attP
actggggtaacctttgagttctctcagtt

SEQ ID NO: 59 - DNA, 29 bp, synthetic construct
  source 1..29: mol_type = other DNA
  misc_feature 1..29: note = attL
ccagggcgtgcccttgagttctctcagtt

SEQ ID NO: 60 - DNA, 29 bp, synthetic construct
  source 1..29: mol_type = other DNA
  misc_feature 1..29: note = attR
actggggtaacctttgggctccccgggcg

SEQ ID NO: 61 - DNA, 78 bp, synthetic construct
  source 1..78: mol_type = other DNA
  misc_feature 1..78: note = Oligonucleotide confers streptomnycin resistance and the silent mutations create a KpnI restriction site in the rps12 plastid gene
gtcttagtaagagggggaagggttaaggatttatccggtgtgaggtaccacattgttcgaggaaccctagatgctgtc